(19) United States
(12) Patent Application Publication
(10) Pub. No.: US 2025/0098533 A1
(43) Pub. Date: Mar. 20, 2025

(54) ORGANIC COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Tiantian MA, Xi'an (CN); Kongyan ZHANG, Xi'an (CN); Xinxuan LI, Xi'an (CN)

(21) Appl. No.: 18/280,216

(22) PCT Filed: Feb. 16, 2023
(86) PCT No.: PCT/CN2023/076637

§ 371 Date: Sep. 2, 2023

(30) Foreign Application Priority Data
May 12, 2022 (CN) 202210513155.X

(51) Int. Cl. H10K 85/60; C07D 209/82; C07D 405/14; C07D 409/14; H10K 50/12; H10K 50/15; H10K 50/17

(52) U.S. Cl. CPC H10K 85/6572; C07D 209/82; C07D 405/14; C07D 409/14; H10K 85/6574; H10K 85/6576; H10K 50/12; H10K 50/15; H10K 50/17

(57) ABSTRACT

The present disclosure relates to an organic compound, an organic electroluminescent device and an electronic apparatus. The organic compound of the present disclosure has a structure shown in a formula 1, and when the organic compound is applied in an organic electroluminescent device, the performance of the device can be significantly improved.

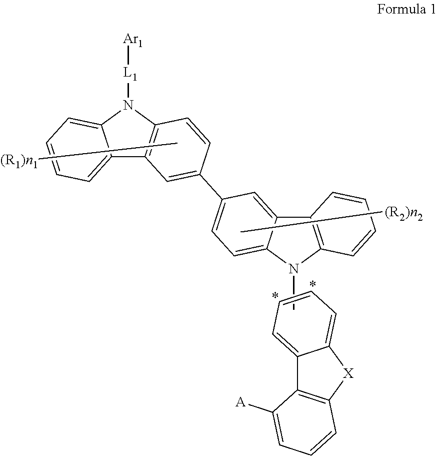

### CROSS REFERENCE TO RELATED APPLICATIONS

This application claims the priority of Chinese patent application No. 202210513155.X, filed on May 12, 2022, the contents of which are incorporated herein by reference in its entirety as part of the present disclosure.

### TECHNICAL FIELD

The present disclosure belongs to the technical field of organic materials, and particularly relates to an organic compound, and an organic electroluminescent device and an electronic apparatus including the same.

### BACKGROUND

Organic electroluminescent materials (OLED) have the advantages of ultra thinness, self-illumination, wide viewing angle, fast response, high luminous efficiency, good temperature adaptability, simple production process, low driving voltage, low energy consumption, and the like as a new-generation display technology, and have been widely used in the industries such as flat panel display, flexible display, solid state lighting, and vehicle display.

Currently, for green organic electroluminescent devices, a phosphorescent organic electroluminescent device is a main development direction, and is mainly used in display devices such as mobile phones, vehicles, and the like. However, with respect to the green organic electroluminescent devices, there are still problems such as decreased luminous efficiency and shortened service life, resulting in a degradation in device performance. Thus, these efficiency or service life problems must be solved for phosphorescent host materials, and there is a constant need to develop new materials for organic light-emitting devices which are highly efficient, long in service life and suitable for mass production.

It should be noted that the information disclosed in the above Background section is merely used to enhance an understanding of the background of the present disclosure, and thus may include information that does not constitute the prior art known to those of ordinary skill in the art.

### SUMMARY

In view of the above problems existing in the prior art, an object of the present disclosure is to provide an organic compound, and an organic electroluminescent device and an electronic apparatus including the same. The organic compound can improve the performance of the organic electroluminescent device and the electronic apparatus, such as reducing the driving voltage of the device, and improving the efficiency and service life of the device.

In a first aspect of the present disclosure, provided is an organic compound, having a structure shown in a formula 1:

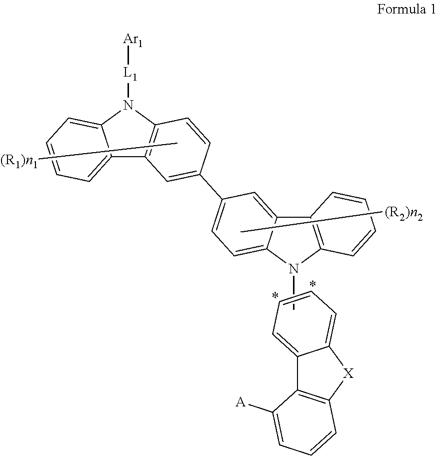

wherein * denotes a connection site;
X is selected from C(R₃R₄), O or S;
a group A is selected from substituted or unsubstituted aryl with 6 to 12 carbon atoms;
L₁ is selected from a single bond, substituted or unsubstituted arylene with 6 to 30 carbon atoms, and substituted or unsubstituted heteroarylene with 3 to 30 carbon atoms;
Ar₁ is selected from substituted or unsubstituted aryl with 6 to 30 carbon atoms, and substituted or unsubstituted heteroaryl with 3 to 30 carbon atoms;
each R₁ and each R₂ are the same or different, and are respectively and independently selected from deuterium, a halogen group, cyano, alkyl with 1 to 10 carbon atoms, deuterated alkyl with 1 to 10 carbon atoms, haloalkyl with 1 to 10 carbon atoms, aryl with 6 to 20 carbon atoms, deuterated aryl with 6 to 20 carbon atoms, and heteroaryl with 3 to 20 carbon atoms;
n₁ is the number of R₁, and is selected from 0, 1, 2, 3, 4, 5, 6 or 7, and when n₁ is greater than 1, any two R₁ are the same or different;
n₂ is the number of R₂, and is selected from 0, 1, 2, 3, 4, 5, 6 or 7, and when n₂ is greater than 1, any two R₂ are the same or different;
R₃ and R₄ are respectively and independently selected from hydrogen, deuterium, alkyl with 1 to 10 carbon atoms or deuterated alkyl with 1 to 10 carbon atoms;
substituents in the group A are the same or different, and are respectively and independently selected from deuterium, a halogen group, cyano, alkyl with 1 to 4 carbon atoms, deuterated alkyl with 1 to 4 carbon atoms, haloalkyl with 1 to 4 carbon atoms or phenyl; and
substituents in L₁ and Ar₁ are the same or different, and are respectively and independently selected from deuterium, a halogen group, cyano, alkyl with 1 to 10 carbon atoms, deuterated alkyl with 1 to 10 carbon atoms, haloalkyl with 1 to 10 carbon atoms, aryl with 6 to 20 carbon atoms, heteroaryl with 3 to 20 carbon atoms, cycloalkyl with 3 to 20 carbon atoms, deuterated aryl with 6 to 20 carbon atoms, haloaryl with 6 to 20 carbon atoms, and triarylsilyl with 18 to 24 carbon atoms.

In a second aspect of the present disclosure, provided is an organic electroluminescent device, including an anode and a cathode which are oppositely disposed, and a functional layer disposed between the anode and the cathode; wherein the functional layer includes the above organic compound.

In a third aspect of the present disclosure, provided is an electronic apparatus, including the organic electroluminescent device according to the second aspect.

The present disclosure provides the organic compound including 3,3-bicarbazole and a dibenzo five-membered ring group having an aryl substituent at a specific position, which are bonded at a specific connection position. Such compounds have enhanced hole mobility and energy transport efficiency and are suitable for being used as a hole-type host material in an organic electroluminescent device. Aryl substitution at the specific position of the dibenzo five-membered ring enhances the steric hindrance effect of the molecule and improves the film-forming properties of the material, so that the efficiency and the service life of the device can be further improved.

Other features and advantages of the present disclosure will be described in detail in the subsequent specific embodiments.

### BRIEF DESCRIPTION OF THE DRAWINGS

The accompanying drawings are used to provide a further understanding of the present disclosure and constitute a part of the description, and are used to explain the present disclosure together with the following specific embodiments, but do not constitute limitations on the present disclosure.

FIG. 1 is a structural schematic diagram of an organic electroluminescent device according to the present disclosure.

FIG. 2 is a structural schematic diagram of an electronic apparatus according to the present disclosure.

### REFERENCE SIGNS

100, anode; 200, cathode; 300, functional layer; 310, hole injection layer;
320, hole transport layer; 330, hole auxiliary layer; 340, organic light-emitting layer;
350, electron transport layer; 360, electron injection layer; 400, electronic apparatus.

### DETAILED DESCRIPTION

Examples will now be described more fully with reference to the accompanying drawings. However, the examples can be implemented in a variety of forms, and should not be understood as a limitation to the instances set forth here; and on the contrary, these examples are provided such that the present disclosure will be more comprehensive and complete, and the concepts of the examples are comprehensively conveyed to those skilled in the art. The described features, structures, or characteristics may be incorporated in one or more embodiments in any suitable manner. In the following description, many specific details are provided to give a sufficient understanding of the examples of the present disclosure.

In a first aspect of the present disclosure, provided is an organic compound, having a structure shown in a formula 1:

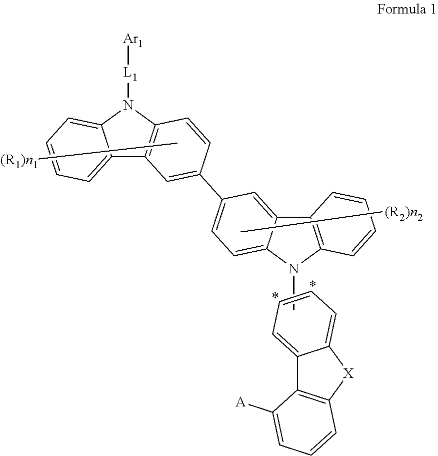

wherein * denotes a connection site;

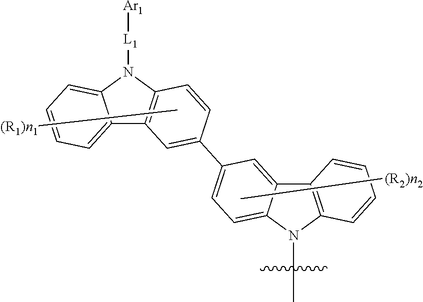

i.e., * denotes a site where is connected to

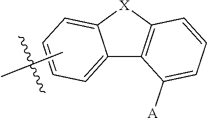

in the formula 1;
X is selected from C(R₃R₄), O or S;
a group A is selected from substituted or unsubstituted aryl with 6 to 12 carbon atoms;
L₁ is selected from a single bond, substituted or unsubstituted arylene with 6 to 30 carbon atoms, and substituted or unsubstituted heteroarylene with 3 to 30 carbon atoms;
Ar₁ is selected from substituted or unsubstituted aryl with 6 to 30 carbon atoms, and substituted or unsubstituted heteroaryl with 3 to 30 carbon atoms;
each R₁ and each R₂ are the same or different, and are respectively and independently selected from deuterium, a halogen group, cyano, alkyl with 1 to 10 carbon atoms, deuterated alkyl with 1 to 10 carbon atoms, haloalkyl with 1 to 10 carbon atoms, aryl with 6 to 20 carbon atoms, deuterated aryl with 6 to 20 carbon atoms, and heteroaryl with 3 to 20 carbon atoms;
n₁ is the number of R₁, and is selected from 0, 1, 2, 3, 4, 5, 6 or 7, and when n₁ is greater than 1, any two R₁ are the same or different;
n₂ is the number of R₂, and is selected from 0, 1, 2, 3, 4, 5, 6 or 7, and when n₂ is greater than 1, any two R₂ are the same or different;
R₃ and R₄ are respectively and independently selected from hydrogen, deuterium, alkyl with 1 to 10 carbon atoms or deuterated alkyl with 1 to 10 carbon atoms;
substituents in the group A are the same or different, and are respectively and independently selected from deuterium, a halogen group, cyano, alkyl with 1 to 4 carbon atoms, deuterated alkyl with 1 to 4 carbon atoms, haloalkyl with 1 to 4 carbon atoms or phenyl; and
substituents in L₁ and Ar₁ are the same or different, and are respectively and independently selected from deuterium, a halogen group, cyano, alkyl with 1 to 10 carbon atoms, deuterated alkyl with 1 to 10 carbon atoms, haloalkyl with 1 to 10 carbon atoms, aryl with 6 to 20 carbon atoms, heteroaryl with 3 to 20 carbon atoms, cycloalkyl with 3 to 20 carbon atoms, deuterated aryl with 6 to 20 carbon atoms, haloaryl with 6 to 20 carbon atoms, and triarylsilyl with 18 to 24 carbon atoms.

In the present disclosure, fluorenyl may be substituted by 1 or 2 substituents, where in the case that the above fluorenyl is substituted, it may be

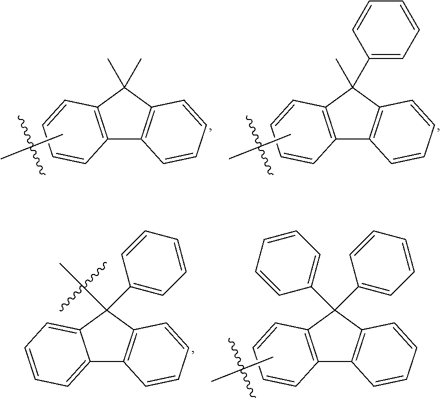

or the like, but is not limited thereto.

In the present disclosure, the adopted description modes “each . . . is independently”, “ . . . is respectively and independently” and “ . . . is each independently selected from” can be interchanged, and should be understood in a broad sense, which means that in different groups, specific options expressed between the same symbols do not influence each other, or in a same group, specific options expressed between the same symbols do not influence each other. For example, the meaning of

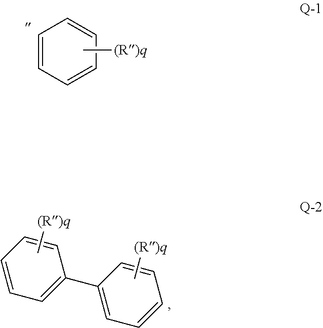

wherein each q is independently 0, 1, 2 or 3, and each R″ is independently selected from hydrogen, deuterium, fluorine and chlorine” is as follows: a formula Q-1 represents that q substituents R″ exist on a benzene ring, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, the term such as “substituted or unsubstituted” means that a functional group described behind the term may have or do not have a substituent (in the following, the substituent is collectively referred to as Rc in order to facilitate description). For example, the “substituted or unsubstituted aryl” refers to aryl having the substituent Rc or unsubstituted aryl. The above substituent, i.e., Rc, may be, for example, deuterium, a halogen group, cyano, alkyl, deuteroalkyl, haloalkyl, aryl, heteroaryl, cycloalkyl, deuteroaryl, haloaryl, triarylsilyl, or the like.

In the present disclosure, the number of carbon atoms of a substituted or unsubstituted functional group refers to the total number of all carbon atoms. For example, if L₁ is substituted arylene with 12 carbon atoms, the number of all carbon atoms of the arylene and substituents on the arylene is 12.

In the present disclosure, aryl refers to an optional functional group or substituent derived from an aromatic carbocyclic ring. The aryl may be monocyclic aryl (e.g., phenyl) or polycyclic aryl, in other words, the aryl can be monocyclic aryl, fused aryl, two or more monocyclic aryl conjugatedly connected through carbon-carbon bonds, monocyclic aryl and fused aryl which are conjugatedly connected through a carbon-carbon bond, or two or more fused aryl conjugatedly connected through carbon-carbon bonds. That is, unless otherwise noted, two or more aromatic groups conjugatedly connected through carbon-carbon bonds can also be regarded as the aryl of the present disclosure. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl, spirobifluorenyl, and the like. In the present disclosure, the arylene involved refers to a divalent group formed by further loss of one hydrogen atom from aryl.

In the present disclosure, terphenyl includes

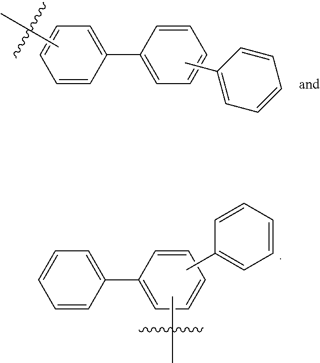

In the present disclosure, the substituted aryl may be that one or two or more hydrogen atoms in the aryl are substituted by groups such as a deuterium atom, a halogen group, cyano, aryl, heteroaryl, alkyl, cycloalkyl, and the like. It should be understood that the number of carbon atoms of the substituted aryl refers to the total number of carbon atoms of the aryl and substituents on the aryl, for example, a substituted aryl with 18 carbon atoms means that the total number of carbon atoms of the aryl and substituents is 18.

In the present disclosure, heteroaryl refers to a monovalent aromatic ring or its derivative containing 1, 2, 3, 4, 5, 6 or 7 heteroatoms in the ring, and the heteroatom may be at least one of B, O, N, P, Si, Se, and S. The heteroaryl may be monocyclic heteroaryl or polycyclic heteroaryl, in other words, the heteroaryl may be a single aromatic ring system or a plurality of aromatic ring systems conjugatedly connected through carbon-carbon bonds, and any one aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silafluorenyl, dibenzofuranyl, as well as N-phenylcarbazolyl, N-pyridylcarbazolyl, N-methylcarbazolyl and the like, but is not limited thereto. The thienyl, furyl, phenanthrolinyl and the like are heteroaryl of the single aromatic ring system, and N-phenylcarbazolyl and N-pyridylcarbazolyl are heteroaryl of the plurality of aromatic ring systems conjugatedly connected through carbon-carbon bonds. In the present disclosure, the heteroarylene involved refers to a divalent group formed by further loss of one hydrogen atom from heteroaryl.

In the present disclosure, the substituted heteroaryl may be that one or two or more hydrogen atoms in the heteroaryl are substituted by groups such as a deuterium atom, a halogen group, cyano, aryl, heteroaryl, alkyl, cycloalkyl, and the like. It should be understood that the number of carbon atoms of the substituted heteroaryl refers to the total number of carbon atoms of heteroaryl and substituents on the heteroaryl.

In the present disclosure, the number of carbon atoms of the substituted or unsubstituted aryl may be 6 to 30, for example, the number of carbon atoms may be 6, 7, 8, 9, 10, 11, 12, 13, 14, 15, 16, 17, 18, 19, 20, 21, 22, 23, 24, 25, 26, 27, 28, 29 or 30.

In the present disclosure, specific examples of aryl as a substituent include, but are not limited to, phenyl, biphenyl, naphthyl, fluorenyl, phenanthryl, anthryl, and chrysenyl.

In the present disclosure, the number of carbon atoms of the substituted or unsubstituted heteroaryl may be from 3 to 30, for example, the number of carbon atoms may be 3, 4, 5, 6, 7, 8, 9, 10, 11, 12, 13, 14, 15, 16, 17, 18, 19, 20, 21, 22, 23, 24, 25, 26, 27, 28, 29, or 30.

In the present disclosure, specific examples of heteroaryl as a substituent include, but are not limited to, carbazolyl, dibenzofuranyl, dibenzothienyl, quinolyl, quinazolinyl, quinoxalinyl, isoquinolyl, and N-phenylcarbazolyl.

In the present disclosure, an unpositioned connecting bond refers to a single bond

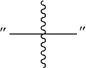

extending from a ring system, which means that one end of the connecting bond can be connected with any position in the ring system through which the bond penetrates, and the other end of the connecting bond is connected with the remaining part of a compound molecule.

For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1) to (f-10):

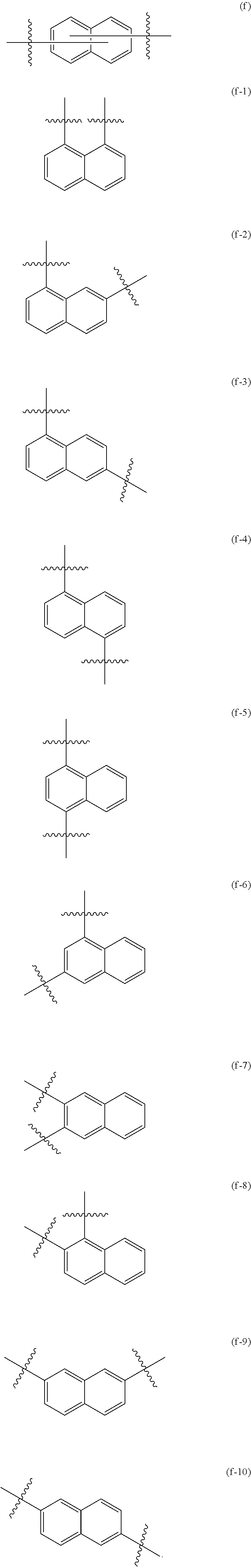

For example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by formulae (X′-1) to (X′-4):

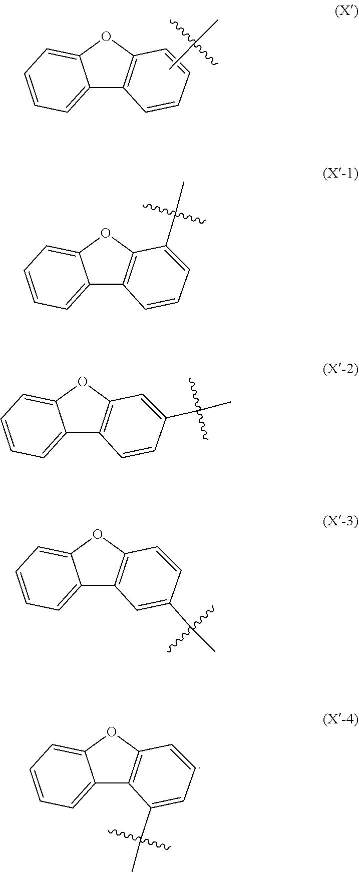

In the present disclosure, the alkyl with 1 to 10 carbon atoms may include linear alkyl with 1 to 10 carbon atoms and branched alkyl with 3 to 10 carbon atoms. The number of carbon atoms of the alkyl may be, for example, 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, n-octyl, 2-ethylhexyl, nonyl, decyl, 3,7-dimethyloctyl, and the like.

In the present disclosure, the halogen group may be, for example, fluorine, chlorine, bromine or iodine.

In the present disclosure, the number of carbon atoms of cycloalkyl with 3 to 10 carbon atoms may be, for example, 3, 4, 5, 6, 7, 8, or 10. Specific examples of cycloalkyl include, but are not limited to, cyclopentyl and cyclohexyl.

In the present disclosure, specific examples of the triarylsilyl with 18 to 24 carbon atoms include, but are not limited to, triphenylsilyl and the like.

In the present disclosure, specific examples of the deuterated alkyl with 1 to 10 carbon atoms include, but are not limited to, trideuteromethyl.

In the present disclosure, specific examples of the deuterated aryl with 6 to 20 carbon atoms include, but are not limited to, monodeuterophenyl, dideutrophenyl, trideuterophenyl, tetradeuterophenyl, and pentadeuterophenyl.

In the present disclosure, specific examples of the haloaryl with 6 to 20 carbon atoms include, but are not limited to, monofluorophenyl, difluorophenyl, trifluorophenyl, tetrafluorophenyl, and pentafluorophenyl.

In some embodiments of the present disclosure, the organic compound has a structure represented by a formula 1-1, a formula 1-2, a formula 1-3, a formula 1-4, a formula 1-5, or a formula 1-6:

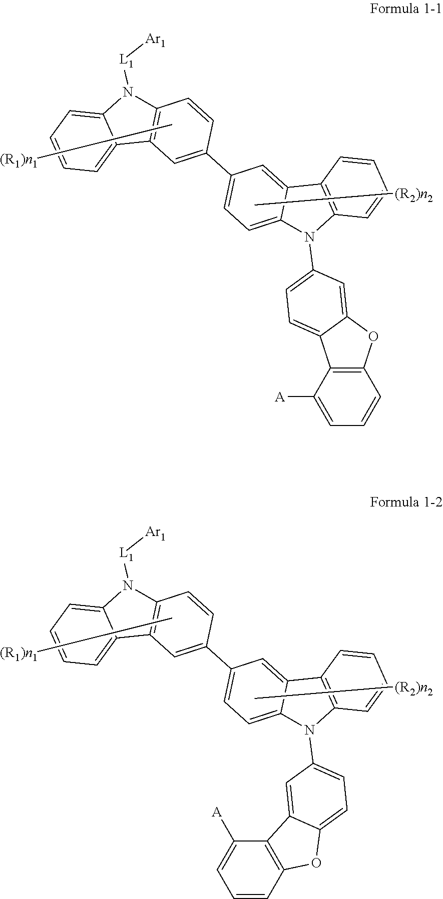

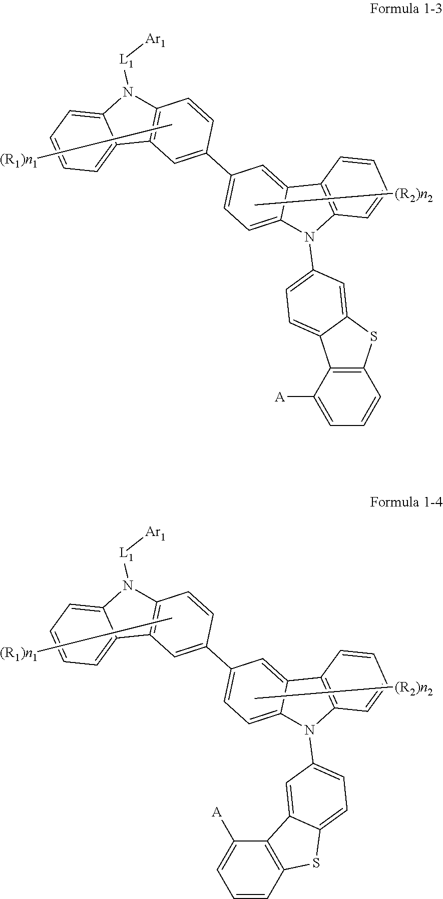

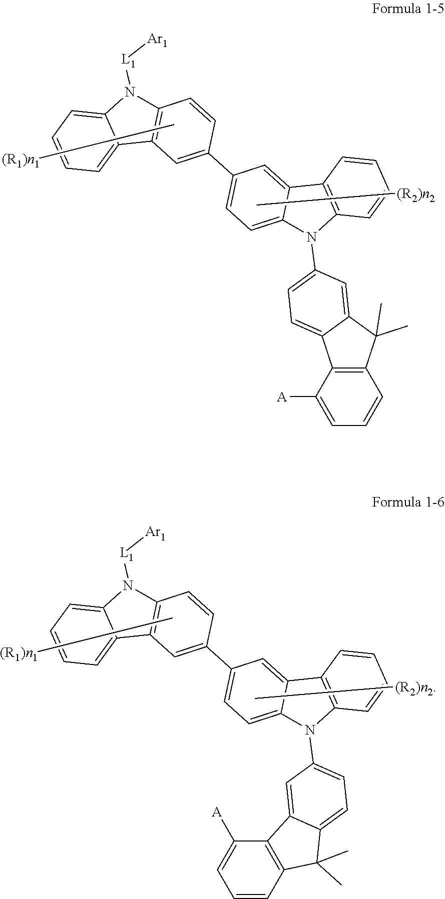

In some embodiments of the present disclosure, the group A is selected from substituted or unsubstituted phenyl, substituted or unsubstituted biphenyl, and substituted or unsubstituted naphthyl.

Optionally, substituents in the group A are the same or different, and are respectively and independently selected from deuterium, fluorine, cyano, methyl, ethyl, n-propyl, isopropyl, tert-butyl or phenyl.

In other embodiments of the present disclosure, the group A is selected from

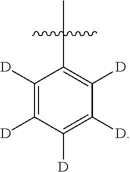

In some embodiments of the present disclosure, X is selected from O or S, and the group A is

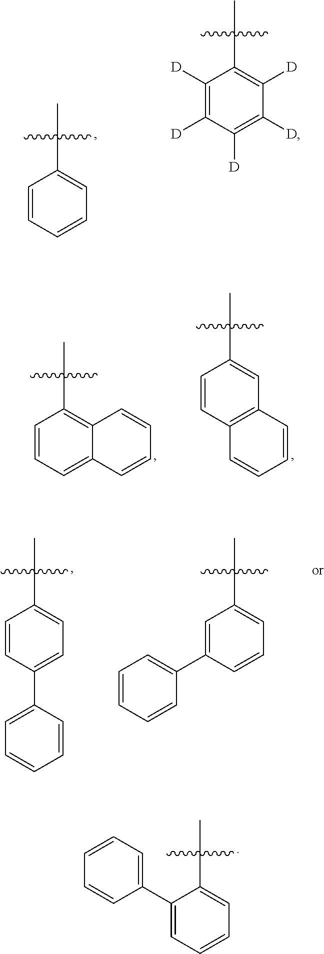

In some embodiments, L₁ is selected from a single bond, substituted or unsubstituted arylene with 6 to 12 carbon atoms, and substituted or unsubstituted heteroarylene with 12 to 20 carbon atoms.

Optionally, substituents in L₁ are the same or different, and are respectively and independently selected from deuterium, a halogen group, cyano, alkyl with 1 to 5 carbon atoms, phenyl, or pentadeuterophenyl.

In other embodiments of the present disclosure, L₁ is selected from a single bond, substituted or unsubstituted phenylene, substituted or unsubstituted naphthylene, substituted or unsubstituted biphenylene, substituted or unsubstituted carbazolylene, substituted or unsubstituted dibenzofurylene, and substituted or unsubstituted dibenzothenylene.

Optionally, substituents in L₁ are the same or different, and are respectively and independently selected from deuterium, fluorine, cyano, methyl, ethyl, n-propyl, isopropyl, tert-butyl, phenyl or pentadeuterophenyl.

In some embodiments of the present disclosure, L₁ is selected from a single bond, and a substituted or unsubstituted group Q, wherein the unsubstituted group Q is selected from the group consisting of:

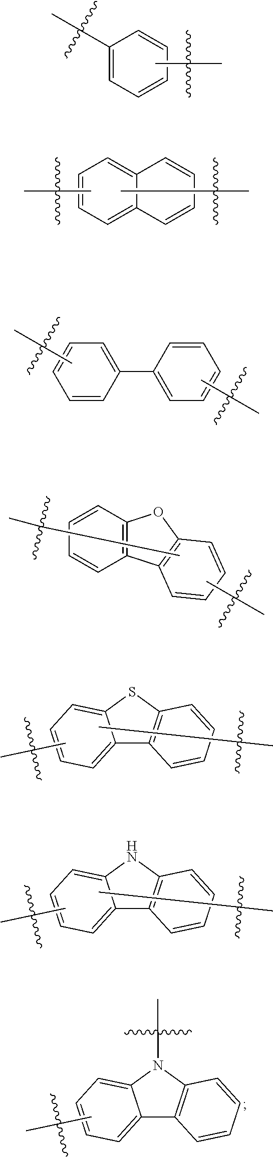

and
the substituted group Q has one or two or more substituents respectively and independently selected from deuterium, fluorine, cyano, methyl, ethyl, n-propyl, isopropyl, tert-butyl, phenyl or pentadeuterophenyl, and when the number of the substituents of the group Q is greater than 1, the substituents are the same or different.

Optionally, L₁ is selected from a single bond or the group consisting of:

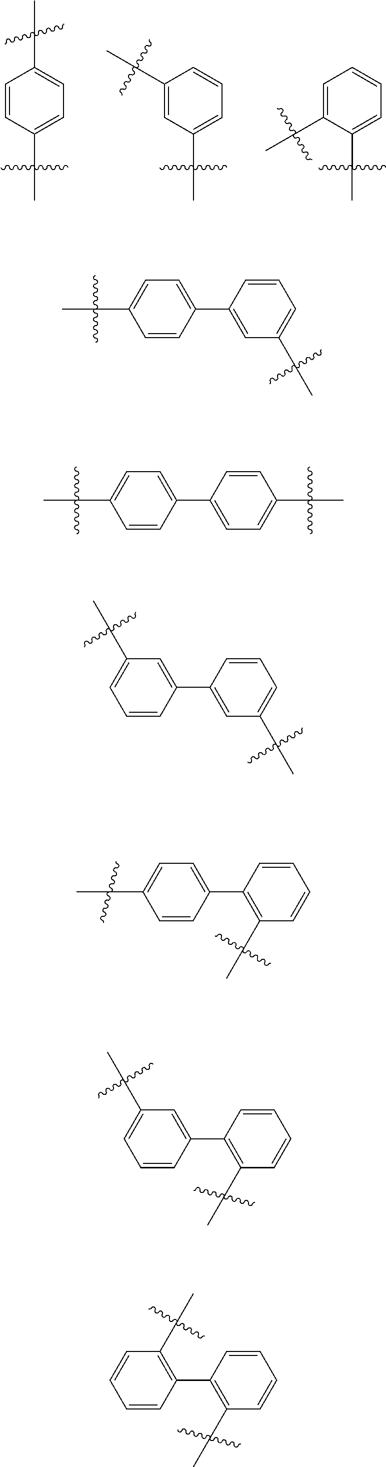

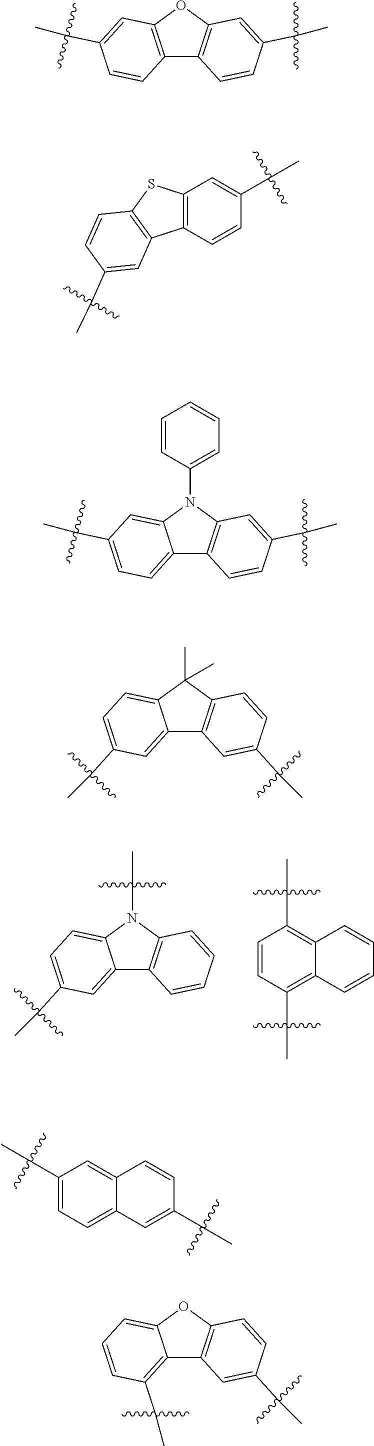

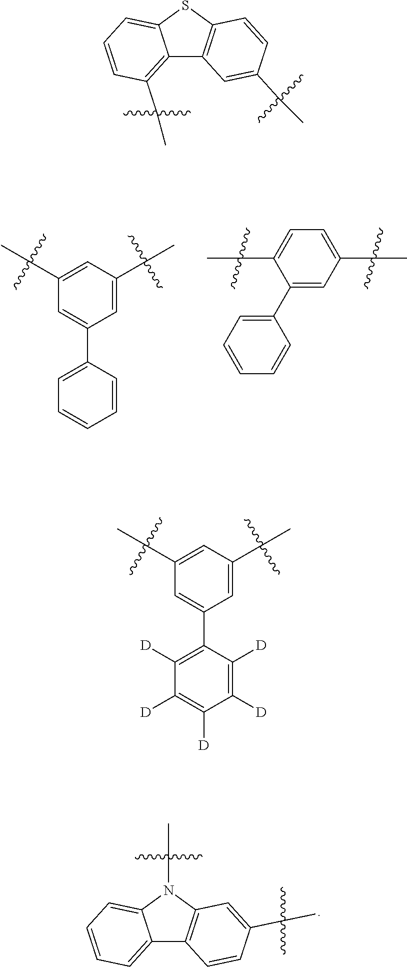 [SMILES: *c1ccc2sc3cccc(*)c3c2c1 ; *c1cc(*)cc(-c2ccccc2)c1 ; *c1ccc(*)c(-c2ccccc2)c1 ; *c1cc(*)cc(-c2c([2H])c([2H])c([2H])c([2H])c2[2H])c1 ; *c1ccc2c3ccccc3n(*)c2c1]

In some embodiments of the present disclosure, Ar₁ is selected from substituted or unsubstituted aryl with 6 to 25 carbon atoms, and substituted or unsubstituted heteroaryl with 12 to 20 carbon atoms.

Optionally, substituents in Ar₁ are the same or different, and are respectively and independently selected from deuterium, a halogen group, cyano, alkyl with 1 to 5 carbon atoms, phenyl, or pentadeuterophenyl.

Optionally, Ar₁ is selected from substituted or unsubstituted aryl with 6 to 20 carbon atoms, and substituted or unsubstituted heteroaryl with 12 to 18 carbon atoms.

In other embodiments of the present disclosure, Ar₁ is selected from substituted or unsubstituted phenyl, substituted or unsubstituted biphenyl, substituted or unsubstituted naphthyl, substituted or unsubstituted fluorenyl, substituted or unsubstituted terphenyl, substituted or unsubstituted dibenzofuranyl, substituted or unsubstituted dibenzothienyl, substituted or unsubstituted carbazolyl, and substituted or unsubstituted phenanthryl.

Optionally, substituents in Ar₁ are the same or different, and are respectively and independently selected from deuterium, fluorine, cyano, methyl, ethyl, n-propyl, isopropyl, tert-butyl, phenyl or pentadeuterophenyl.

In some embodiments of the present disclosure, Ar₁ is selected from a substituted or unsubstituted group W, wherein the unsubstituted group W is selected from the group consisting of:

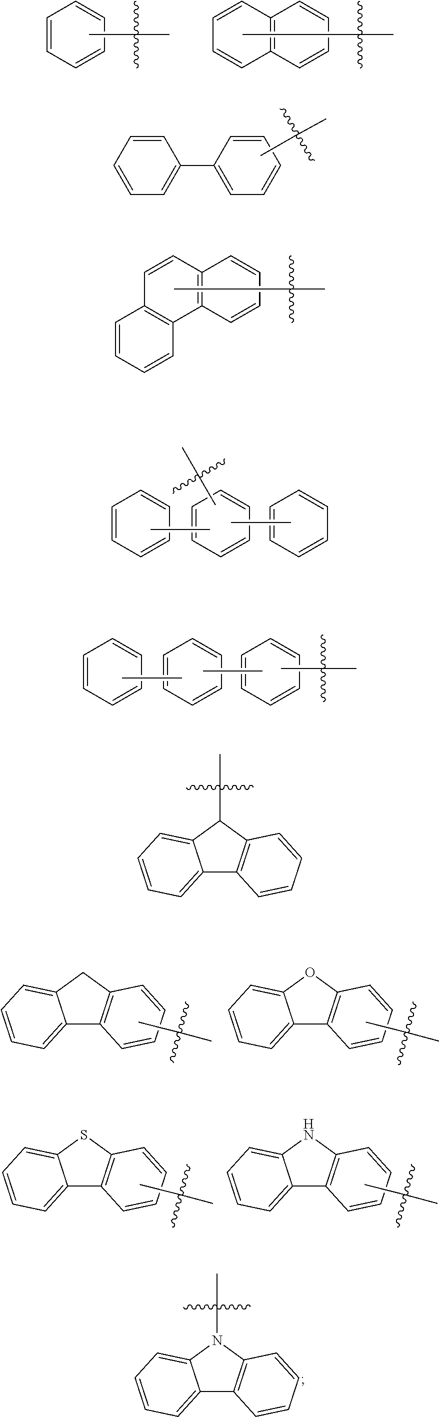

and
the substituted group W has one or two or more substituents, the substituents in the substituted group W are respectively and independently selected from the group consisting of deuterium, fluorine, cyano, methyl, ethyl, n-propyl, isopropyl, tert-butyl, phenyl or pentadeuterophenyl, and when the number of the substituents on the group W is greater than 1, the substituents are the same or different.

Optionally, Ar₁ is selected from the group consisting of:

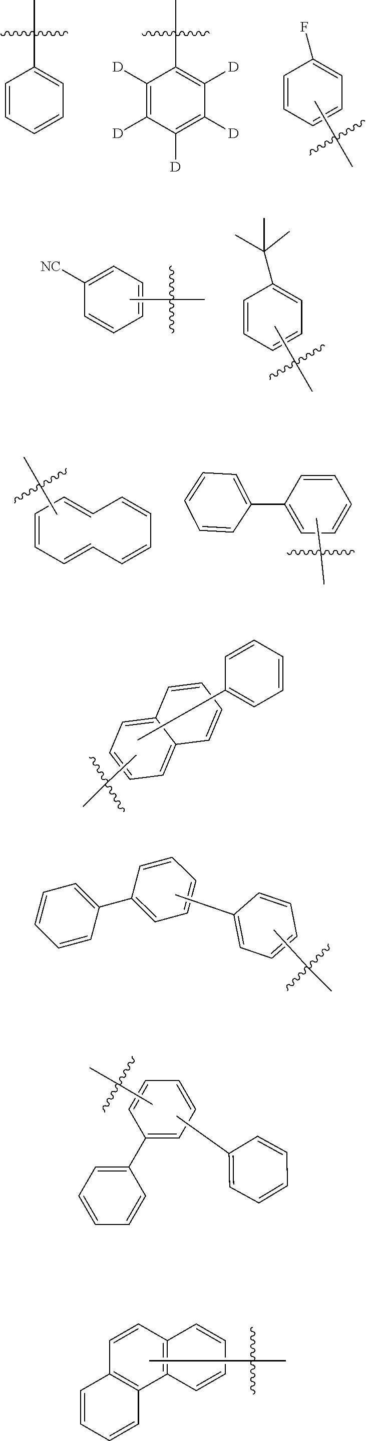

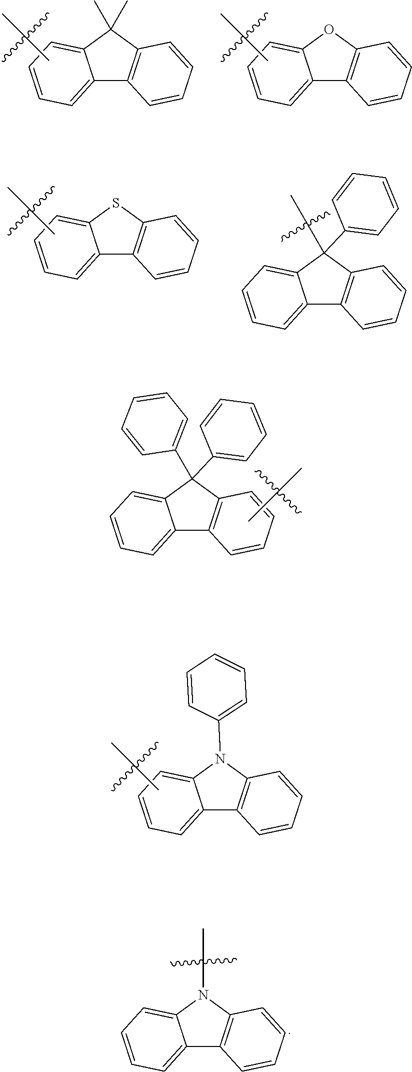

In particular, Ar₁ is selected from the group consisting of:

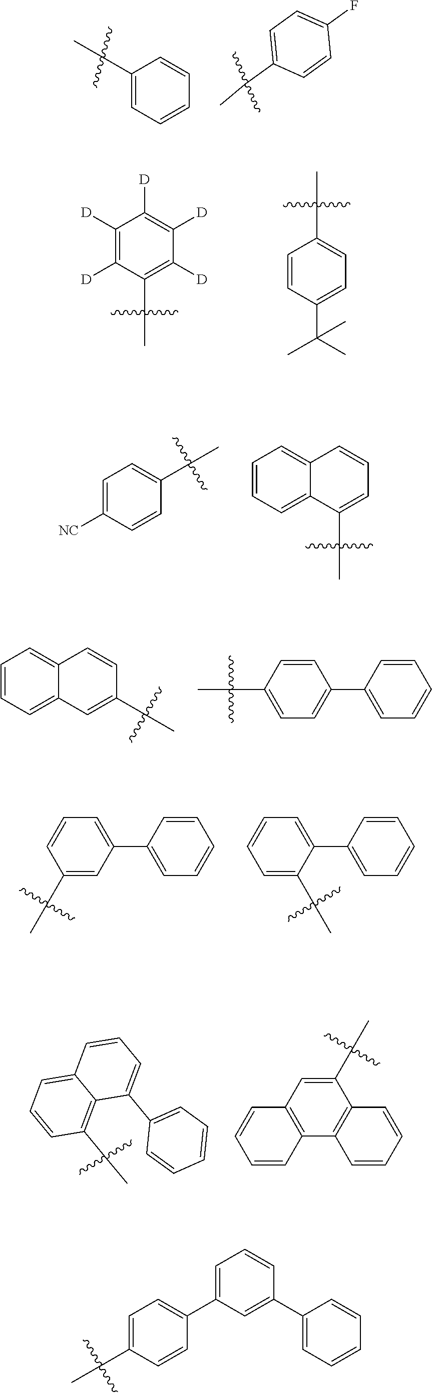

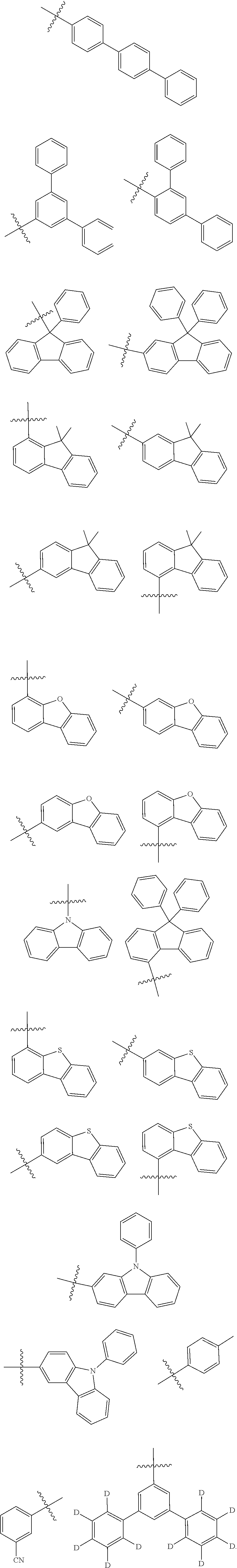

In some embodiments of the present disclosure,

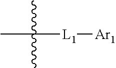

is selected from a substituted or unsubstituted group V, wherein the unsubstituted group V is selected from the group consisting of:

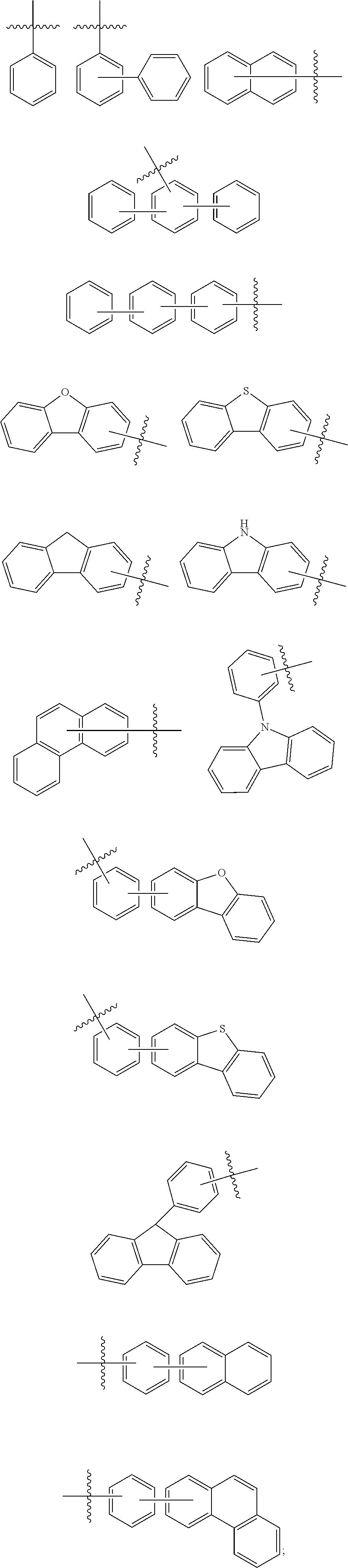

and

the substituted group V has one or two or more substituents, the substituents in substituted group V are respectively and independently selected from the group consisting of deuterium, fluorine, cyano, methyl, ethyl, n-propyl, isopropyl, tert-butyl, phenyl or pentadeuterophenyl, and when the number of the substituents on the group V is greater than 1, the substituents are the same or different.

Optionally,

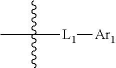

is selected from the group consisting of:

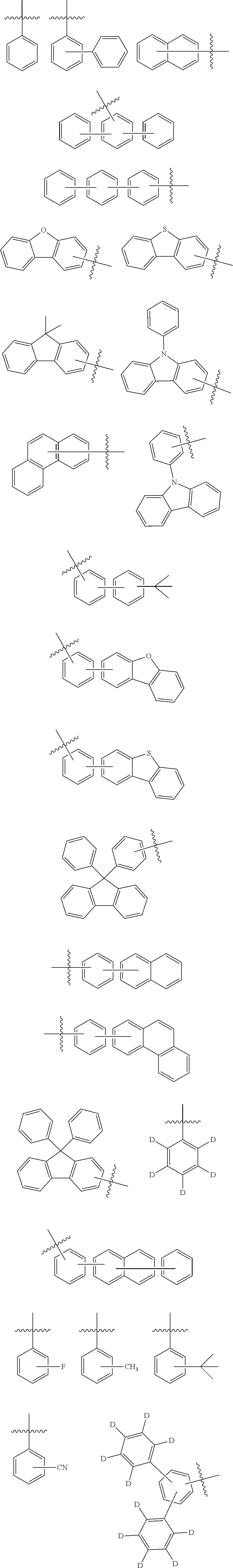

Further optionally,

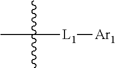

is selected from the group consisting of:

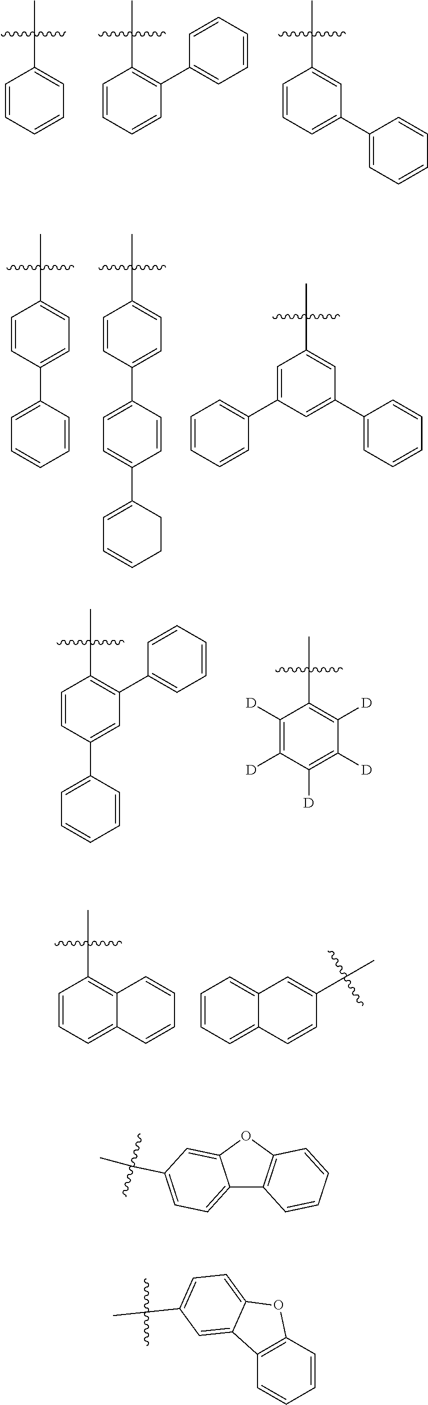

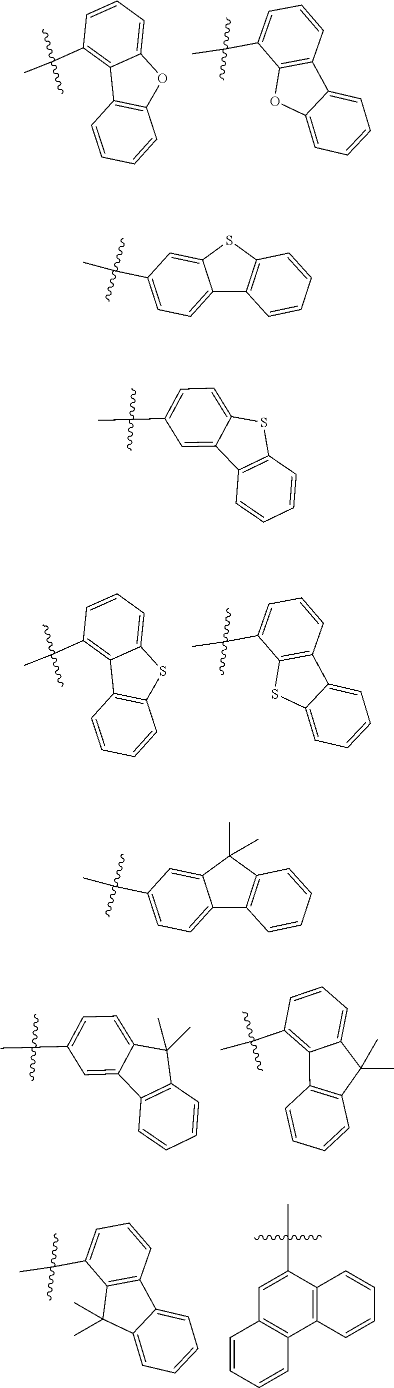

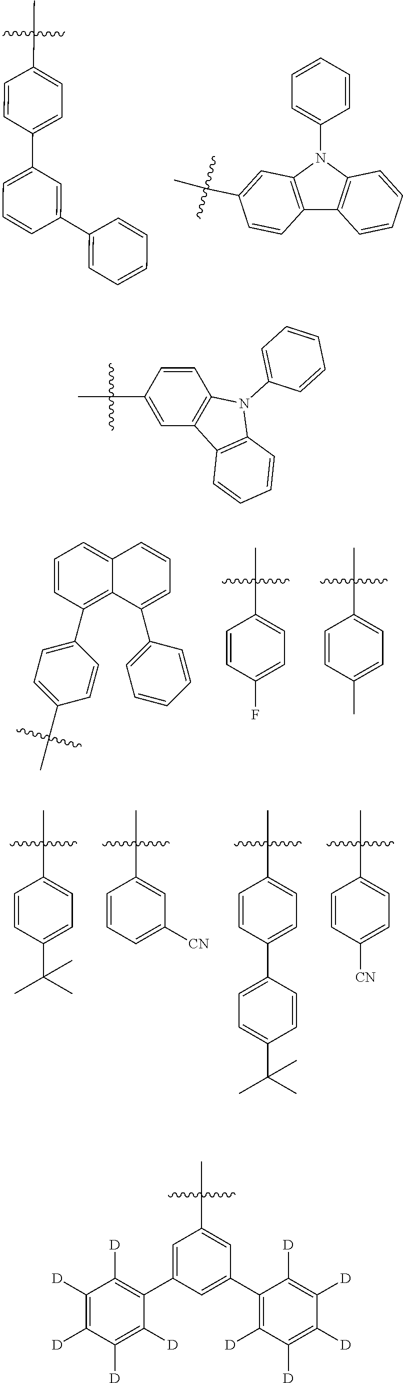

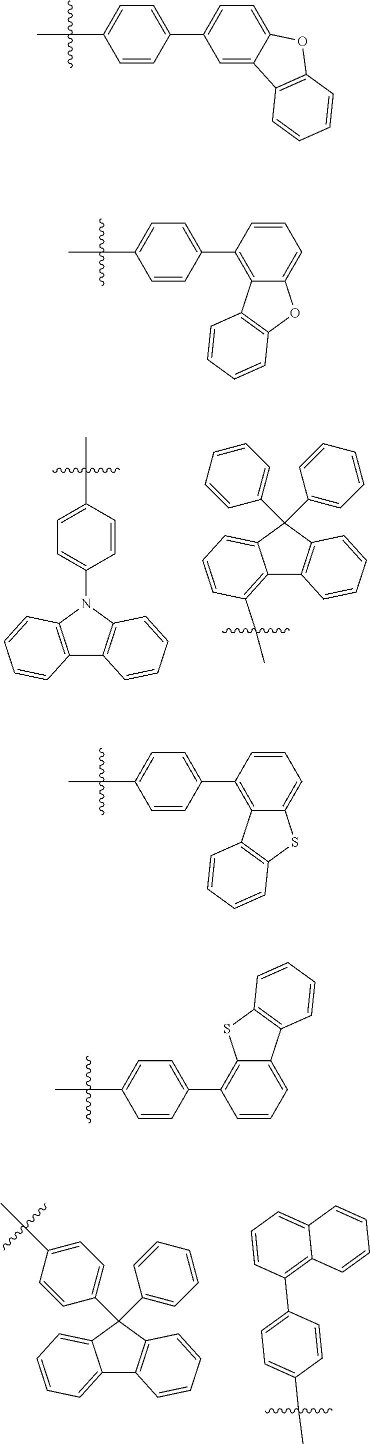

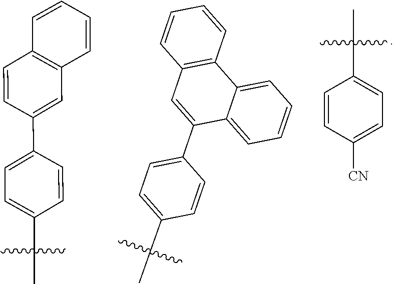

In some embodiments of the present disclosure, each R₁ and each R₂ are the same or different, and are respectively and independently selected from deuterium, a halogen group, cyano, alkyl with 1 to 5 carbon atoms, aryl with 6 to 12 carbon atoms, and deuterated aryl with 6 to 12 carbon atoms.

Optionally, each R₁ and each R₂ are the same or different, and are respectively and independently selected from deuterium, fluorine, cyano, methyl, ethyl, n-propyl, isopropyl, tert-butyl, phenyl, naphthyl, biphenyl or pentadeuterophenyl.

Further optionally, each R₁ and each R₂ are the same or different, and are respectively and independently selected from deuterium, phenyl or pentadeuterophenyl.

In other embodiments of the present disclosure, each R₁ and each R₂ are the same or different, and are respectively and independently selected from deuterium, fluorine, cyano, methyl, ethyl, n-propyl, isopropyl, tert-butyl or the group consisting of:

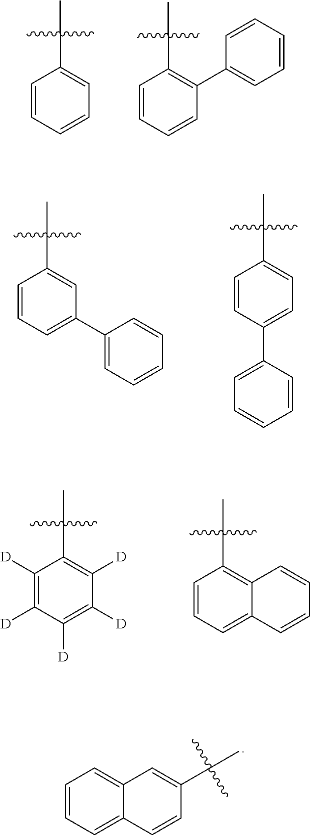

In other embodiments of the present disclosure, R₃ and R₄ are the same or different, and are respectively and independently selected from hydrogen, deuterium or methyl.

Optionally, the organic compound is selected from the following compounds:

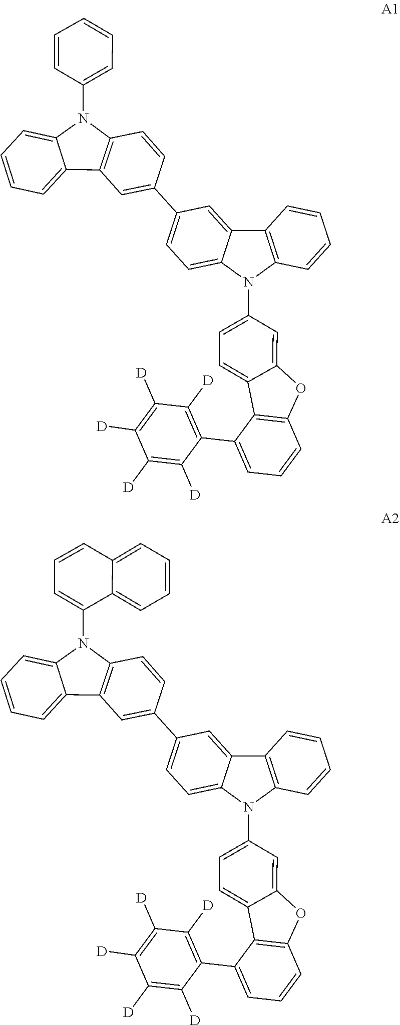 [SMILES: [2H]c1c([2H])c([2H])c(-c2cccc3oc4cc(-n5c6ccccc6c6cc(-c7ccc8c(c7)c7ccccc7n8-c7ccccc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cccc8ccccc78)ccc65)ccc4c23)c([2H])c1[2H]]

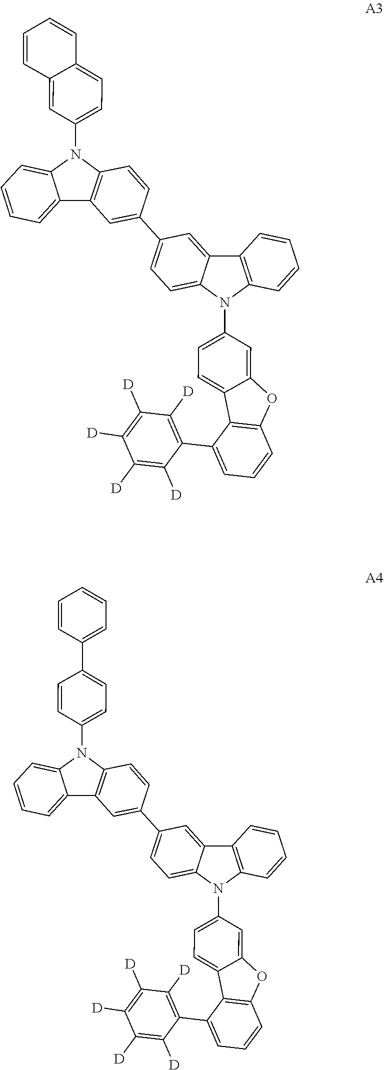 [SMILES: [2H]c1c([2H])c([2H])c(-c2cccc3oc4cc(-n5c6ccccc6c6cc(-c7ccc8c(c7)c7ccccc7n8-c7ccc8ccccc8c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(-c8ccccc8)cc7)ccc65)ccc4c23)c([2H])c1[2H]]

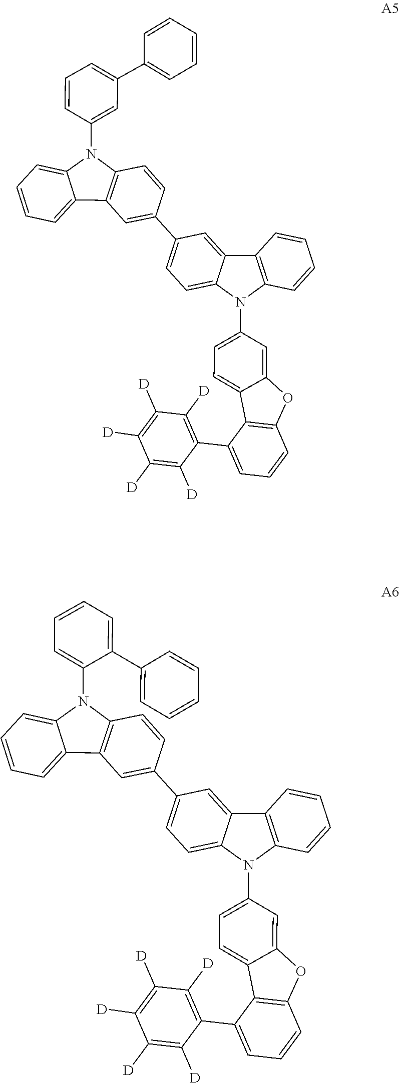 [SMILES: [2H]c1c([2H])c([2H])c(-c2cccc3oc4cc(-n5c6ccccc6c6cc(-c7ccc8c(c7)c7ccccc7n8-c7cccc(-c8ccccc8)c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccccc7-c7ccccc7)ccc65)ccc4c23)c([2H])c1[2H]]

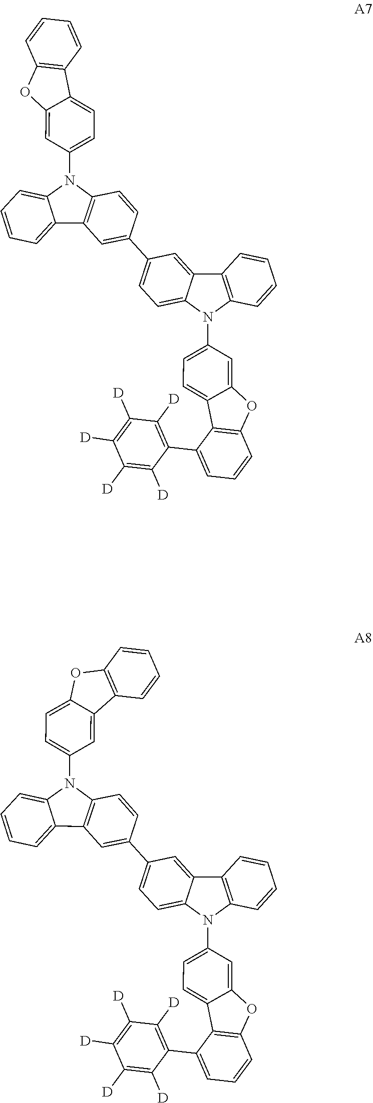 [SMILES: [2H]c1c([2H])c([2H])c(-c2cccc3oc4cc(-n5c6ccccc6c6cc(-c7ccc8c(c7)c7ccccc7n8-c7ccc8c(c7)oc7ccccc78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc8oc9ccccc9c8c7)ccc65)ccc4c23)c([2H])c1[2H]]

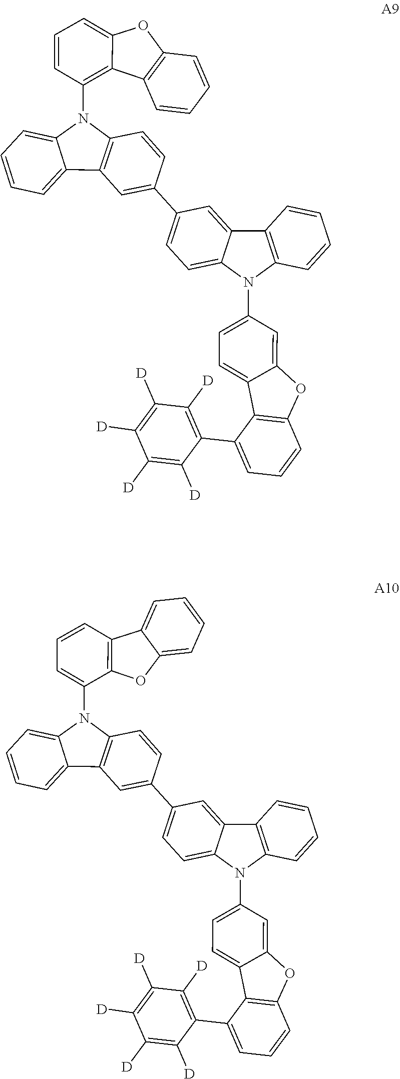 [SMILES: [2H]c1c([2H])c([2H])c(-c2cccc3oc4cc(-n5c6ccccc6c6cc(-c7ccc8c(c7)c7ccccc7n8-c7cccc8oc9ccccc9c78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cccc8c7oc7ccccc78)ccc65)ccc4c23)c([2H])c1[2H]]

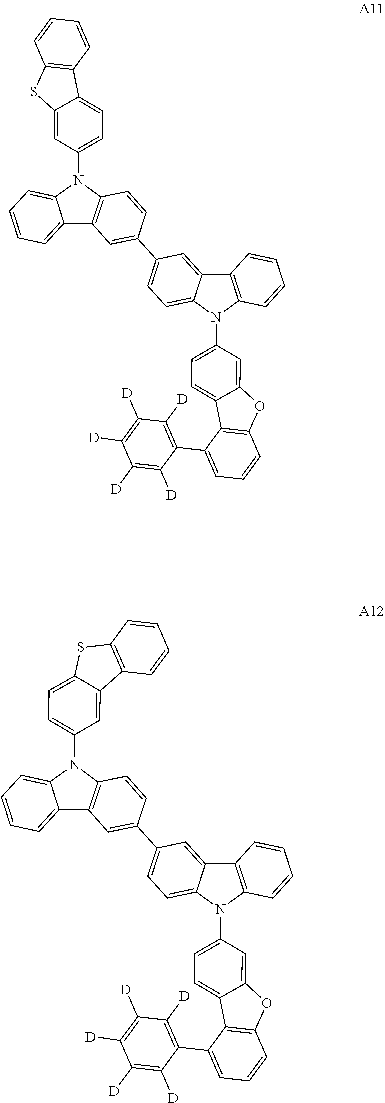 [SMILES: [2H]c1c([2H])c([2H])c(-c2cccc3oc4cc(-n5c6ccccc6c6cc(-c7ccc8c(c7)c7ccccc7n8-c7ccc8c(c7)sc7ccccc78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc8sc9ccccc9c8c7)ccc65)ccc4c23)c([2H])c1[2H]]

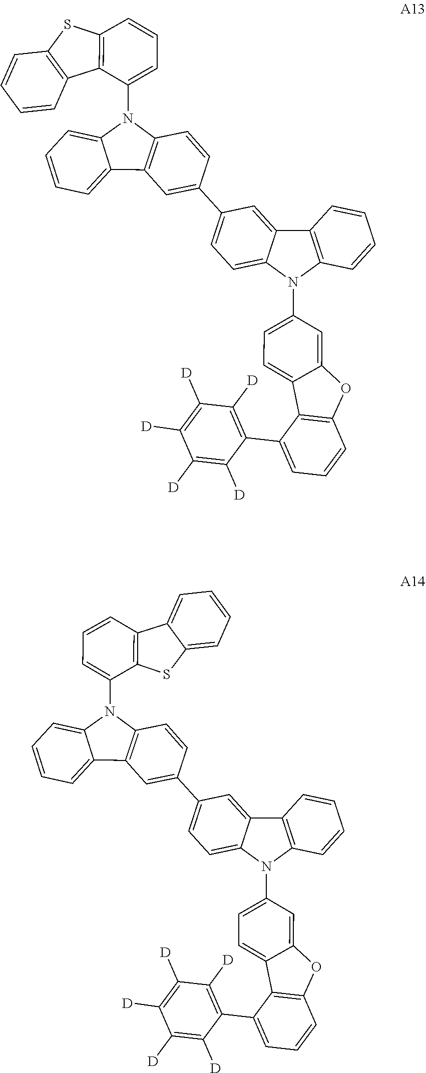 [SMILES: [2H]c1c([2H])c([2H])c(-c2cccc3oc4cc(-n5c6ccccc6c6cc(-c7ccc8c(c7)c7ccccc7n8-c7cccc8sc9ccccc9c78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cccc8c7sc7ccccc78)ccc65)ccc4c23)c([2H])c1[2H]]

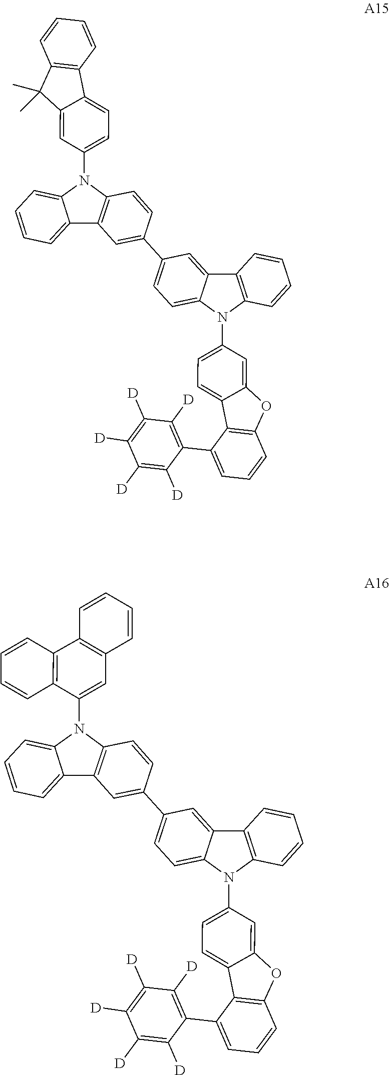 [SMILES: [2H]c1c([2H])c([2H])c(-c2cccc3oc4cc(-n5c6ccccc6c6cc(-c7ccc8c(c7)c7ccccc7n8-c7ccc8c(c7)C(C)(C)c7ccccc7-8)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cc8ccccc8c8ccccc78)ccc65)ccc4c23)c([2H])c1[2H]]

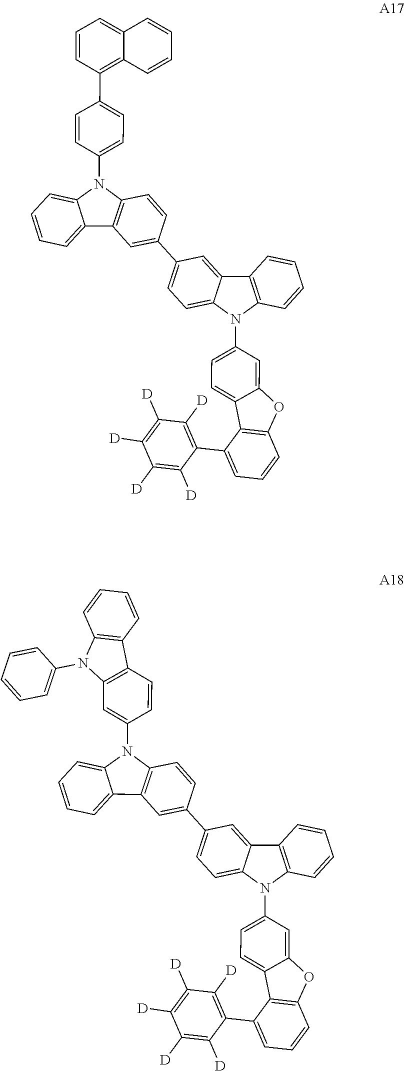 [SMILES: [2H]c1c([2H])c([2H])c(-c2cccc3oc4cc(-n5c6ccccc6c6cc(-c7ccc8c(c7)c7ccccc7n8-c7ccc(-c8cccc9ccccc89)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc8c9ccccc9n(-c9ccccc9)c8c7)ccc65)ccc4c23)c([2H])c1[2H]]

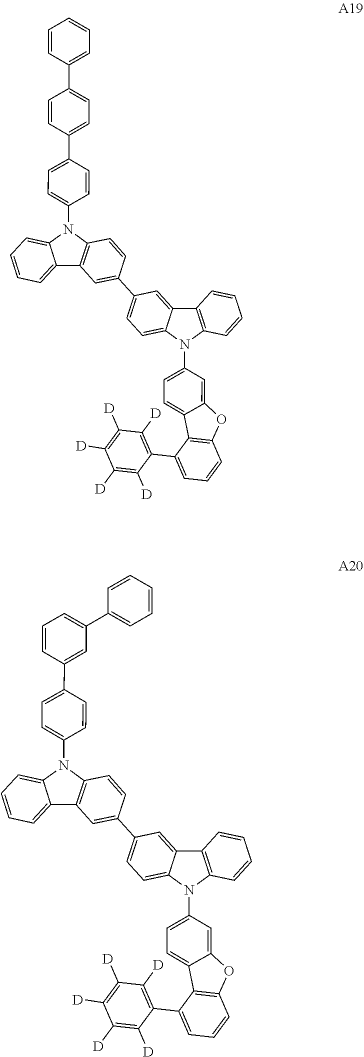 [SMILES: [2H]c1c([2H])c([2H])c(-c2cccc3oc4cc(-n5c6ccccc6c6cc(-c7ccc8c(c7)c7ccccc7n8-c7ccc(-c8ccc(-c9ccccc9)cc8)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(-c8cccc(-c9ccccc9)c8)cc7)ccc65)ccc4c23)c([2H])c1[2H]]

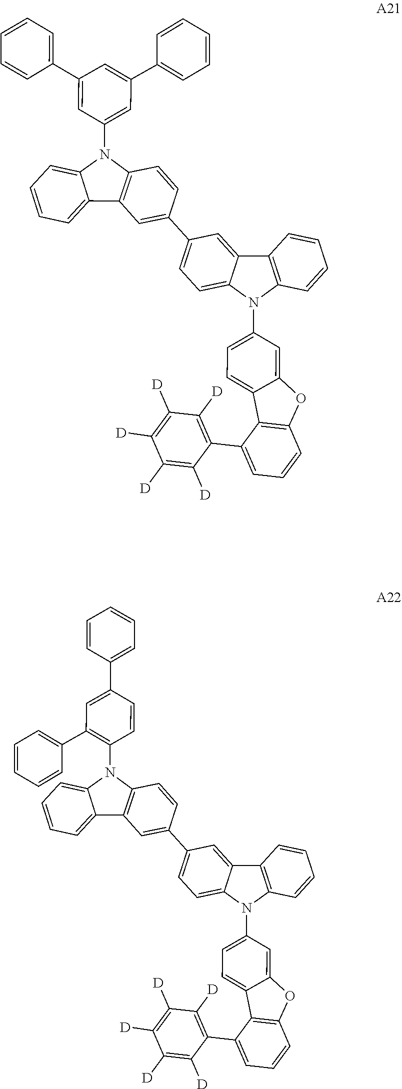 [SMILES: [2H]c1c([2H])c([2H])c(-c2cccc3oc4cc(-n5c6ccccc6c6cc(-c7ccc8c(c7)c7ccccc7n8-c7cc(-c8ccccc8)cc(-c8ccccc8)c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(-c8ccccc8)cc7-c7ccccc7)ccc65)ccc4c23)c([2H])c1[2H]]

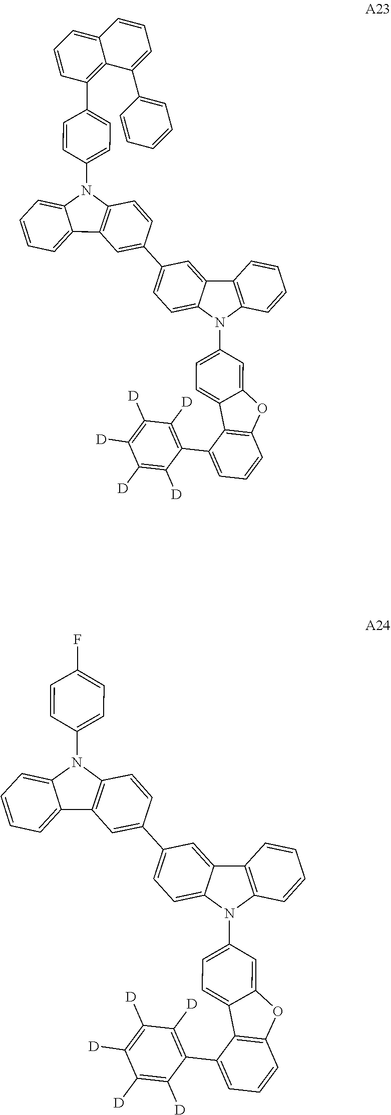 [SMILES: [2H]c1c([2H])c([2H])c(-c2cccc3oc4cc(-n5c6ccccc6c6cc(-c7ccc8c(c7)c7ccccc7n8-c7ccc(-c8cccc9cccc(-c%10ccccc%10)c89)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(F)cc7)ccc65)ccc4c23)c([2H])c1[2H]]

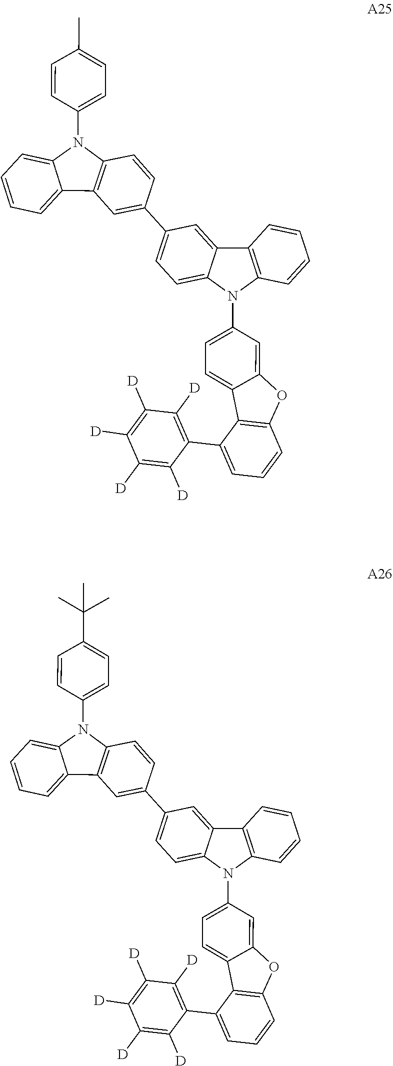 [SMILES: [2H]c1c([2H])c([2H])c(-c2cccc3oc4cc(-n5c6ccccc6c6cc(-c7ccc8c(c7)c7ccccc7n8-c7ccc(C)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(C(C)(C)C)cc7)ccc65)ccc4c23)c([2H])c1[2H]]

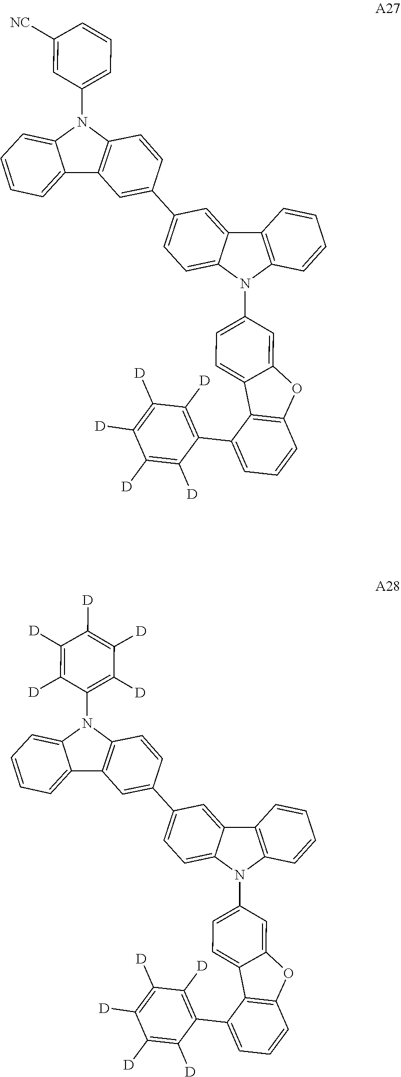 [SMILES: [2H]c1c([2H])c([2H])c(-c2cccc3oc4cc(-n5c6ccccc6c6cc(-c7ccc8c(c7)c7ccccc7n8-c7cccc(C#N)c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7c([2H])c([2H])c([2H])c([2H])c7[2H])ccc65)ccc4c23)c([2H])c1[2H]]

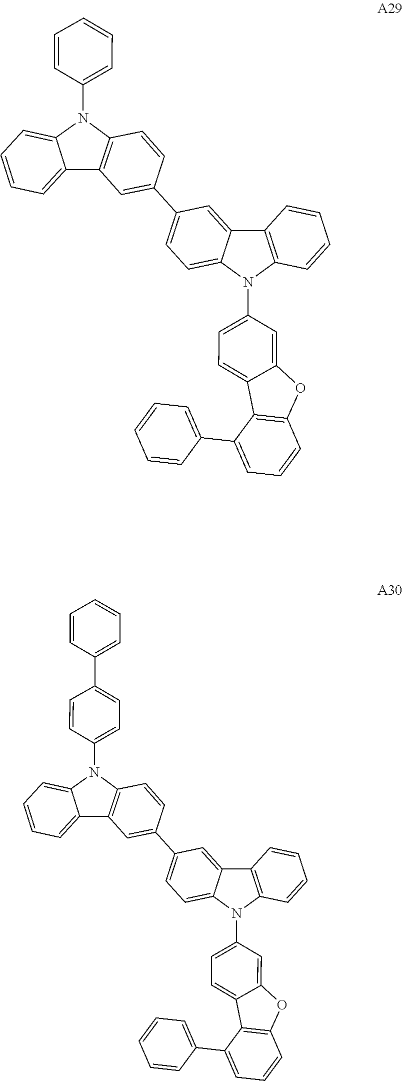 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc6c(c5)oc5cccc(-c7ccccc7)c56)ccc43)cc2)cc1]

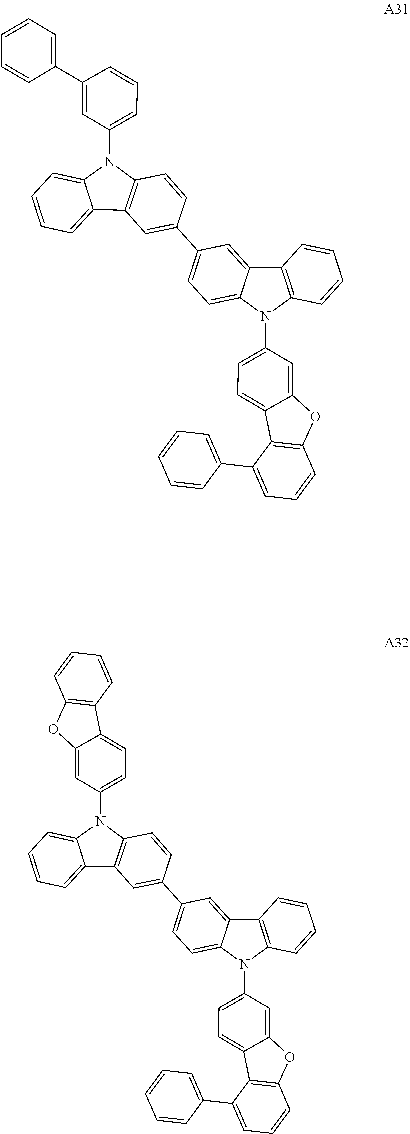 [SMILES: c1ccc(-c2cccc(-n3c4ccccc4c4cc(-c5ccc6c(c5)c5ccccc5n6-c5ccc6c(c5)oc5cccc(-c7ccccc7)c56)ccc43)c2)cc1 ; c1ccc(-c2cccc3oc4cc(-n5c6ccccc6c6cc(-c7ccc8c(c7)c7ccccc7n8-c7ccc8c(c7)oc7ccccc78)ccc65)ccc4c23)cc1]

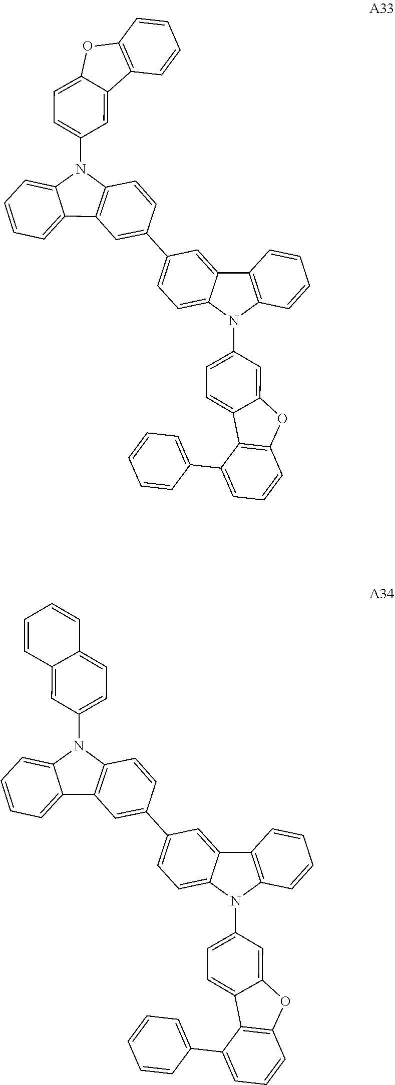 [SMILES: c1ccc(-c2cccc3oc4cc(-n5c6ccccc6c6cc(-c7ccc8c(c7)c7ccccc7n8-c7ccc8oc9ccccc9c8c7)ccc65)ccc4c23)cc1 ; c1ccc(-c2cccc3oc4cc(-n5c6ccccc6c6cc(-c7ccc8c(c7)c7ccccc7n8-c7ccc8ccccc8c7)ccc65)ccc4c23)cc1]

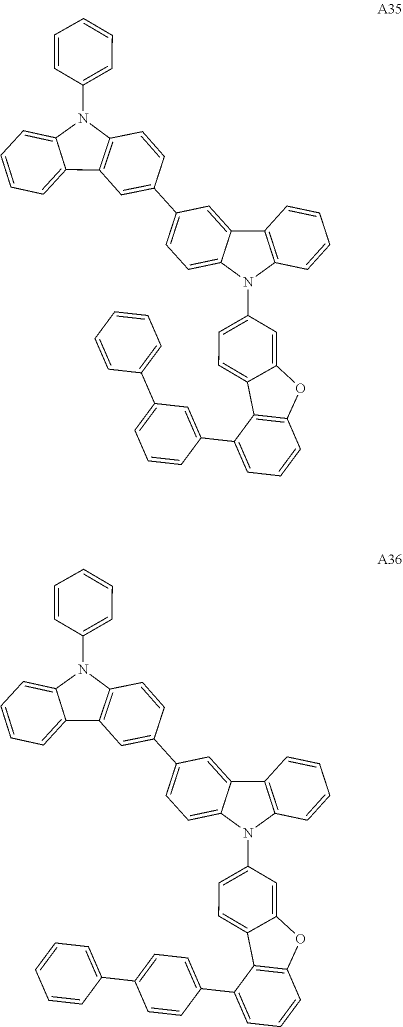 [SMILES: c1ccc(-c2cccc(-c3cccc4oc5cc(-n6c7ccccc7c7cc(-c8ccc9c(c8)c8ccccc8n9-c8ccccc8)ccc76)ccc5c34)c2)cc1 ; c1ccc(-c2ccc(-c3cccc4oc5cc(-n6c7ccccc7c7cc(-c8ccc9c(c8)c8ccccc8n9-c8ccccc8)ccc76)ccc5c34)cc2)cc1]

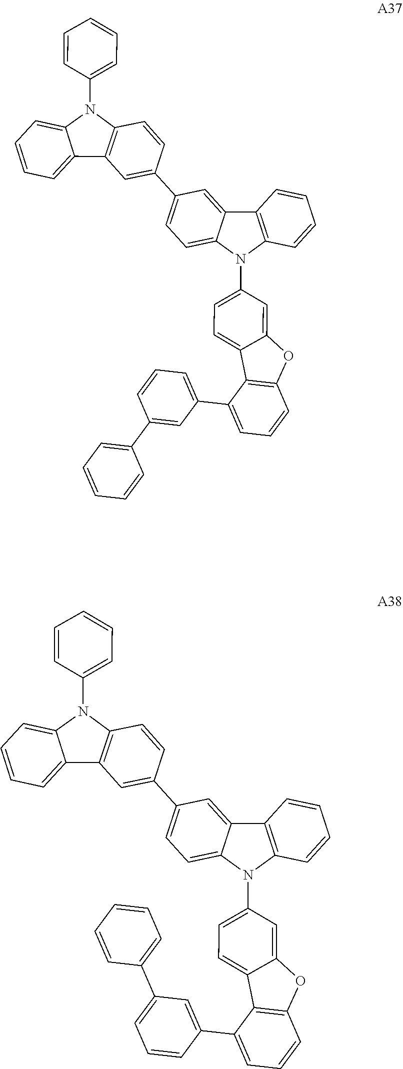 [SMILES: c1ccc(-c2cccc(-c3cccc4oc5cc(-n6c7ccccc7c7cc(-c8ccc9c(c8)c8ccccc8n9-c8ccccc8)ccc76)ccc5c34)c2)cc1 ; c1ccc(-c2cccc(-c3cccc4oc5cc(-n6c7ccccc7c7cc(-c8ccc9c(c8)c8ccccc8n9-c8ccccc8)ccc76)ccc5c34)c2)cc1]

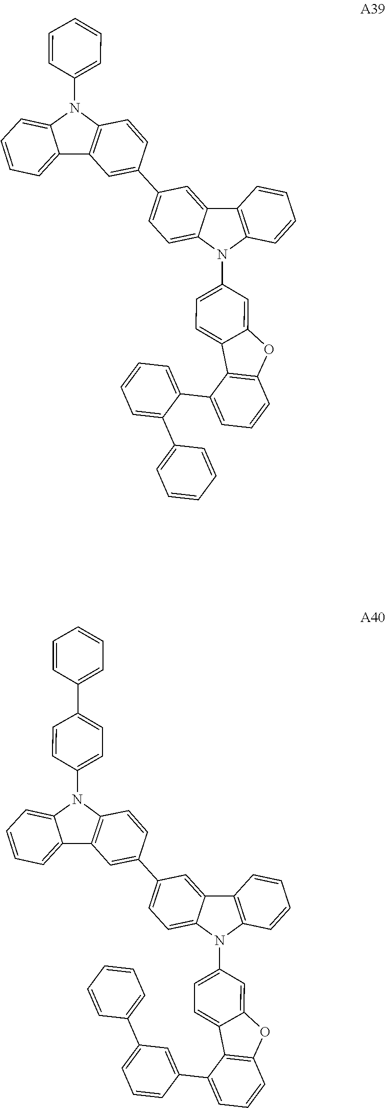 [SMILES: c1ccc(-c2ccccc2-c2cccc3oc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc6c(c5)oc5cccc(-c7cccc(-c8ccccc8)c7)c56)ccc43)cc2)cc1]

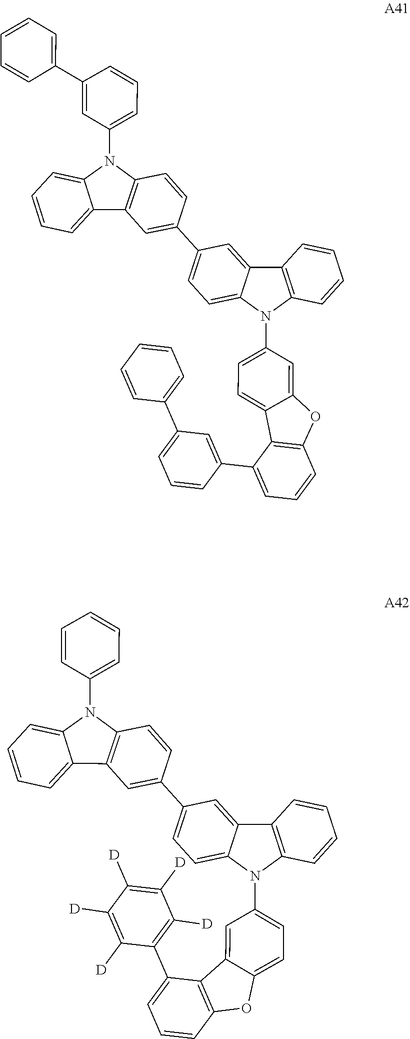 [SMILES: c1ccc(-c2cccc(-c3cccc4oc5cc(-n6c7ccccc7c7cc(-c8ccc9c(c8)c8ccccc8n9-c8cccc(-c9ccccc9)c8)ccc76)ccc5c34)c2)cc1 ; [2H]c1c([2H])c([2H])c(-c2cccc3oc4ccc(-n5c6ccccc6c6cc(-c7ccc8c(c7)c7ccccc7n8-c7ccccc7)ccc65)cc4c23)c([2H])c1[2H]]

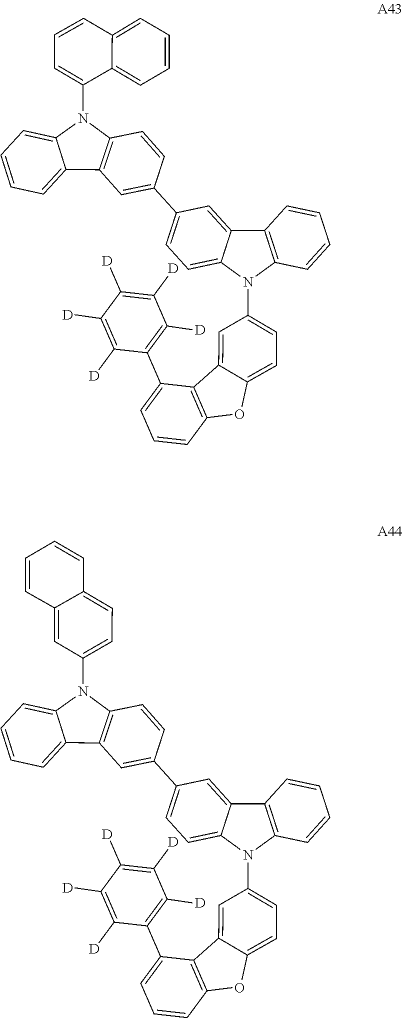 [SMILES: [2H]c1c([2H])c([2H])c(-c2cccc3oc4ccc(-n5c6ccccc6c6cc(-c7ccc8c(c7)c7ccccc7n8-c7cccc8ccccc78)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8ccccc8c7)ccc65)cc4c23)c([2H])c1[2H]]

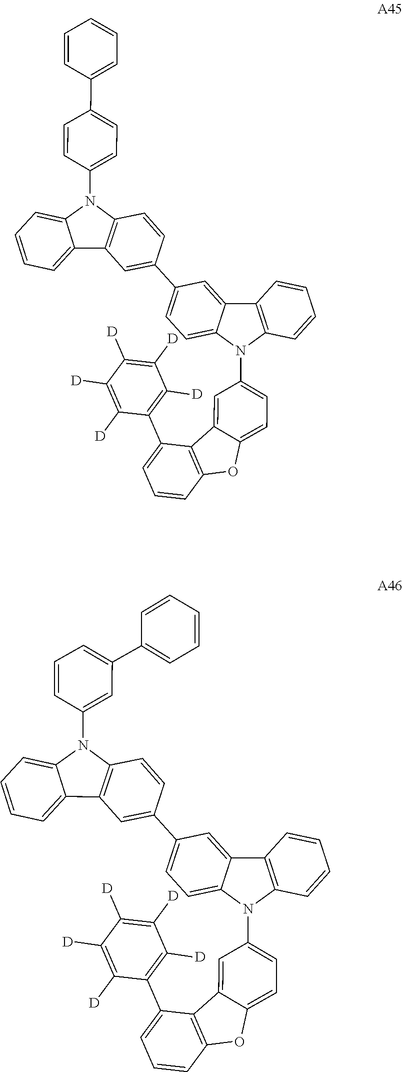 [SMILES: [2H]c1c([2H])c([2H])c(-c2cccc3oc4ccc(-n5c6ccccc6c6cc(-c7ccc8c(c7)c7ccccc7n8-c7ccc(-c8ccccc8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7cccc(-c8ccccc8)c7)ccc65)cc4c23)c([2H])c1[2H]]

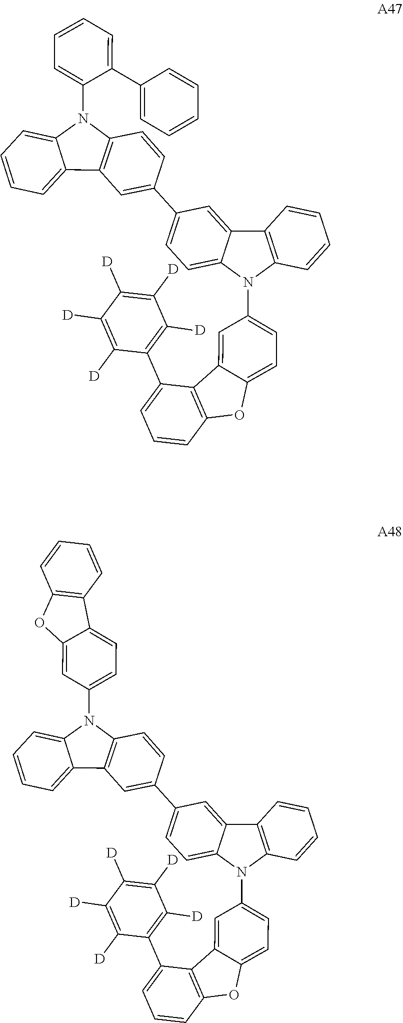 [SMILES: [2H]c1c([2H])c([2H])c(-c2cccc3oc4ccc(-n5c6ccccc6c6cc(-c7ccc8c(c7)c7ccccc7n8-c7ccccc7-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)oc7ccccc78)ccc65)cc4c23)c([2H])c1[2H]]

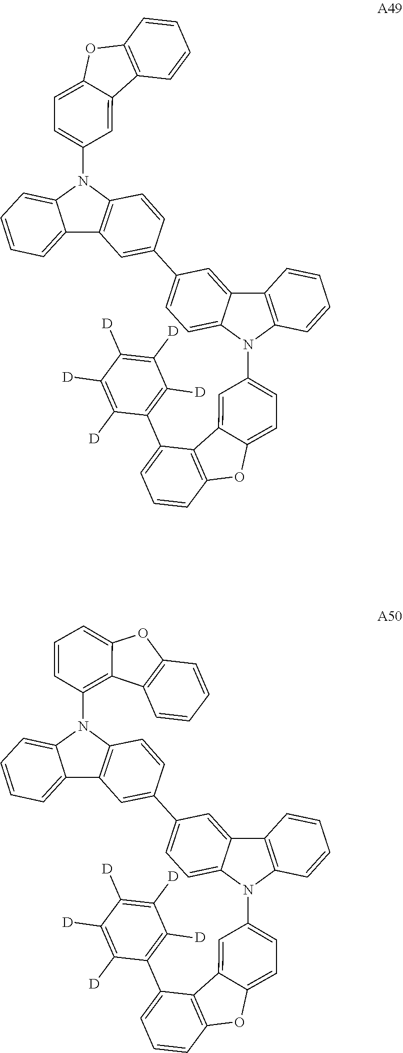 [SMILES: [2H]c1c([2H])c([2H])c(-c2cccc3oc4ccc(-n5c6ccccc6c6cc(-c7ccc8c(c7)c7ccccc7n8-c7ccc8oc9ccccc9c8c7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7cccc8oc9ccccc9c78)ccc65)cc4c23)c([2H])c1[2H]]

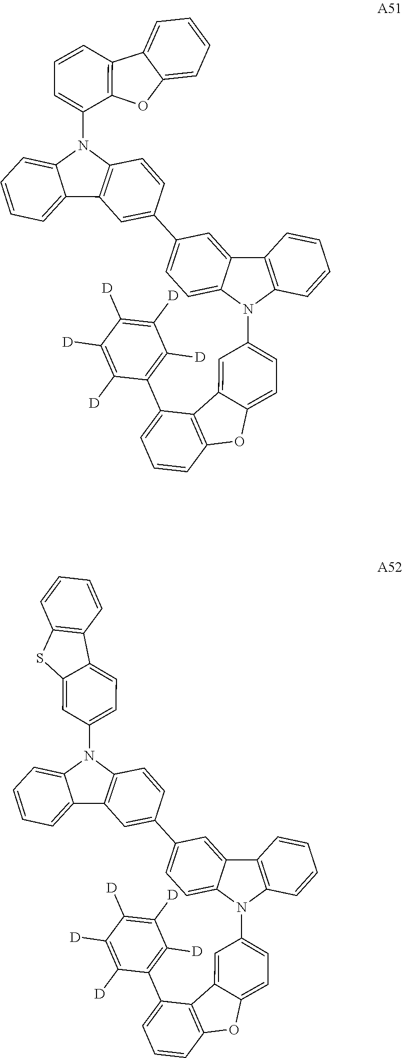 [SMILES: [2H]c1c([2H])c([2H])c(-c2cccc3oc4ccc(-n5c6ccccc6c6cc(-c7ccc8c(c7)c7ccccc7n8-c7cccc8c7oc7ccccc78)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)sc7ccccc78)ccc65)cc4c23)c([2H])c1[2H]]

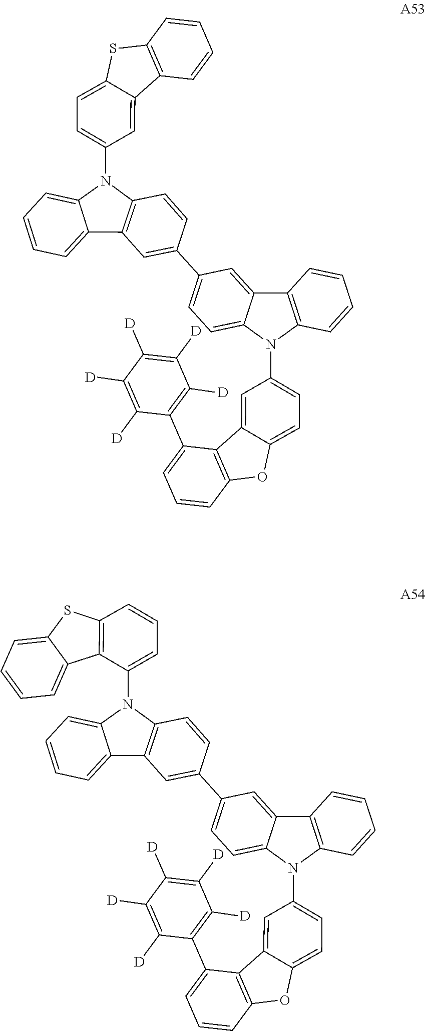 [SMILES: [2H]c1c([2H])c([2H])c(-c2cccc3oc4ccc(-n5c6ccccc6c6cc(-c7ccc8c(c7)c7ccccc7n8-c7ccc8sc9ccccc9c8c7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7cccc8sc9ccccc9c78)ccc65)cc4c23)c([2H])c1[2H]]

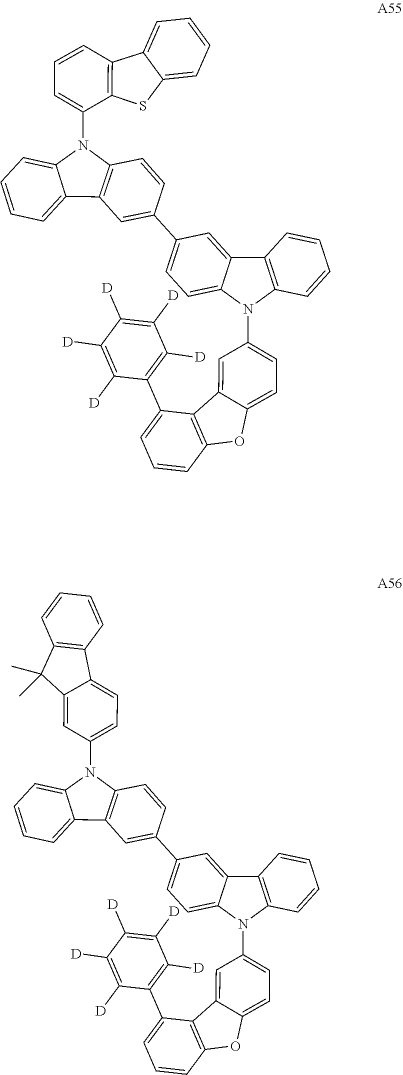 [SMILES: [2H]c1c([2H])c([2H])c(-c2cccc3oc4ccc(-n5c6ccccc6c6cc(-c7ccc8c(c7)c7ccccc7n8-c7cccc8c7sc7ccccc78)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)C(C)(C)c7ccccc7-8)ccc65)cc4c23)c([2H])c1[2H]]

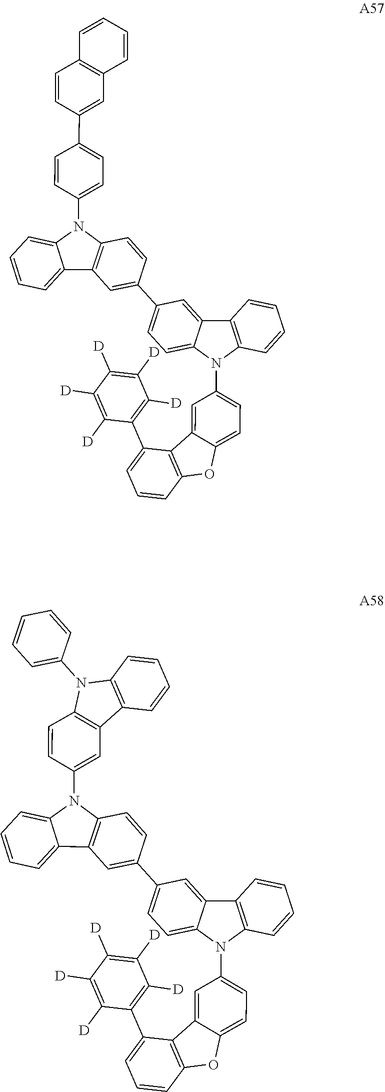 [SMILES: [2H]c1c([2H])c([2H])c(-c2cccc3oc4ccc(-n5c6ccccc6c6cc(-c7ccc8c(c7)c7ccccc7n8-c7ccc(-c8ccc9ccccc9c8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)c7ccccc7n8-c7ccccc7)ccc65)cc4c23)c([2H])c1[2H]]

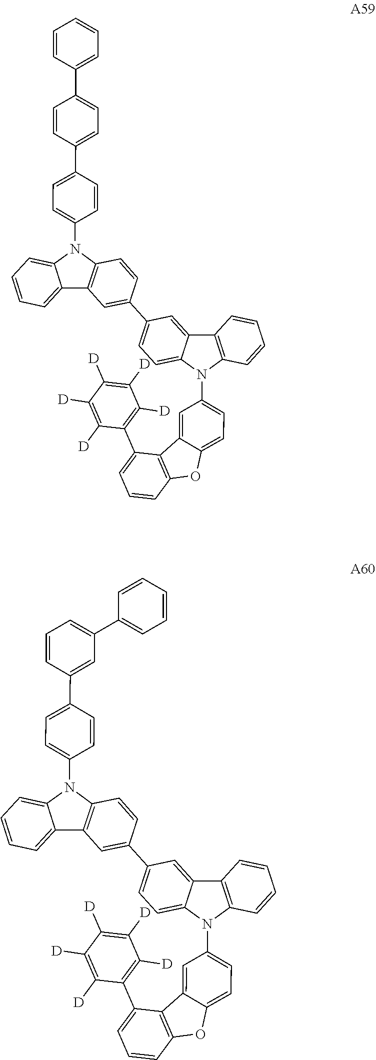 [SMILES: [2H]c1c([2H])c([2H])c(-c2cccc3oc4ccc(-n5c6ccccc6c6cc(-c7ccc8c(c7)c7ccccc7n8-c7ccc(-c8ccc(-c9ccccc9)cc8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(-c8cccc(-c9ccccc9)c8)cc7)ccc65)cc4c23)c([2H])c1[2H]]

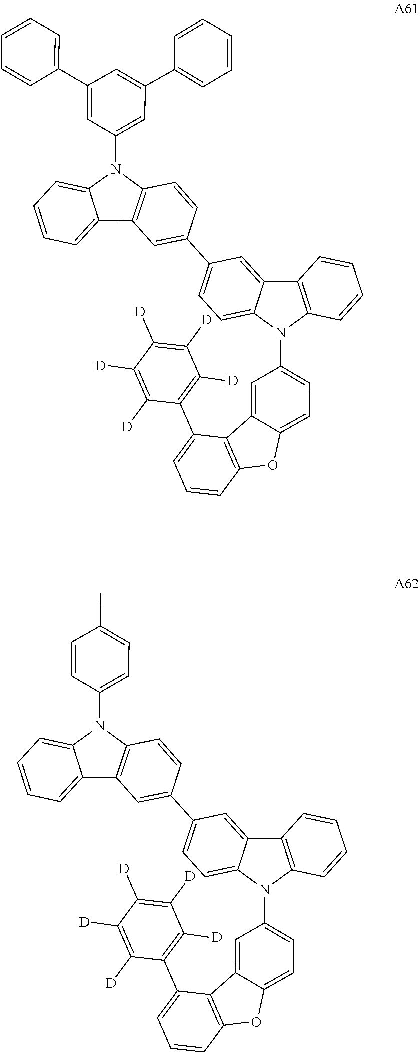 [SMILES: [2H]c1c([2H])c([2H])c(-c2cccc3oc4ccc(-n5c6ccccc6c6cc(-c7ccc8c(c7)c7ccccc7n8-c7cc(-c8ccccc8)cc(-c8ccccc8)c7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(C)cc7)ccc65)cc4c23)c([2H])c1[2H]]

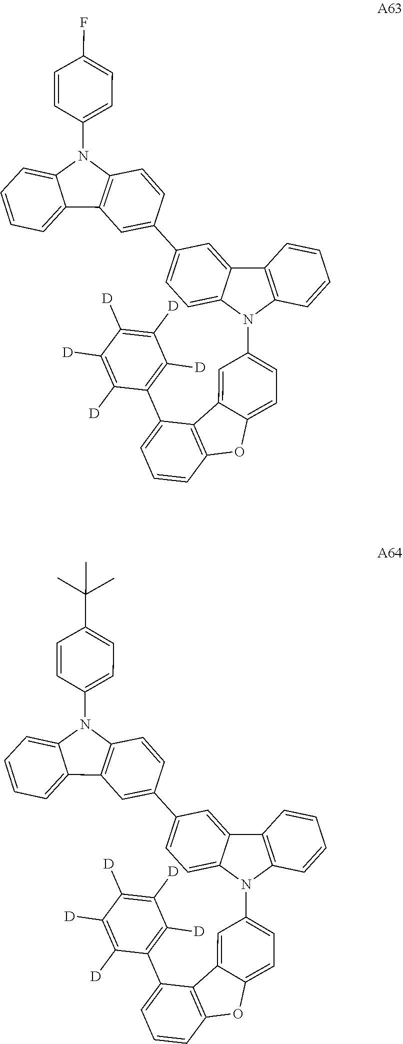 [SMILES: [2H]c1c([2H])c([2H])c(-c2cccc3oc4ccc(-n5c6ccccc6c6cc(-c7ccc8c(c7)c7ccccc7n8-c7ccc(F)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(C(C)(C)C)cc7)ccc65)cc4c23)c([2H])c1[2H]]

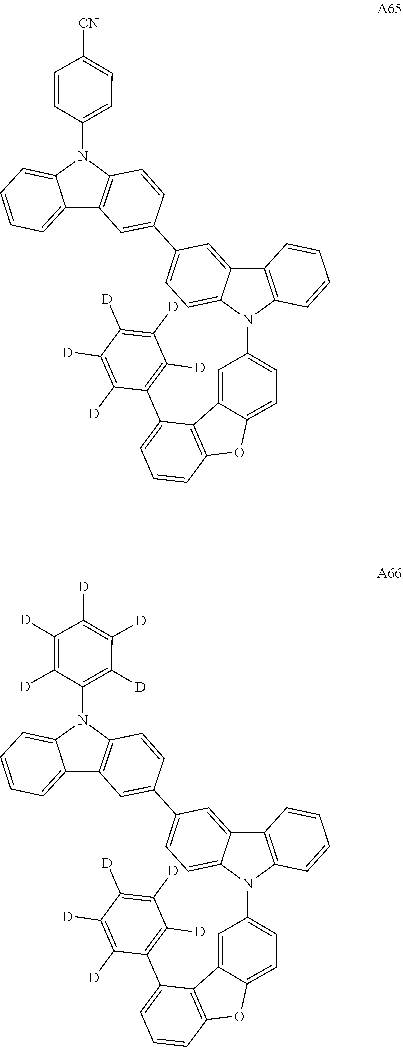 [SMILES: [2H]c1c([2H])c([2H])c(-c2cccc3oc4ccc(-n5c6ccccc6c6cc(-c7ccc8c(c7)c7ccccc7n8-c7ccc(C#N)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7c([2H])c([2H])c([2H])c([2H])c7[2H])ccc65)cc4c23)c([2H])c1[2H]]

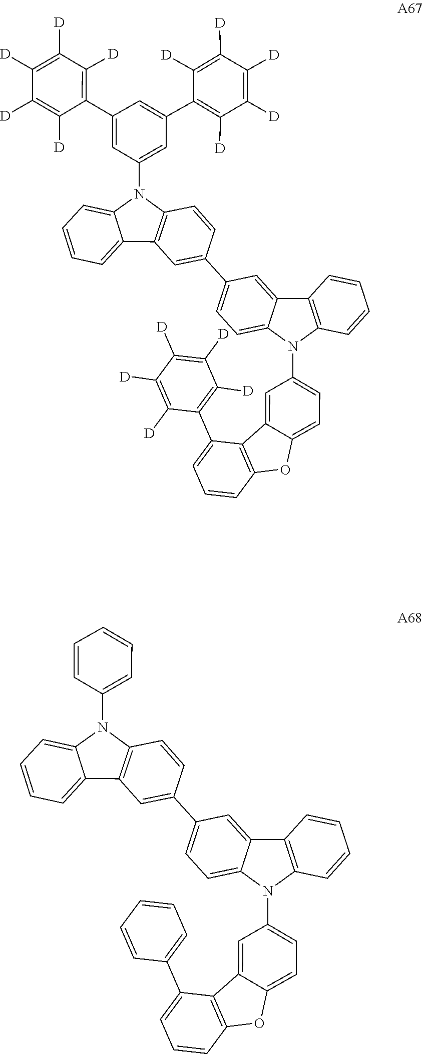 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccc6oc7cccc(-c8c([2H])c([2H])c([2H])c([2H])c8[2H])c7c6c5)ccc43)c2)c([2H])c1[2H] ; c1ccc(-c2cccc3oc4ccc(-n5c6ccccc6c6cc(-c7ccc8c(c7)c7ccccc7n8-c7ccccc7)ccc65)cc4c23)cc1]

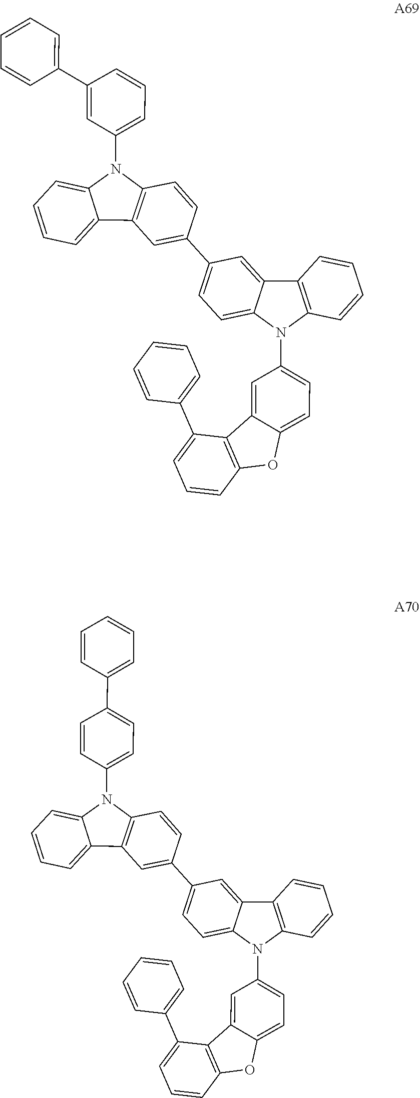 [SMILES: c1ccc(-c2cccc(-n3c4ccccc4c4cc(-c5ccc6c(c5)c5ccccc5n6-c5ccc6oc7cccc(-c8ccccc8)c7c6c5)ccc43)c2)cc1 ; c1ccc(-c2ccc(-n3c4ccccc4c4cc(-c5ccc6c(c5)c5ccccc5n6-c5ccc6oc7cccc(-c8ccccc8)c7c6c5)ccc43)cc2)cc1]

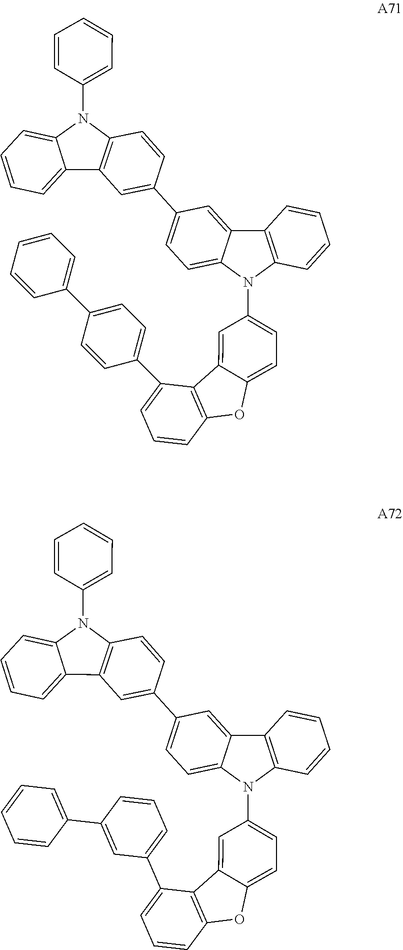 [SMILES: c1ccc(-c2ccc(-c3cccc4oc5ccc(-n6c7ccccc7c7cc(-c8ccc9c(c8)c8ccccc8n9-c8ccccc8)ccc76)cc5c34)cc2)cc1 ; c1ccc(-c2cccc(-c3cccc4oc5ccc(-n6c7ccccc7c7cc(-c8ccc9c(c8)c8ccccc8n9-c8ccccc8)ccc76)cc5c34)c2)cc1]

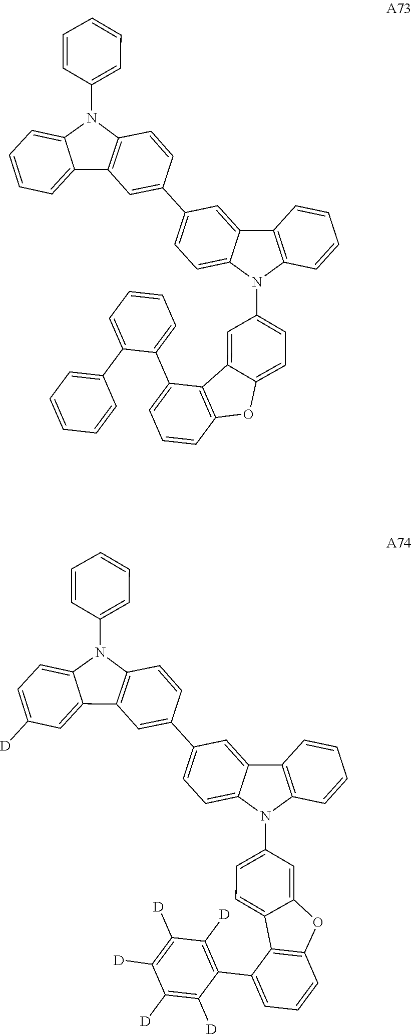 [SMILES: c1ccc(-c2ccccc2-c2cccc3oc4ccc(-n5c6ccccc6c6cc(-c7ccc8c(c7)c7ccccc7n8-c7ccccc7)ccc65)cc4c23)cc1 ; [2H]c1ccc2c(c1)c1cc(-c3ccc4c(c3)c3ccccc3n4-c3ccc4c(c3)oc3cccc(-c5c([2H])c([2H])c([2H])c([2H])c5[2H])c34)ccc1n2-c1ccccc1]

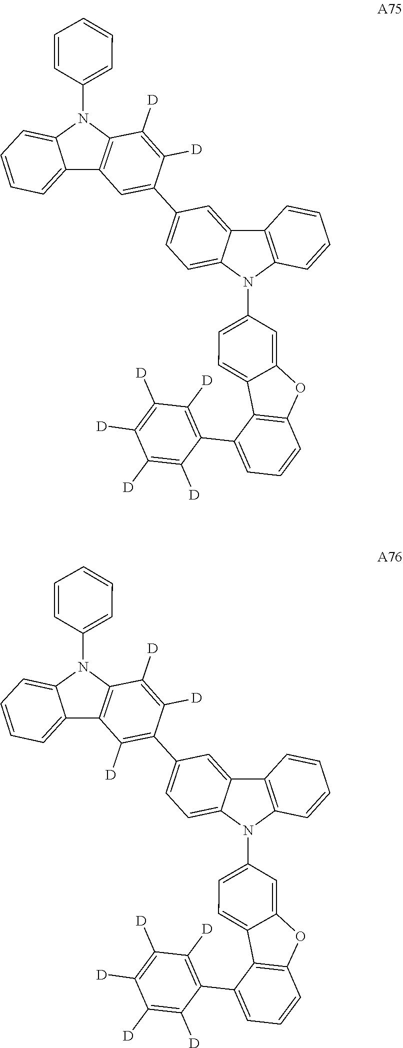 [SMILES: [2H]c1c([2H])c([2H])c(-c2cccc3oc4cc(-n5c6ccccc6c6cc(-c7cc8c9ccccc9n(-c9ccccc9)c8c([2H])c7[2H])ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7ccccc7)ccc65)ccc4c23)c([2H])c1[2H]]

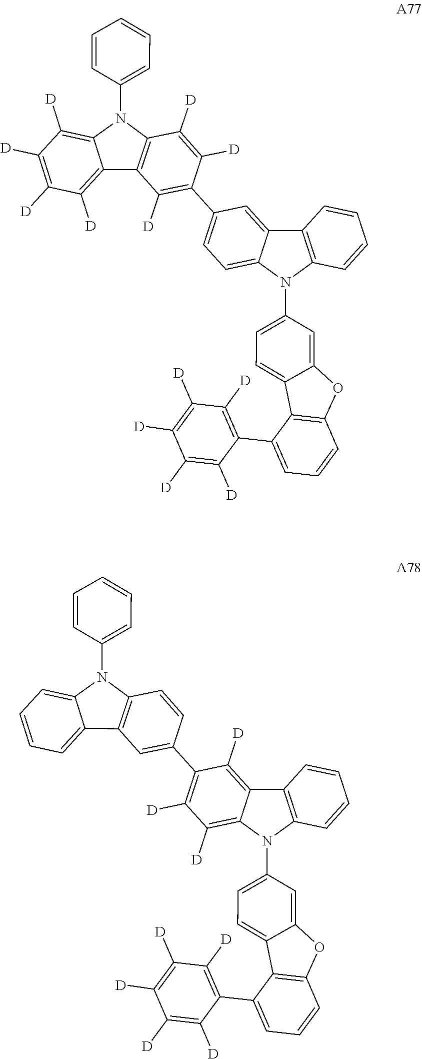 [SMILES: [2H]c1c([2H])c([2H])c(-c2cccc3oc4cc(-n5c6ccccc6c6cc(-c7c([2H])c([2H])c8c(c7[2H])c7c([2H])c([2H])c([2H])c([2H])c7n8-c7ccccc7)ccc65)ccc4c23)c([2H])c1[2H] ; [2H]c1c([2H])c([2H])c(-c2cccc3oc4cc(-n5c6ccccc6c6c([2H])c(-c7ccc8c(c7)c7ccccc7n8-c7ccccc7)c([2H])c([2H])c65)ccc4c23)c([2H])c1[2H]]

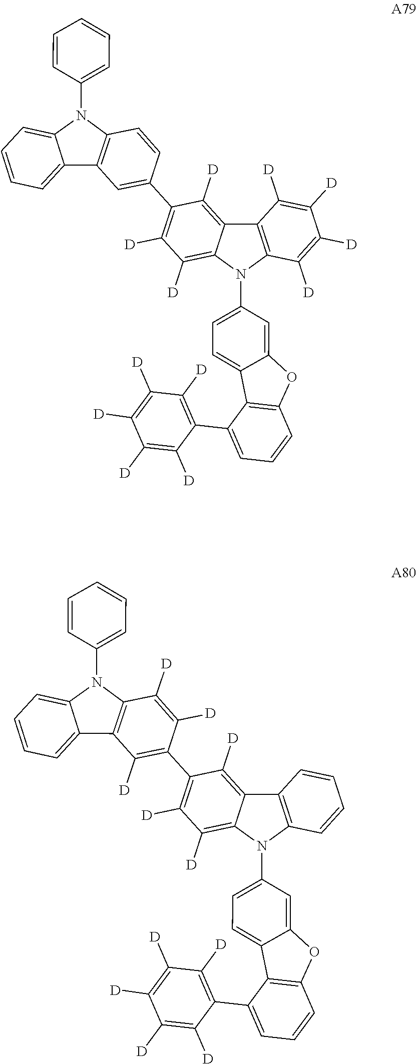 [SMILES: [2H]c1c([2H])c([2H])c(-c2cccc3oc4cc(-n5c6c([2H])c([2H])c([2H])c([2H])c6c6c([2H])c(-c7ccc8c(c7)c7ccccc7n8-c7ccccc7)c([2H])c([2H])c65)ccc4c23)c([2H])c1[2H] ; [2H]c1c([2H])c([2H])c(-c2cccc3oc4cc(-n5c6ccccc6c6c([2H])c(-c7c([2H])c([2H])c8c(c7[2H])c7ccccc7n8-c7ccccc7)c([2H])c([2H])c65)ccc4c23)c([2H])c1[2H]]

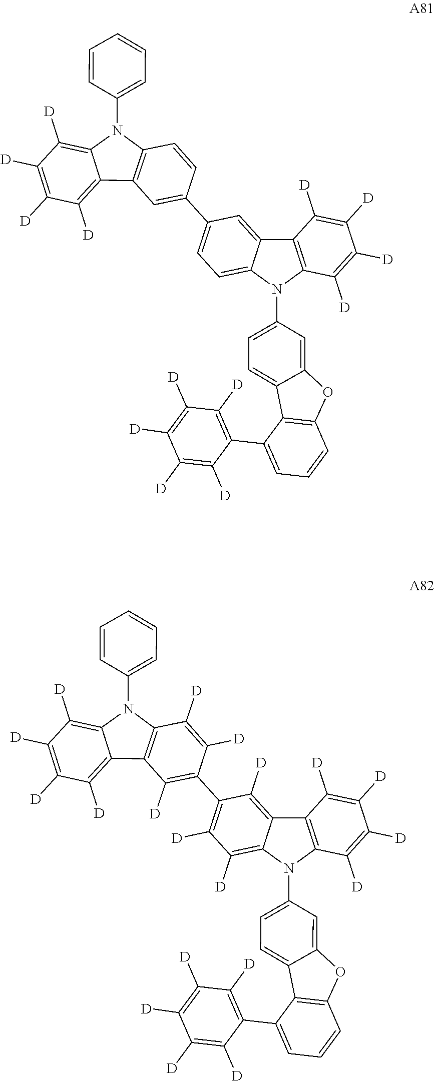 [SMILES: [2H]c1c([2H])c([2H])c(-c2cccc3oc4cc(-n5c6ccc(-c7ccc8c(c7)c7c([2H])c([2H])c([2H])c([2H])c7n8-c7ccccc7)cc6c6c([2H])c([2H])c([2H])c([2H])c65)ccc4c23)c([2H])c1[2H] ; [2H]c1c([2H])c([2H])c(-c2cccc3oc4cc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)ccc4c23)c([2H])c1[2H]]

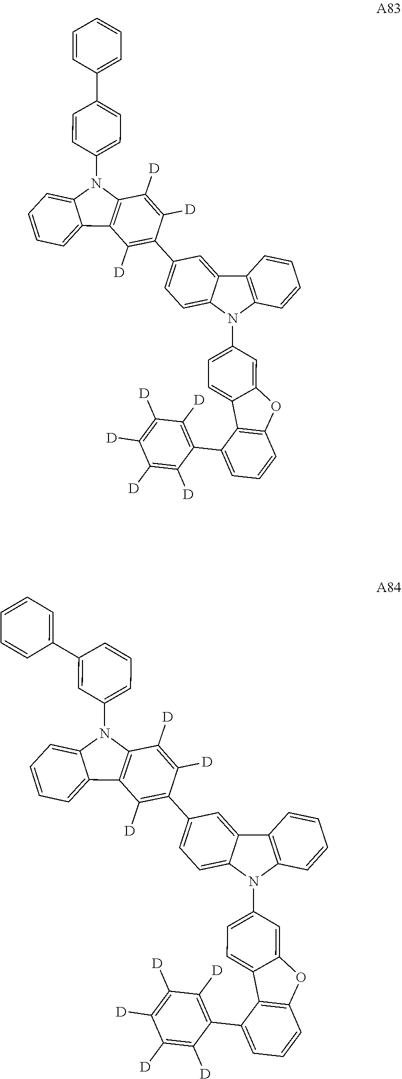 [SMILES: [2H]c1c([2H])c([2H])c(-c2cccc3oc4cc(-n5c6ccccc6c6cc(-c7c([2H])c([2H])c8c(c7[2H])c7ccccc7n8-c7ccc(-c8ccccc8)cc7)ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7cccc(-c8ccccc8)c7)ccc65)ccc4c23)c([2H])c1[2H]]

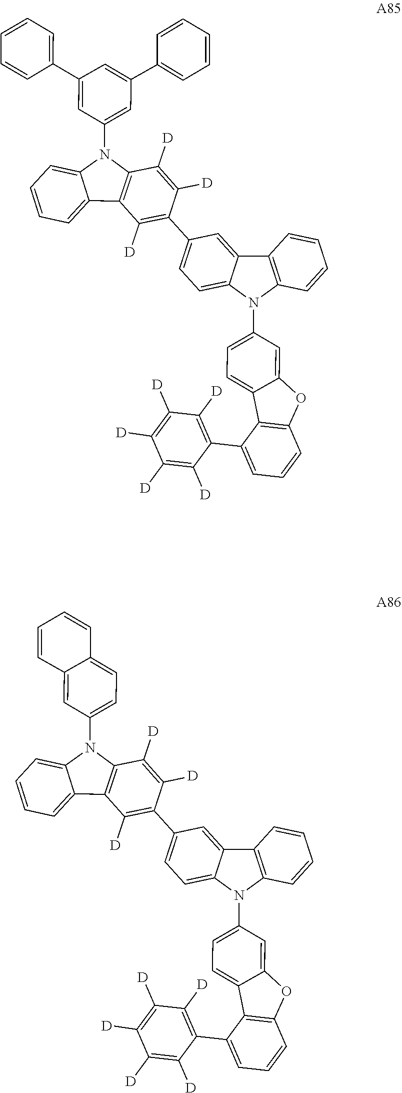 [SMILES: [2H]c1c([2H])c([2H])c(-c2cccc3oc4cc(-n5c6ccccc6c6cc(-c7c([2H])c([2H])c8c(c7[2H])c7ccccc7n8-c7cc(-c8ccccc8)cc(-c8ccccc8)c7)ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7ccc8ccccc8c7)ccc65)ccc4c23)c([2H])c1[2H]]

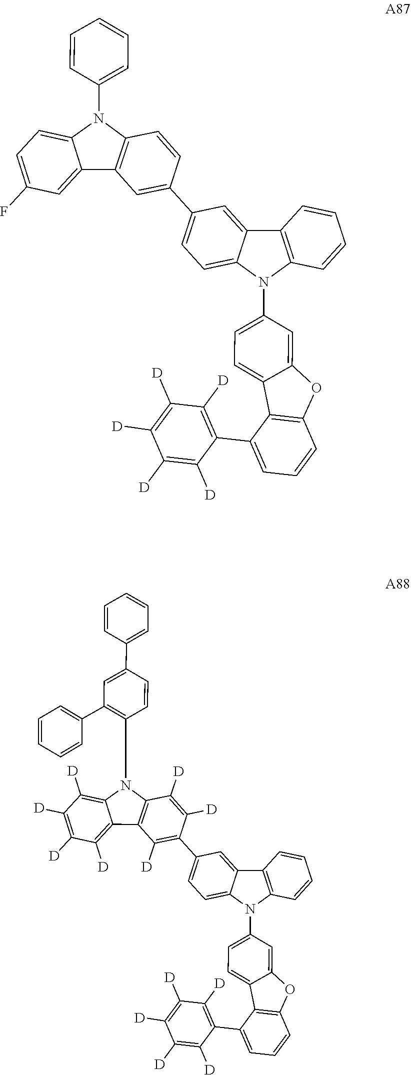 [SMILES: [2H]c1c([2H])c([2H])c(-c2cccc3oc4cc(-n5c6ccccc6c6cc(-c7ccc8c(c7)c7cc(F)ccc7n8-c7ccccc7)ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7c([2H])c([2H])c([2H])c([2H])c7n8-c7ccc(-c8ccccc8)cc7-c7ccccc7)ccc65)ccc4c23)c([2H])c1[2H]]

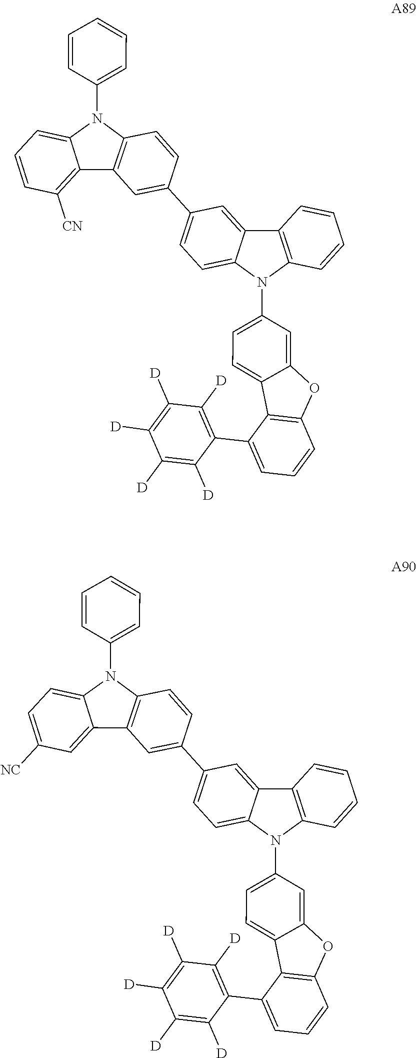 [SMILES: [2H]c1c([2H])c([2H])c(-c2cccc3oc4cc(-n5c6ccccc6c6cc(-c7ccc8c(c7)c7c(C#N)cccc7n8-c7ccccc7)ccc65)ccc4c23)c([2H])c1[2H] ; [2H]c1c([2H])c([2H])c(-c2cccc3oc4cc(-n5c6ccccc6c6cc(-c7ccc8c(c7)c7cc(C#N)ccc7n8-c7ccccc7)ccc65)ccc4c23)c([2H])c1[2H]]

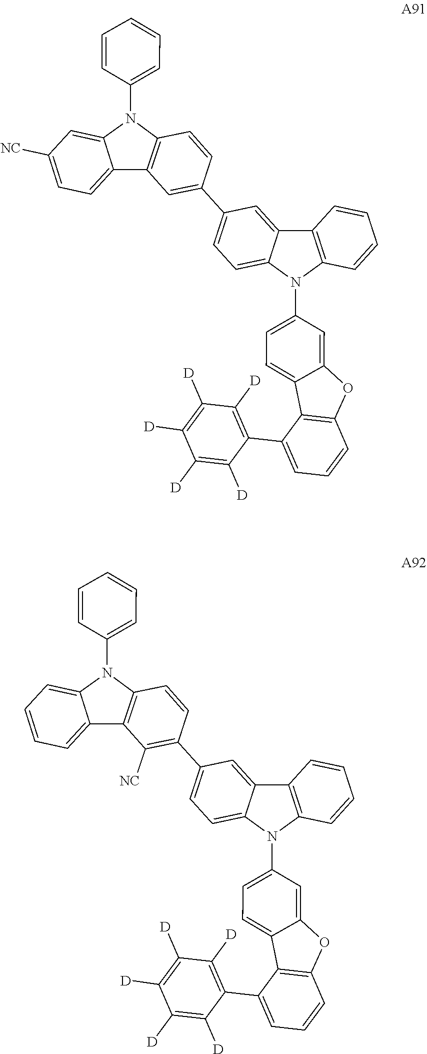 [SMILES: [2H]c1c([2H])c([2H])c(-c2cccc3oc4cc(-n5c6ccccc6c6cc(-c7ccc8c(c7)c7ccc(C#N)cc7n8-c7ccccc7)ccc65)ccc4c23)c([2H])c1[2H] ; [2H]c1c([2H])c([2H])c(-c2cccc3oc4cc(-n5c6ccccc6c6cc(-c7ccc8c(c7C#N)c7ccccc7n8-c7ccccc7)ccc65)ccc4c23)c([2H])c1[2H]]

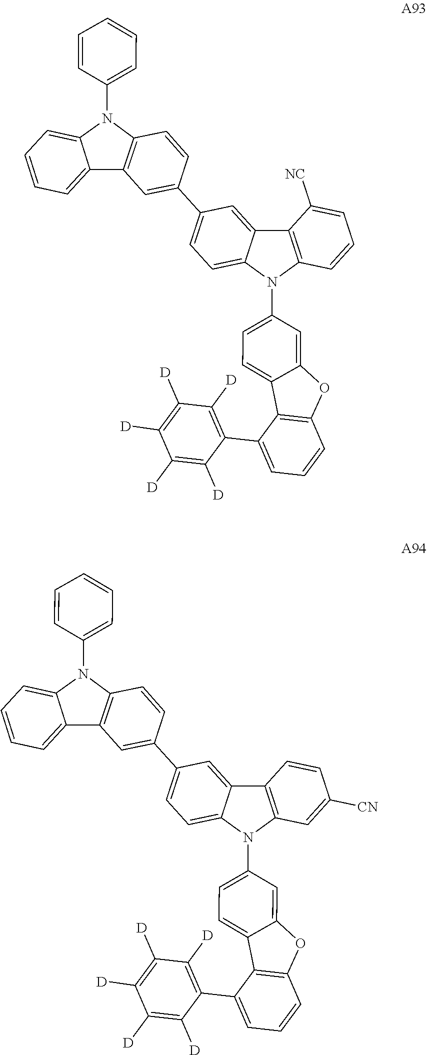 [SMILES: [2H]c1c([2H])c([2H])c(-c2cccc3oc4cc(-n5c6ccc(-c7ccc8c(c7)c7ccccc7n8-c7ccccc7)cc6c6c(C#N)cccc65)ccc4c23)c([2H])c1[2H] ; [2H]c1c([2H])c([2H])c(-c2cccc3oc4cc(-n5c6ccc(-c7ccc8c(c7)c7ccccc7n8-c7ccccc7)cc6c6ccc(C#N)cc65)ccc4c23)c([2H])c1[2H]]

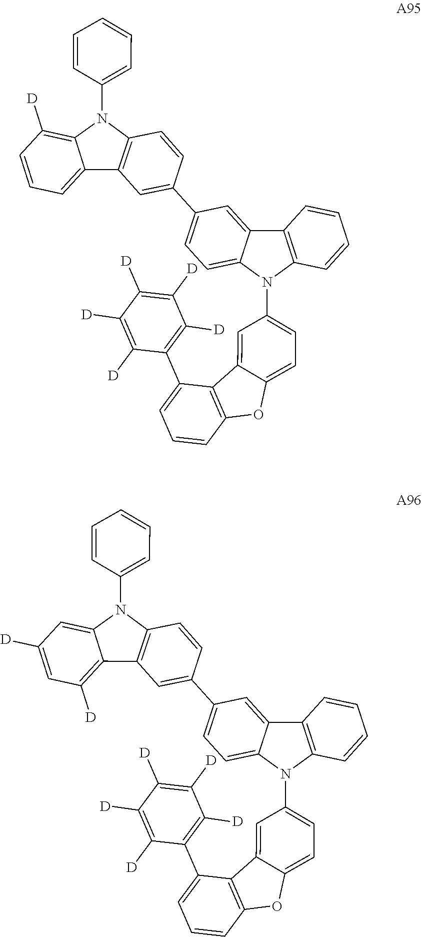 [SMILES: [2H]c1c([2H])c([2H])c(-c2cccc3oc4ccc(-n5c6ccccc6c6cc(-c7ccc8c(c7)c7cccc([2H])c7n8-c7ccccc7)ccc65)cc4c23)c([2H])c1[2H] ; [2H]c1cc([2H])c2c3cc(-c4ccc5c(c4)c4ccccc4n5-c4ccc5oc6cccc(-c7c([2H])c([2H])c([2H])c([2H])c7[2H])c6c5c4)ccc3n(-c3ccccc3)c2c1]

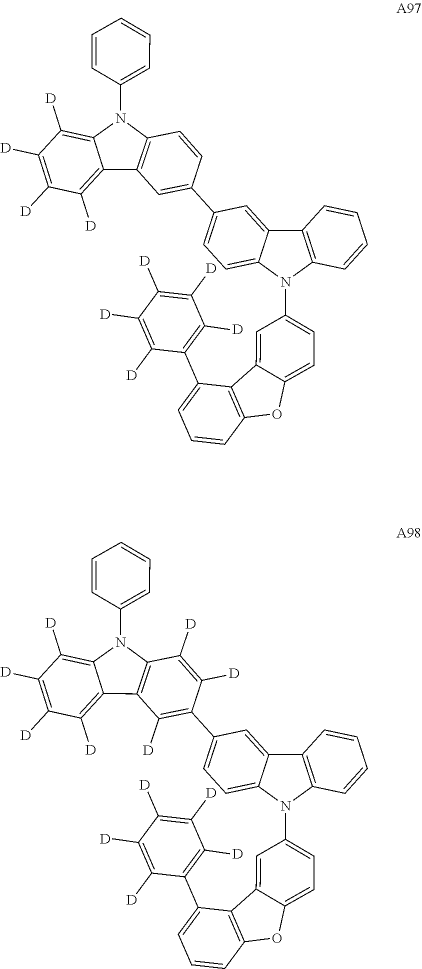 [SMILES: [2H]c1c([2H])c([2H])c(-c2cccc3oc4ccc(-n5c6ccccc6c6cc(-c7ccc8c(c7)c7c([2H])c([2H])c([2H])c([2H])c7n8-c7ccccc7)ccc65)cc4c23)c([2H])c1[2H] ; [2H]c1c([2H])c([2H])c(-c2cccc3oc4ccc(-n5c6ccccc6c6cc(-c7c([2H])c([2H])c8c(c7[2H])c7c([2H])c([2H])c([2H])c([2H])c7n8-c7ccccc7)ccc65)cc4c23)c([2H])c1[2H]]

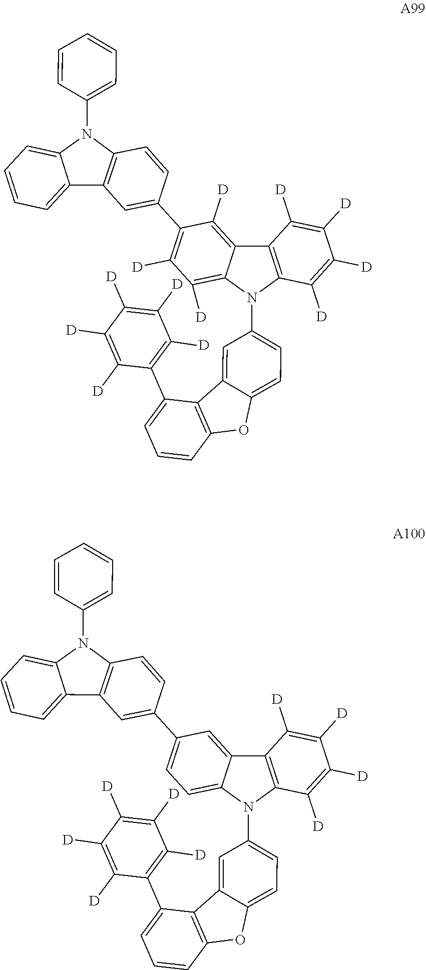 [SMILES: [2H]c1c([2H])c([2H])c(-c2cccc3oc4ccc(-n5c6c([2H])c([2H])c([2H])c([2H])c6c6c([2H])c(-c7ccc8c(c7)c7ccccc7n8-c7ccccc7)c([2H])c([2H])c65)cc4c23)c([2H])c1[2H] ; [2H]c1c([2H])c([2H])c(-c2cccc3oc4ccc(-n5c6ccc(-c7ccc8c(c7)c7ccccc7n8-c7ccccc7)cc6c6c([2H])c([2H])c([2H])c([2H])c65)cc4c23)c([2H])c1[2H]]

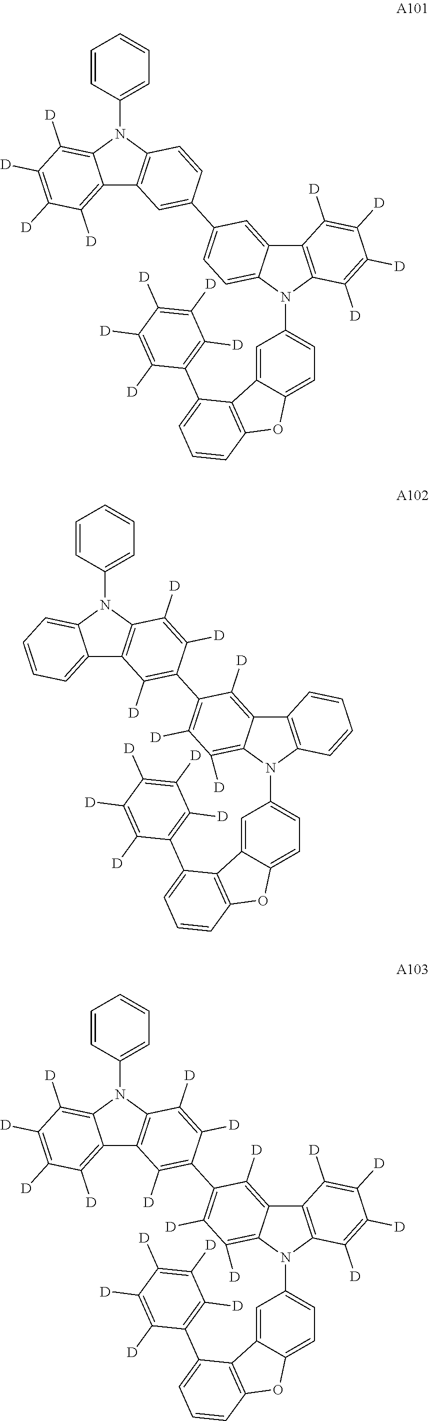

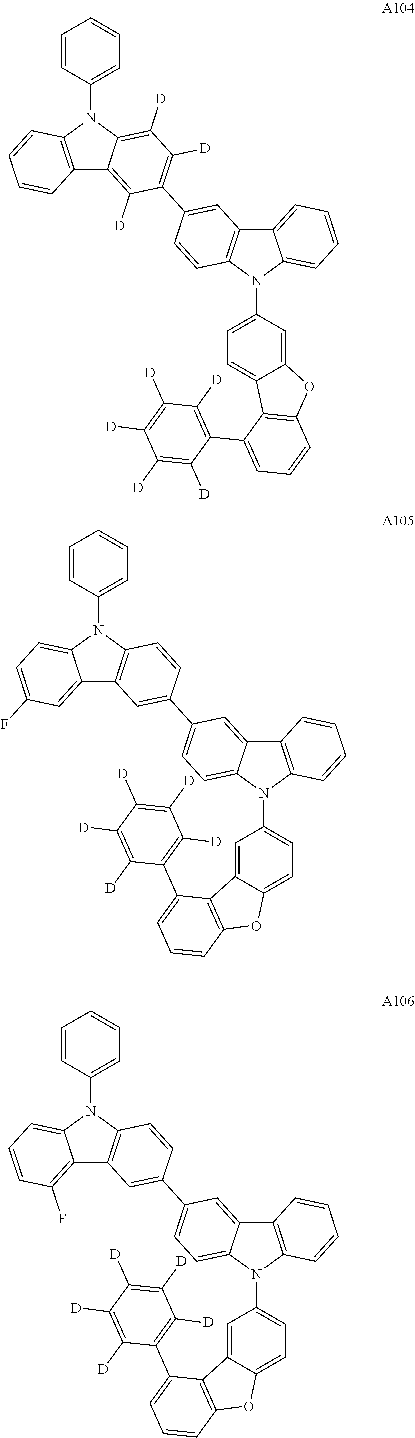 [SMILES: [2H]c1c([2H])c([2H])c(-c2cccc3oc4cc(-n5c6ccccc6c6cc(-c7c([2H])c([2H])c8c(c7[2H])c7ccccc7n8-c7ccccc7)ccc65)ccc4c23)c([2H])c1[2H] ; [2H]c1c([2H])c([2H])c(-c2cccc3oc4ccc(-n5c6ccccc6c6cc(-c7ccc8c(c7)c7cc(F)ccc7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7c(F)cccc7n8-c7ccccc7)ccc65)cc4c23)c([2H])c1[2H]]

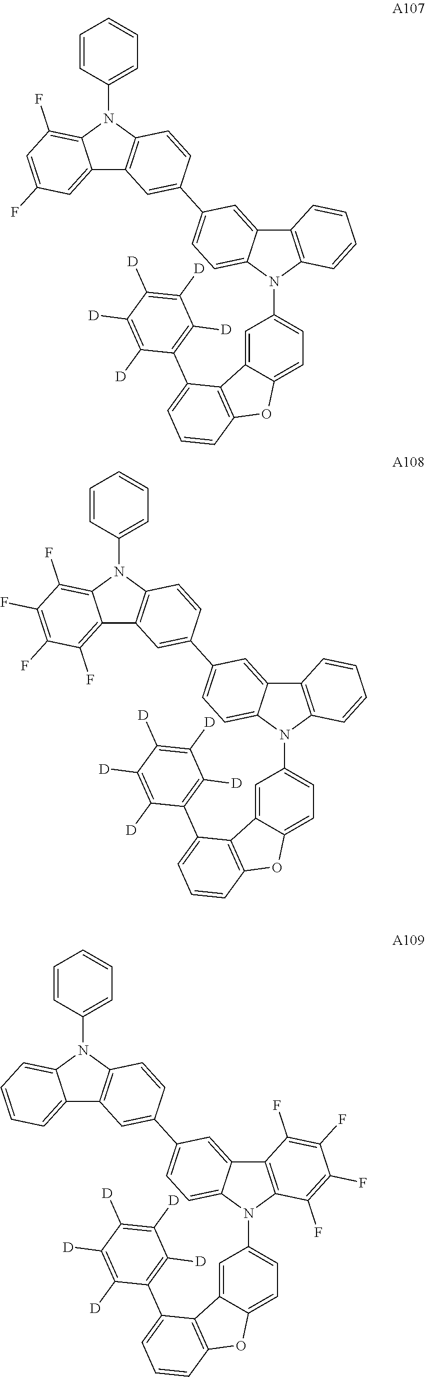 [SMILES: [2H]c1c([2H])c([2H])c(-c2cccc3oc4ccc(-n5c6ccccc6c6cc(-c7ccc8c(c7)c7cc(F)cc(F)c7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7c(F)c(F)c(F)c(F)c7n8-c7ccccc7)ccc65)cc4c23)c([2H])c1[2H] ; [2H]c1c([2H])c([2H])c(-c2cccc3oc4ccc(-n5c6ccc(-c7ccc8c(c7)c7ccccc7n8-c7ccccc7)cc6c6c(F)c(F)c(F)c(F)c65)cc4c23)c([2H])c1[2H]]

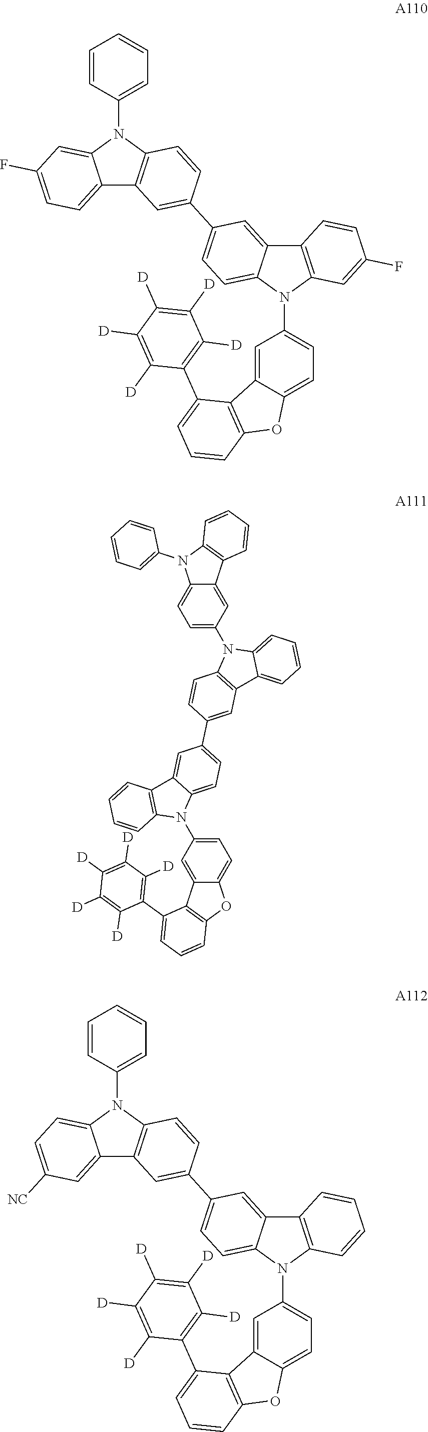 [SMILES: [2H]c1c([2H])c([2H])c(-c2cccc3oc4ccc(-n5c6ccc(-c7ccc8c(c7)c7ccc(F)cc7n8-c7ccccc7)cc6c6ccc(F)cc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)c7ccccc7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7cc(C#N)ccc7n8-c7ccccc7)ccc65)cc4c23)c([2H])c1[2H]]

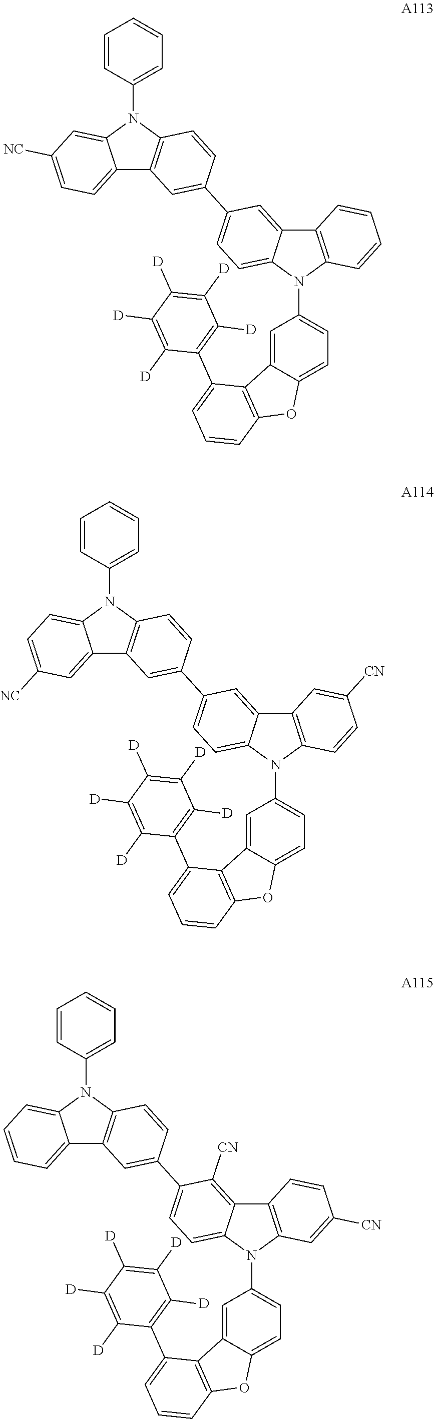 [SMILES: [2H]c1c([2H])c([2H])c(-c2cccc3oc4ccc(-n5c6ccccc6c6cc(-c7ccc8c(c7)c7ccc(C#N)cc7n8-c7ccccc7)ccc65)cc4c23)c([2H])c1[2H] ; [2H]c1c([2H])c([2H])c(-c2cccc3oc4ccc(-n5c6ccc(C#N)cc6c6cc(-c7ccc8c(c7)c7cc(C#N)ccc7n8-c7ccccc7)ccc65)cc4c23)c([2H])c1[2H] ; [2H]c1c([2H])c([2H])c(-c2cccc3oc4ccc(-n5c6cc(C#N)ccc6c6c(C#N)c(-c7ccc8c(c7)c7ccccc7n8-c7ccccc7)ccc65)cc4c23)c([2H])c1[2H]]

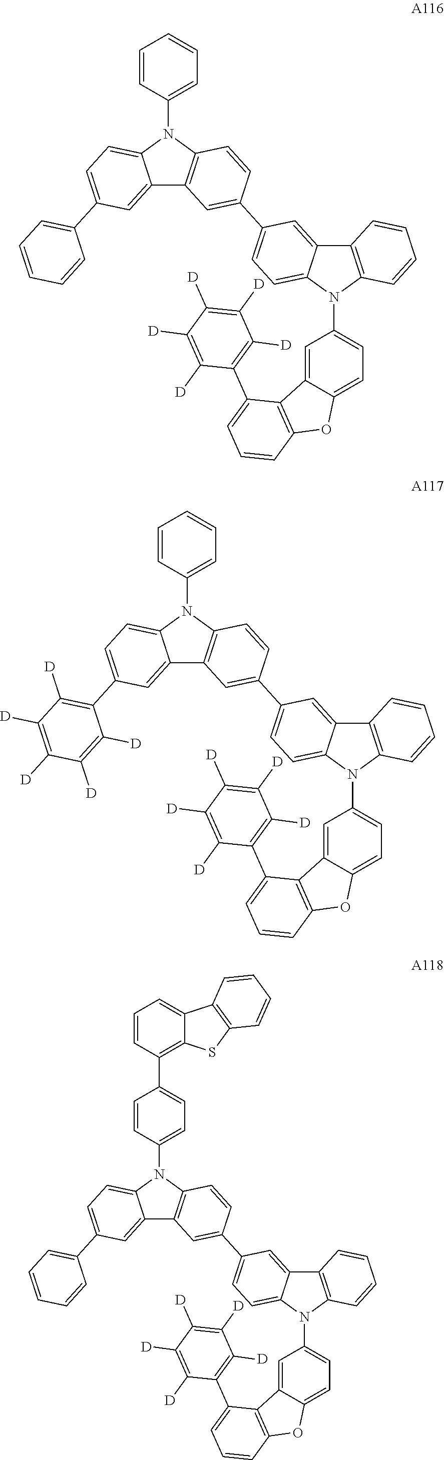

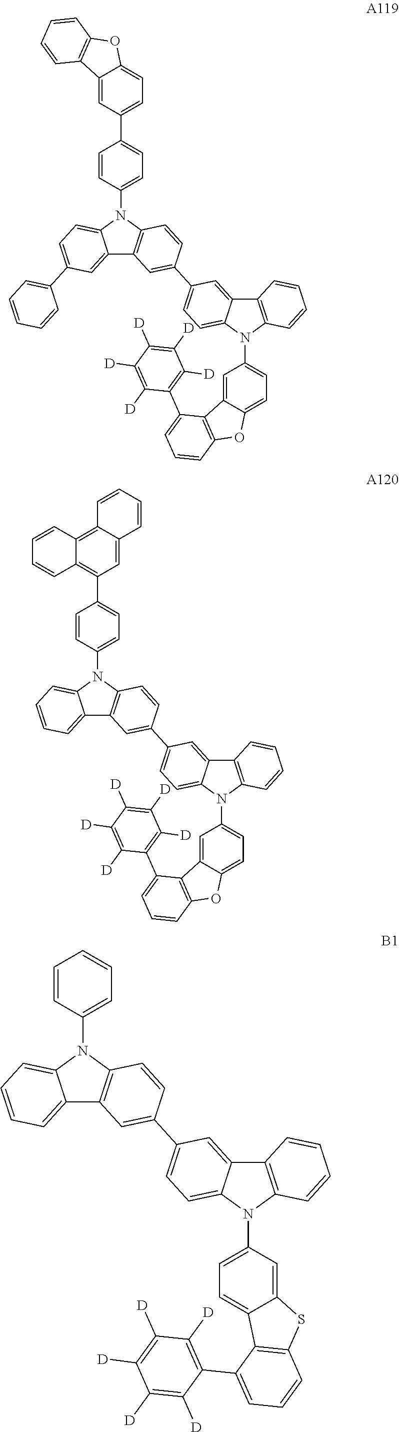 [SMILES: [2H]c1c([2H])c([2H])c(-c2cccc3oc4ccc(-n5c6ccccc6c6cc(-c7ccc8c(c7)c7cc(-c9ccccc9)ccc7n8-c7ccc(-c8ccc9oc%10ccccc%10c9c8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(-c8cc9ccccc9c9ccccc89)cc7)ccc65)cc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccccc7)ccc65)ccc4c23)c([2H])c1[2H]]

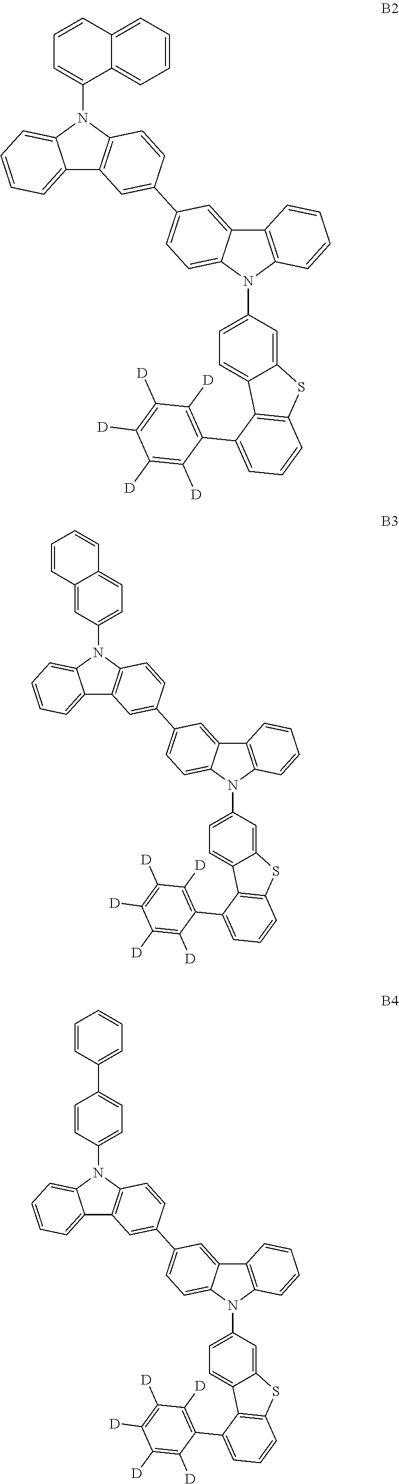 [SMILES: [2H]c1c([2H])c([2H])c(-c2cccc3sc4cc(-n5c6ccccc6c6cc(-c7ccc8c(c7)c7ccccc7n8-c7cccc8ccccc78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc8ccccc8c7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc(-c8ccccc8)cc7)ccc65)ccc4c23)c([2H])c1[2H]]

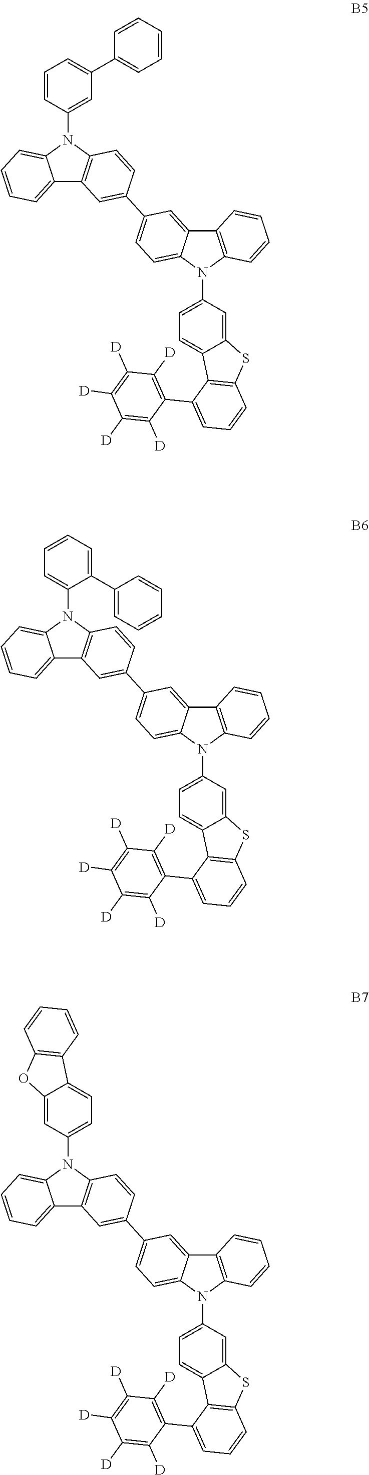 [SMILES: [2H]c1c([2H])c([2H])c(-c2cccc3sc4cc(-n5c6ccccc6c6cc(-c7ccc8c(c7)c7ccccc7n8-c7cccc(-c8ccccc8)c7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccccc7-c7ccccc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc8c(c7)oc7ccccc78)ccc65)ccc4c23)c([2H])c1[2H]]

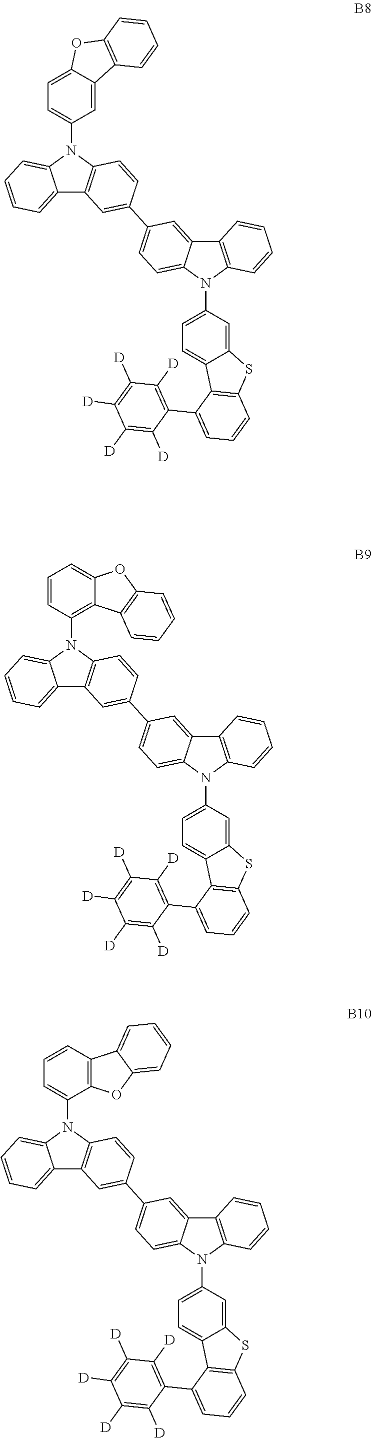 [SMILES: [2H]c1c([2H])c([2H])c(-c2cccc3sc4cc(-n5c6ccccc6c6cc(-c7ccc8c(c7)c7ccccc7n8-c7ccc8oc9ccccc9c8c7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc8oc9ccccc9c78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc8c7oc7ccccc78)ccc65)ccc4c23)c([2H])c1[2H]]

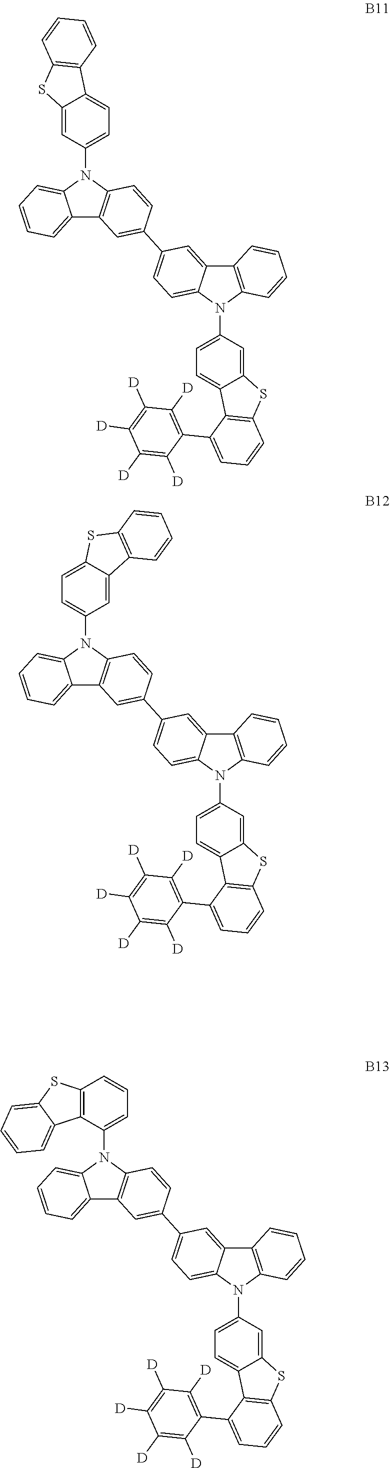

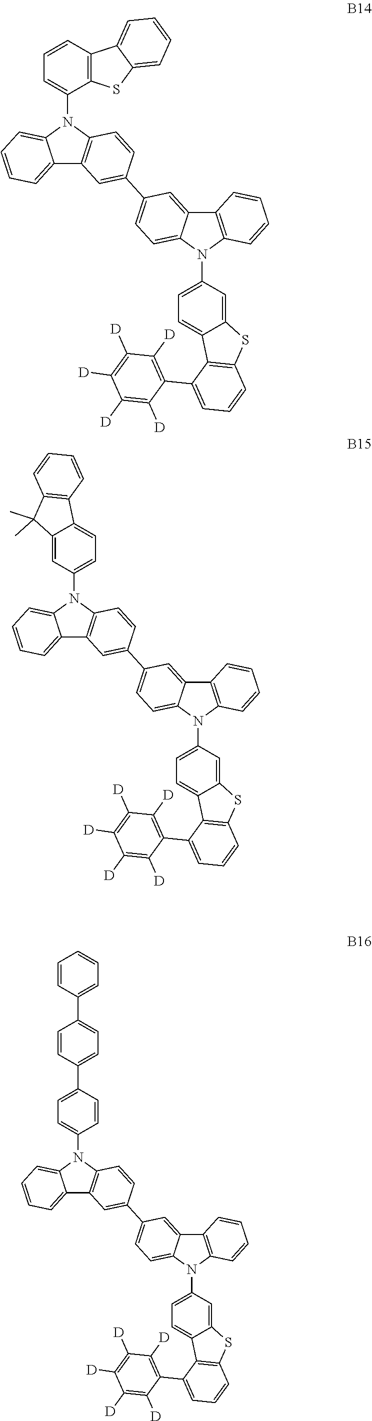 [SMILES: [2H]c1c([2H])c([2H])c(-c2cccc3sc4cc(-n5c6ccccc6c6cc(-c7ccc8c(c7)c7ccccc7n8-c7cccc8c7sc7ccccc78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc8c(c7)C(C)(C)c7ccccc7-8)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc(-c8ccc(-c9ccccc9)cc8)cc7)ccc65)ccc4c23)c([2H])c1[2H]]

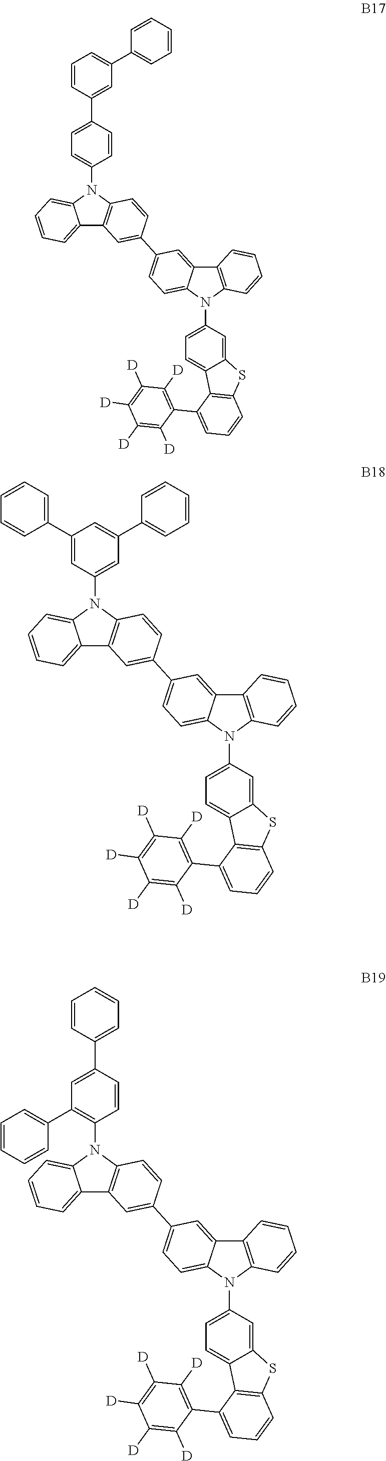

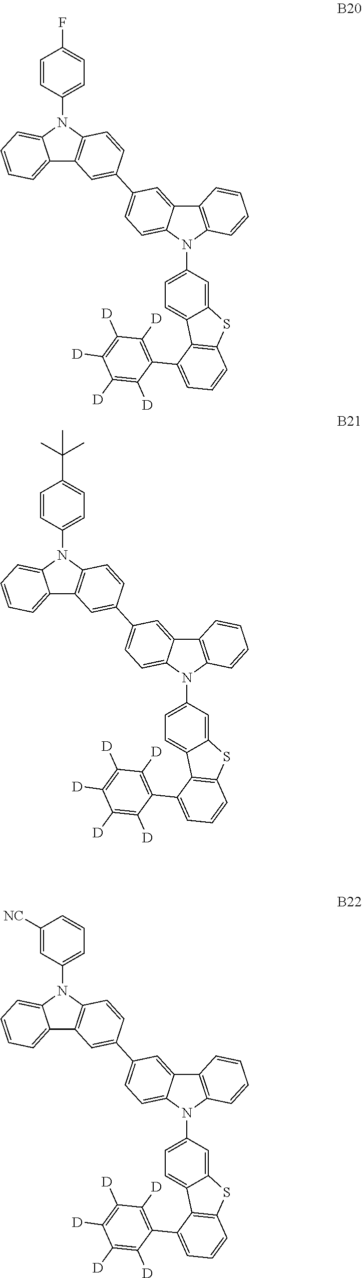 [SMILES: [2H]c1c([2H])c([2H])c(-c2cccc3sc4cc(-n5c6ccccc6c6cc(-c7ccc8c(c7)c7ccccc7n8-c7ccc(F)cc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc(C(C)(C)C)cc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc(C#N)c7)ccc65)ccc4c23)c([2H])c1[2H]]

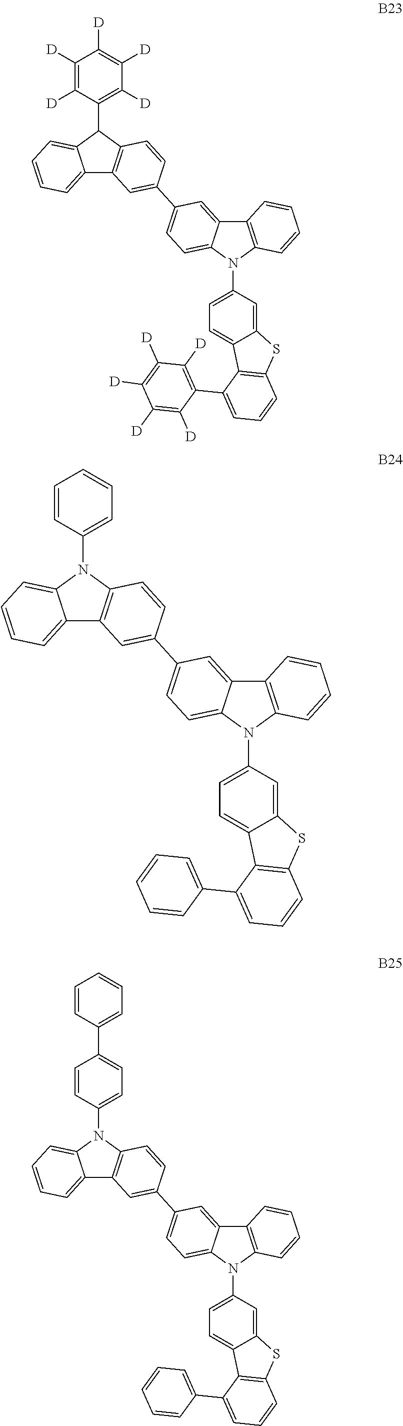 [SMILES: [2H]c1c([2H])c([2H])c(-c2cccc3sc4cc(-n5c6ccccc6c6cc(-c7ccc8c(c7)-c7ccccc7C8c7c([2H])c([2H])c([2H])c([2H])c7[2H])ccc65)ccc4c23)c([2H])c1[2H] ; c1ccc(-c2cccc3sc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc6c(c5)sc5cccc(-c7ccccc7)c56)ccc43)cc2)cc1]

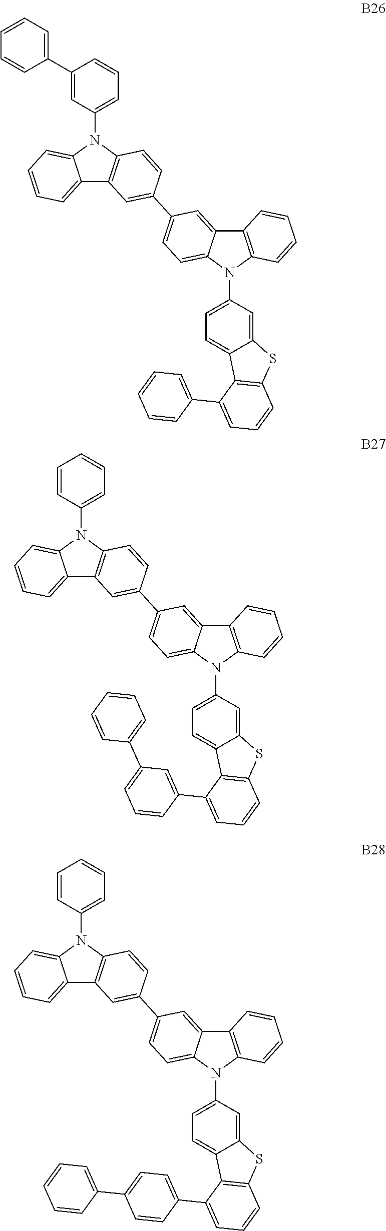 [SMILES: c1ccc(-c2cccc(-n3c4ccccc4c4cc(-c5ccc6c(c5)c5ccccc5n6-c5ccc6c(c5)sc5cccc(-c7ccccc7)c56)ccc43)c2)cc1 ; c1ccc(-c2cccc(-c3cccc4sc5cc(-n6c7ccccc7c7cc(-c8ccc9c(c8)c8ccccc8n9-c8ccccc8)ccc76)ccc5c34)c2)cc1 ; c1ccc(-c2ccc(-c3cccc4sc5cc(-n6c7ccccc7c7cc(-c8ccc9c(c8)c8ccccc8n9-c8ccccc8)ccc76)ccc5c34)cc2)cc1]

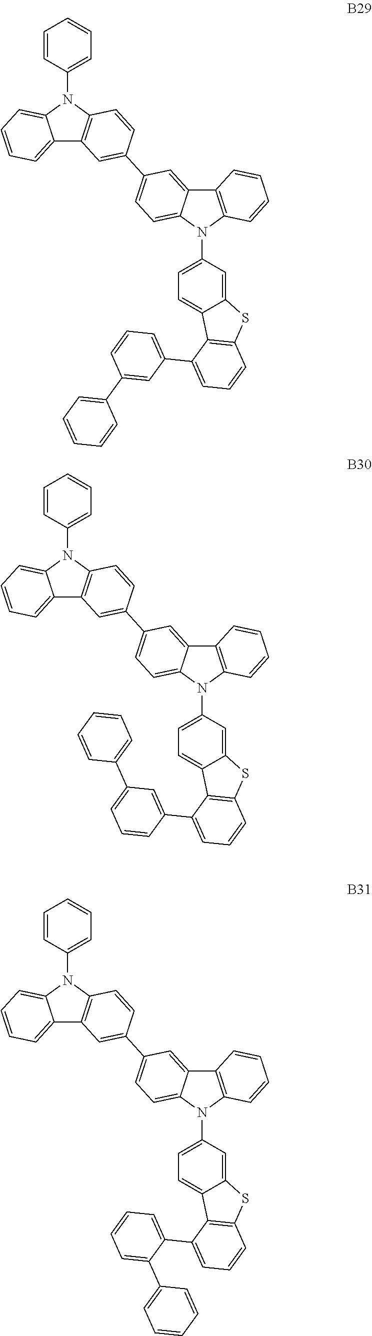

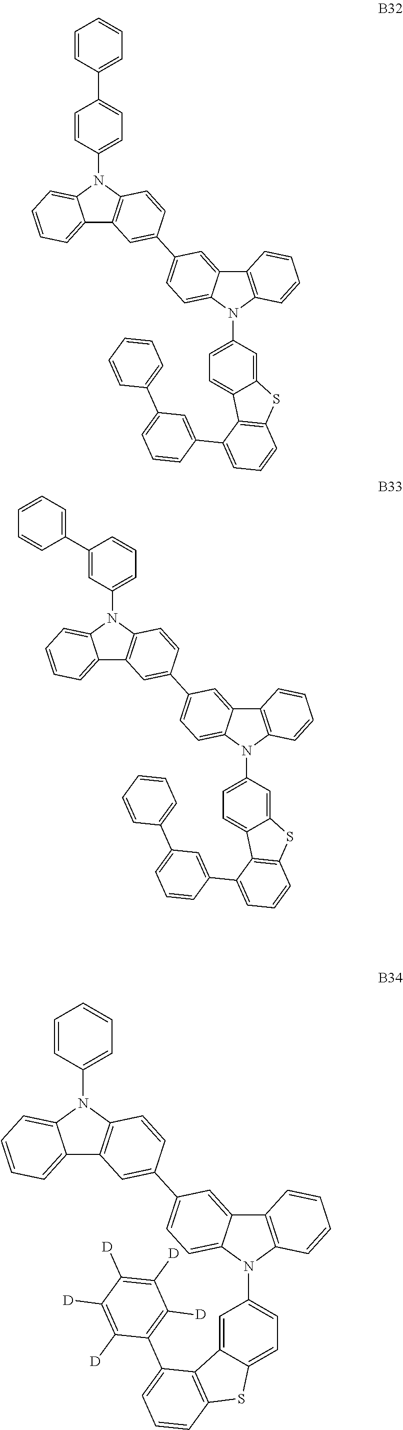 [SMILES: c1ccc(-c2ccc(-n3c4ccccc4c4cc(-c5ccc6c(c5)c5ccccc5n6-c5ccc6c(c5)sc5cccc(-c7cccc(-c8ccccc8)c7)c56)ccc43)cc2)cc1 ; c1ccc(-c2cccc(-c3cccc4sc5cc(-n6c7ccccc7c7cc(-c8ccc9c(c8)c8ccccc8n9-c8cccc(-c9ccccc9)c8)ccc76)ccc5c34)c2)cc1 ; [2H]c1c([2H])c([2H])c(-c2cccc3sc4ccc(-n5c6ccccc6c6cc(-c7ccc8c(c7)c7ccccc7n8-c7ccccc7)ccc65)cc4c23)c([2H])c1[2H]]

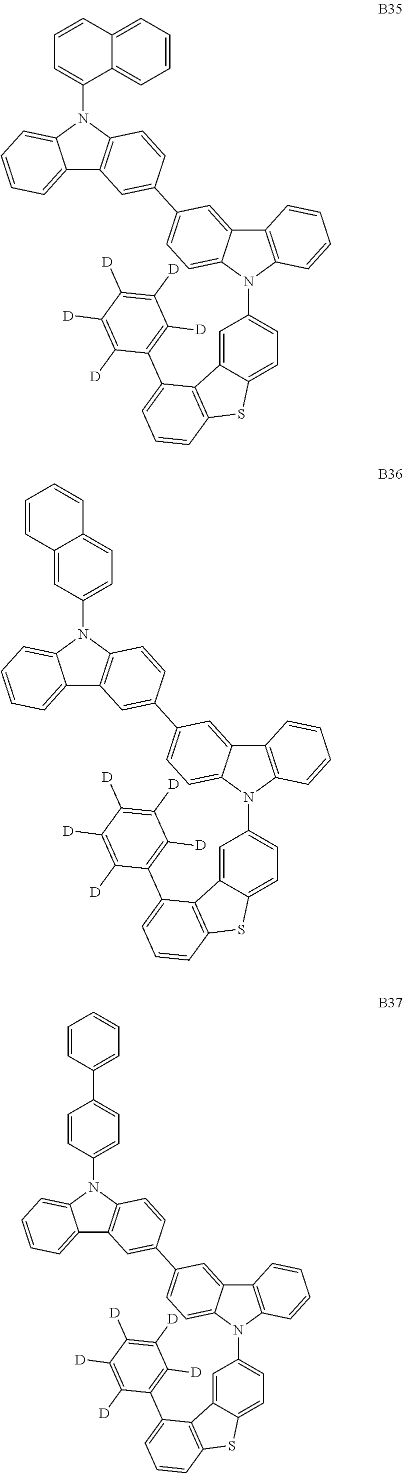 [SMILES: [2H]c1c([2H])c([2H])c(-c2cccc3sc4ccc(-n5c6ccccc6c6cc(-c7ccc8c(c7)c7ccccc7n8-c7cccc8ccccc78)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8ccccc8c7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc(-c8ccccc8)cc7)ccc65)cc4c23)c([2H])c1[2H]]

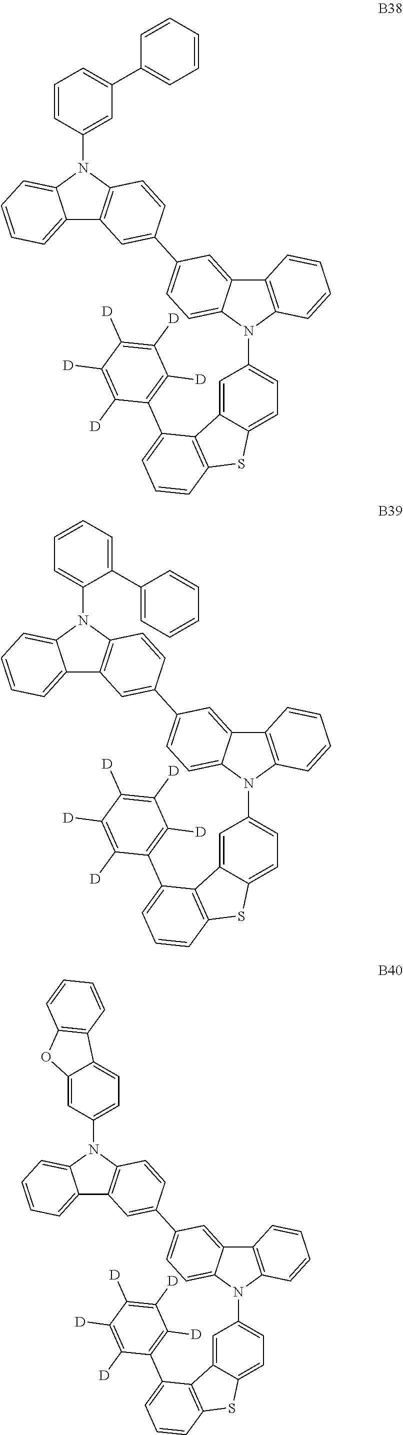 [SMILES: [2H]c1c([2H])c([2H])c(-c2cccc3sc4ccc(-n5c6ccccc6c6cc(-c7ccc8c(c7)c7ccccc7n8-c7cccc(-c8ccccc8)c7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccccc7-c7ccccc7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8c(c7)oc7ccccc78)ccc65)cc4c23)c([2H])c1[2H]]

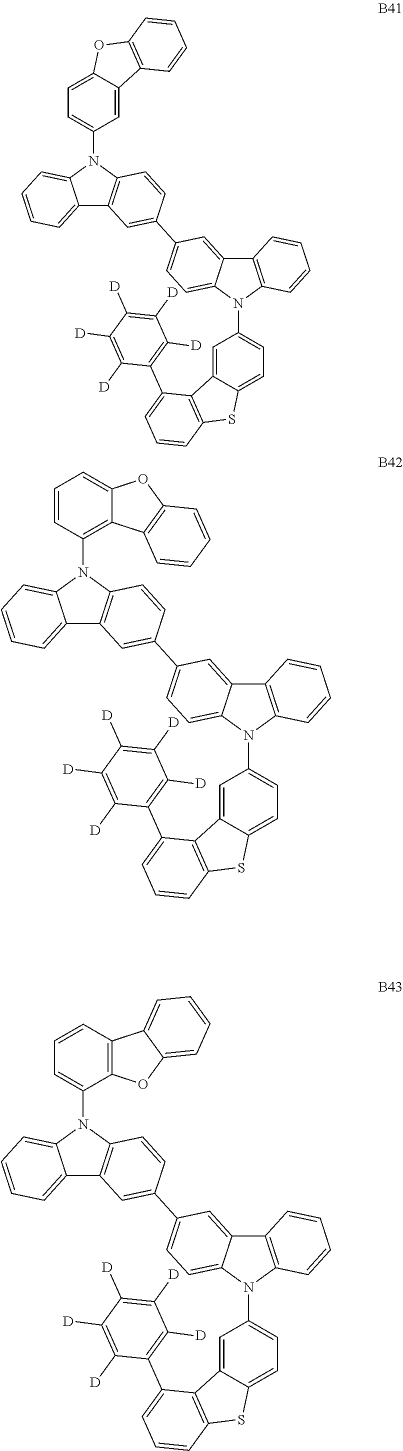

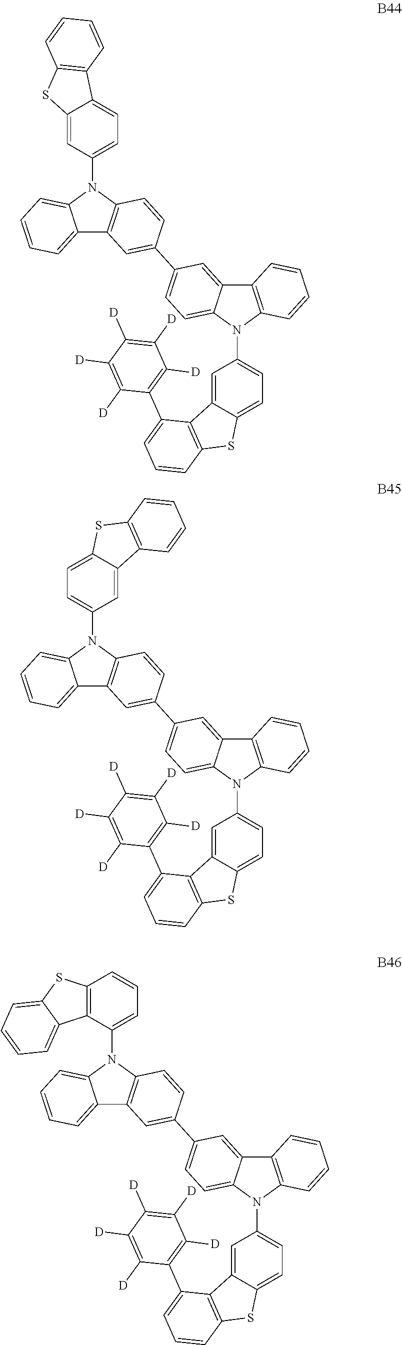

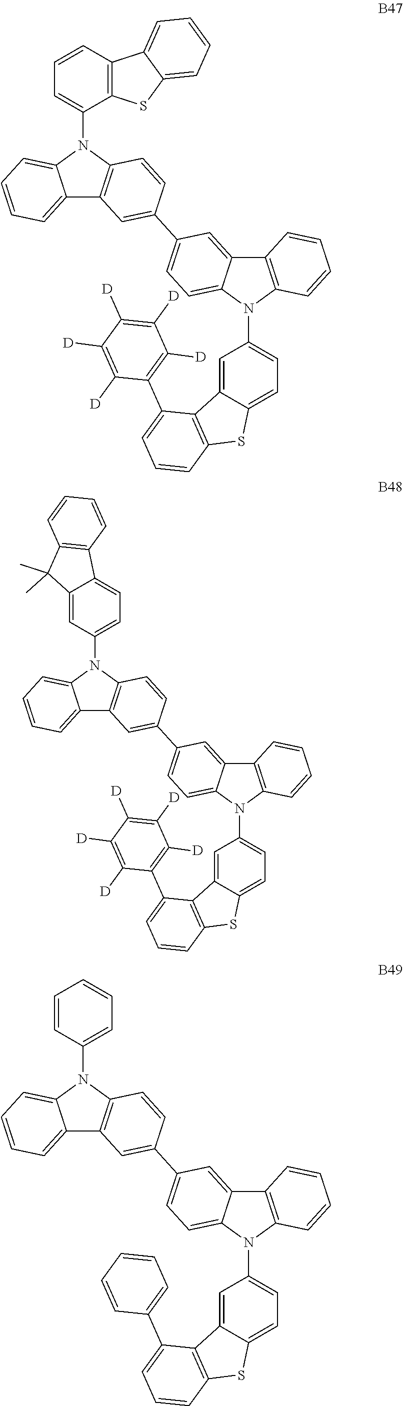 [SMILES: [2H]c1c([2H])c([2H])c(-c2cccc3sc4ccc(-n5c6ccccc6c6cc(-c7ccc8c(c7)c7ccccc7n8-c7cccc8c7sc7ccccc78)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8c(c7)C(C)(C)c7ccccc7-8)ccc65)cc4c23)c([2H])c1[2H] ; c1ccc(-c2cccc3sc4ccc(-n5c6ccccc6c6cc(-c7ccc8c(c7)c7ccccc7n8-c7ccccc7)ccc65)cc4c23)cc1]

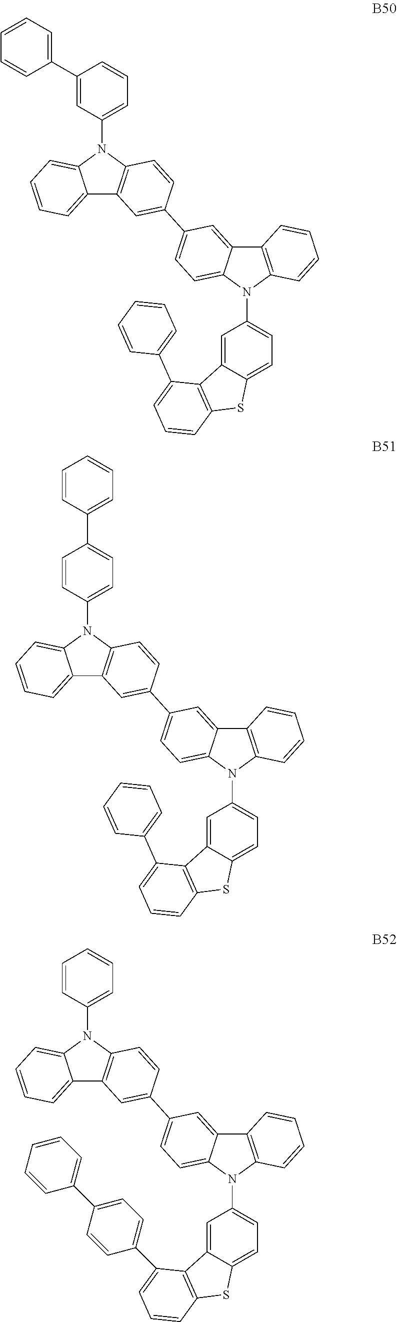 [SMILES: c1ccc(-c2cccc(-n3c4ccccc4c4cc(-c5ccc6c(c5)c5ccccc5n6-c5ccc6sc7cccc(-c8ccccc8)c7c6c5)ccc43)c2)cc1 ; c1ccc(-c2ccc(-n3c4ccccc4c4cc(-c5ccc6c(c5)c5ccccc5n6-c5ccc6sc7cccc(-c8ccccc8)c7c6c5)ccc43)cc2)cc1 ; c1ccc(-c2ccc(-c3cccc4sc5ccc(-n6c7ccccc7c7cc(-c8ccc9c(c8)c8ccccc8n9-c8ccccc8)ccc76)cc5c34)cc2)cc1]

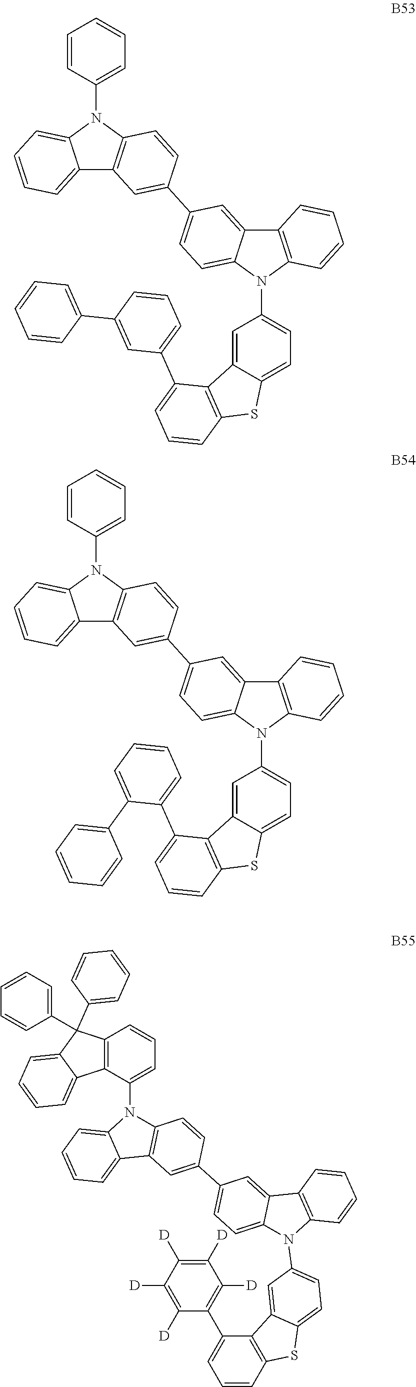 [SMILES: c1ccc(-c2cccc(-c3cccc4sc5ccc(-n6c7ccccc7c7cc(-c8ccc9c(c8)c8ccccc8n9-c8ccccc8)ccc76)cc5c34)c2)cc1 ; c1ccc(-c2ccccc2-c2cccc3sc4ccc(-n5c6ccccc6c6cc(-c7ccc8c(c7)c7ccccc7n8-c7ccccc7)ccc65)cc4c23)cc1 ; [2H]c1c([2H])c([2H])c(-c2cccc3sc4ccc(-n5c6ccccc6c6cc(-c7ccc8c(c7)c7ccccc7n8-c7cccc8c7-c7ccccc7C8(c7ccccc7)c7ccccc7)ccc65)cc4c23)c([2H])c1[2H]]

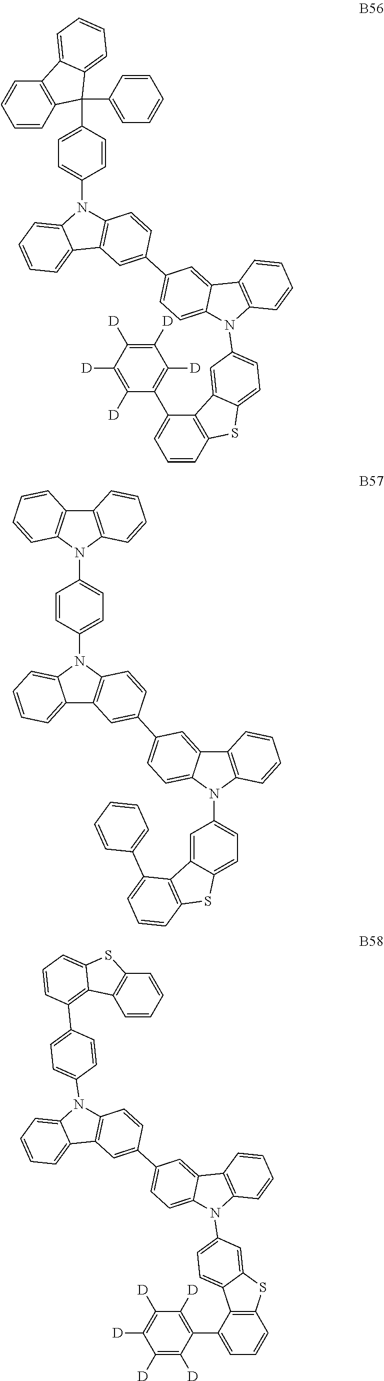 [SMILES: [2H]c1c([2H])c([2H])c(-c2cccc3sc4ccc(-n5c6ccccc6c6cc(-c7ccc8c(c7)c7ccccc7n8-c7ccc(C8(c9ccccc9)c9ccccc9-c9ccccc98)cc7)ccc65)cc4c23)c([2H])c1[2H] ; c1ccc(-c2cccc3sc4ccc(-n5c6ccccc6c6cc(-c7ccc8c(c7)c7ccccc7n8-c7ccc(-n8c9ccccc9c9ccccc98)cc7)ccc65)cc4c23)cc1 ; [2H]c1c([2H])c([2H])c(-c2cccc3sc4cc(-n5c6ccccc6c6cc(-c7ccc8c(c7)c7ccccc7n8-c7ccc(-c8cccc9sc%10ccccc%10c89)cc7)ccc65)ccc4c23)c([2H])c1[2H]]

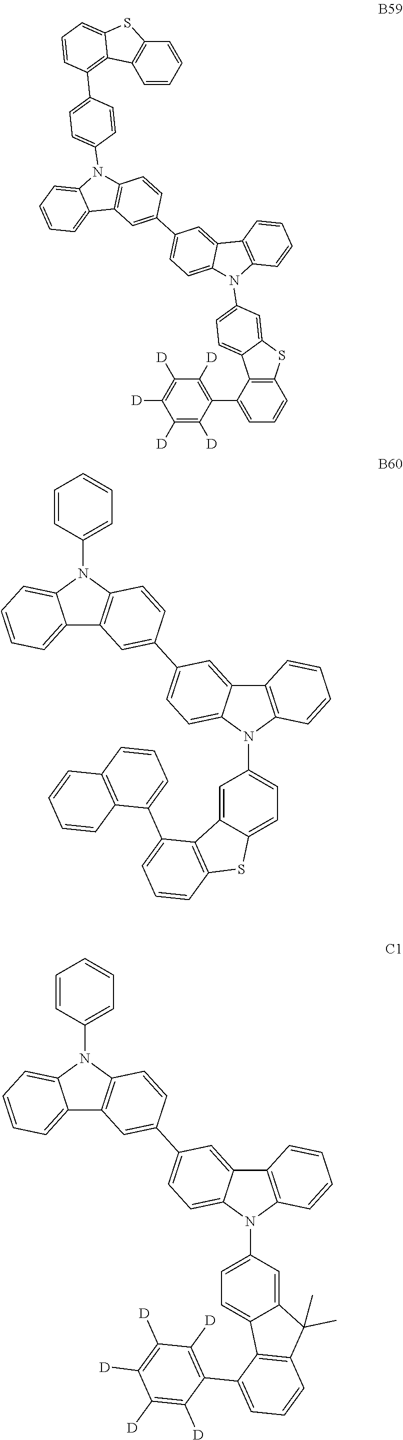 [SMILES: [2H]c1c([2H])c([2H])c(-c2cccc3sc4cc(-n5c6ccccc6c6cc(-c7ccc8c(c7)c7ccccc7n8-c7ccc(-c8cccc9sc%10ccccc%10c89)cc7)ccc65)ccc4c23)c([2H])c1[2H] ; c1ccc(-n2c3ccccc3c3cc(-c4ccc5c(c4)c4ccccc4n5-c4ccc5sc6cccc(-c7cccc8ccccc78)c6c5c4)ccc32)cc1 ; [2H]c1c([2H])c([2H])c(-c2cccc3c2-c2ccc(-n4c5ccccc5c5cc(-c6ccc7c(c6)c6ccccc6n7-c6ccccc6)ccc54)cc2C3(C)C)c([2H])c1[2H]]

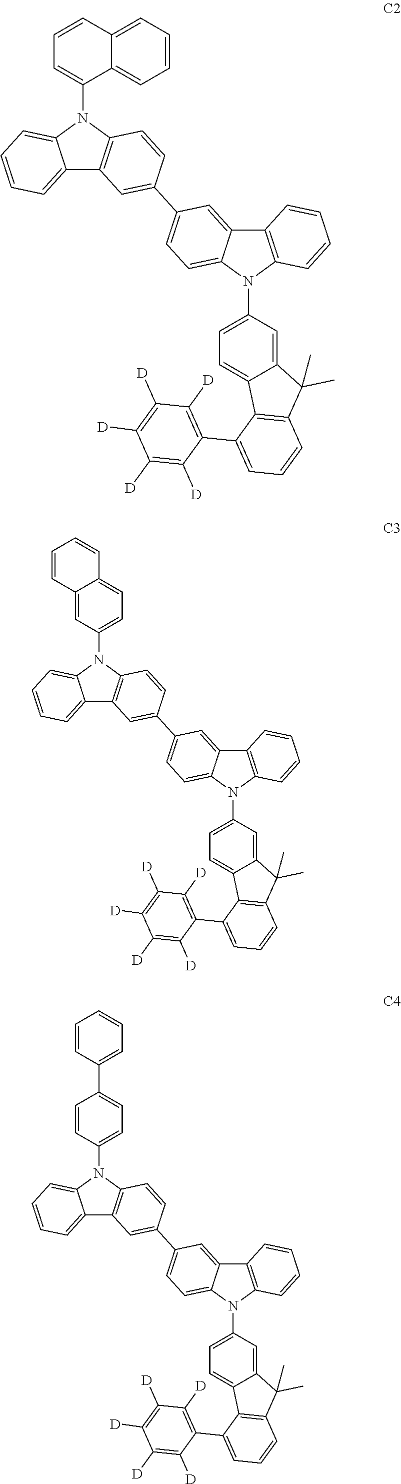 [SMILES: [2H]c1c([2H])c([2H])c(-c2cccc3c2-c2ccc(-n4c5ccccc5c5cc(-c6ccc7c(c6)c6ccccc6n7-c6cccc7ccccc67)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc7ccccc7c6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc(-c7ccccc7)cc6)ccc54)cc2C3(C)C)c([2H])c1[2H]]

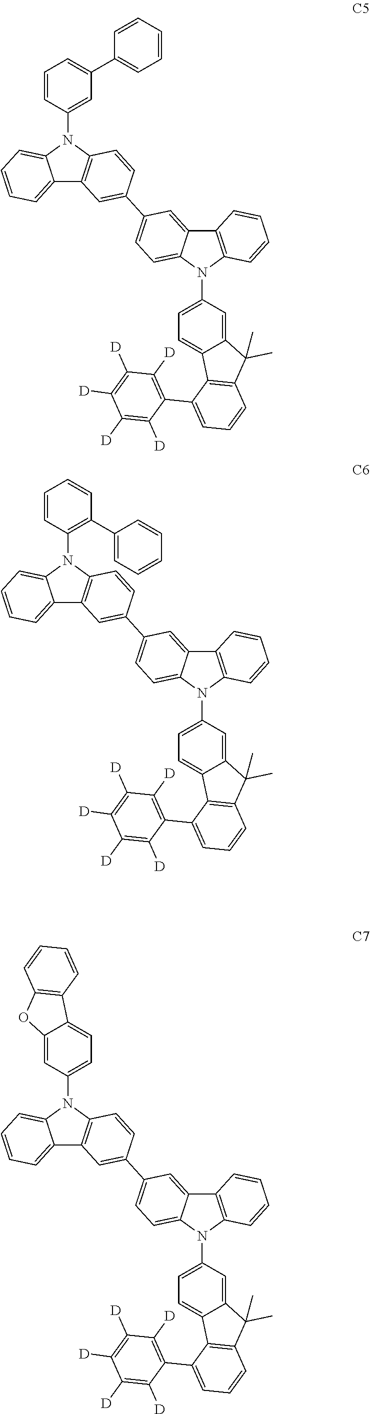 [SMILES: [2H]c1c([2H])c([2H])c(-c2cccc3c2-c2ccc(-n4c5ccccc5c5cc(-c6ccc7c(c6)c6ccccc6n7-c6cccc(-c7ccccc7)c6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccccc6-c6ccccc6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc7c(c6)oc6ccccc67)ccc54)cc2C3(C)C)c([2H])c1[2H]]

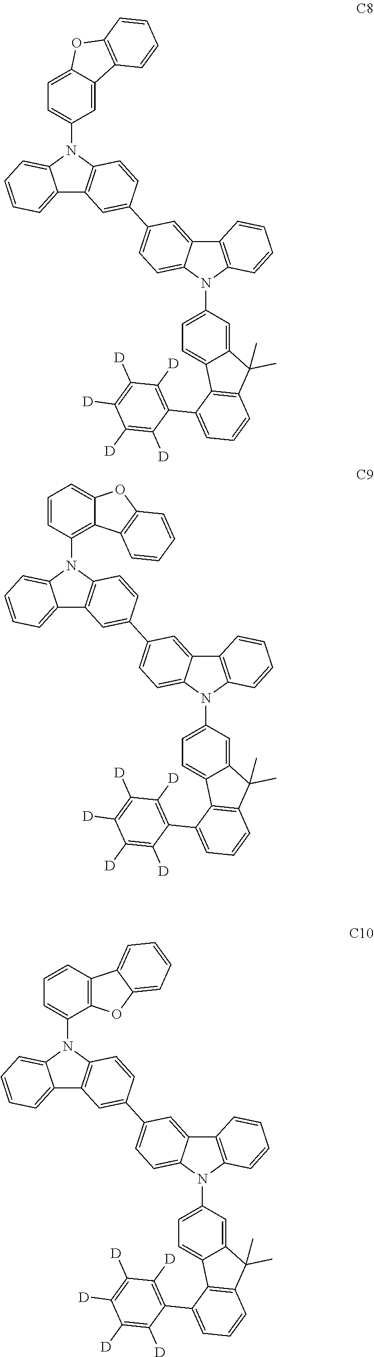 [SMILES: [2H]c1c([2H])c([2H])c(-c2cccc3c2-c2ccc(-n4c5ccccc5c5cc(-c6ccc7c(c6)c6ccccc6n7-c6ccc7oc8ccccc8c7c6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6cccc7oc8ccccc8c67)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6cccc7c6oc6ccccc67)ccc54)cc2C3(C)C)c([2H])c1[2H]]

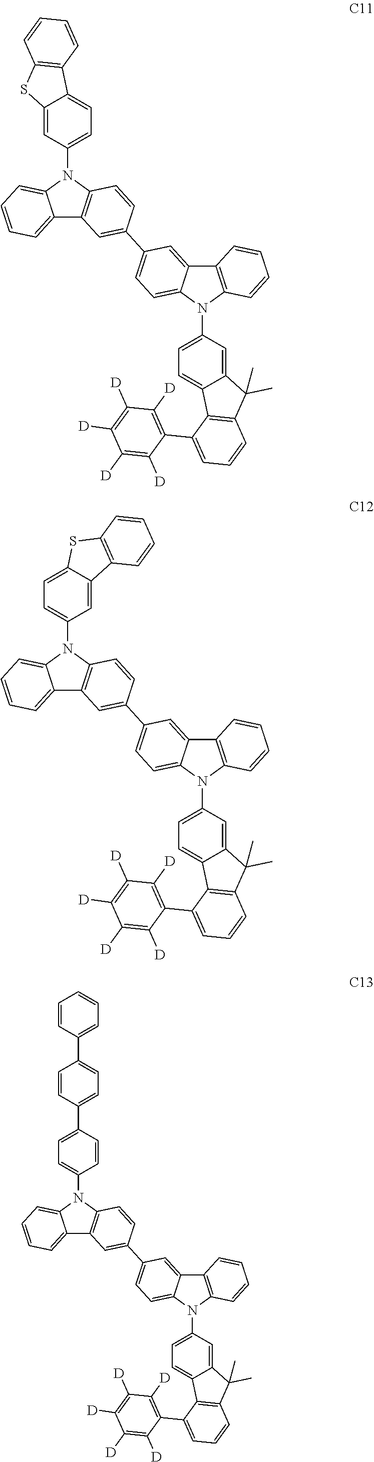 [SMILES: [2H]c1c([2H])c([2H])c(-c2cccc3c2-c2ccc(-n4c5ccccc5c5cc(-c6ccc7c(c6)c6ccccc6n7-c6ccc7c(c6)sc6ccccc67)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc7sc8ccccc8c7c6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc(-c7ccc(-c8ccccc8)cc7)cc6)ccc54)cc2C3(C)C)c([2H])c1[2H]]

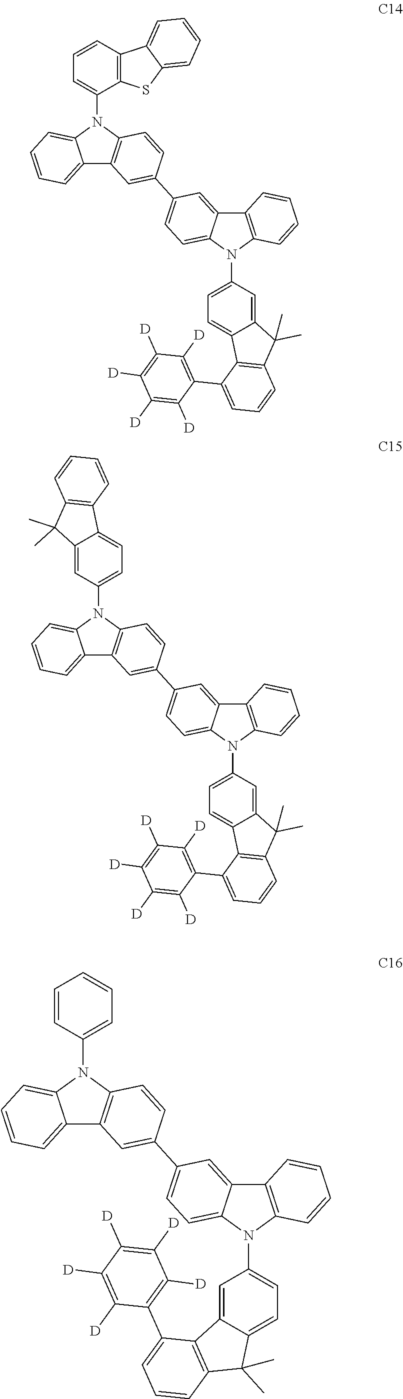

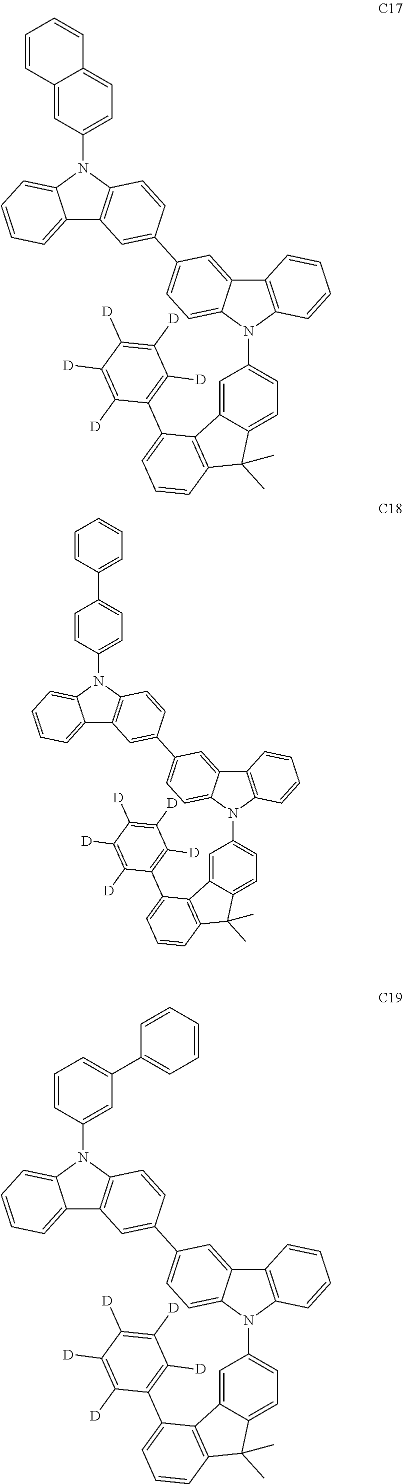 [SMILES: [2H]c1c([2H])c([2H])c(-c2cccc3c2-c2cc(-n4c5ccccc5c5cc(-c6ccc7c(c6)c6ccccc6n7-c6ccc7ccccc7c6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccc(-c7ccccc7)cc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6cccc(-c7ccccc7)c6)ccc54)ccc2C3(C)C)c([2H])c1[2H]]

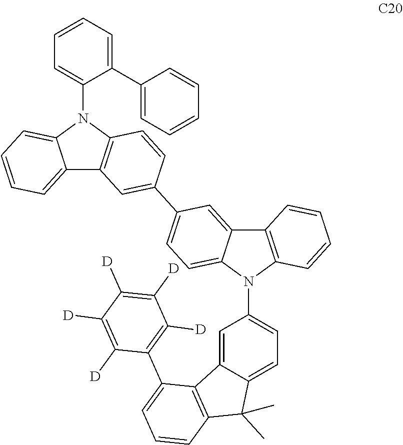 [SMILES: [2H]c1c([2H])c([2H])c(-c2cccc3c2-c2cc(-n4c5ccccc5c5cc(-c6ccc7c(c6)c6ccccc6n7-c6ccccc6-c6ccccc6)ccc54)ccc2C3(C)C)c([2H])c1[2H]]

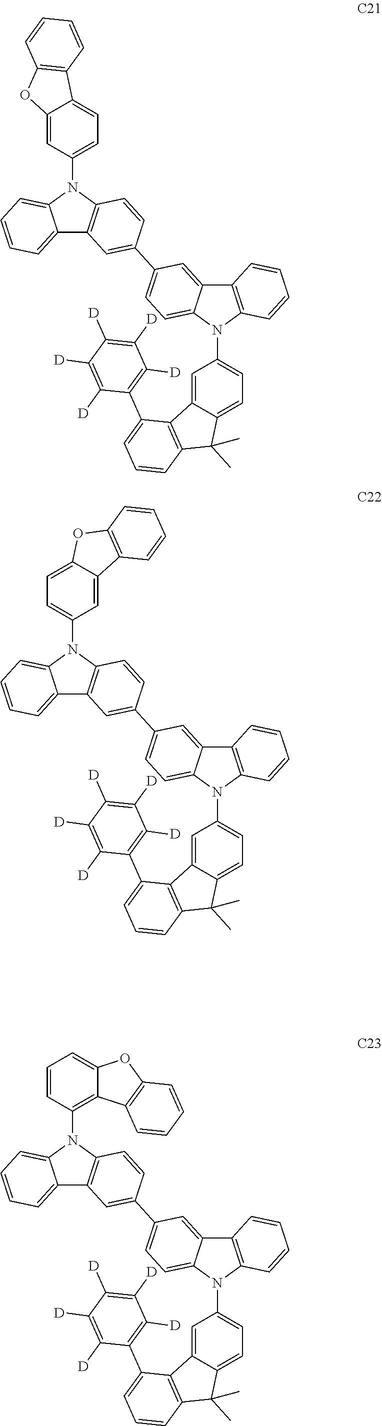

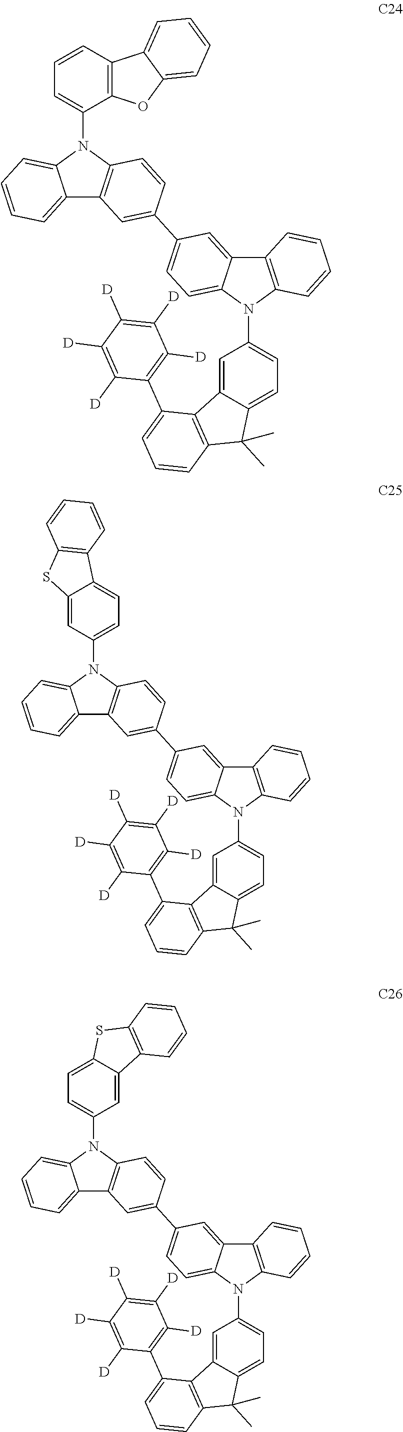 [SMILES: [2H]c1c([2H])c([2H])c(-c2cccc3c2-c2cc(-n4c5ccccc5c5cc(-c6ccc7c(c6)c6ccccc6n7-c6cccc7c6oc6ccccc67)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccc7c(c6)sc6ccccc67)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccc7sc8ccccc8c7c6)ccc54)ccc2C3(C)C)c([2H])c1[2H]]

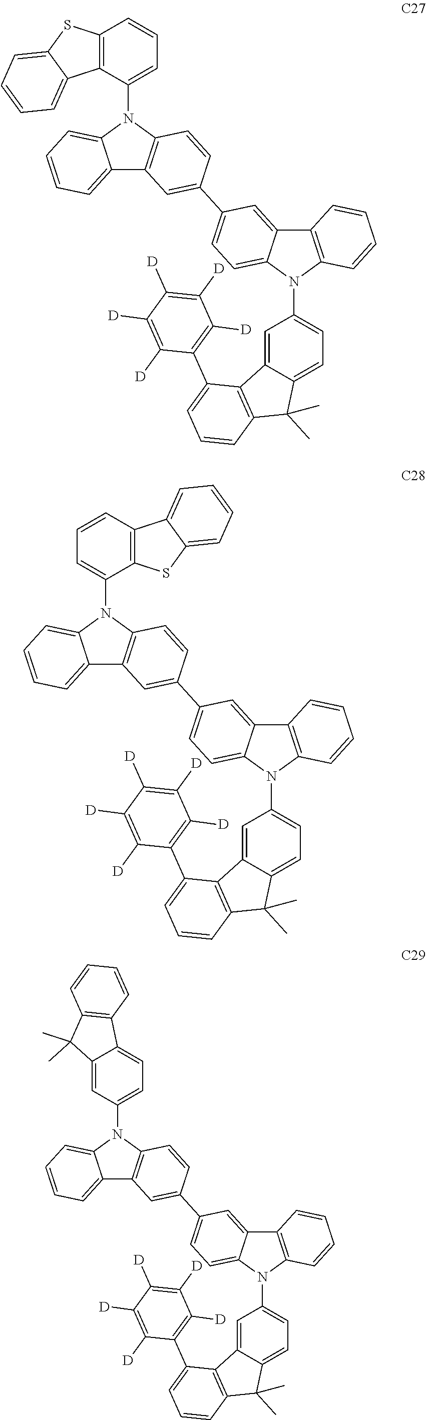

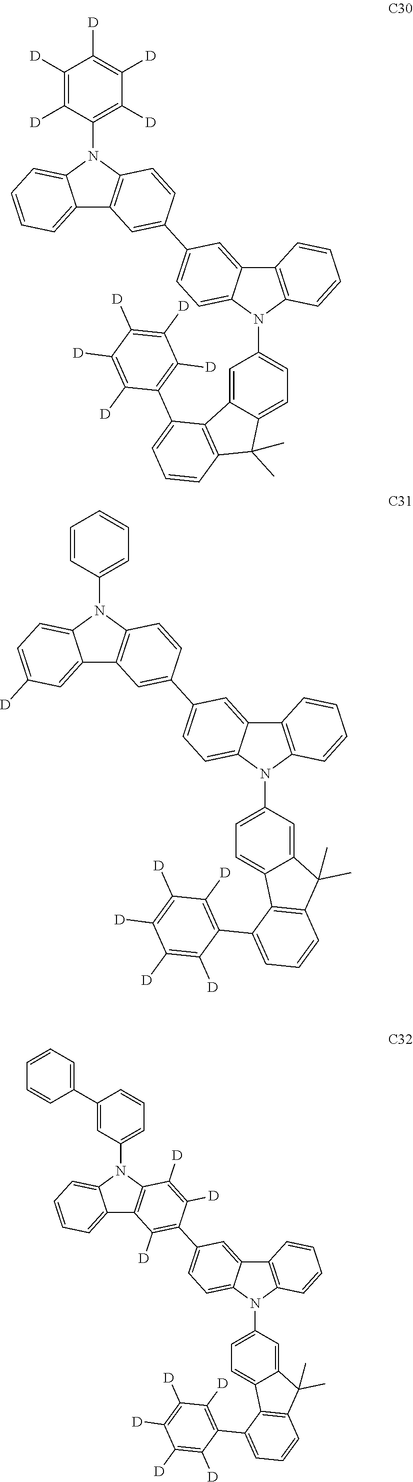 [SMILES: [2H]c1c([2H])c([2H])c(-c2cccc3c2-c2cc(-n4c5ccccc5c5cc(-c6ccc7c(c6)c6ccccc6n7-c6c([2H])c([2H])c([2H])c([2H])c6[2H])ccc54)ccc2C3(C)C)c([2H])c1[2H] ; [2H]c1ccc2c(c1)c1cc(-c3ccc4c(c3)c3ccccc3n4-c3ccc4c(c3)C(C)(C)c3cccc(-c5c([2H])c([2H])c([2H])c([2H])c5[2H])c3-4)ccc1n2-c1ccccc1 ; [2H]c1c([2H])c([2H])c(-c2cccc3c2-c2ccc(-n4c5ccccc5c5cc(-c6c([2H])c([2H])c7c(c6[2H])c6ccccc6n7-c6cccc(-c7ccccc7)c6)ccc54)cc2C3(C)C)c([2H])c1[2H]]

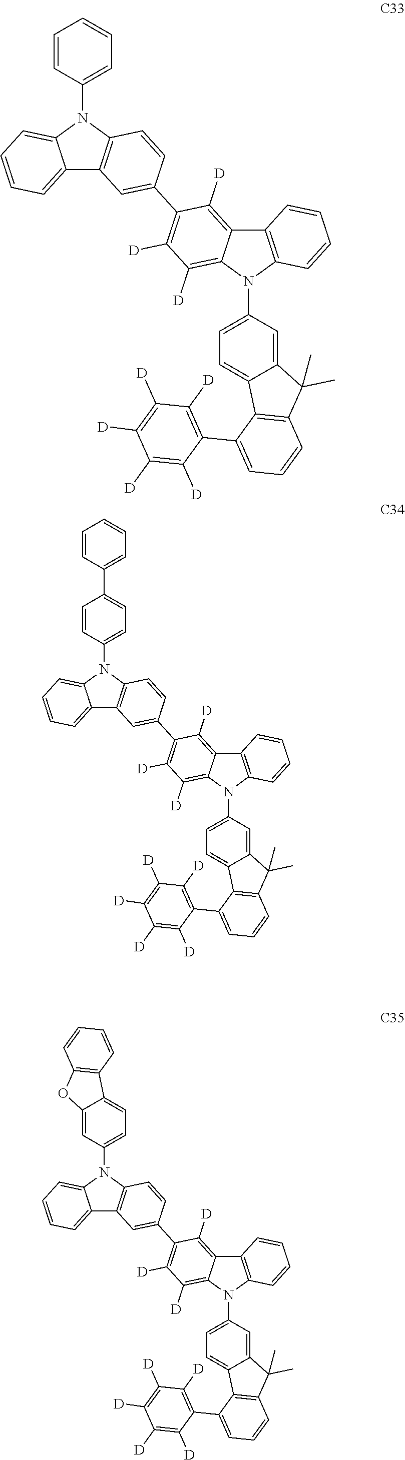 [SMILES: [2H]c1c([2H])c([2H])c(-c2cccc3c2-c2ccc(-n4c5ccccc5c5c([2H])c(-c6ccc7c(c6)c6ccccc6n7-c6ccccc6)c([2H])c([2H])c54)cc2C3(C)C)c([2H])c1[2H] ; [2H]c1c([2H])c([2H])c(-c2cccc3c2-c2ccc(-n4c5ccccc5c5c([2H])c(-c6ccc7c(c6)c6ccccc6n7-c6ccc(-c7ccccc7)cc6)c([2H])c([2H])c54)cc2C3(C)C)c([2H])c1[2H] ; [2H]c1c([2H])c([2H])c(-c2cccc3c2-c2ccc(-n4c5ccccc5c5c([2H])c(-c6ccc7c(c6)c6ccccc6n7-c6ccc7c(c6)oc6ccccc67)c([2H])c([2H])c54)cc2C3(C)C)c([2H])c1[2H]]

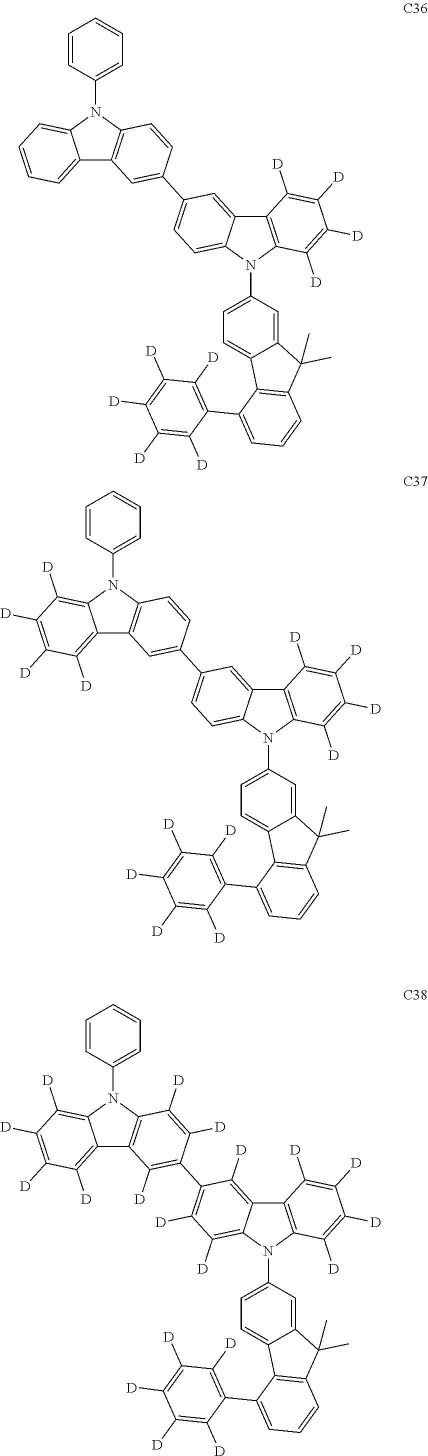

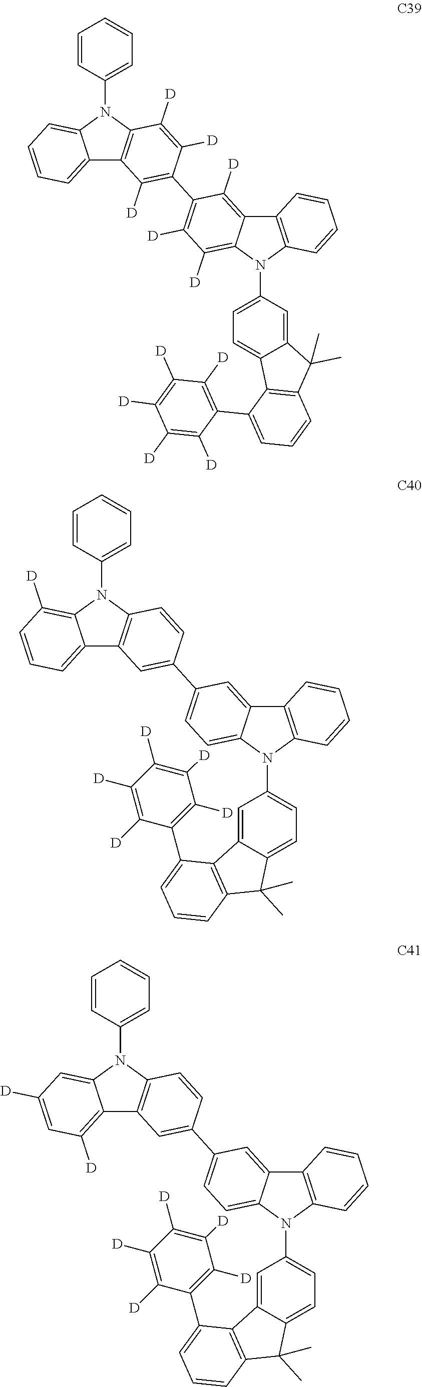 [SMILES: [2H]c1c([2H])c([2H])c(-c2cccc3c2-c2ccc(-n4c5ccccc5c5c([2H])c(-c6c([2H])c([2H])c7c(c6[2H])c6ccccc6n7-c6ccccc6)c([2H])c([2H])c54)cc2C3(C)C)c([2H])c1[2H] ; [2H]c1c([2H])c([2H])c(-c2cccc3c2-c2cc(-n4c5ccccc5c5cc(-c6ccc7c(c6)c6cccc([2H])c6n7-c6ccccc6)ccc54)ccc2C3(C)C)c([2H])c1[2H] ; [2H]c1cc([2H])c2c3cc(-c4ccc5c(c4)c4ccccc4n5-c4ccc5c(c4)-c4c(-c6c([2H])c([2H])c([2H])c([2H])c6[2H])cccc4C5(C)C)ccc3n(-c3ccccc3)c2c1]

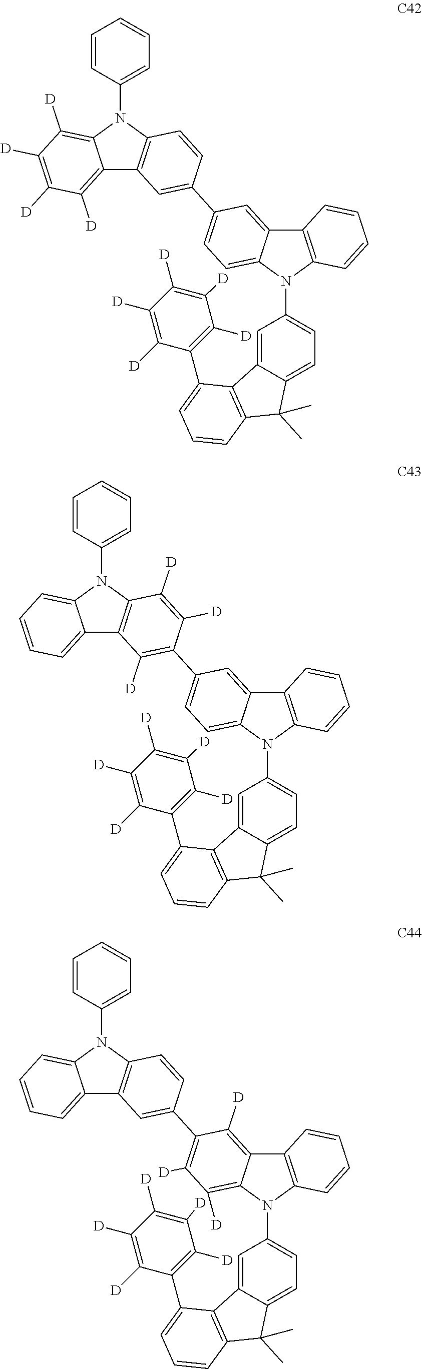 [SMILES: [2H]c1c([2H])c([2H])c(-c2cccc3c2-c2cc(-n4c5ccccc5c5cc(-c6ccc7c(c6)c6c([2H])c([2H])c([2H])c([2H])c6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6c([2H])c([2H])c7c(c6[2H])c6ccccc6n7-c6ccccc6)ccc54)ccc2C3(C)C)c([2H])c1[2H] ; [2H]c1c([2H])c([2H])c(-c2cccc3c2-c2cc(-n4c5ccccc5c5c([2H])c(-c6ccc7c(c6)c6ccccc6n7-c6ccccc6)c([2H])c([2H])c54)ccc2C3(C)C)c([2H])c1[2H]]

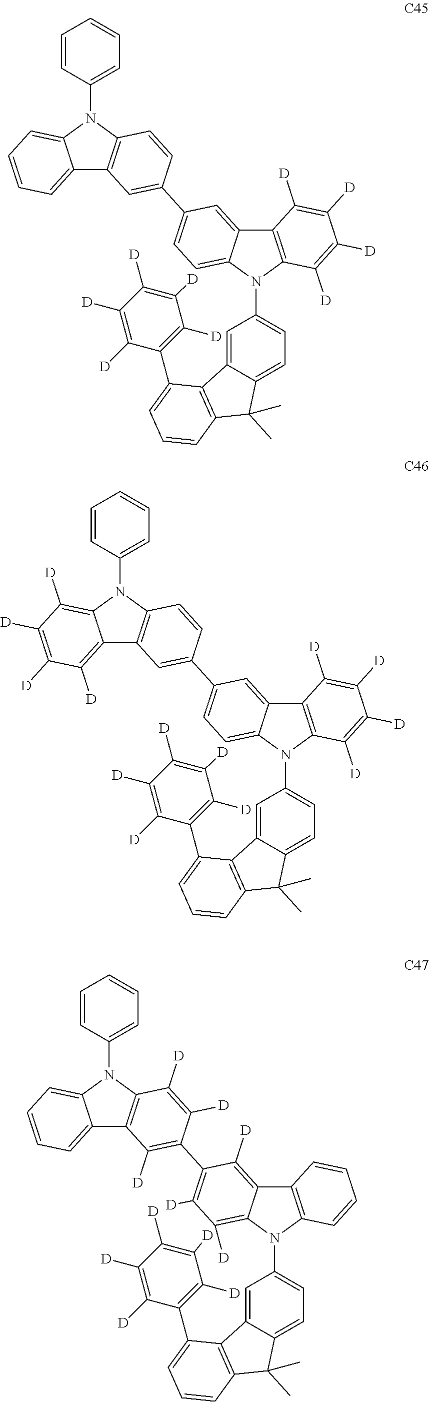 [SMILES: [2H]c1c([2H])c([2H])c(-c2cccc3c2-c2cc(-n4c5ccc(-c6ccc7c(c6)c6ccccc6n7-c6ccccc6)cc5c5c([2H])c([2H])c([2H])c([2H])c54)ccc2C3(C)C)c([2H])c1[2H] ; [2H]c1c([2H])c([2H])c(-c2cccc3c2-c2cc(-n4c5ccc(-c6ccc7c(c6)c6c([2H])c([2H])c([2H])c([2H])c6n7-c6ccccc6)cc5c5c([2H])c([2H])c([2H])c([2H])c54)ccc2C3(C)C)c([2H])c1[2H] ; [2H]c1c([2H])c([2H])c(-c2cccc3c2-c2cc(-n4c5ccccc5c5c([2H])c(-c6c([2H])c([2H])c7c(c6[2H])c6ccccc6n7-c6ccccc6)c([2H])c([2H])c54)ccc2C3(C)C)c([2H])c1[2H]]

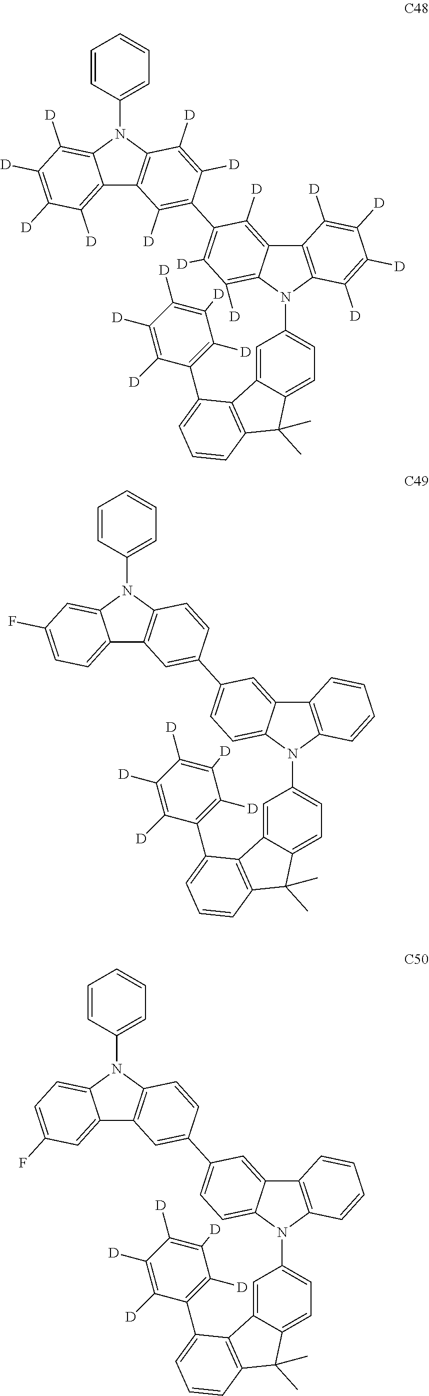 [SMILES: [2H]c1c([2H])c([2H])c(-c2cccc3c2-c2cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccc(F)cc6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6cc(F)ccc6n7-c6ccccc6)ccc54)ccc2C3(C)C)c([2H])c1[2H]]

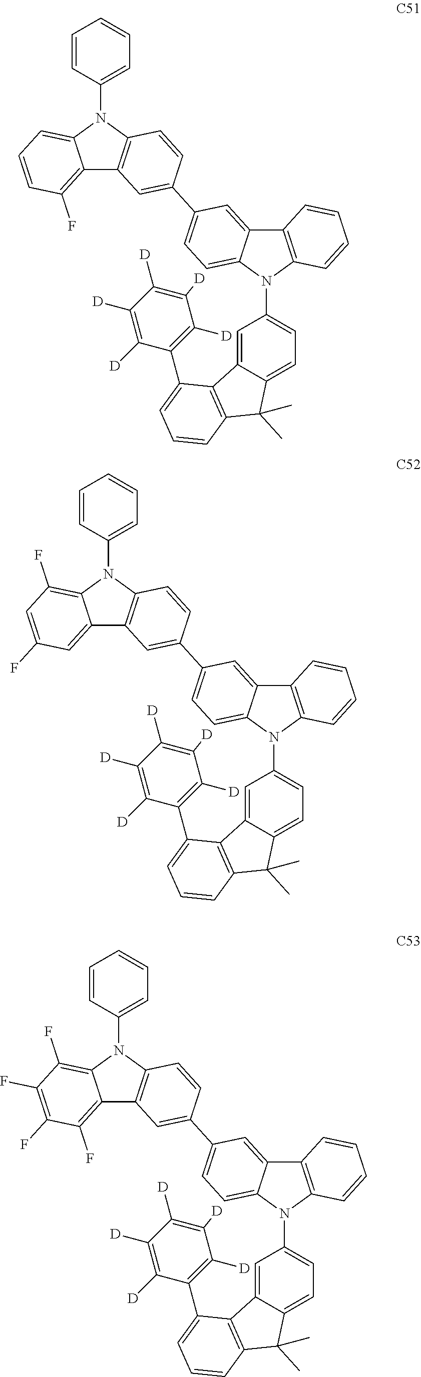 [SMILES: [2H]c1c([2H])c([2H])c(-c2cccc3c2-c2cc(-n4c5ccccc5c5cc(-c6ccc7c(c6)c6c(F)cccc6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6cc(F)cc(F)c6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6c(F)c(F)c(F)c(F)c6n7-c6ccccc6)ccc54)ccc2C3(C)C)c([2H])c1[2H]]

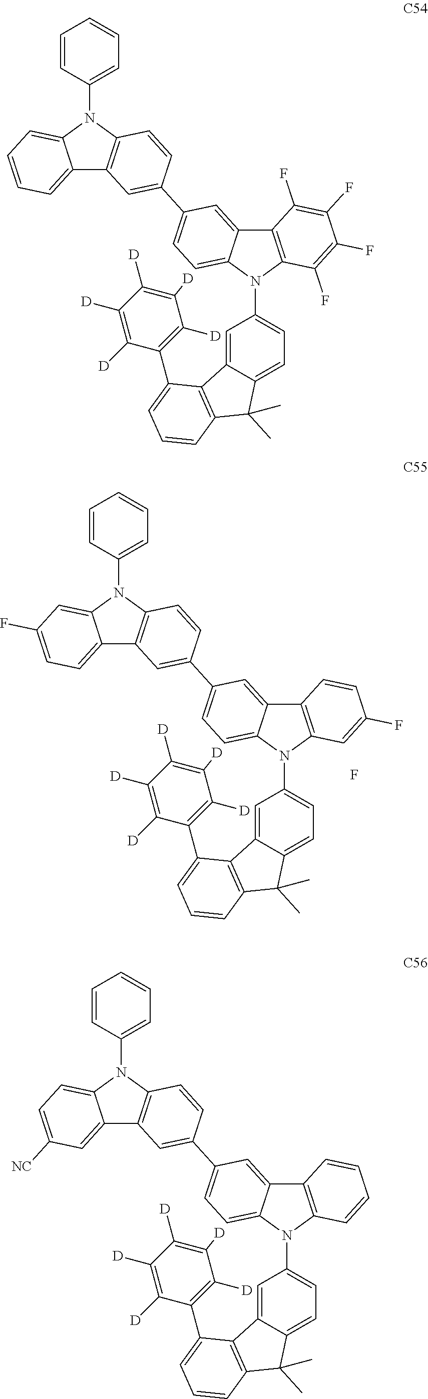

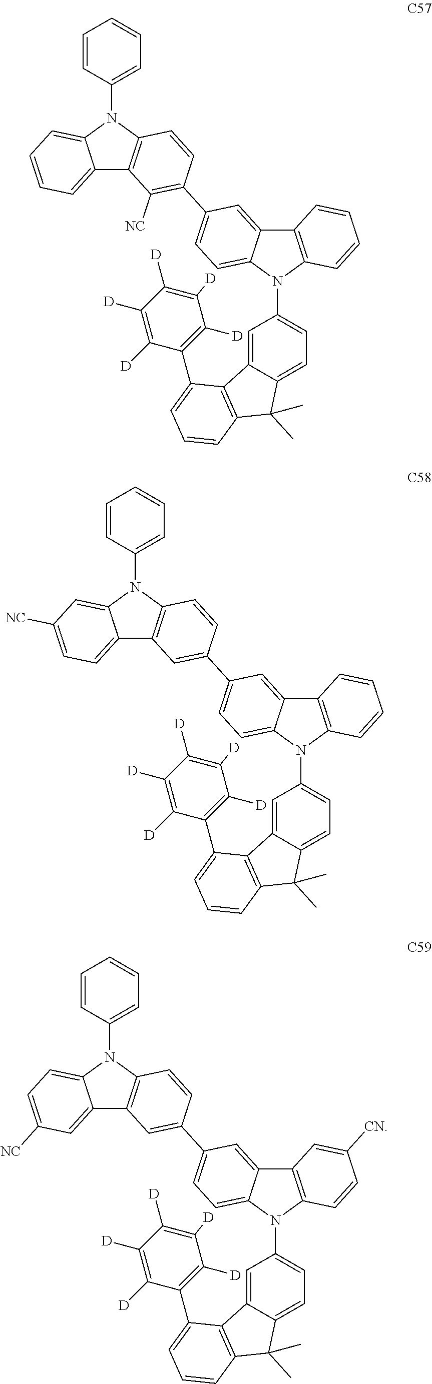 [SMILES: [2H]c1c([2H])c([2H])c(-c2cccc3c2-c2cc(-n4c5ccccc5c5cc(-c6ccc7c(c6C#N)c6ccccc6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccc(C#N)cc6n7-c6ccccc6)ccc54)ccc2C3(C)C)c([2H])c1[2H] ; [2H]c1c([2H])c([2H])c(-c2cccc3c2-c2cc(-n4c5ccc(C#N)cc5c5cc(-c6ccc7c(c6)c6cc(C#N)ccc6n7-c6ccccc6)ccc54)ccc2C3(C)C)c([2H])c1[2H]]

In a second aspect of the present disclosure, the present disclosure provides an organic electroluminescent device, including an anode and a cathode which are oppositely disposed, and a functional layer disposed between the anode and the cathode; wherein the functional layer includes the organic compound of the present disclosure.

Optionally, the functional layer includes an organic light-emitting layer.

Further optionally, the organic light-emitting layer includes the organic compound of the present disclosure.

In some embodiments of the present disclosure, the organic electroluminescent device is a phosphorescent device.

In some specific embodiments of the present disclosure, the organic electroluminescent device is a green organic electroluminescent device.

In some embodiments of the present disclosure, the organic electroluminescent device sequentially includes an anode (an ITO substrate), a hole transport layer, a hole auxiliary layer, an organic light-emitting layer, an electron transport layer, an electron injection layer, a cathode (a Mg—Ag mixture), and an organic capping layer.

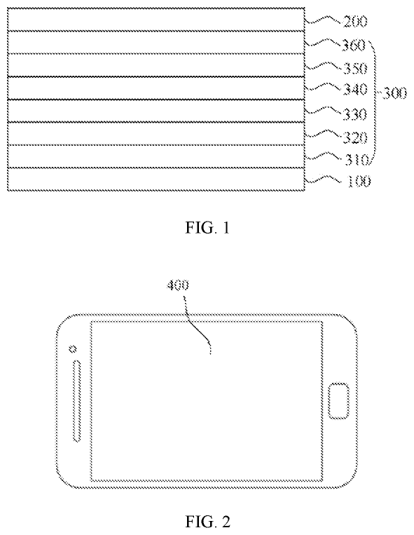

In one specific embodiment of the present disclosure, as shown in FIG. 1, the organic electroluminescent device of the present disclosure includes an anode 100, a cathode 200 disposed opposite to the anode 100, and at least one functional layer 300 between the anode layer and the cathode layer, wherein the functional layer 300 includes a hole injection layer 310, a hole transport layer 320, a hole auxiliary layer 330, an organic light-emitting layer 340, an electron transport layer 350, and an electron injection layer 360 which are sequentially stacked.

Optionally, the anode 100 includes the following anode materials which are preferably materials having a large work function that facilitate hole injection into the organic layer. Specific examples of the anode materials include metals such as nickel, platinum, vanadium, chromium, copper, zinc, and gold, or their alloy; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene] (PEDT), polypyrrole, and polyaniline, but are not limited thereto. In one specific embodiment of the present disclosure, an ITO substrate is selected as the anode.

Optionally, the hole transport layer 320 can include one or more hole transport materials, and the hole transport materials can be selected from a carbazole polymer, carbazole connected triarylamine compounds or other types of compounds, which are not specially limited in the present disclosure. For example, in some embodiments of the present disclosure, the hole transport layer 320 consists of HT-4.

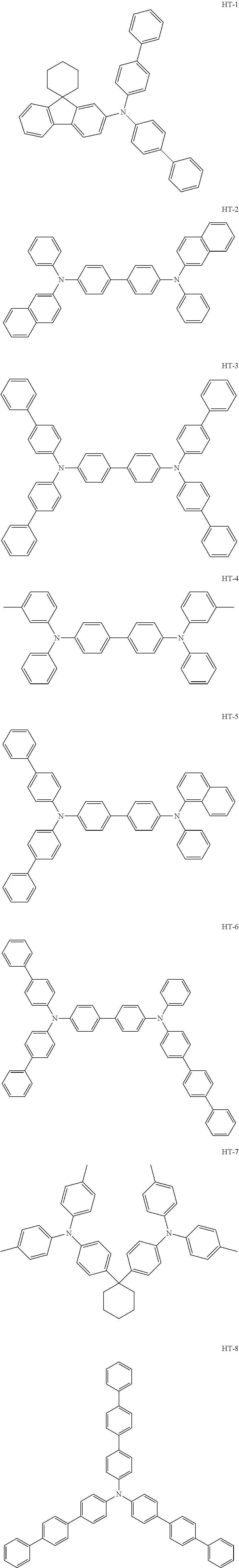

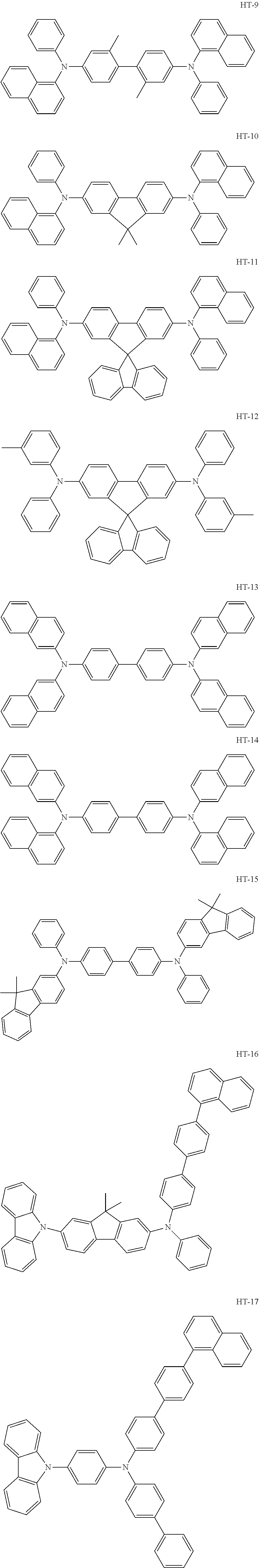

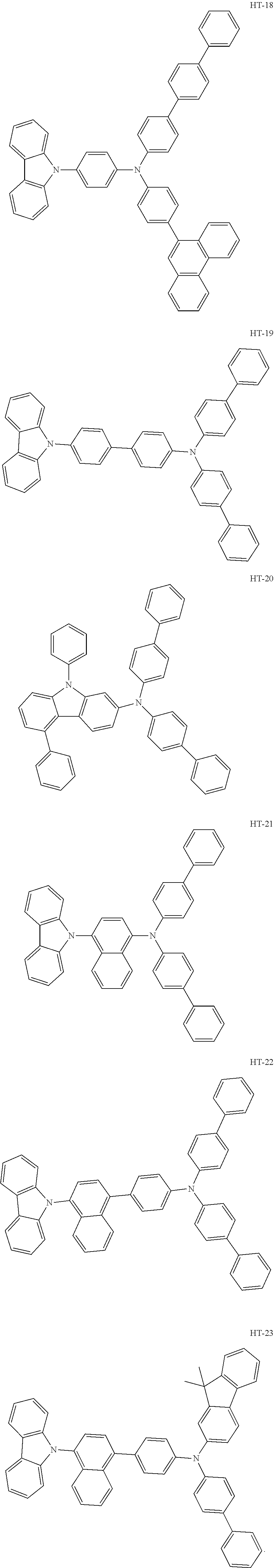

Optionally, the hole auxiliary layer 330 may include one or more hole transport materials, and the hole transport materials may be selected from a carbazole polymer, carbazole connected triarylamine compounds or other types of compounds, which are not specially limited in the present disclosure. For example, in some embodiments of the present disclosure, the hole auxiliary layer 330 consists of HT-20.

Optionally, the hole injection layer 310 may also be arranged between the anode 100 and the hole transport layer 320 to enhance the ability to inject holes into the hole transport layer 320. The hole injection layer 310 can be made of a benzidine derivative, a starburst arylamine compound, a phthalocyanine derivative or other materials, which is not specially limited in the present disclosure. A material of the hole injection layer 310 may be selected from, for example, the following compounds or any combination of them;

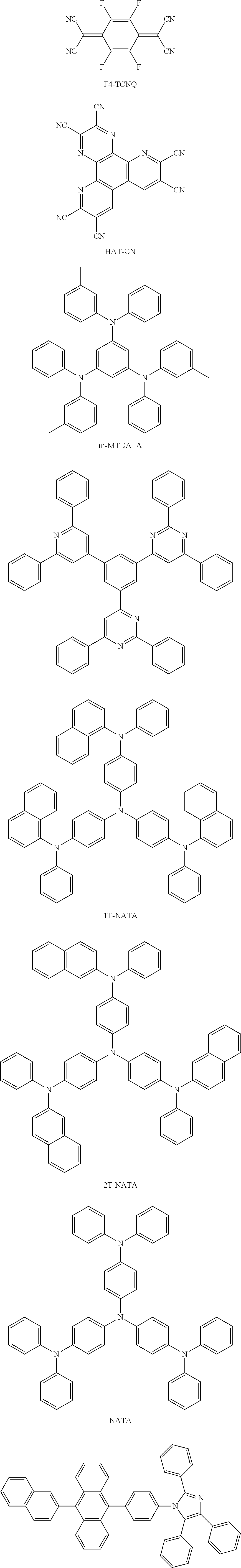

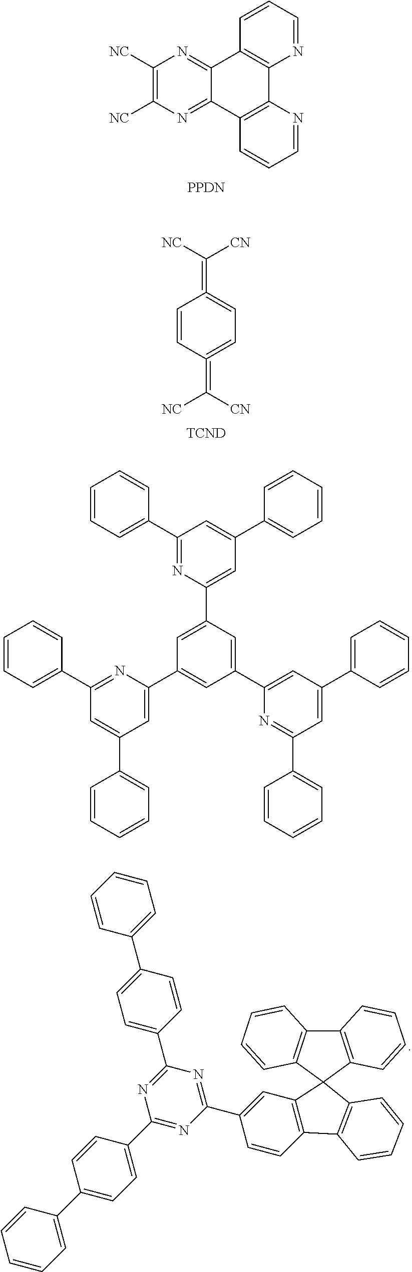

In one embodiment of the present disclosure, the hole injection layer 310 is composed of HAT-CN.

Optionally, the organic light-emitting layer 340 may consist of a single light-emitting material or may include a host material and a guest material. Optionally, the organic light-emitting layer 340 is composed of the host material and the guest material, holes injected into the organic light-emitting layer 340 and electrons injected into the organic light-emitting layer 330 may be recombined in the organic light-emitting layer 340 to form excitons, the excitons transfer energy to the host material, and the host material transfers energy to the guest material, and then the guest material can emit light.

The host material of the organic light-emitting layer 340 may be a mixed host material in which a hole-type host material and an electron-type host material are contained. The electron-type host material may be a triazine material, a quinoline material, or the like, which is not particularly limited in the present disclosure. For example, specific examples of the electron-type host material include, but are not limited to,

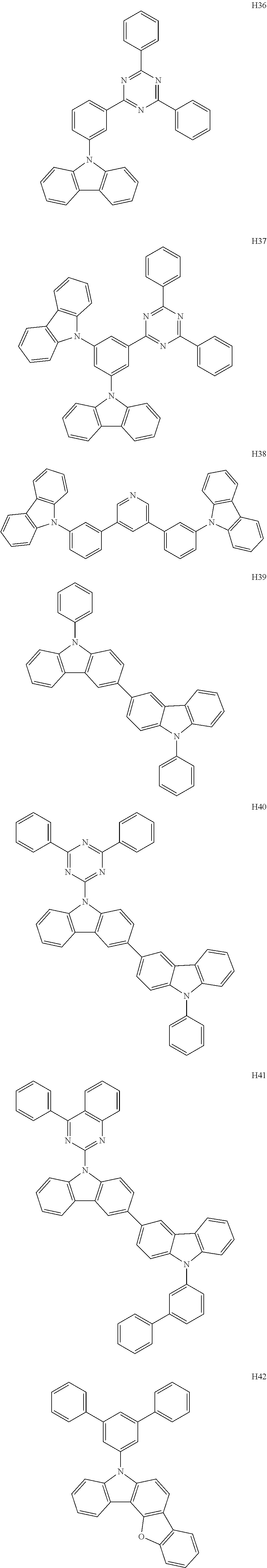

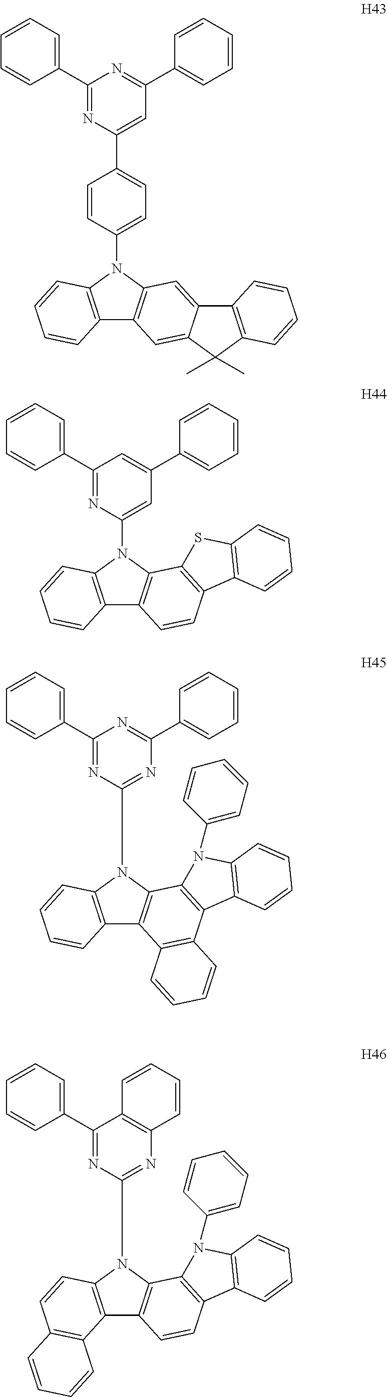

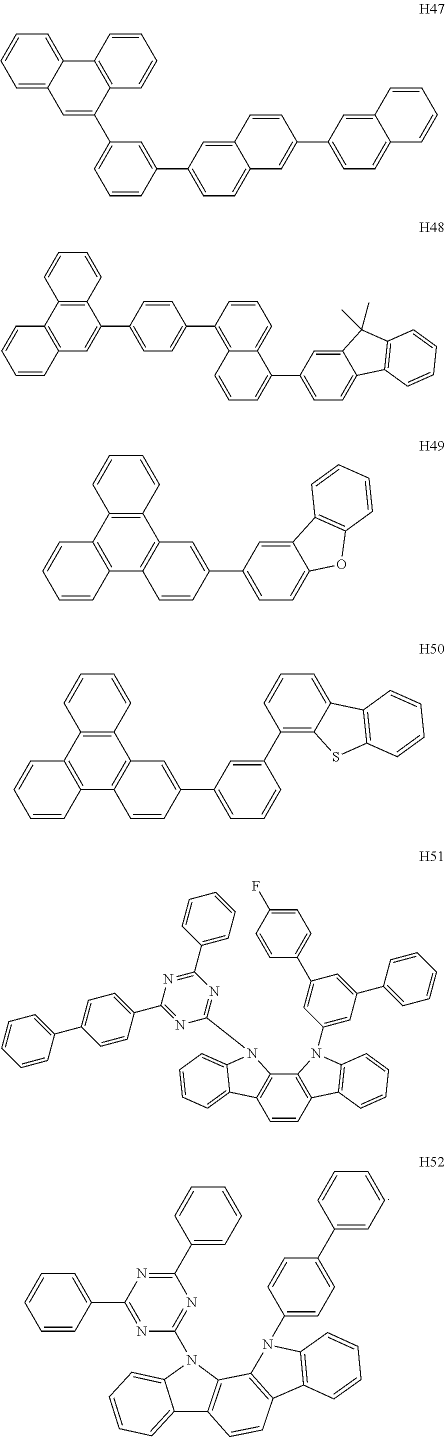

In one specific embodiment of the present disclosure, the electron-type host material of the organic light-emitting layer is

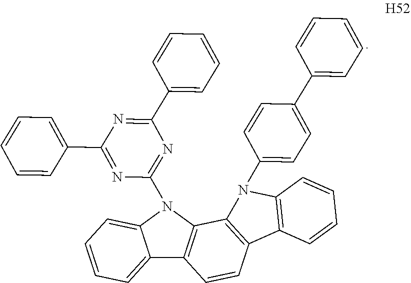 [SMILES: c1ccc(-c2ccc(-n3c4ccccc4c4ccc5c6ccccc6n(-c6nc(-c7ccccc7)nc(-c7ccccc7)n6)c5c43)cc2)cc1]

In one specific embodiment of the present disclosure, the hole-type host material of the organic light-emitting layer is the organic compound of the present disclosure.

The guest material of the organic light-emitting layer 340 may be a compound having a condensed aryl ring or its derivative, a compound having a heteroaryl ring or its derivative, an aromatic amine derivative, or other materials, which is not particularly limited in the present disclosure. The guest material is also referred to as a doping material or a dopant. The guest material can be divided into fluorescent dopants and phosphorescent dopants according to luminescence types. For example, specific examples of green phosphorescent dopants include, but are not limited to,

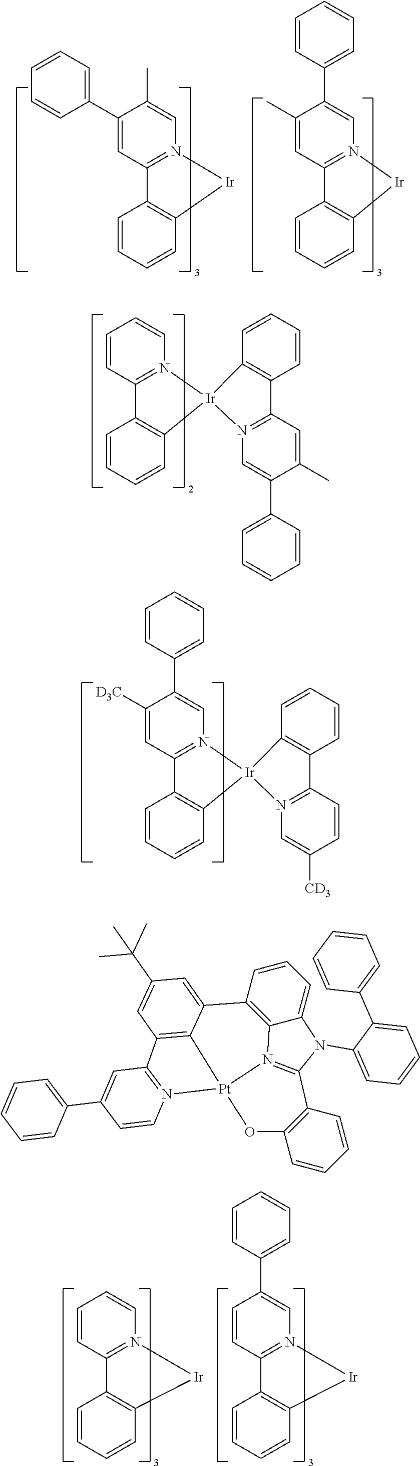

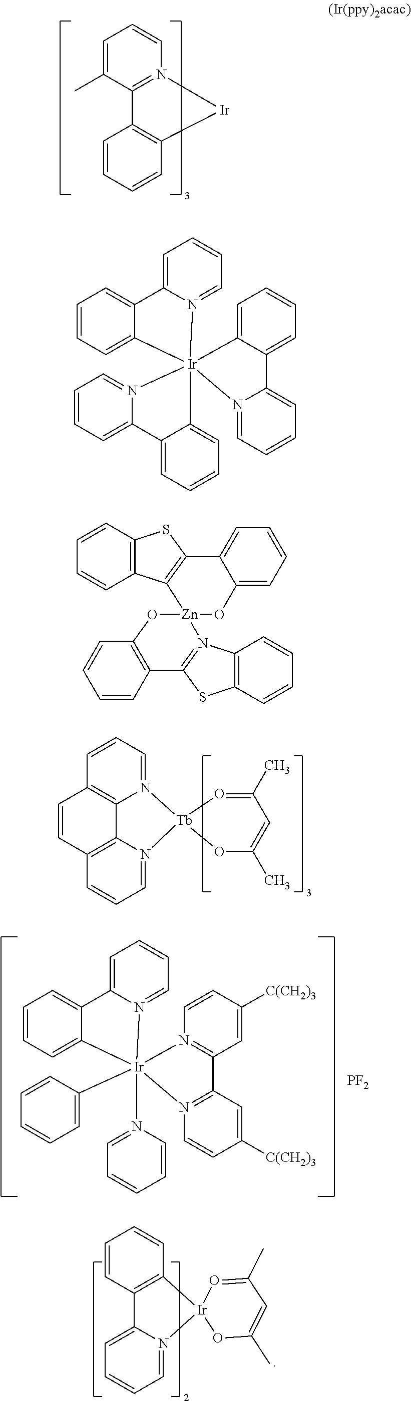

In one embodiment of the present disclosure, the organic electroluminescent device is a green organic electroluminescent device, the host material of the organic light-emitting layer 340 is the organic compound of the present disclosure and H52, and the guest material of the organic light-emitting layer 340 is Ir(ppy)₂acac.

The electron transport layer 350 can be of a single-layer structure or a multi-layer structure, and can include one or more electron transport materials, and the electron transport materials can be selected from a benzimidazole derivative, an oxadiazole derivative, a quinoxaline derivative or other electron transport materials, which are not specially limited in the present disclosure. For example, in some embodiments of the present disclosure, the electron transport layer 350 may be composed of ET-01 and LiQ. A material of the electron transport layer 350 includes, but is not limited to, the following compounds:

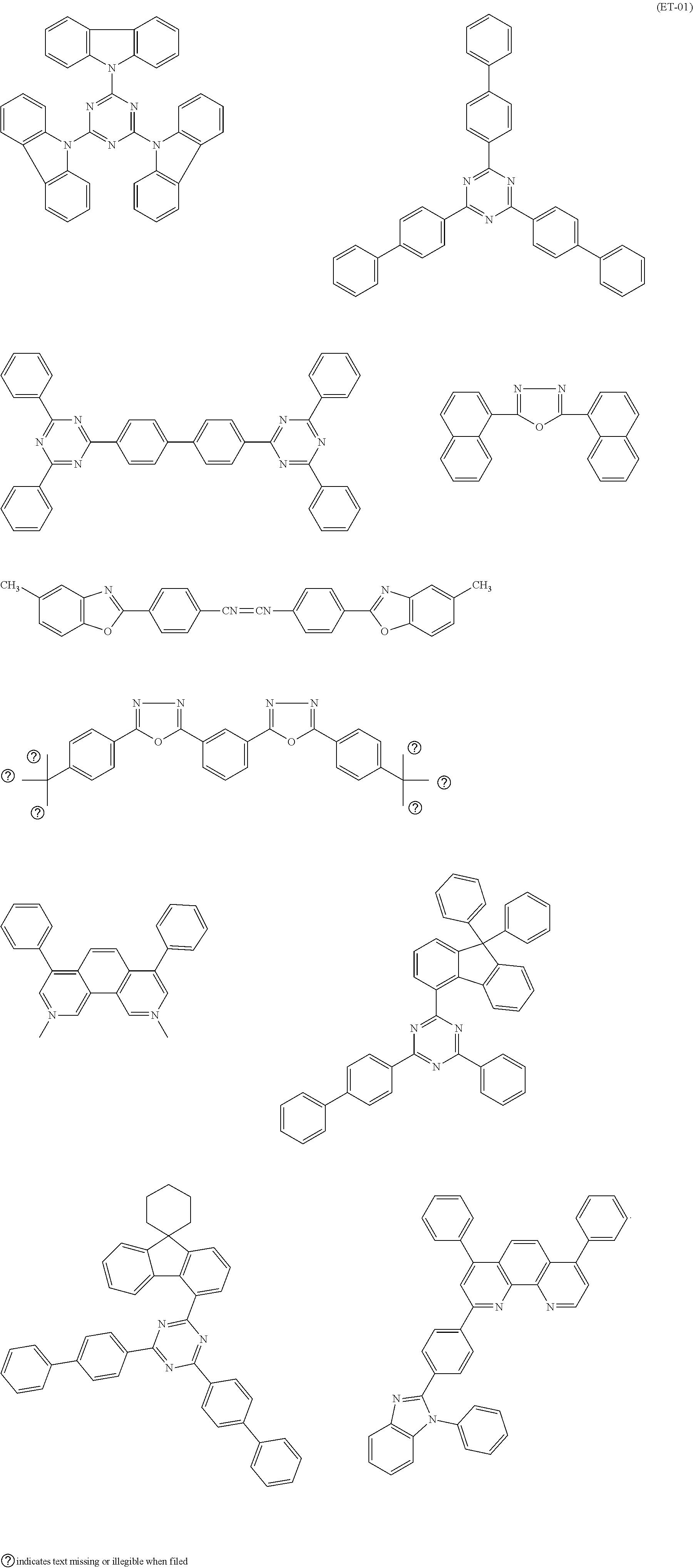

In one embodiment of the present disclosure, the electron transport layer 350 may consist of ET-01 (having a structure shown above) and LiQ.

Optionally, the cathode 200 includes the following cathode materials which are materials having a small work function that facilitate electron injection into the organic layer. Specific examples of the cathode materials include metals such as magnesium, calcium, sodium, potassium, titanium, indium, yttrium, lithium, gadolinium, aluminum, silver, tin and lead, or their alloy; or multilayer materials such as LiF/Al, Liq/Al, LiO₂/Al, LiF/Ca, LiF/Al, and BaF₂/Ca, but are not limited thereto. A metal electrode including silver and magnesium as the cathode is preferably included.

Optionally, the electron injection layer 360 may also be disposed between the cathode 200 and the electron transport layer 350 to enhance the ability to inject electrons into the electron transport layer 350. The electron injection layer 360 may include an inorganic material such as an alkali metal sulfide and an alkali metal halide, or may include a complex of an alkali metal and an organic substance. In some embodiments of the present disclosure, the electron injection layer 360 may include LiQ.

The present disclosure also provides an electronic apparatus, including the organic electroluminescent device described in the present disclosure.

For example, as shown in FIG. 2, the electronic apparatus provided by the present disclosure is a first electronic apparatus 400 including any one of the organic electroluminescent devices described in the above embodiments of the organic electroluminescent device. The electronic apparatus can be a display device, a lighting device, an optical communication device or other types of electronic devices, and may include, for example, but is not limited to, a computer screen, a mobile phone screen, a television, electronic paper, an emergency lighting lamp, an optical module and the like. Since the first electronic apparatus 400 is provided with the above-described organic electroluminescent device, the first electronic apparatus 400 has the same beneficial effects, which will not be repeated here.

The present disclosure will be described in detail with reference to the examples, but the following description is intended to explain the present disclosure and is not intended to limit the scope of the present disclosure in any way.

### Synthesis of an Intermediate Sub A-1

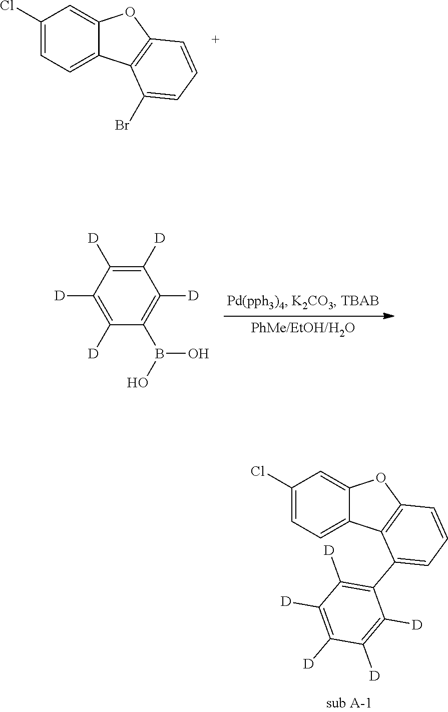 [SMILES: Clc1ccc2c(c1)oc1cccc(Br)c12 ; [2H]c1c([2H])c([2H])c(B(O)O)c([2H])c1[2H] ; [2H]c1c([2H])c([2H])c(-c2cccc3oc4cc(Cl)ccc4c23)c([2H])c1[2H]]

1-Bromo-7-chlorodibenzofuran (50.0 g, 177.6 mmol), deuterated benzeneboronic acid (22.5 g, 177.6 mmol), tetrakis(triphenylphosphine) palladium (2.0 g, 1.7 mmol), potassium carbonate (49.1 g, 355.2 mmol) and tetrabutylammonium bromide (0.5 g, 1.7 mmol) were added into a three-necked flask, toluene (400 mL), ethanol (100 mL) and deionized water (100 mL) were added into the three-necked flask, under nitrogen protection, the mixture was heated to 76° C., and stirred with heating under reflux for 18 h. The reaction solution was cooled to room temperature, stopped stirring, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain sub A-1 (31.2 g, 62%) as a white product.

Referring to the synthesis method of sub A-1, intermediate compounds sub A-X shown in Table 1 below were synthesized by using a reactant A in Table 1 below instead of 1-bromo-7-chlorodibenzofuran and a reactant B in Table 1 below instead of deuterated benzeneboronic acid:

TABLE 1

| | | | | Yield |
|---|---|---|---|---|
| sub A-X | Reactant A | Reactant B | Structure | (%) |
| sub A-2 | 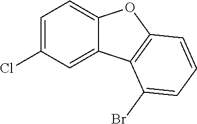 [SMILES: Clc1ccc2oc3cccc(Br)c3c2c1] | 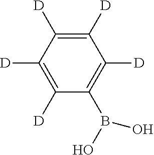 [SMILES: [2H]c1c([2H])c([2H])c(B(O)O)c([2H])c1[2H]] | 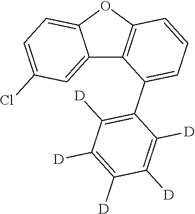 [SMILES: [2H]c1c([2H])c([2H])c(-c2cccc3oc4ccc(Cl)cc4c23)c([2H])c1[2H]] | 65 |
| sub A-3 | | 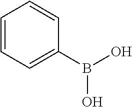 | 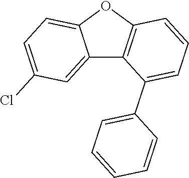 [SMILES: Clc1ccc2oc3cccc(-c4ccccc4)c3c2c1] | 63 |
| sub A-4 | | 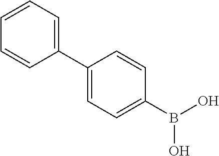 [SMILES: OB(O)c1ccc(-c2ccccc2)cc1] | 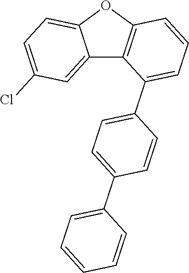 [SMILES: Clc1ccc2oc3cccc(-c4ccc(-c5ccccc5)cc4)c3c2c1] | 59 |
| sub A-5 | 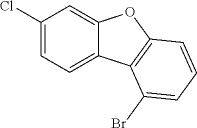 [SMILES: Clc1ccc2c(c1)oc1cccc(Br)c12] | 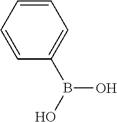 | 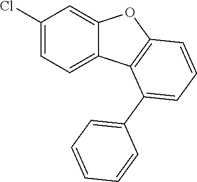 [SMILES: Clc1ccc2c(c1)oc1cccc(-c3ccccc3)c12] | 71 |
| sub A-6 | 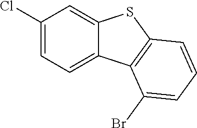 [SMILES: Clc1ccc2c(c1)sc1cccc(Br)c12] | 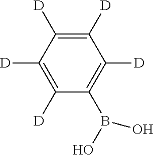 [SMILES: [2H]c1c([2H])c([2H])c(B(O)O)c([2H])c1[2H]] | 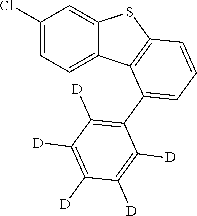 [SMILES: [2H]c1c([2H])c([2H])c(-c2cccc3sc4cc(Cl)ccc4c23)c([2H])c1[2H]] | 75 |
| sub A-7 | 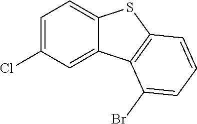 [SMILES: Clc1ccc2sc3cccc(Br)c3c2c1] | 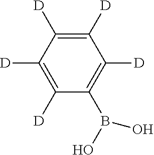 [SMILES: [2H]c1c([2H])c([2H])c(B(O)O)c([2H])c1[2H]] | 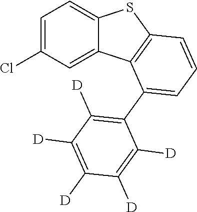 [SMILES: [2H]c1c([2H])c([2H])c(-c2cccc3sc4ccc(Cl)cc4c23)c([2H])c1[2H]] | 62 |
| sub A-8 | 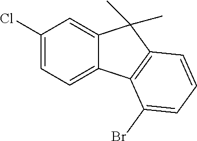 [SMILES: CC1(C)c2cc(Cl)ccc2-c2c(Br)cccc21] | 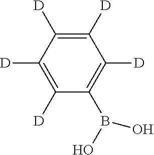 [SMILES: [2H]c1c([2H])c([2H])c(B(O)O)c([2H])c1[2H]] | 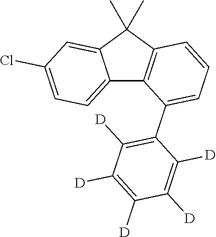 [SMILES: [2H]c1c([2H])c([2H])c(-c2cccc3c2-c2ccc(Cl)cc2C3(C)C)c([2H])c1[2H]] | 73 |

### Synthesis of an Intermediate Sub B-2

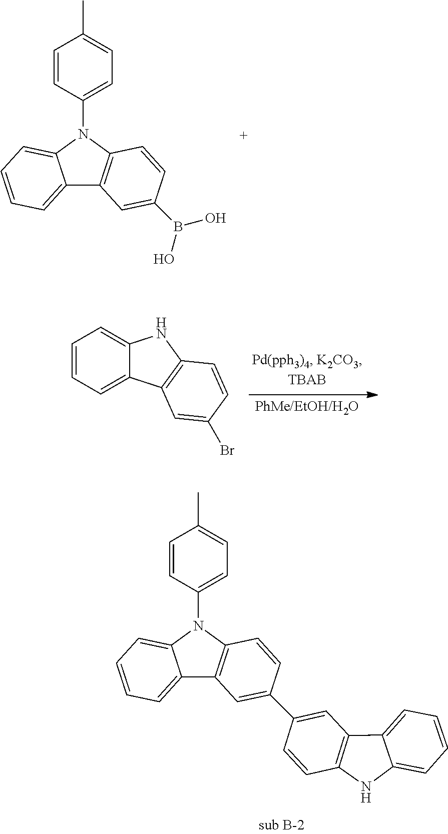 [SMILES: Cc1ccc(-n2c3ccccc3c3cc(B(O)O)ccc32)cc1 ; Brc1ccc2[nH]c3ccccc3c2c1 ; Cc1ccc(-n2c3ccccc3c3cc(-c4ccc5[nH]c6ccccc6c5c4)ccc32)cc1]

9-p-tolyl-9-carbazole-3-boronic acid (31.2 g, 103.6 mmol), 3-bromocarbazole (25.0 g, 105.5 mmol), tetrakis(triphenylphosphine) palladium (1.1 g, 1.0 mmol), potassium carbonate (28.0 g, 203.1 mmol) and tetrabutylammonium bromide (0.3 g, 1.0 mmol) were added into a three-necked flask, toluene (200 mL), ethanol (100 mL) and deionized water (50 mL) were added into the three-necked flask, under nitrogen protection, and the mixture was heated to 76° C., and stirred with heating under reflux for 18 h. The reaction solution was cooled to room temperature, stopped stirring, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain sub B-2 (27.4 g, 64%) as a white product.

Referring to the synthesis method of sub B-2, intermediates sub B-X shown in Table 2 below were synthesized by using a reactant C in Table 2 below instead of 9-p-tolyl-9-carbazole-3-boronic acid:

TABLE 2

| sub | | | Yield |
|---|---|---|---|
| B-X | Reactant C | Structure | (%) |
| sub B-3 | 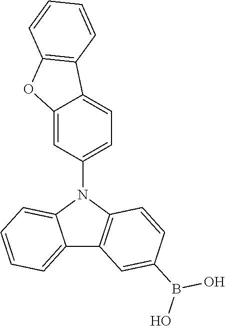 [SMILES: OB(O)c1ccc2c(c1)c1ccccc1n2-c1ccc2c(c1)oc1ccccc12] | 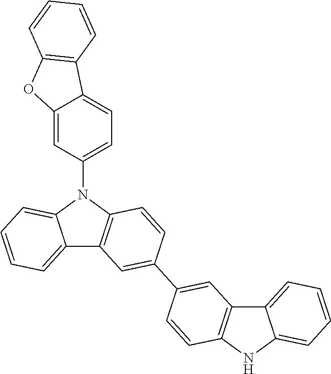 [SMILES: c1ccc2c(c1)[nH]c1ccc(-c3ccc4c(c3)c3ccccc3n4-c3ccc4c(c3)oc3ccccc34)cc12] | 55 |
| sub B-4 | 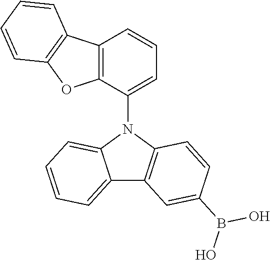 [SMILES: OB(O)c1ccc2c(c1)c1ccccc1n2-c1cccc2c1oc1ccccc12] | 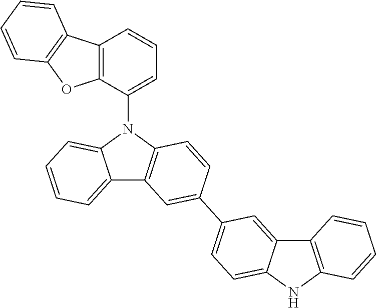 [SMILES: c1ccc2c(c1)[nH]c1ccc(-c3ccc4c(c3)c3ccccc3n4-c3cccc4c3oc3ccccc34)cc12] | 62 |
| sub B-5 | 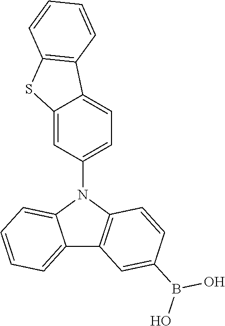 [SMILES: OB(O)c1ccc2c(c1)c1ccccc1n2-c1ccc2c(c1)sc1ccccc12] | 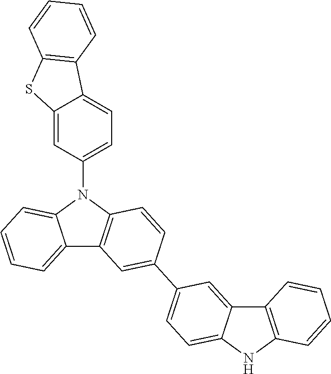 [SMILES: c1ccc2c(c1)[nH]c1ccc(-c3ccc4c(c3)c3ccccc3n4-c3ccc4c(c3)sc3ccccc34)cc12] | 61 |
| sub B-6 | 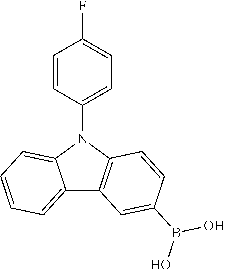 [SMILES: OB(O)c1ccc2c(c1)c1ccccc1n2-c1ccc(F)cc1] | 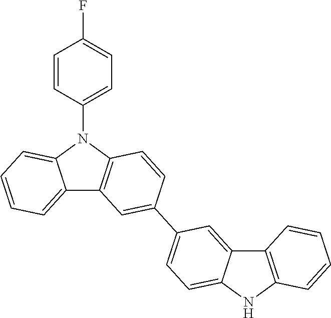 [SMILES: Fc1ccc(-n2c3ccccc3c3cc(-c4ccc5[nH]c6ccccc6c5c4)ccc32)cc1] | 60 |
| sub B-7 | 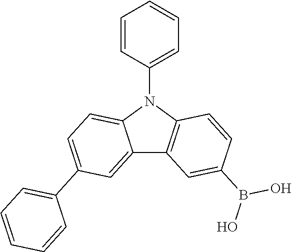 [SMILES: OB(O)c1ccc2c(c1)c1cc(-c3ccccc3)ccc1n2-c1ccccc1] | 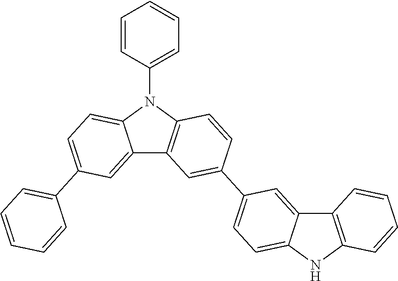 [SMILES: c1ccc(-c2ccc3c(c2)c2cc(-c4ccc5[nH]c6ccccc6c5c4)ccc2n3-c2ccccc2)cc1] | 52 |
| sub B-8 | 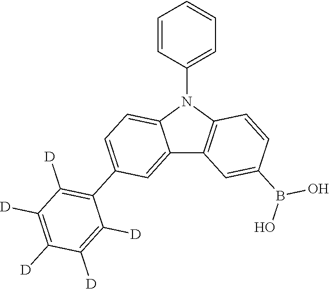 [SMILES: [2H]c1c([2H])c([2H])c(-c2ccc3c(c2)c2cc(B(O)O)ccc2n3-c2ccccc2)c([2H])c1[2H]] | 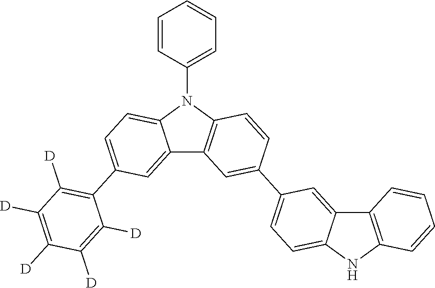 [SMILES: [2H]c1c([2H])c([2H])c(-c2ccc3c(c2)c2cc(-c4ccc5[nH]c6ccccc6c5c4)ccc2n3-c2ccccc2)c([2H])c1[2H]] | 58 |
| sub B-9 | 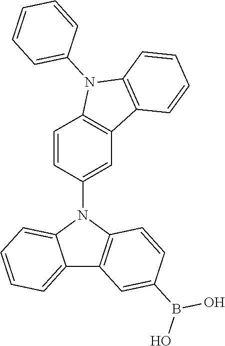 [SMILES: OB(O)c1ccc2c(c1)c1ccccc1n2-c1ccc2c(c1)c1ccccc1n2-c1ccccc1] | 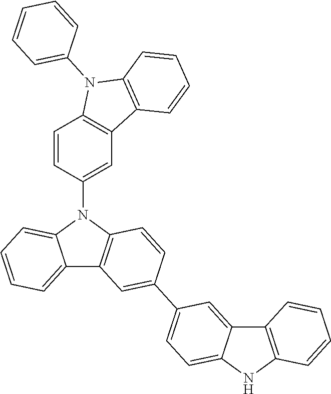 [SMILES: c1ccc(-n2c3ccccc3c3cc(-n4c5ccccc5c5cc(-c6ccc7[nH]c8ccccc8c7c6)ccc54)ccc32)cc1] | 63 |
| sub B-10 | 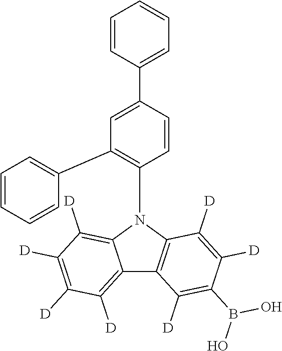 [SMILES: [2H]c1c([2H])c([2H])c2c(c1[2H])c1c([2H])c(B(O)O)c([2H])c([2H])c1n2-c1ccc(-c2ccccc2)cc1-c1ccccc1] | 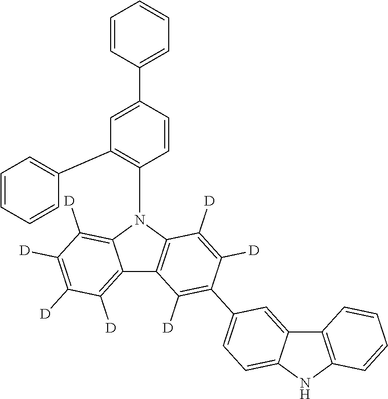 [SMILES: [2H]c1c([2H])c([2H])c2c(c1[2H])c1c([2H])c(-c3ccc4[nH]c5ccccc5c4c3)c([2H])c([2H])c1n2-c1ccc(-c2ccccc2)cc1-c1ccccc1] | 55 |
| sub B-11 | 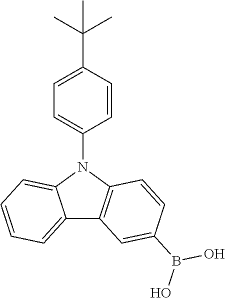 [SMILES: CC(C)(C)c1ccc(-n2c3ccccc3c3cc(B(O)O)ccc32)cc1] | 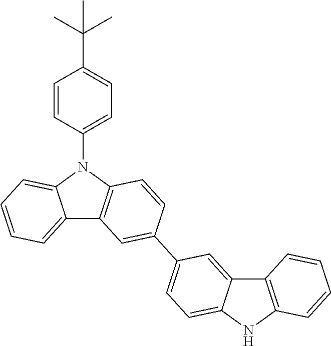 [SMILES: CC(C)(C)c1ccc(-n2c3ccccc3c3cc(-c4ccc5[nH]c6ccccc6c5c4)ccc32)cc1] | 61 |

### Synthesis of a Compound A1

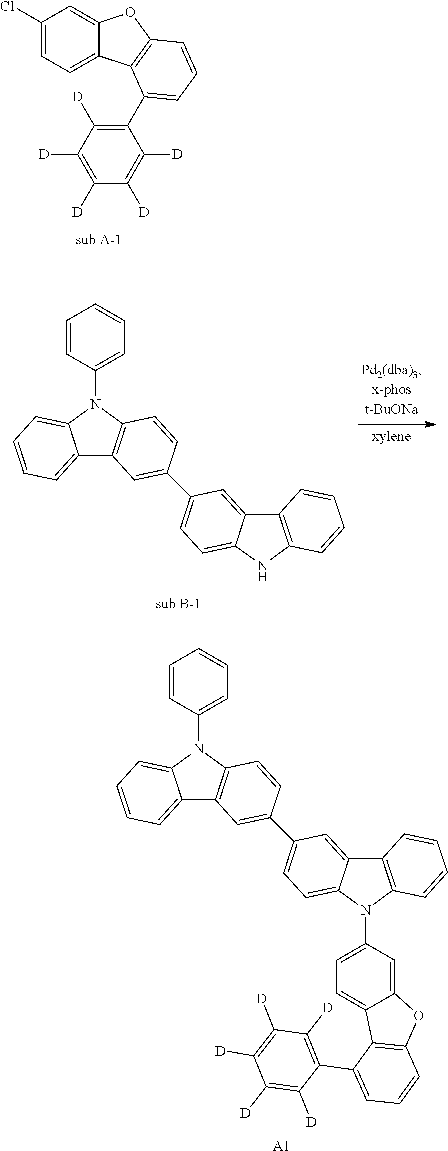

sub A-1 (7.1 g, 24.9 mmol), sub B-1 (10.0 g, 24.4 mmol), tris(dibenzylideneacetone) dipalladium (0.20 g, 0.21 mmol), 2-dicyclohexylphosphino-2′,4′,6′-triisopropylbiphenyl (0.20 g, 0.4 mmol) and sodium tert-butoxide (3.5 g, 36.7 mmol) were added to xylene (150 mL), and the mixture was heated to 140° C. and stirred for 4 h under nitrogen protection. Then, the reaction solution was cooled to room temperature, the reaction solution was washed with water, dried by adding anhydrous magnesium sulfate, and filtered, and a solvent was removed from the obtained filtrate under reduced pressure; and a crude product was purified by recrystallization using a dichloromethane/n-heptane system to obtain the compound A1 (11.5 g, yield: 72%). Mass spectrum: m/z=656.27 [M+H]⁺. Referring to the synthesis method of the compound A1, compounds shown in Table 3 below were synthesized by using a reactant D in Table 3 below instead of sub A-1 and a reactant E instead of sub B-1:

TABLE 3

| Compound | Reactant D | Reactant E |
|---|---|---|
| A4 | 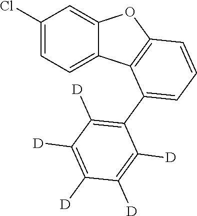 [SMILES: [2H]c1c([2H])c([2H])c(-c2cccc3oc4cc(Cl)ccc4c23)c([2H])c1[2H]] | 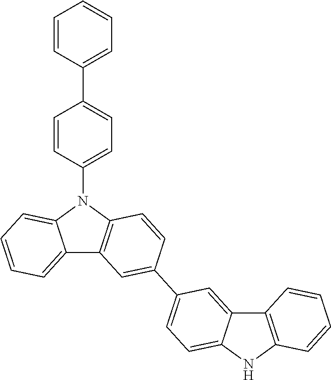 [SMILES: c1ccc(-c2ccc(-n3c4ccccc4c4cc(-c5ccc6[nH]c7ccccc7c6c5)ccc43)cc2)cc1] |
| A5 | 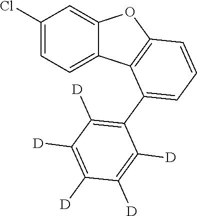 [SMILES: [2H]c1c([2H])c([2H])c(-c2cccc3oc4cc(Cl)ccc4c23)c([2H])c1[2H]] | 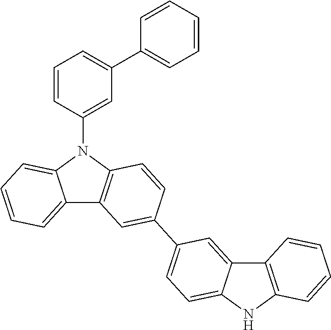 [SMILES: c1ccc(-c2cccc(-n3c4ccccc4c4cc(-c5ccc6[nH]c7ccccc7c6c5)ccc43)c2)cc1] |
| A6 | 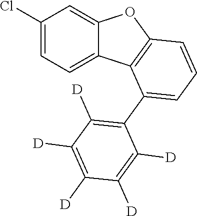 [SMILES: [2H]c1c([2H])c([2H])c(-c2cccc3oc4cc(Cl)ccc4c23)c([2H])c1[2H]] | 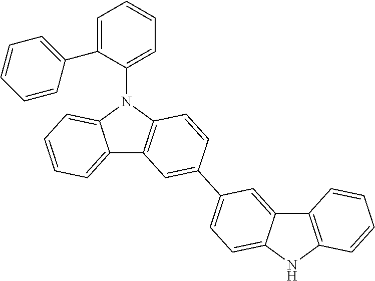 [SMILES: c1ccc(-c2ccccc2-n2c3ccccc3c3cc(-c4ccc5[nH]c6ccccc6c5c4)ccc32)cc1] |
| A3 | 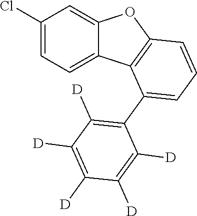 [SMILES: [2H]c1c([2H])c([2H])c(-c2cccc3oc4cc(Cl)ccc4c23)c([2H])c1[2H]] | 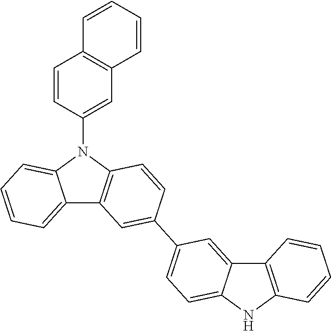 [SMILES: c1ccc2cc(-n3c4ccccc4c4cc(-c5ccc6[nH]c7ccccc7c6c5)ccc43)ccc2c1] |
| A8 | 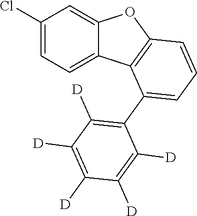 [SMILES: [2H]c1c([2H])c([2H])c(-c2cccc3oc4cc(Cl)ccc4c23)c([2H])c1[2H]] | 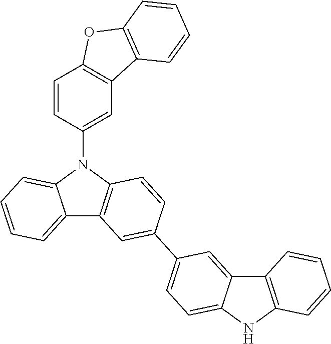 [SMILES: c1ccc2c(c1)[nH]c1ccc(-c3ccc4c(c3)c3ccccc3n4-c3ccc4oc5ccccc5c4c3)cc12] |
| A42 | 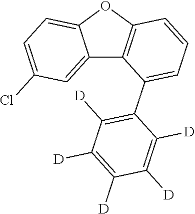 [SMILES: [2H]c1c([2H])c([2H])c(-c2cccc3oc4ccc(Cl)cc4c23)c([2H])c1[2H]] | 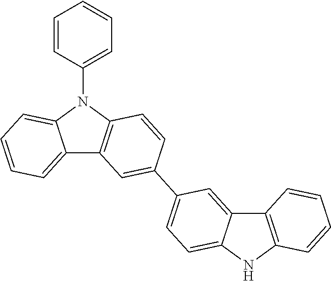 [SMILES: c1ccc(-n2c3ccccc3c3cc(-c4ccc5[nH]c6ccccc6c5c4)ccc32)cc1] |
| A45 | 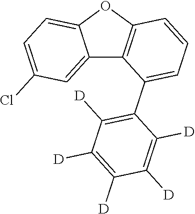 [SMILES: [2H]c1c([2H])c([2H])c(-c2cccc3oc4ccc(Cl)cc4c23)c([2H])c1[2H]] | 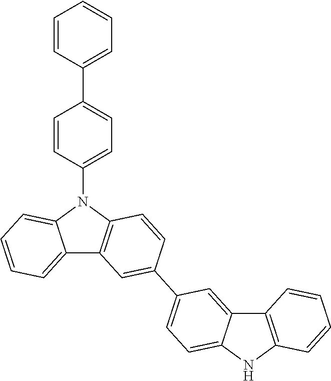 [SMILES: c1ccc(-c2ccc(-n3c4ccccc4c4cc(-c5ccc6[nH]c7ccccc7c6c5)ccc43)cc2)cc1] |
| A46 | 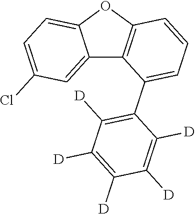 [SMILES: [2H]c1c([2H])c([2H])c(-c2cccc3oc4ccc(Cl)cc4c23)c([2H])c1[2H]] | 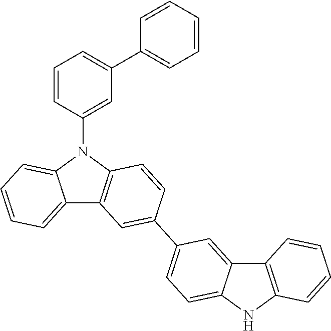 [SMILES: c1ccc(-c2cccc(-n3c4ccccc4c4cc(-c5ccc6[nH]c7ccccc7c6c5)ccc43)c2)cc1] |
| A49 | 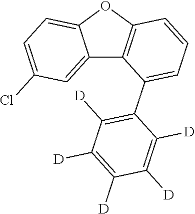 [SMILES: [2H]c1c([2H])c([2H])c(-c2cccc3oc4ccc(Cl)cc4c23)c([2H])c1[2H]] | 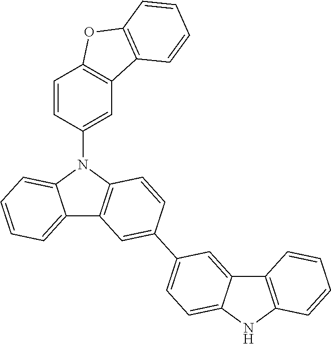 [SMILES: c1ccc2c(c1)[nH]c1ccc(-c3ccc4c(c3)c3ccccc3n4-c3ccc4oc5ccccc5c4c3)cc12] |
| A62 | 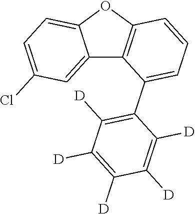 [SMILES: [2H]c1c([2H])c([2H])c(-c2cccc3oc4ccc(Cl)cc4c23)c([2H])c1[2H]] | 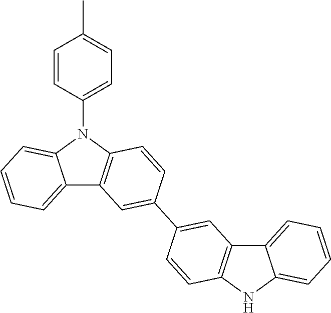 [SMILES: Cc1ccc(-n2c3ccccc3c3cc(-c4ccc5[nH]c6ccccc6c5c4)ccc32)cc1] |
| A48 | 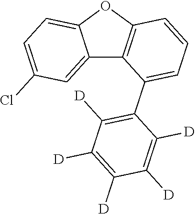 [SMILES: [2H]c1c([2H])c([2H])c(-c2cccc3oc4ccc(Cl)cc4c23)c([2H])c1[2H]] | 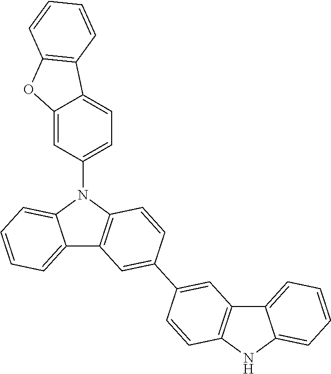 [SMILES: c1ccc2c(c1)[nH]c1ccc(-c3ccc4c(c3)c3ccccc3n4-c3ccc4c(c3)oc3ccccc34)cc12] |
| A51 | 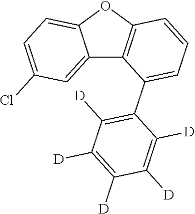 [SMILES: [2H]c1c([2H])c([2H])c(-c2cccc3oc4ccc(Cl)cc4c23)c([2H])c1[2H]] | 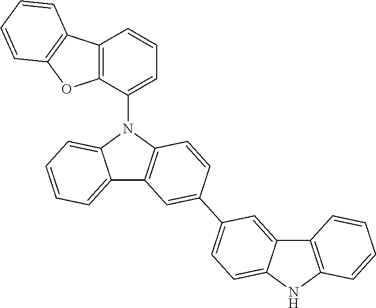 [SMILES: c1ccc2c(c1)[nH]c1ccc(-c3ccc4c(c3)c3ccccc3n4-c3cccc4c3oc3ccccc34)cc12] |
| A52 | 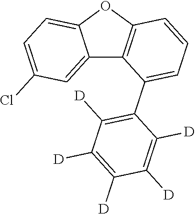 [SMILES: [2H]c1c([2H])c([2H])c(-c2cccc3oc4ccc(Cl)cc4c23)c([2H])c1[2H]] | 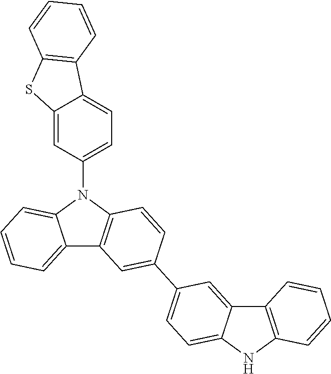 [SMILES: c1ccc2c(c1)[nH]c1ccc(-c3ccc4c(c3)c3ccccc3n4-c3ccc4c(c3)sc3ccccc34)cc12] |
| A63 | 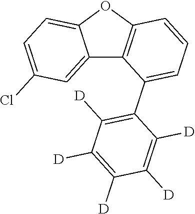 [SMILES: [2H]c1c([2H])c([2H])c(-c2cccc3oc4ccc(Cl)cc4c23)c([2H])c1[2H]] | 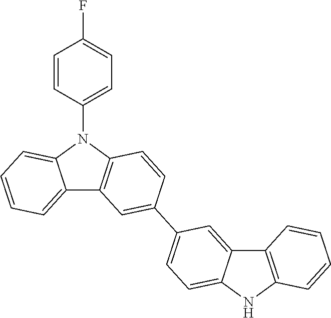 [SMILES: Fc1ccc(-n2c3ccccc3c3cc(-c4ccc5[nH]c6ccccc6c5c4)ccc32)cc1] |
| A68 | 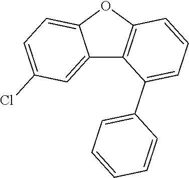 [SMILES: Clc1ccc2oc3cccc(-c4ccccc4)c3c2c1] | 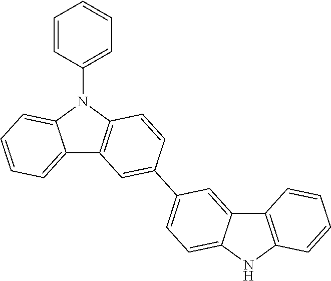 [SMILES: c1ccc(-n2c3ccccc3c3cc(-c4ccc5[nH]c6ccccc6c5c4)ccc32)cc1] |
| A71 | 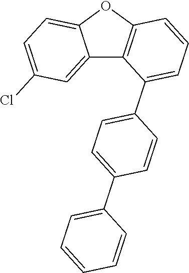 [SMILES: Clc1ccc2oc3cccc(-c4ccc(-c5ccccc5)cc4)c3c2c1] | 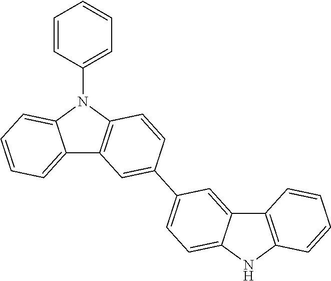 [SMILES: c1ccc(-n2c3ccccc3c3cc(-c4ccc5[nH]c6ccccc6c5c4)ccc32)cc1] |
| A30 | 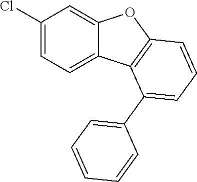 [SMILES: Clc1ccc2c(c1)oc1cccc(-c3ccccc3)c12] | 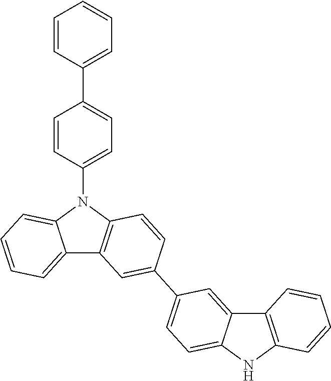 [SMILES: c1ccc(-c2ccc(-n3c4ccccc4c4cc(-c5ccc6[nH]c7ccccc7c6c5)ccc43)cc2)cc1] |
| B1 | 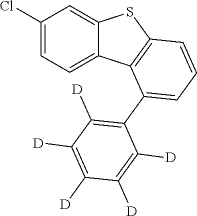 [SMILES: [2H]c1c([2H])c([2H])c(-c2cccc3sc4cc(Cl)ccc4c23)c([2H])c1[2H]] | 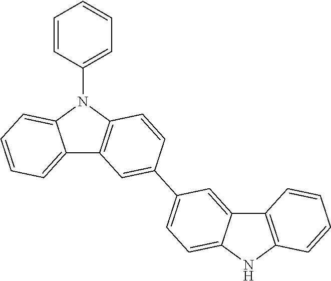 [SMILES: c1ccc(-n2c3ccccc3c3cc(-c4ccc5[nH]c6ccccc6c5c4)ccc32)cc1] |
| B34 | 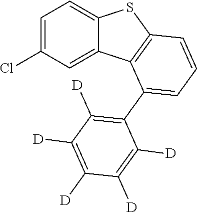 [SMILES: [2H]c1c([2H])c([2H])c(-c2cccc3sc4ccc(Cl)cc4c23)c([2H])c1[2H]] | 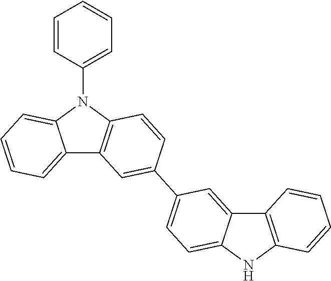 [SMILES: c1ccc(-n2c3ccccc3c3cc(-c4ccc5[nH]c6ccccc6c5c4)ccc32)cc1] |
| C1 | 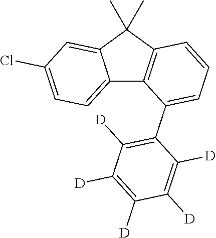 [SMILES: [2H]c1c([2H])c([2H])c(-c2cccc3c2-c2ccc(Cl)cc2C3(C)C)c([2H])c1[2H]] | 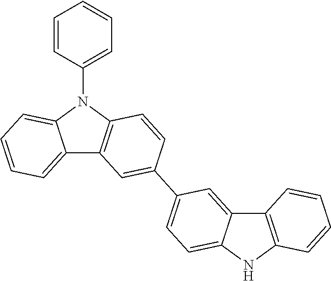 [SMILES: c1ccc(-n2c3ccccc3c3cc(-c4ccc5[nH]c6ccccc6c5c4)ccc32)cc1] |
| A116 | 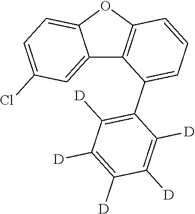 [SMILES: [2H]c1c([2H])c([2H])c(-c2cccc3oc4ccc(Cl)cc4c23)c([2H])c1[2H]] | 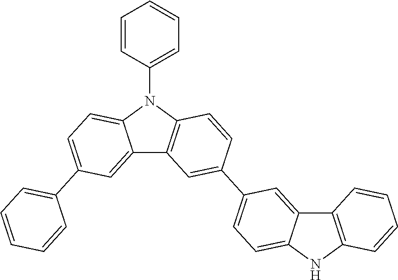 [SMILES: c1ccc(-c2ccc3c(c2)c2cc(-c4ccc5[nH]c6ccccc6c5c4)ccc2n3-c2ccccc2)cc1] |
| A117 | 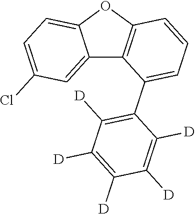 [SMILES: [2H]c1c([2H])c([2H])c(-c2cccc3oc4ccc(Cl)cc4c23)c([2H])c1[2H]] | 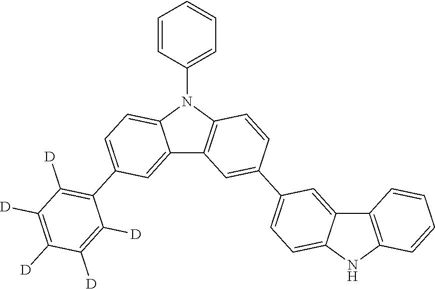 [SMILES: [2H]c1c([2H])c([2H])c(-c2ccc3c(c2)c2cc(-c4ccc5[nH]c6ccccc6c5c4)ccc2n3-c2ccccc2)c([2H])c1[2H]] |
| A111 | 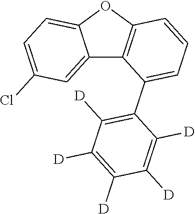 [SMILES: [2H]c1c([2H])c([2H])c(-c2cccc3oc4ccc(Cl)cc4c23)c([2H])c1[2H]] | 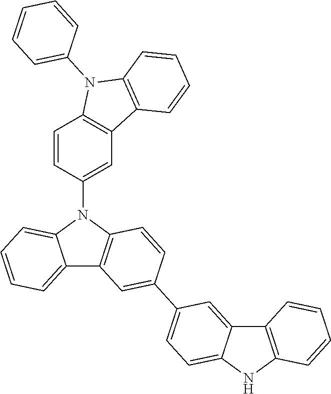 [SMILES: c1ccc(-n2c3ccccc3c3cc(-n4c5ccccc5c5cc(-c6ccc7[nH]c8ccccc8c7c6)ccc54)ccc32)cc1] |
| A88 | 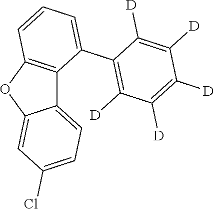 [SMILES: [2H]c1c([2H])c([2H])c(-c2cccc3oc4cc(Cl)ccc4c23)c([2H])c1[2H]] | 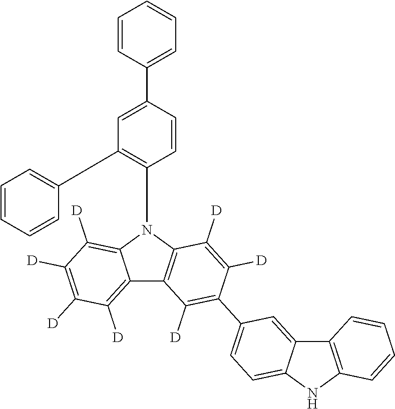 [SMILES: [2H]c1c([2H])c([2H])c2c(c1[2H])c1c([2H])c(-c3ccc4[nH]c5ccccc5c4c3)c([2H])c([2H])c1n2-c1ccc(-c2ccccc2)cc1-c1ccccc1] |
| A64 | 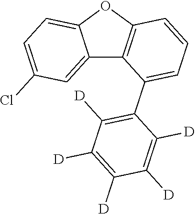 [SMILES: [2H]c1c([2H])c([2H])c(-c2cccc3oc4ccc(Cl)cc4c23)c([2H])c1[2H]] | 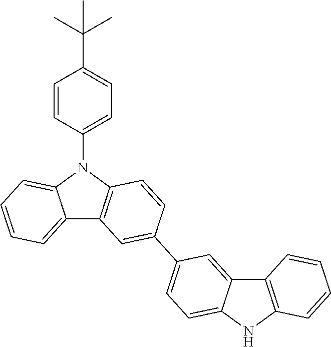 [SMILES: CC(C)(C)c1ccc(-n2c3ccccc3c3cc(-c4ccc5[nH]c6ccccc6c5c4)ccc32)cc1] |

| Compound | Structure | Yield (%) |
|---|---|---|
| A4 | 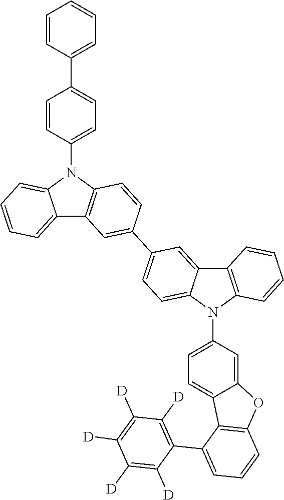 [SMILES: [2H]c1c([2H])c([2H])c(-c2cccc3oc4cc(-n5c6ccccc6c6cc(-c7ccc8c(c7)c7ccccc7n8-c7ccc(-c8ccccc8)cc7)ccc65)ccc4c23)c([2H])c1[2H]] | 71 |
| A5 | 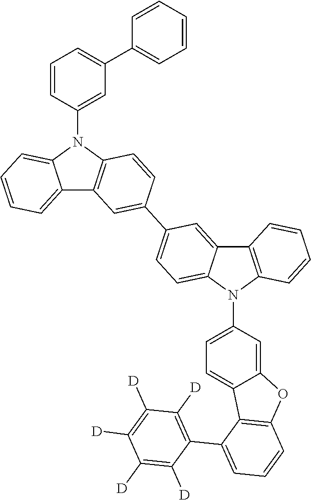 [SMILES: [2H]c1c([2H])c([2H])c(-c2cccc3oc4cc(-n5c6ccccc6c6cc(-c7ccc8c(c7)c7ccccc7n8-c7cccc(-c8ccccc8)c7)ccc65)ccc4c23)c([2H])c1[2H]] | 62 |
| A6 | 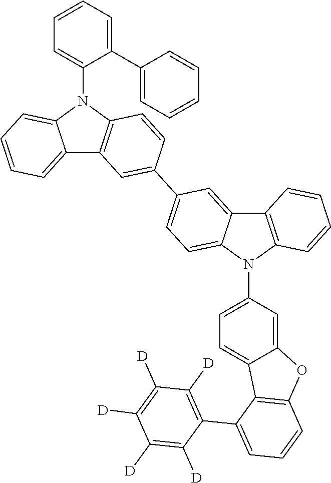 [SMILES: [2H]c1c([2H])c([2H])c(-c2cccc3oc4cc(-n5c6ccccc6c6cc(-c7ccc8c(c7)c7ccccc7n8-c7ccccc7-c7ccccc7)ccc65)ccc4c23)c([2H])c1[2H]] | 60 |
| A3 | 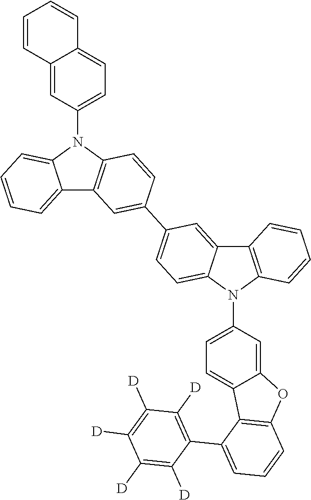 [SMILES: [2H]c1c([2H])c([2H])c(-c2cccc3oc4cc(-n5c6ccccc6c6cc(-c7ccc8c(c7)c7ccccc7n8-c7ccc8ccccc8c7)ccc65)ccc4c23)c([2H])c1[2H]] | 68 |
| A8 | 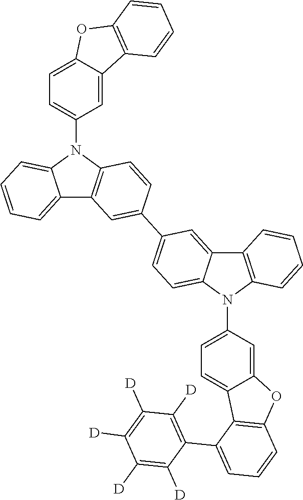 [SMILES: [2H]c1c([2H])c([2H])c(-c2cccc3oc4cc(-n5c6ccccc6c6cc(-c7ccc8c(c7)c7ccccc7n8-c7ccc8oc9ccccc9c8c7)ccc65)ccc4c23)c([2H])c1[2H]] | 58 |
| A42 | 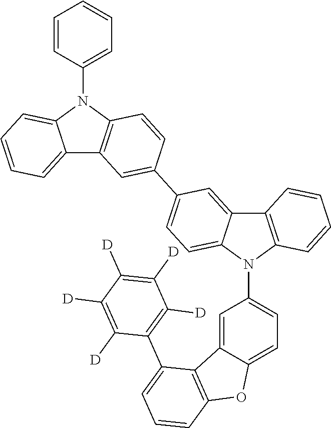 [SMILES: [2H]c1c([2H])c([2H])c(-c2cccc3oc4ccc(-n5c6ccccc6c6cc(-c7ccc8c(c7)c7ccccc7n8-c7ccccc7)ccc65)cc4c23)c([2H])c1[2H]] | 75 |
| A45 | 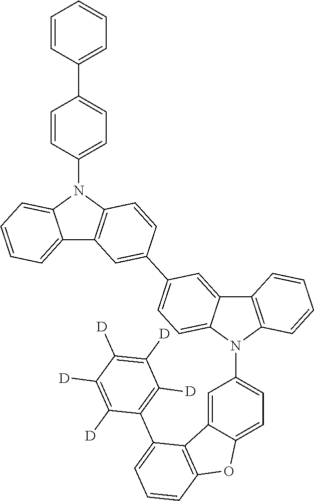 [SMILES: [2H]c1c([2H])c([2H])c(-c2cccc3oc4ccc(-n5c6ccccc6c6cc(-c7ccc8c(c7)c7ccccc7n8-c7ccc(-c8ccccc8)cc7)ccc65)cc4c23)c([2H])c1[2H]] | 66 |
| A46 | 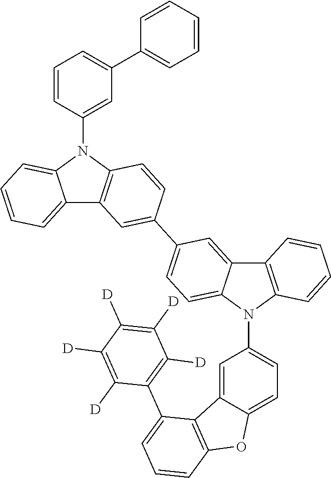 [SMILES: [2H]c1c([2H])c([2H])c(-c2cccc3oc4ccc(-n5c6ccccc6c6cc(-c7ccc8c(c7)c7ccccc7n8-c7cccc(-c8ccccc8)c7)ccc65)cc4c23)c([2H])c1[2H]] | 64 |
| A49 | 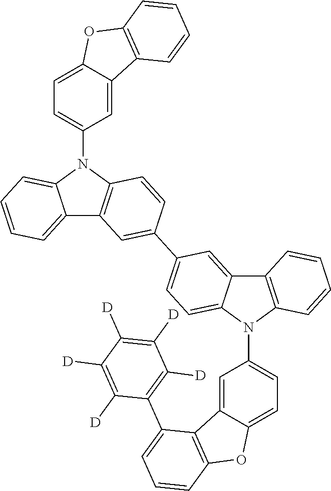 [SMILES: [2H]c1c([2H])c([2H])c(-c2cccc3oc4ccc(-n5c6ccccc6c6cc(-c7ccc8c(c7)c7ccccc7n8-c7ccc8oc9ccccc9c8c7)ccc65)cc4c23)c([2H])c1[2H]] | 61 |
| A62 | 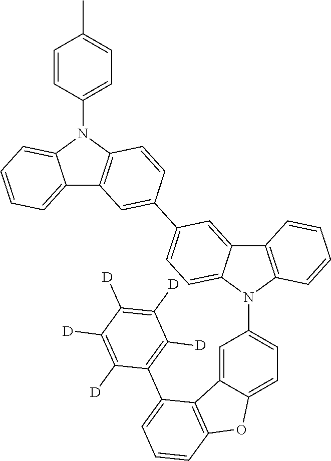 [SMILES: [2H]c1c([2H])c([2H])c(-c2cccc3oc4ccc(-n5c6ccccc6c6cc(-c7ccc8c(c7)c7ccccc7n8-c7ccc(C)cc7)ccc65)cc4c23)c([2H])c1[2H]] | 57 |
| A48 | 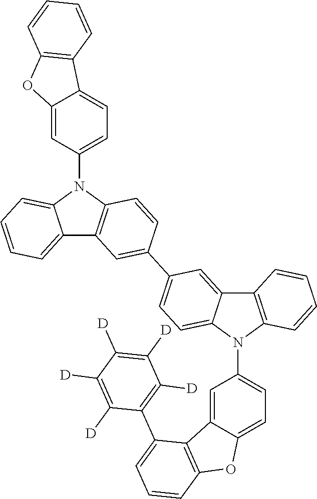 [SMILES: [2H]c1c([2H])c([2H])c(-c2cccc3oc4ccc(-n5c6ccccc6c6cc(-c7ccc8c(c7)c7ccccc7n8-c7ccc8c(c7)oc7ccccc78)ccc65)cc4c23)c([2H])c1[2H]] | 61 |
| A51 | 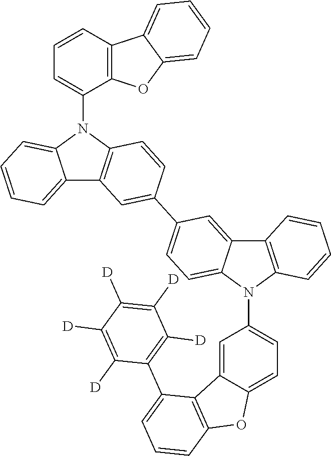 [SMILES: [2H]c1c([2H])c([2H])c(-c2cccc3oc4ccc(-n5c6ccccc6c6cc(-c7ccc8c(c7)c7ccccc7n8-c7cccc8c7oc7ccccc78)ccc65)cc4c23)c([2H])c1[2H]] | 58 |
| A52 | 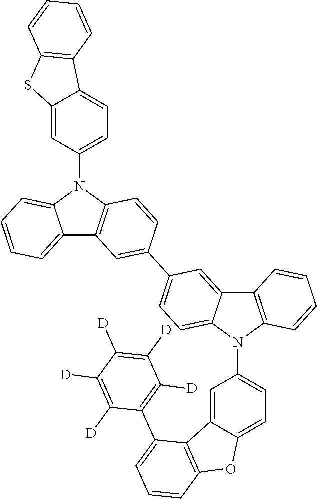 [SMILES: [2H]c1c([2H])c([2H])c(-c2cccc3oc4ccc(-n5c6ccccc6c6cc(-c7ccc8c(c7)c7ccccc7n8-c7ccc8c(c7)sc7ccccc78)ccc65)cc4c23)c([2H])c1[2H]] | 56 |
| A63 | 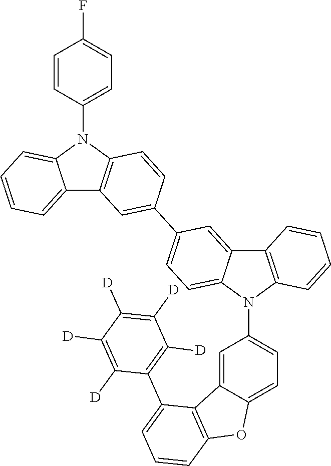 [SMILES: [2H]c1c([2H])c([2H])c(-c2cccc3oc4ccc(-n5c6ccccc6c6cc(-c7ccc8c(c7)c7ccccc7n8-c7ccc(F)cc7)ccc65)cc4c23)c([2H])c1[2H]] | 61 |
| A68 | 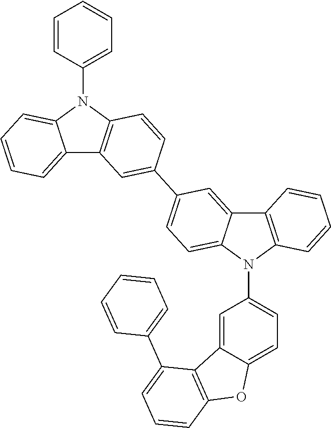 [SMILES: c1ccc(-c2cccc3oc4ccc(-n5c6ccccc6c6cc(-c7ccc8c(c7)c7ccccc7n8-c7ccccc7)ccc65)cc4c23)cc1] | 68 |
| A71 | 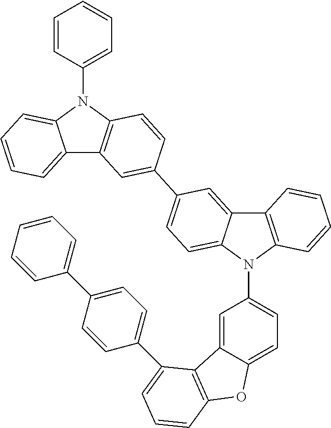 [SMILES: c1ccc(-c2ccc(-c3cccc4oc5ccc(-n6c7ccccc7c7cc(-c8ccc9c(c8)c8ccccc8n9-c8ccccc8)ccc76)cc5c34)cc2)cc1] | 55 |
| A30 | 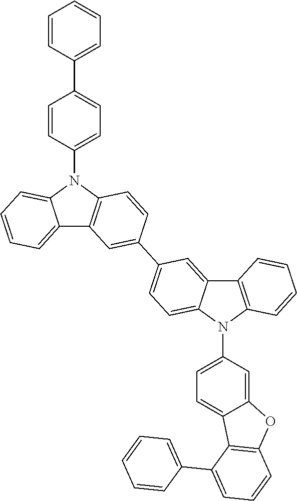 [SMILES: c1ccc(-c2ccc(-n3c4ccccc4c4cc(-c5ccc6c(c5)c5ccccc5n6-c5ccc6c(c5)oc5cccc(-c7ccccc7)c56)ccc43)cc2)cc1] | 62 |
| B1 | 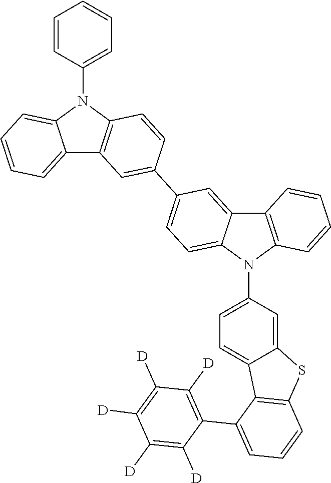 [SMILES: [2H]c1c([2H])c([2H])c(-c2cccc3sc4cc(-n5c6ccccc6c6cc(-c7ccc8c(c7)c7ccccc7n8-c7ccccc7)ccc65)ccc4c23)c([2H])c1[2H]] | 67 |
| B34 | 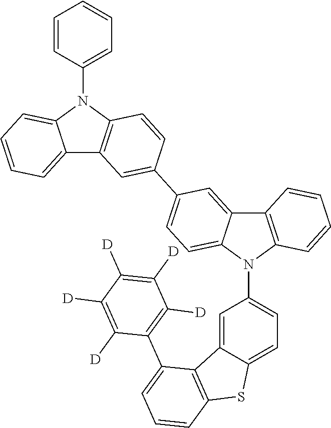 [SMILES: [2H]c1c([2H])c([2H])c(-c2cccc3sc4ccc(-n5c6ccccc6c6cc(-c7ccc8c(c7)c7ccccc7n8-c7ccccc7)ccc65)cc4c23)c([2H])c1[2H]] | 69 |
| C1 | 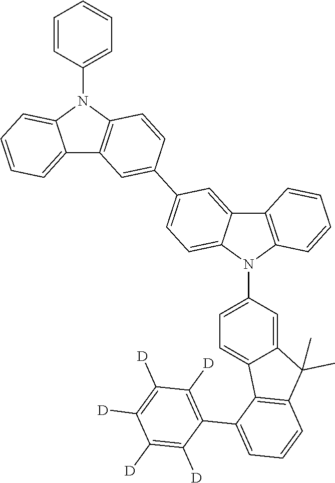 [SMILES: [2H]c1c([2H])c([2H])c(-c2cccc3c2-c2ccc(-n4c5ccccc5c5cc(-c6ccc7c(c6)c6ccccc6n7-c6ccccc6)ccc54)cc2C3(C)C)c([2H])c1[2H]] | 67 |
| A116 | 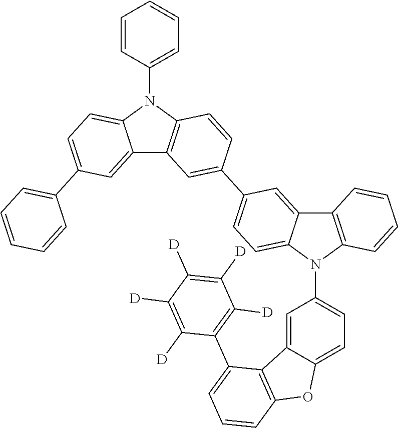 [SMILES: [2H]c1c([2H])c([2H])c(-c2cccc3oc4ccc(-n5c6ccccc6c6cc(-c7ccc8c(c7)c7cc(-c9ccccc9)ccc7n8-c7ccccc7)ccc65)cc4c23)c([2H])c1[2H]] | 54 |
| A117 | 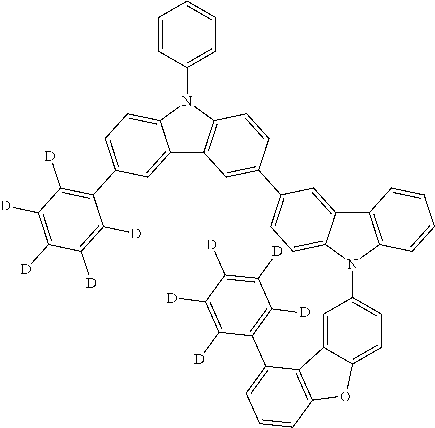 [SMILES: [2H]c1c([2H])c([2H])c(-c2ccc3c(c2)c2cc(-c4ccc5c(c4)c4ccccc4n5-c4ccc5oc6cccc(-c7c([2H])c([2H])c([2H])c([2H])c7[2H])c6c5c4)ccc2n3-c2ccccc2)c([2H])c1[2H]] | 60 |
| A111 | 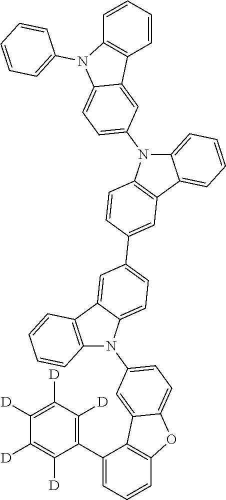 | 59 |
| A88 | 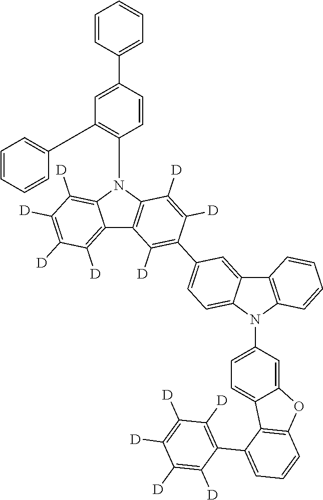 [SMILES: [2H]c1c([2H])c([2H])c(-c2cccc3oc4cc(-n5c6ccccc6c6cc(-c7c([2H])c([2H])c8c(c7[2H])c7c([2H])c([2H])c([2H])c([2H])c7n8-c7ccc(-c8ccccc8)cc7-c7ccccc7)ccc65)ccc4c23)c([2H])c1[2H]] | 63 |
| A64 | 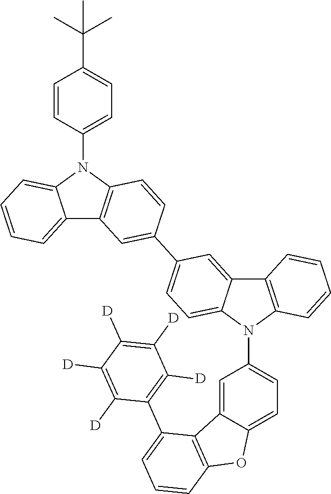 [SMILES: [2H]c1c([2H])c([2H])c(-c2cccc3oc4ccc(-n5c6ccccc6c6cc(-c7ccc8c(c7)c7ccccc7n8-c7ccc(C(C)(C)C)cc7)ccc65)cc4c23)c([2H])c1[2H]] | 57 |

### Synthesis of an Intermediate Sub 1-I-B1

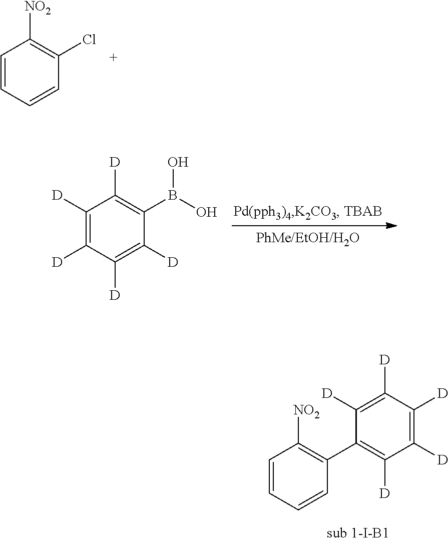 [SMILES: O=[N+]([O-])c1ccccc1Cl ; [2H]c1c([2H])c([2H])c(B(O)O)c([2H])c1[2H] ; [2H]c1c([2H])c([2H])c(-c2ccccc2[N+](=O)[O-])c([2H])c1[2H]]

o-Chloronitrobenzene (50.0 g, 317.3 mmol), deuterated benzeneboronic acid (40.2 g, 317.3 mmol), tetrakis(triphenylphosphine) palladium (3.6 g, 3.1 mmol), potassium carbonate (87.7 g, 634.7 mmol) and tetrabutylammonium bromide (1.0 g, 3.1 mmol) were added into a three-necked flask, toluene (400 mL), ethanol (200 mL) and deionized water (100 mL) were added into the three-necked flask, and the mixture was heated to 76° C., and stirred with heating under reflux for 18 h under nitrogen protection. The reaction solution was cooled to room temperature, stopped stirring, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain the intermediate sub 1-I-B1 (42.7 g, 66%).

### Synthesis of an Intermediate Sub 1-II-B1

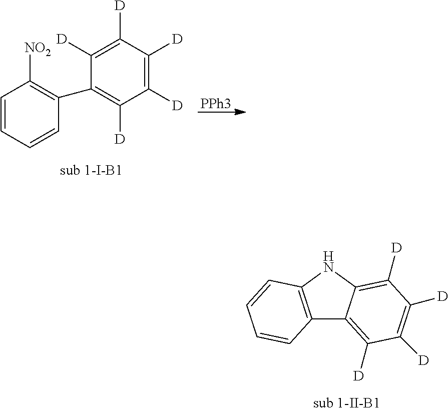 [SMILES: [2H]c1c([2H])c([2H])c(-c2ccccc2[N+](=O)[O-])c([2H])c1[2H] ; [2H]c1c([2H])c([2H])c2c([nH]c3ccccc32)c1[2H] ; "PPh3"]

The intermediate sub 1-I-B1 (40.0 g, 195.8 mmol), triphenylphosphine (102.7 g, 391.6 mmol) and o-dichlorobenzene (400 mL) were added into a three-necked flask, and the mixture was heated to 150° C., and stirred with heating under reflux for 18 h under nitrogen protection. The reaction solution was Cooled to room temperature, stopped stirring, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain the intermediate sub 1-II-B1 (19.1 g, 57%).

### Synthesis of an Intermediate Sub 1-III-B1

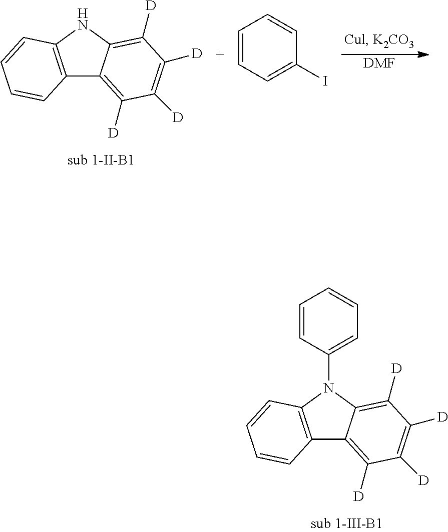

Sub 1-I-B1 (19.0 g, 110.9 mmol), iodobenzene (22.6 g, 110.9 mmol), CuI (4.2 g, 22.1 mmol), K₂CO₃ (33.7 g, 244.1 mmol) and 18-crown-6 (7.9 g, 44.3 mmol) were added into a three-necked flask, and a solvent of dried DMF (200 mL) was added, under nitrogen protection, the mixture was heated to 150° C., and stirred for 17 h while maintaining the temperature; the reaction solution was cooled to room temperature, stopped stirring, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain the intermediate sub 1-III-B1 (21.4 g, 78%).

### Synthesis of an Intermediate Sub 1-IV-B1

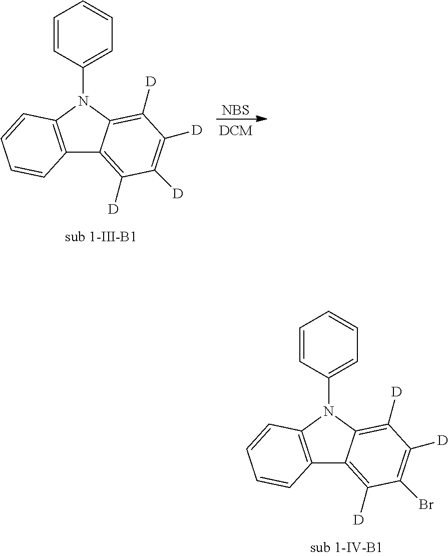

The intermediate sub 1-III-B1 (20.0 g, 80.8 mmol) and dichloromethane (200 mL) were added into a three-necked flask, and N-bromosuccinimide (NBS) (12.9 g, 72.7 mmol) was added under nitrogen protection. A reaction was carried out under stirring overnight, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain the intermediate sub 1-IV-B1 (14.7 g, 56%).

### Synthesis of an Intermediate Sub B-12

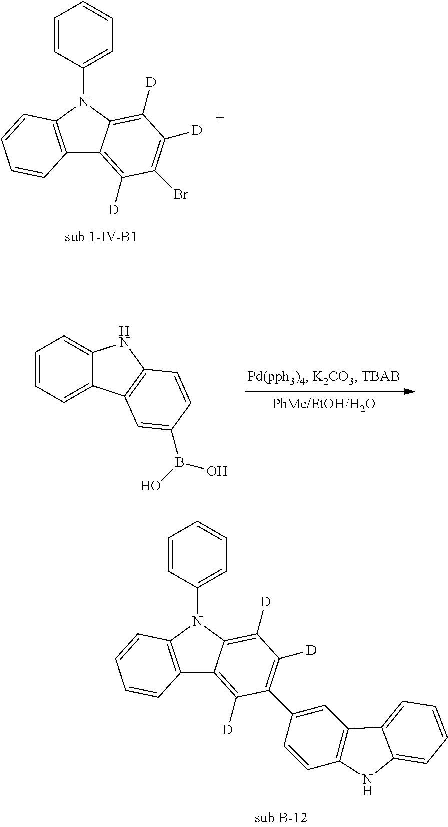 [SMILES: [2H]c1c(Br)c([2H])c2c3ccccc3n(-c3ccccc3)c2c1[2H] ; OB(O)c1ccc2[nH]c3ccccc3c2c1 ; [2H]c1c(-c2ccc3[nH]c4ccccc4c3c2)c([2H])c2c3ccccc3n(-c3ccccc3)c2c1[2H]]

sub 1-IV-B1 (14.0 g, 43.0 mmol), 9H-carbazol-3-ylboronic acid (9.2 g, 43.9 mmol), tetrakis(triphenylphosphine) palladium (0.5 g, 0.4 mmol), potassium carbonate (11.8 g, 86.0 mmol) and tetrabutylammonium bromide (0.1 g, 0.4 mmol) were added into a three-necked flask, toluene (112 mL), ethanol (28 mL) and deionized water (28 mL) were added into the three-necked flask, and the mixture was heated to 76° C., and stirred with heating under reflux for 18 h under nitrogen protection. The reaction solution was cooled to room temperature, stopped stirring, the reaction solution was washed with water, an organic phase was separated, and dried over anhydrous magnesium sulfate, and a solvent was removed under reduced pressure to obtain a crude product; and the crude product was purified by silica gel column chromatography using dichloromethane/n-heptane as a mobile phase to obtain the intermediate sub B-12 (12.2 g, 69%).

### Synthesis of a Compound A104

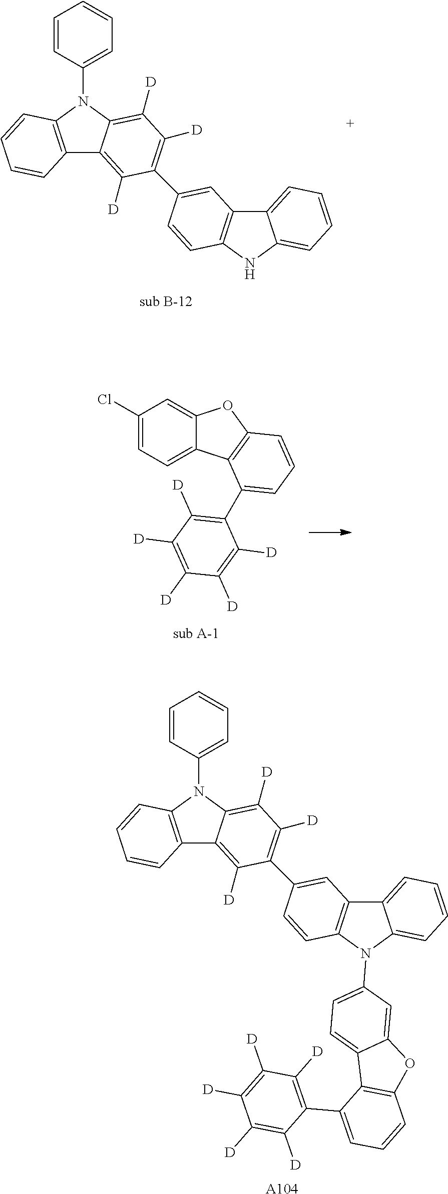

sub B-12 (12.0 g, 29.1 mmol), sub A-1 (8.6 g, 30.6 mmol), tris(dibenzylideneacetone) dipalladium (0.2 g, 0.2 mmol), 2-dicyclohexylphosphino-2′,4′,6′-triisopropylbiphenyl (0.2 g, 0.5 mmol) and sodium tert-butoxide (5.6 g, 58.3 mmol) were added to xylene (120 mL), and the mixture was heated to 140° C. and stirred for 4 h under nitrogen protection. Then, the reaction solution was cooled to room temperature, the reaction solution was washed with water, dried by adding magnesium sulfate, and filtered, a solvent was removed from the obtained filtrate under reduced pressure; and a crude product was purified by recrystallization using a dichloromethane/n-heptane system to obtain the compound A104 (12.2 g, yield: 64%). Mass spectrum: m/z=659.29 [M+H]⁺.

Mass spectrum data for some compounds are shown in Table 4 below.

TABLE 4

| | | | |
|---|---|---|---|
| Compound A1 | m/z = 656.27 [M + H]⁺ | Compound A51 | m/z = 746.28 [M + H]⁺ |
| Compound A4 | m/z = 732.31 [M + H]⁺ | Compound A52 | m/z = 762.26 [M + H]⁺ |
| Compound A5 | m/z = 732.30 [M + H]⁺ | Compound A63 | m/z = 674.27 [M + H]⁺ |
| Compound A6 | m/z = 732.30 [M + H]⁺ | Compound A68 | m/z = 651.24 [M + H]⁺ |
| Compound A3 | m/z = 706.29 [M + H]⁺ | Compound A71 | m/z = 727.27 [M + H]⁺ |
| Compound A8 | m/z = 746.29 [M + H]⁺ | Compound A30 | m/z = 727.27 [M + H]⁺ |
| Compound A42 | m/z = 656.27 [M + H]⁺ | Compound A104 | m/z = 659.30 [M + H]⁺ |
| Compound A45 | m/z = 732.31 [M + H]⁺ | Compound B1 | m/z = 672.25 [M + H]⁺ |
| Compound A46 | m/z = 732.30 [M + H]⁺ | Compound B34 | m/z = 672.24 [M + H]⁺ |
| Compound A49 | m/z = 746.29 [M + H]⁺ | Compound C1 | m/z = 682.33 [M + H]⁺ |
| Compound A62 | m/z = 670.29 [M + H]⁺ | Compound A116 | m/z = 732.31 [M + H]⁺ |
| Compound A48 | m/z = 746.28 [M + H]⁺ | Compound A117 | m/z = 732.34 [M + H]⁺ |
| Compound A111 | m/z = 821.33 [M + H]⁺ | Compound A88 | m/z = 815.38 [M + H]⁺ |
| Compound A64 | m/z = 712.34 [M + H]⁺ | | |

NMR data of some compounds are shown in Table 5 below

TABLE 5

| Compound | NMR data |
|---|---|
| A1 | ¹HNMR (400 MHZ, CD₂Cl₂): δ8.49 (d, 2H), δ8.23 (t, 2H), |
| | δ7.76-7.87 (m, 3H), δ7.58-7.70 (m, 8H), δ7.56 (d, 1H), |
| | δ7.38-7.54 (m, 6H), δ7.26-7.36 (m, 2H), δ7.20 (t, 1H). |
| A42 | ¹HNMR (400 MHZ, CD₂Cl₂): δ8.47 (d, 2H), δ8.22 (t, 2H), |
| | δ7.77-7.84 (m, 3H), δ7.73 (d, 1H), 7.55-7.68 (m, 7H), |
| | δ7.35-7.54 (m, 7H), δ7.26-7.33 (m, 3H). |

Manufacture and evaluation of organic electroluminescent device

### Example 1: Manufacture of Green Organic Electroluminescent Device

An anode was prepared by the following processes: an ITO substrate with a thickness of 1300 Å (manufactured by Corning) was cut into a size of 40 mm×40 mm×0.7 mm to be prepared into an experimental substrate with a cathode, an anode and an insulating layer pattern by adopting a photoetching process, and surface treatment was performed by utilizing ultraviolet ozone and O₂:N₂ plasma to increase the work function of the anode (the experiment substrate), and remove scum.

HAT-CN was vacuum evaporated on the experimental substrate (the anode) to form a hole injection layer (HIL) having a thickness of 150 Å, and HT-4 was evaporated on the hole injection layer to form a hole transport layer having a thickness of 1052 Å.

HT-20 was vacuum evaporated on the hole transport layer to form a hole auxiliary layer having a thickness of 355 Å.

A compound A1, H52 and Ir(ppy)₂(acac) were co-evaporated on the hole auxiliary layer at a film thickness ratio of 50%:40%:10% to form an organic light-emitting layer (EML) having a thickness of 400 Å.

ET-01 and LiQ were mixed in a weight ratio of 1:1 and evaporated to form an electron transport layer (ETL) having a thickness of 350 Å, LiQ was evaporated on the electron transport layer to form an electron injection layer (EIL) having a thickness of 12 Å, and then magnesium (Mg) and silver (Ag) were vacuum evaporated on the electron injection layer at an evaporation rate of 1:9 to form a cathode having a thickness of 120 Å.

In addition, HT-3 having a thickness of 700 Å was evaporated on the above cathode to form an organic capping layer (CPL), thus completing manufacturing of the organic electroluminescent device.

### Examples 2 to 27

An organic electroluminescent device was manufactured with reference to the method in Example 1 except that compounds shown in Table 6 below were used instead of the compound A1 when the organic light-emitting layer was formed.

### Comparative Examples 1 to 4

An organic electroluminescent device was manufactured with reference to the method in Example 1 except that a compound I, a compound II, a compound III, or a compound IV was used instead of the compound A1 when the organic light-emitting layer was formed.

The structures of materials used in the examples and the comparative examples are shown below:

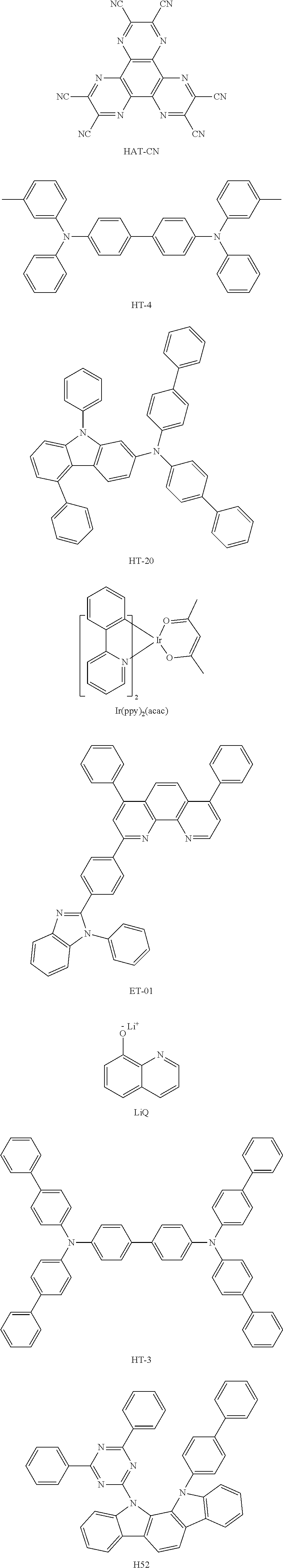

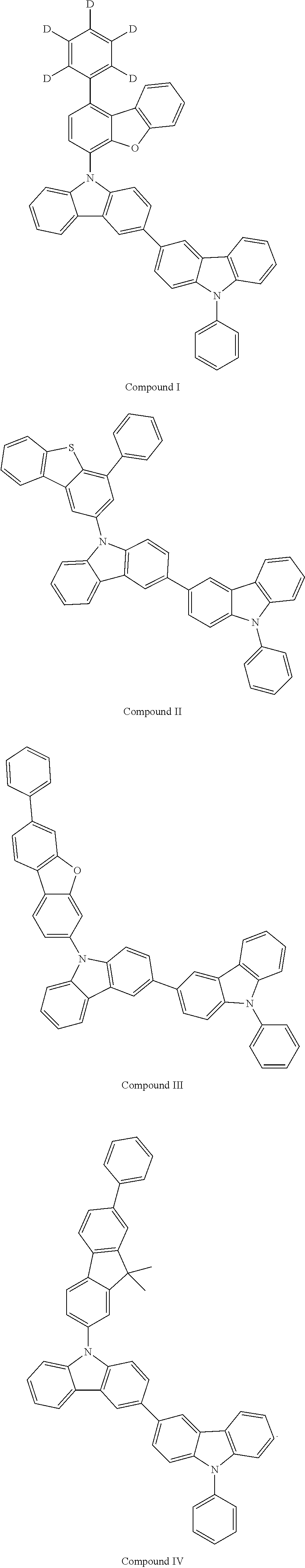 [SMILES: [2H]c1c([2H])c([2H])c(-c2ccc(-n3c4ccccc4c4cc(-c5ccc6c(c5)c5ccccc5n6-c5ccccc5)ccc43)c3oc4ccccc4c23)c([2H])c1[2H] ; c1ccc(-c2cc(-n3c4ccccc4c4cc(-c5ccc6c(c5)c5ccccc5n6-c5ccccc5)ccc43)cc3c2sc2ccccc23)cc1 ; c1ccc(-c2ccc3c(c2)oc2cc(-n4c5ccccc5c5cc(-c6ccc7c(c6)c6ccccc6n7-c6ccccc6)ccc54)ccc23)cc1 ; CC1(C)c2cc(-c3ccccc3)ccc2-c2ccc(-n3c4ccccc4c4cc(-c5ccc6c(c5)c5ccccc5n6-c5ccccc5)ccc43)cc21]

For the organic electroluminescent devices manufactured above, the performance of the devices was analyzed under the condition of 15 mA/cm², and the results are shown in Table 6 below:

TABLE 6

| | | Driving | Current | Chromaticity | Chromaticity | T95 (h) |
|---|---|---|---|---|---|---|
| | | voltage | efficiency | coordinate | coordinate | 15 |
| Example No. | Compound | (V) | (Cd/A) | CIEx | CIEy | mA/cm² |
| Example 1 | Compound | 4.01 | 80.59 | 0.221 | 0.73 | 320 |
| | A1 | | | | | |
| Example 2 | Compound | 4.03 | 80.22 | 0.221 | 0.73 | 318 |
| | A3 | | | | | |
| Example 3 | Compound | 4.04 | 79.41 | 0.221 | 0.73 | 342 |
| | A4 | | | | | |
| Example 4 | Compound | 4.04 | 79.07 | 0.221 | 0.73 | 326 |
| | A5 | | | | | |
| Example 5 | Compound | 4.00 | 79.19 | 0.221 | 0.73 | 335 |
| | A6 | | | | | |
| Example 6 | Compound | 3.95 | 80.89 | 0.221 | 0.73 | 347 |
| | A8 | | | | | |
| Example 7 | Compound | 3.89 | 83.32 | 0.221 | 0.73 | 352 |
| | A42 | | | | | |
| Example 8 | Compound | 3.85 | 83.95 | 0.221 | 0.73 | 351 |
| | A45 | | | | | |
| Example 9 | Compound | 3.91 | 82.12 | 0.221 | 0.73 | 356 |
| | A46 | | | | | |
| Example 10 | Compound | 3.84 | 82.62 | 0.221 | 0.73 | 330 |
| | A49 | | | | | |
| Example 11 | Compound | 3.87 | 82.01 | 0.221 | 0.73 | 360 |
| | A62 | | | | | |
| Example 12 | Compound | 4.01 | 81.75 | 0.221 | 0.73 | 328 |
| | A48 | | | | | |
| Example 13 | Compound | 4.02 | 79.60 | 0.221 | 0.73 | 338 |
| | A51 | | | | | |
| Example 14 | Compound | 3.99 | 81.02 | 0.221 | 0.73 | 323 |
| | A52 | | | | | |
| Example 15 | Compound | 4.02 | 79.76 | 0.221 | 0.73 | 345 |
| | A63 | | | | | |
| Example 16 | Compound | 3.98 | 78.70 | 0.221 | 0.73 | 305 |
| | A68 | | | | | |
| Example 17 | Compound | 4.05 | 79.75 | 0.221 | 0.73 | 307 |
| | A71 | | | | | |
| Example 18 | Compound | 3.99 | 78.83 | 0.221 | 0.73 | 300 |
| | A30 | | | | | |
| Example 19 | Compound | 4.03 | 79.89 | 0.221 | 0.73 | 337 |
| | A104 | | | | | |
| Example 20 | Compound | 3.95 | 79.18 | 0.221 | 0.73 | 343 |
| | B1 | | | | | |
| Example 21 | Compound | 3.96 | 79.14 | 0.221 | 0.73 | 331 |
| | B34 | | | | | |
| Example 22 | Compound | 4.00 | 74.67 | 0.221 | 0.73 | 295 |
| | C1 | | | | | |
| Example 23 | Compound | 3.82 | 80.05 | 0.221 | 0.73 | 353 |
| | A116 | | | | | |
| Example 24 | Compound | 3.90 | 83.80 | 0.221 | 0.73 | 344 |
| | A117 | | | | | |
| Example 25 | Compound | 3.84 | 80.71 | 0.221 | 0.73 | 339 |
| | A111 | | | | | |
| Example 26 | Compound | 3.93 | 82.90 | 0.221 | 0.73 | 346 |
| | A88 | | | | | |
| Example 27 | Compound | 3.87 | 80.74 | 0.221 | 0.73 | 358 |
| | A64 | | | | | |
| Comparative | Compound | 4.13 | 52.30 | 0.221 | 0.73 | 217 |
| example 1 | I | | | | | |
| Comparative | Compound | 4.25 | 51.59 | 0.221 | 0.73 | 220 |
| example 2 | II | | | | | |
| Comparative | Compound | 4.21 | 65.71 | 0.221 | 0.73 | 253 |
| example 3 | III | | | | | |
| Comparative | Compound | 4.17 | 62.57 | 0.221 | 0.73 | 245 |
| example 4 | IV | | | | | |

As can be seen from the above Table 6, Examples 1 to 27 in which the compound of the present disclosure is used as a hole-type host material in a mixed host material of a green light-emitting layer have the advantages that the voltage, current efficiency, and service life of the device are all significantly improved compared with Comparative examples 1 to 4. Specifically, the current efficiency is improved by at least 13.63% and the service life T95 (h) is improved by at least 16.6%. Especially, the device performance is better when a dibenzo five-membered ring is dibenzofuran/dibenzothiophene, and aryl is pentadeuterophenyl.

The compounds of the examples of the present disclosure have the advantages that the current efficiency is improved by at least 42.8%, and the service life T95 (h) is improved by at least 34.1% compared with Comparative examples 1 and 2. The reason may be that aryl (pentadeuterophenyl) and carbazolyl in the compounds in the comparative examples are connected to a same benzene ring in dibenzofuranyl/dibenzothienyl, so that the steric hindrance effect of the compound molecule is weakened, the film-forming properties of the material is reduced, and thus the service life and current efficiency of the device are reduced.

Compared with Comparative examples 3 and 4, the compounds of the examples of the present disclosure have the advantages that the current efficiency is improved by at least 13.63%, and the service life T95 (h) is improved by at least 16.6%. The reason may be that aryl in the organic compound of the present disclosure is connected at a specific position in a dibenzo five-membered ring, and is bonded to 3,3-bicarbazole, so that the compound has enhanced hole mobility and energy transfer efficiency, and is suitable for being used as a hole-type host material in an organic electroluminescent device; and by aryl substitution at the specific position, the steric hindrance effect of the molecule is enhanced, the film-forming properties of the material is improved, so that the efficiency and service life of the device are further improved.

It should be understood that the present disclosure is not limited to the precise structures that have been described above and shown in the drawings, and that various modifications and changes can be made without departing from its scope. The scope of the present disclosure is limited only by the following claims.

1. An organic compound, having a structure shown in a formula 1: 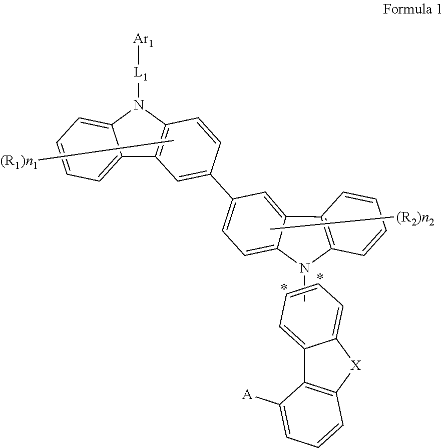
wherein * denotes a connection site;
X is selected from C(R₃R₄), O or S;
a group A is selected from substituted or unsubstituted aryl with 6 to 12 carbon atoms;
L₁ is selected from a single bond, substituted or unsubstituted arylene with 6 to 30 carbon atoms, and substituted or unsubstituted heteroarylene with 3 to 30 carbon atoms;
Ar₁ is selected from substituted or unsubstituted aryl with 6 to 30 carbon atoms, and substituted or unsubstituted heteroaryl with 3 to 30 carbon atoms;
each R₁ and each R₂ are the same or different, and are respectively and independently selected from deuterium, a halogen group, cyano, alkyl with 1 to 10 carbon atoms, deuterated alkyl with 1 to 10 carbon atoms, haloalkyl with 1 to 10 carbon atoms, aryl with 6 to 20 carbon atoms, deuterated aryl with 6 to 20 carbon atoms, and heteroaryl with 3 to 20 carbon atoms;
n₁ is the number of R₁, and is selected from 0, 1, 2, 3, 4, 5, 6 or 7, and when n₁ is greater than 1, any two R₁ are the same or different;
n₂ is the number of R₂, and is selected from 0, 1, 2, 3, 4, 5, 6 or 7, and when n₂ is greater than 1, any two R₂ are the same or different;
R₃ and R₄ are respectively and independently selected from hydrogen, deuterium, alkyl with 1 to 10 carbon atoms or deuterated alkyl with 1 to 10 carbon atoms;
substituents in the group A are the same or different, and are respectively and independently selected from deuterium, a halogen group, cyano, alkyl with 1 to 4 carbon atoms, deuterated alkyl with 1 to 4 carbon atoms, haloalkyl with 1 to 4 carbon atoms or phenyl; and
substituents in L₁ and Ar₁ are the same or different, and are respectively and independently selected from deuterium, a halogen group, cyano, alkyl with 1 to 10 carbon atoms, deuterated alkyl with 1 to 10 carbon atoms, haloalkyl with 1 to 10 carbon atoms, aryl with 6 to 20 carbon atoms, heteroaryl with 3 to 20 carbon atoms, cycloalkyl with 3 to 20 carbon atoms, deuterated aryl with 6 to 20 carbon atoms, haloaryl with 6 to 20 carbon atoms, and triarylsilyl with 18 to 24 carbon atoms.

2. The organic compound according to claim 1, wherein the organic compound has a structure represented by a formula 1-1, a formula 1-2, a formula 1-3, a formula 1-4, a formula 1-5, or a formula 1-6:

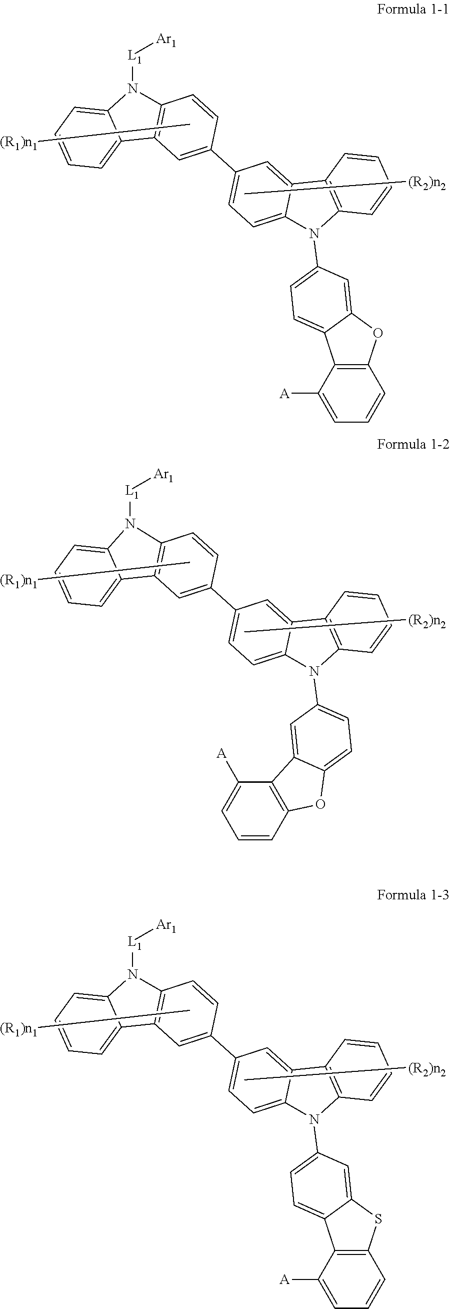

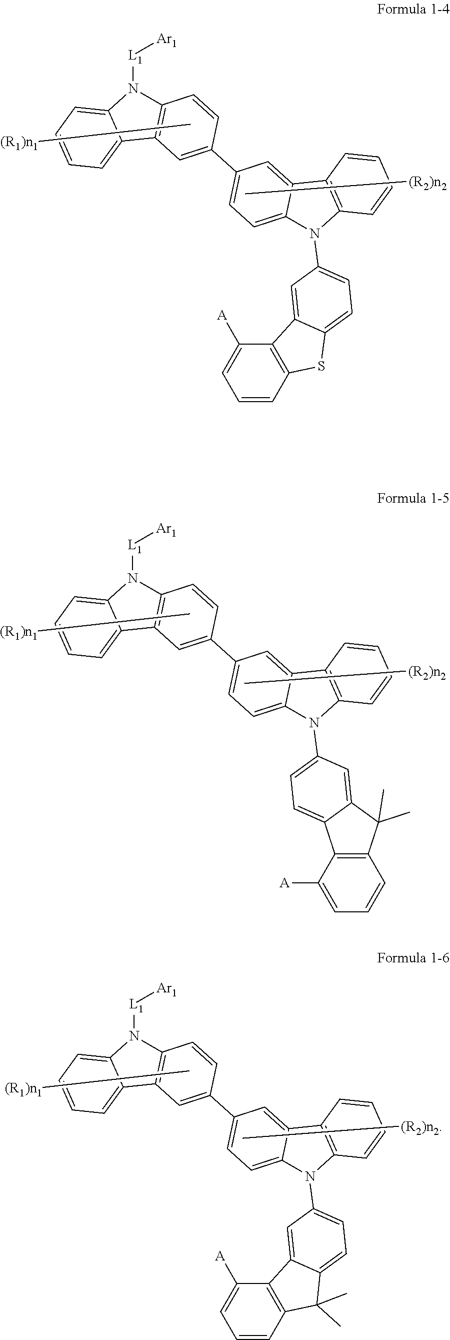

3. The organic compound according to claim 1, wherein the group A is selected from substituted or unsubstituted phenyl, substituted or unsubstituted biphenyl, and substituted or unsubstituted naphthyl; and
preferably, substituents in the group A are the same or different, and are respectively and independently selected from deuterium, fluorine, cyano, methyl, ethyl, n-propyl, tert-butyl or phenyl.

4. The organic compound according to claim 1, wherein the group A is selected from

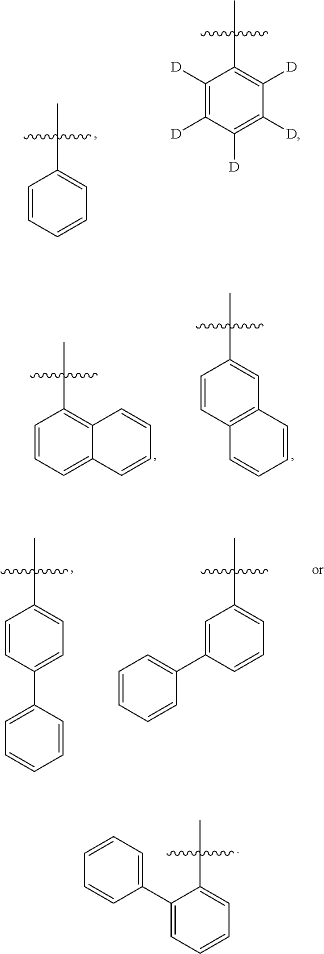

5. The organic compound according to claim 1, wherein L₁ is selected from a single bond, substituted or unsubstituted phenylene, substituted or unsubstituted naphthylene, substituted or unsubstituted biphenylene, substituted or unsubstituted carbazolylene, substituted or unsubstituted dibenzofurylene, and substituted or unsubstituted dibenzothenylene; and
preferably, substituents in L₁ are the same or different, and are respectively and independently selected from deuterium, fluorine, cyano, methyl, ethyl, n-propyl, isopropyl, tert-butyl, phenyl or pentadeuteriophenyl.

6. The organic compound according to claim 1, wherein Ar₁ is selected from substituted or unsubstituted aryl with 6 to 25 carbon atoms, and substituted or unsubstituted heteroaryl with 12 to 20 carbon atoms; and
preferably, substituents in Ar₁ are the same or different, and are respectively and independently selected from deuterium, a halogen group, cyano, alkyl with 1 to 5 carbon atoms, phenyl, or pentadeuterophenyl.

7. The organic compound according to claim 1, wherein Ar₁ is selected from substituted or unsubstituted phenyl, substituted or unsubstituted biphenyl, substituted or unsubstituted naphthyl, substituted or unsubstituted fluorenyl, substituted or unsubstituted terphenyl, substituted or unsubstituted dibenzofuranyl, substituted or unsubstituted dibenzothienyl, and substituted or unsubstituted carbazolyl; and
preferably, substituents in the Ar₁ are the same or different, and are respectively and independently selected from deuterium, fluorine, cyano, methyl, ethyl, n-propyl, isopropyl, tert-butyl, phenyl or pentadeuterophenyl.

8. The organic compound according to claim 1, wherein Ar₁ is selected from substituted or unsubstituted phenanthryl; and
preferably, substituents in Ar₁ are the same or different, and are respectively and independently selected from deuterium, fluorine, cyano, methyl, ethyl, n-propyl, isopropyl, tert-butyl, phenyl or pentadeuterophenyl.

9. The organic compound according to claim 1, wherein 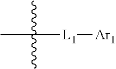
is selected from a substituted or unsubstituted group V, wherein the unsubstituted groups V is selected from the group consisting of: 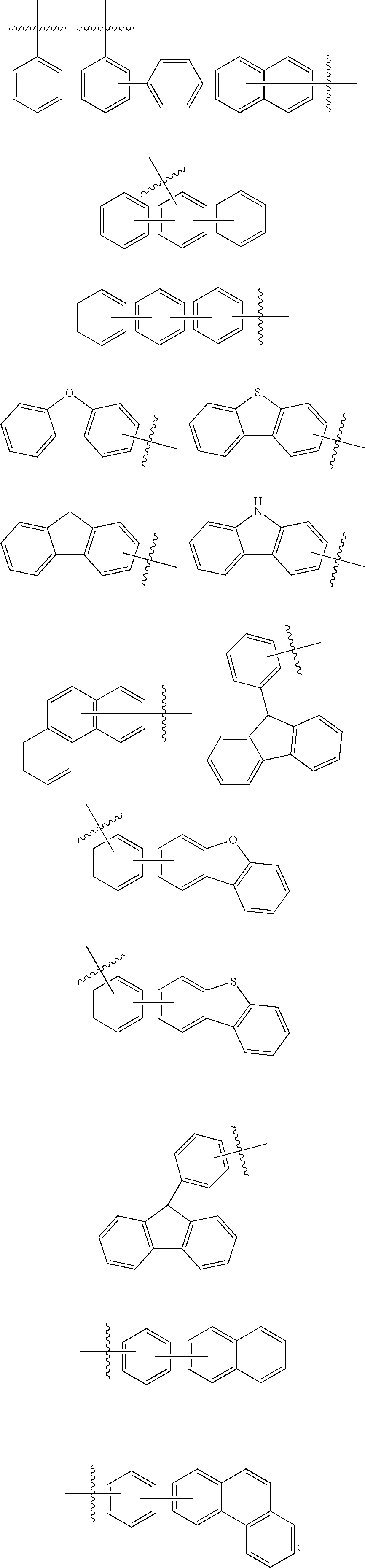
and
the substituted group V has one or two or more substituents, the substituents in substituted group V are respectively and independently selected from the group consisting of deuterium, fluorine, cyano, methyl, ethyl, n-propyl, isopropyl, tert-butyl, phenyl or pentadeuterophenyl, and when the number of the substituents on the group V is greater than 1, the substituents are the same or different.

10. The organic compound according to claim 1, wherein 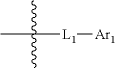
is selected from the group consisting of:

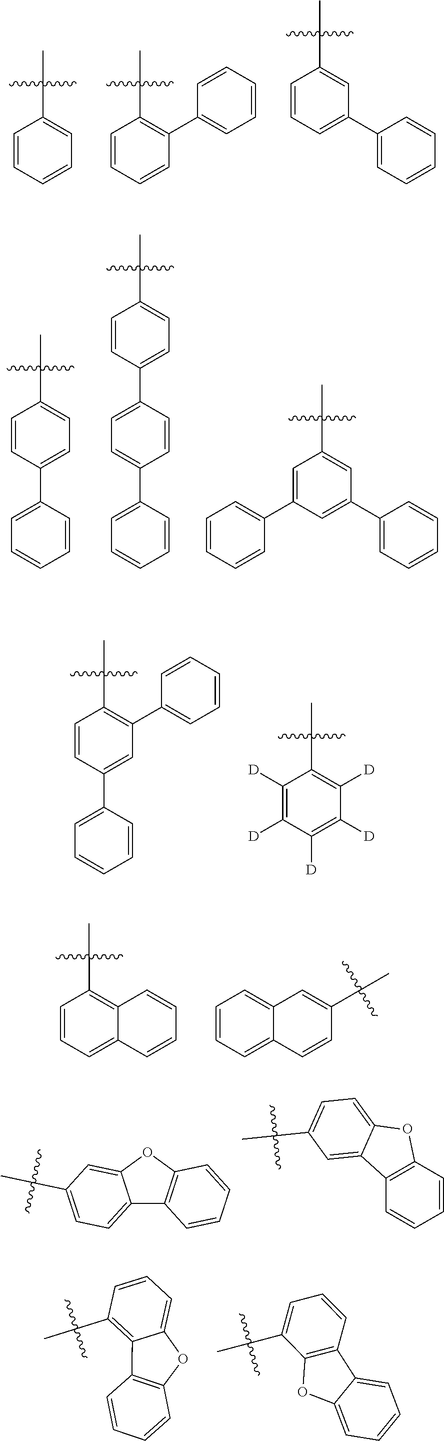

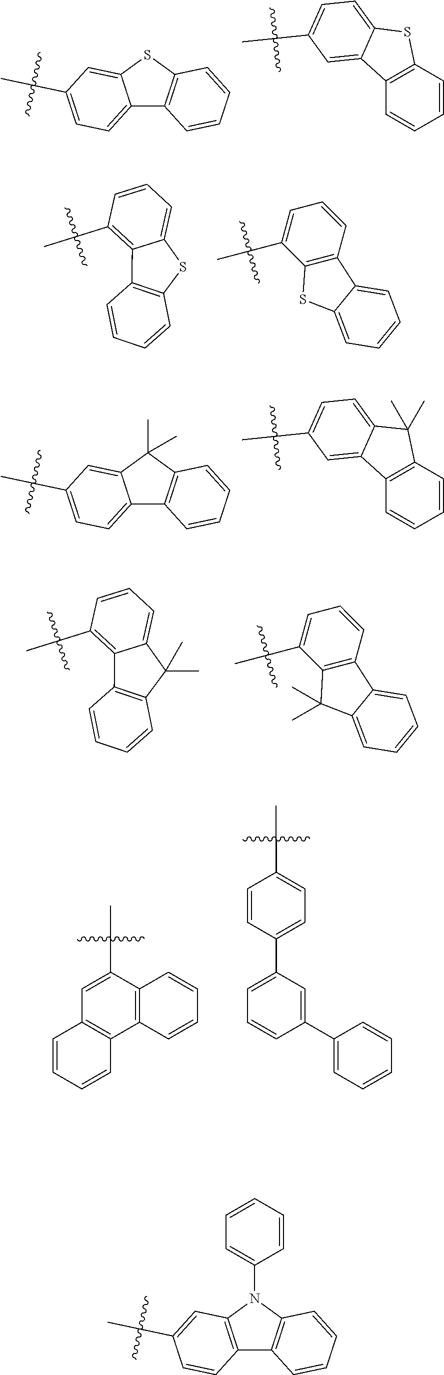

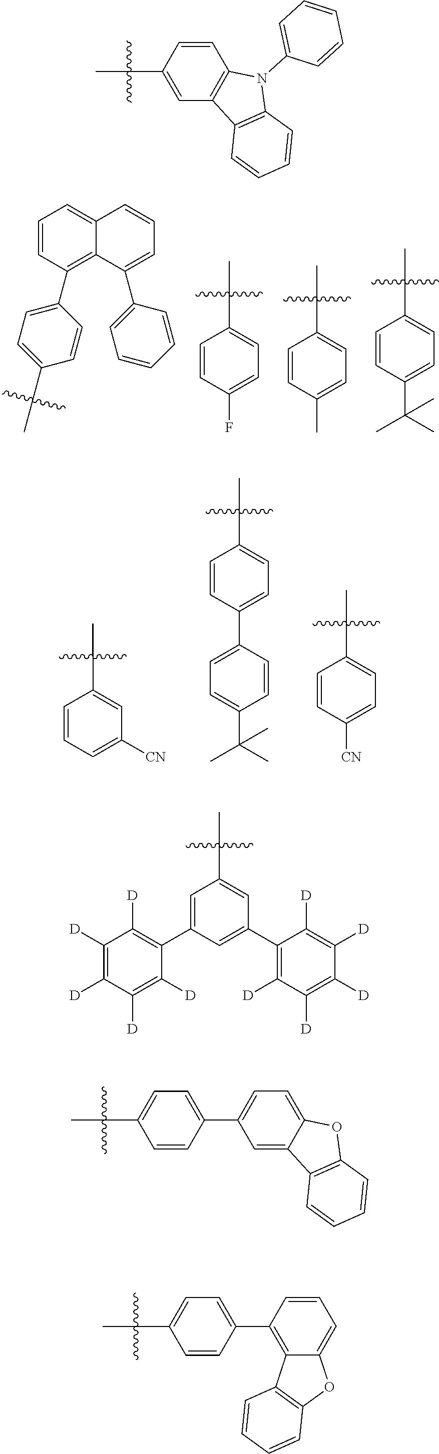

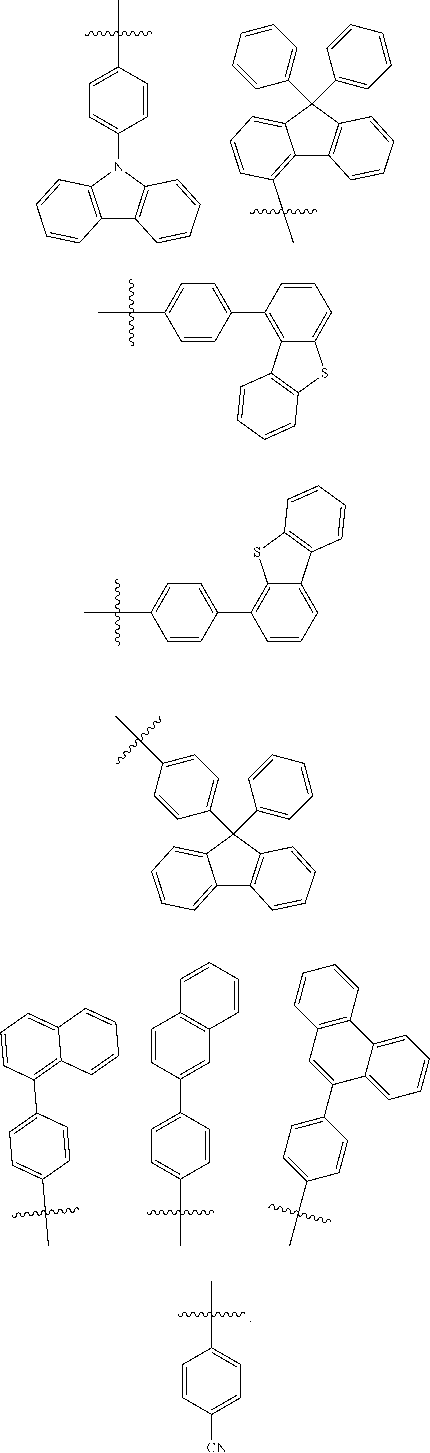

11. The organic compound according to claim 1, wherein each R₁ and each R₂ are the same or different, and are respectively and independently selected from deuterium, fluorine, cyano, methyl, ethyl, n-propyl, isopropyl, tert-butyl, phenyl, naphthyl, biphenyl or pentadeuteriophenyl.

12. The organic compound according to claim 1, wherein R₃ and R₄ are the same or different, and are respectively and independently selected from hydrogen, deuterium or methyl.

13. The organic compound according to claim 1, wherein the organic compound is selected from the following compounds:

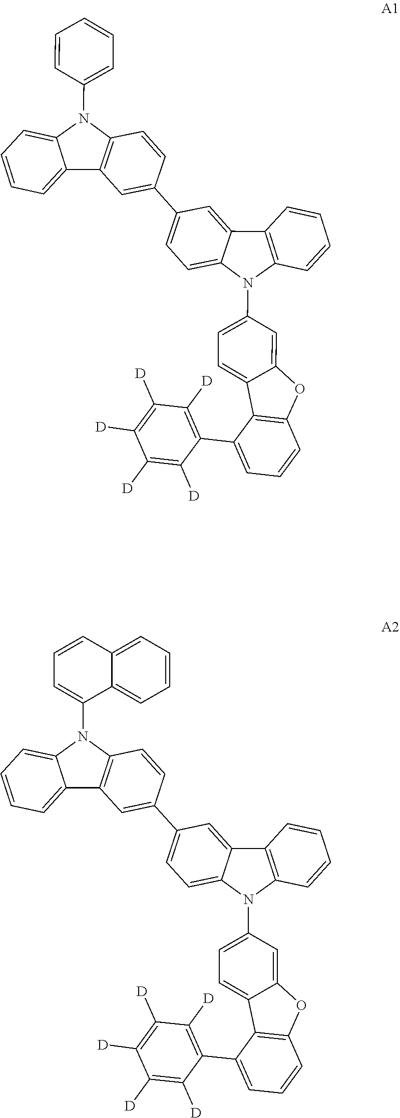 [SMILES: [2H]c1c([2H])c([2H])c(-c2cccc3oc4cc(-n5c6ccccc6c6cc(-c7ccc8c(c7)c7ccccc7n8-c7ccccc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cccc8ccccc78)ccc65)ccc4c23)c([2H])c1[2H]]

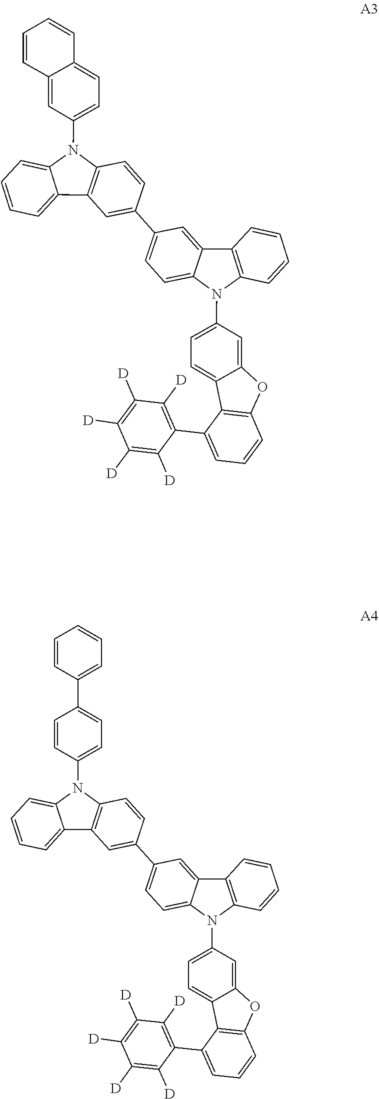 [SMILES: [2H]c1c([2H])c([2H])c(-c2cccc3oc4cc(-n5c6ccccc6c6cc(-c7ccc8c(c7)c7ccccc7n8-c7ccc8ccccc8c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(-c8ccccc8)cc7)ccc65)ccc4c23)c([2H])c1[2H]]

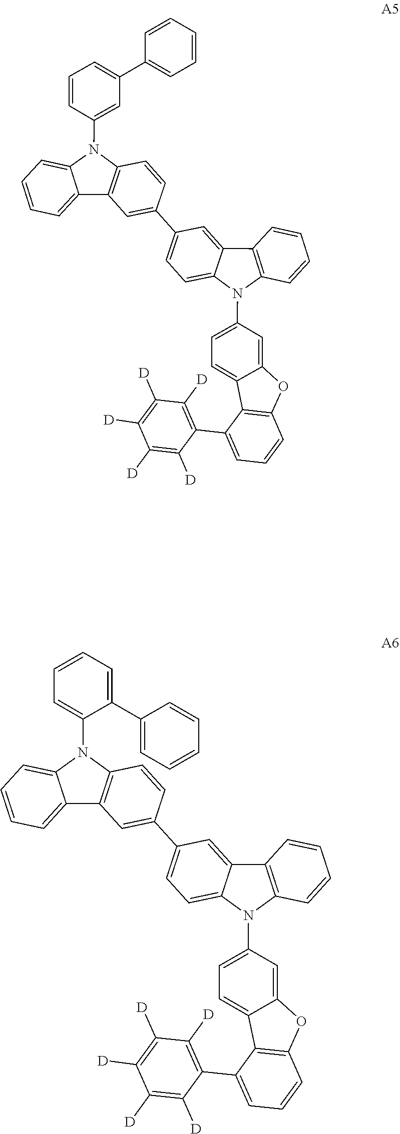 [SMILES: [2H]c1c([2H])c([2H])c(-c2cccc3oc4cc(-n5c6ccccc6c6cc(-c7ccc8c(c7)c7ccccc7n8-c7cccc(-c8ccccc8)c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccccc7-c7ccccc7)ccc65)ccc4c23)c([2H])c1[2H]]

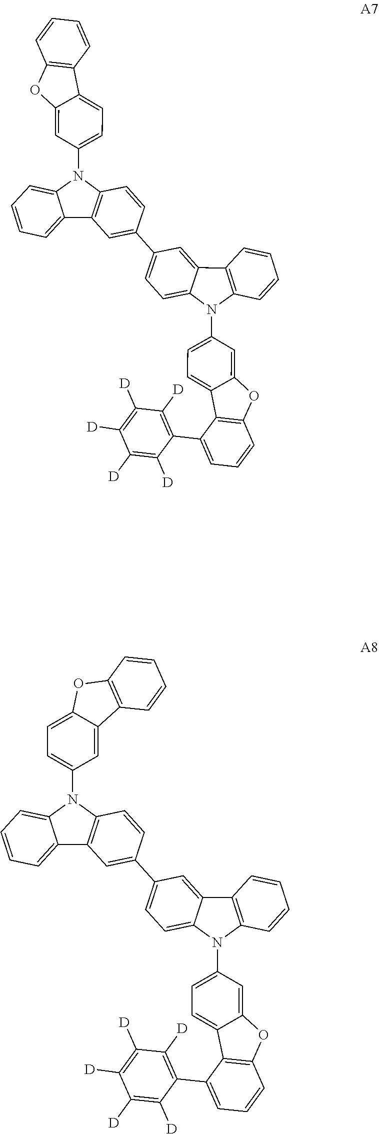 [SMILES: [2H]c1c([2H])c([2H])c(-c2cccc3oc4cc(-n5c6ccccc6c6cc(-c7ccc8c(c7)c7ccccc7n8-c7ccc8c(c7)oc7ccccc78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc8oc9ccccc9c8c7)ccc65)ccc4c23)c([2H])c1[2H]]

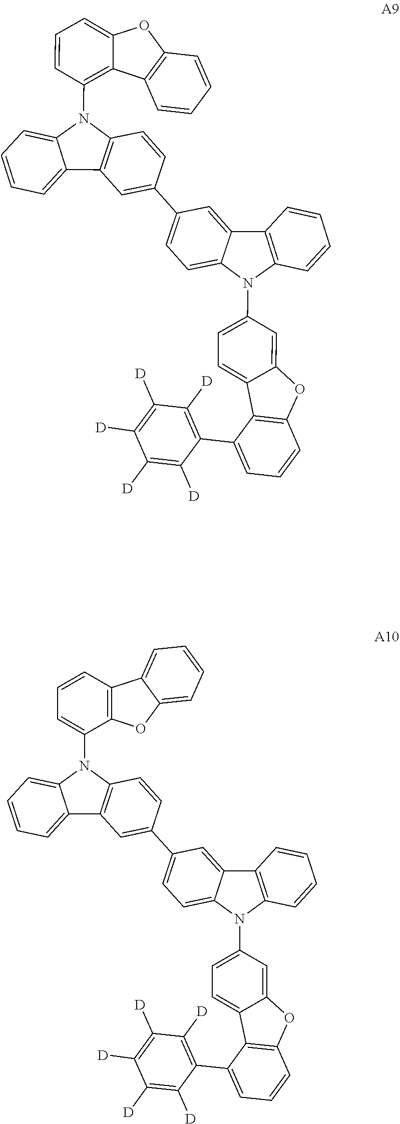 [SMILES: [2H]c1c([2H])c([2H])c(-c2cccc3oc4cc(-n5c6ccccc6c6cc(-c7ccc8c(c7)c7ccccc7n8-c7cccc8oc9ccccc9c78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cccc8c7oc7ccccc78)ccc65)ccc4c23)c([2H])c1[2H]]

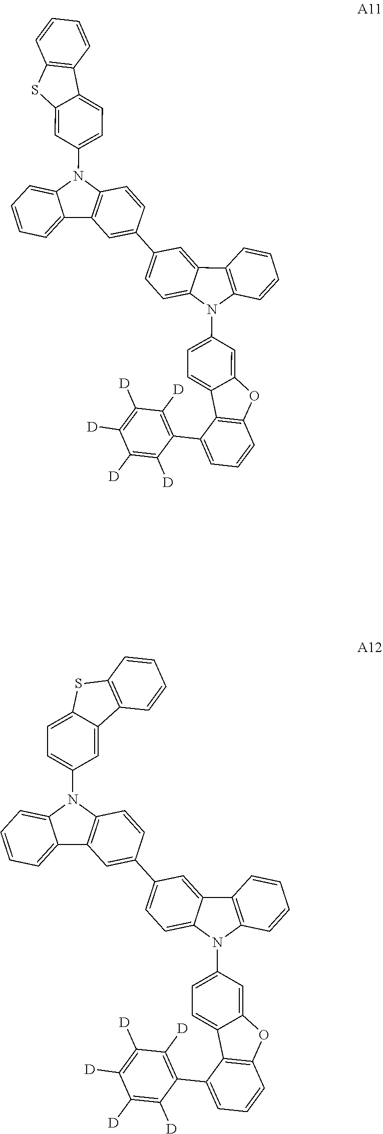 [SMILES: [2H]c1c([2H])c([2H])c(-c2cccc3oc4cc(-n5c6ccccc6c6cc(-c7ccc8c(c7)c7ccccc7n8-c7ccc8c(c7)sc7ccccc78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc8sc9ccccc9c8c7)ccc65)ccc4c23)c([2H])c1[2H]]

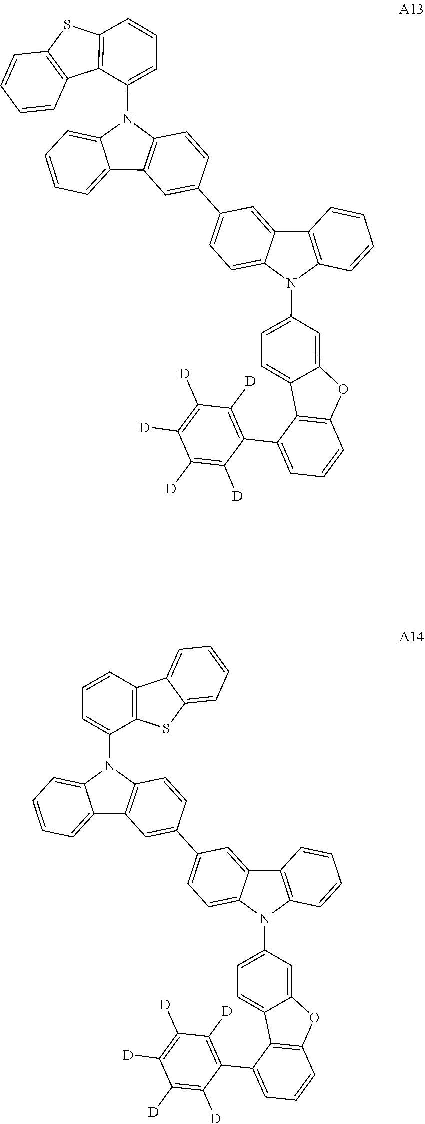 [SMILES: [2H]c1c([2H])c([2H])c(-c2cccc3oc4cc(-n5c6ccccc6c6cc(-c7ccc8c(c7)c7ccccc7n8-c7cccc8sc9ccccc9c78)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cccc8c7sc7ccccc78)ccc65)ccc4c23)c([2H])c1[2H]]

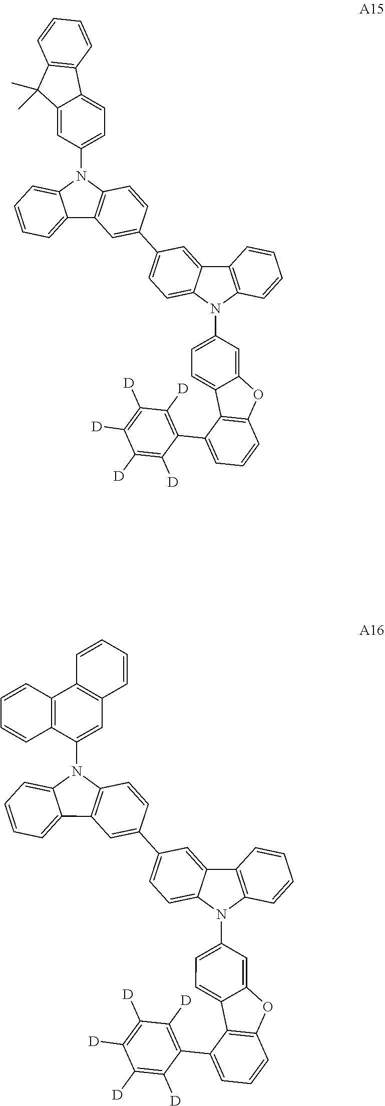 [SMILES: [2H]c1c([2H])c([2H])c(-c2cccc3oc4cc(-n5c6ccccc6c6cc(-c7ccc8c(c7)c7ccccc7n8-c7ccc8c(c7)C(C)(C)c7ccccc7-8)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7cc8ccccc8c8ccccc78)ccc65)ccc4c23)c([2H])c1[2H]]

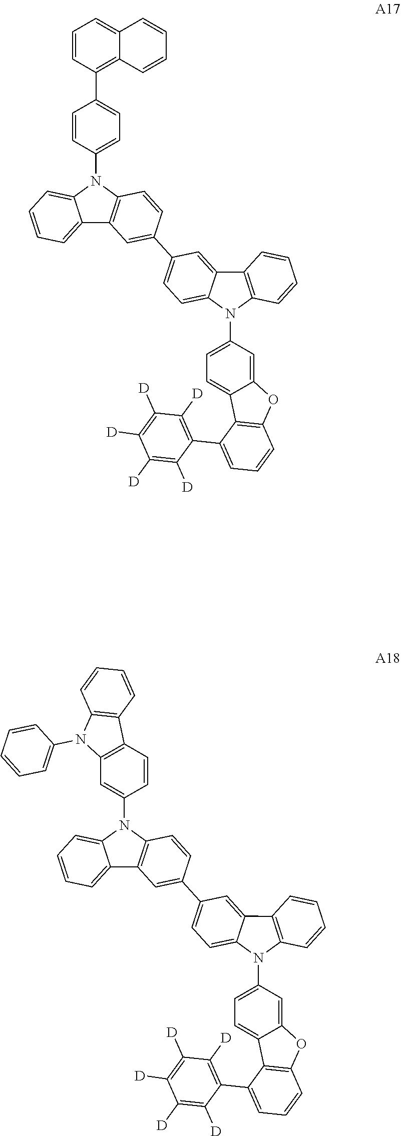 [SMILES: [2H]c1c([2H])c([2H])c(-c2cccc3oc4cc(-n5c6ccccc6c6cc(-c7ccc8c(c7)c7ccccc7n8-c7ccc(-c8cccc9ccccc89)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc8c9ccccc9n(-c9ccccc9)c8c7)ccc65)ccc4c23)c([2H])c1[2H]]

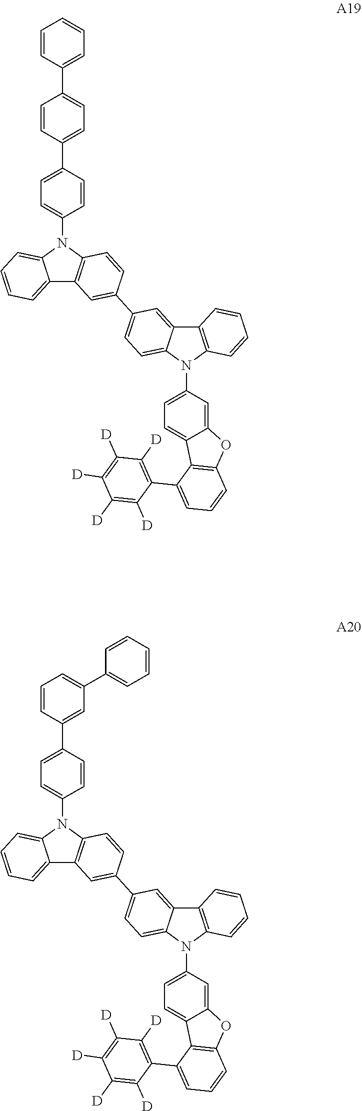 [SMILES: [2H]c1c([2H])c([2H])c(-c2cccc3oc4cc(-n5c6ccccc6c6cc(-c7ccc8c(c7)c7ccccc7n8-c7ccc(-c8ccc(-c9ccccc9)cc8)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(-c8cccc(-c9ccccc9)c8)cc7)ccc65)ccc4c23)c([2H])c1[2H]]

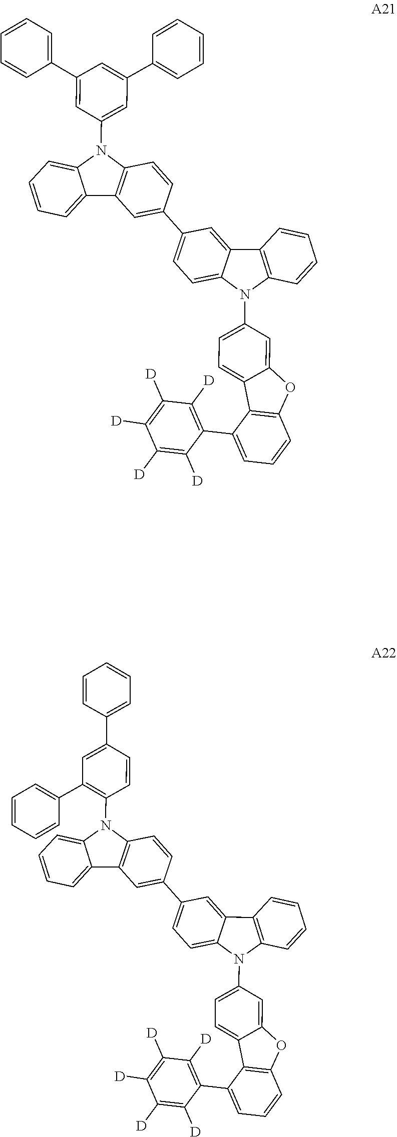 [SMILES: [2H]c1c([2H])c([2H])c(-c2cccc3oc4cc(-n5c6ccccc6c6cc(-c7ccc8c(c7)c7ccccc7n8-c7cc(-c8ccccc8)cc(-c8ccccc8)c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(-c8ccccc8)cc7-c7ccccc7)ccc65)ccc4c23)c([2H])c1[2H]]

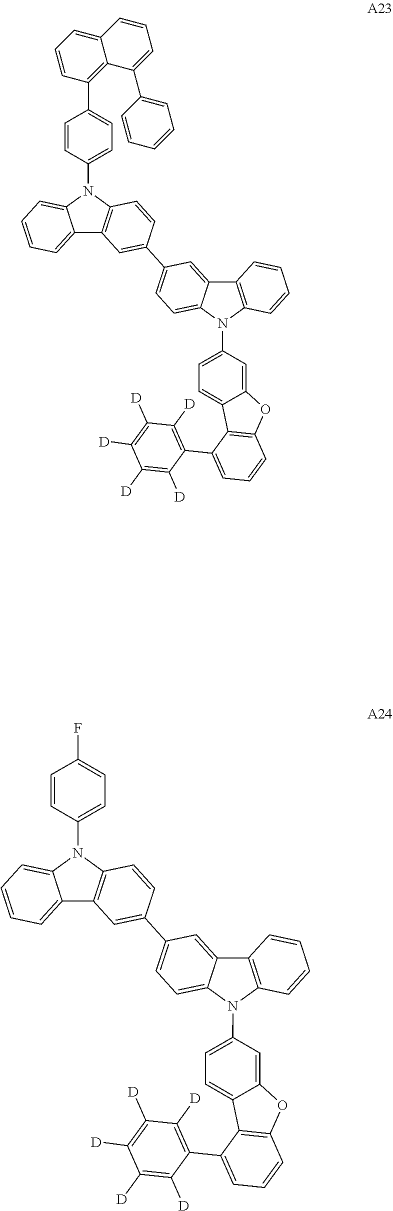 [SMILES: [2H]c1c([2H])c([2H])c(-c2cccc3oc4cc(-n5c6ccccc6c6cc(-c7ccc8c(c7)c7ccccc7n8-c7ccc(-c8cccc9cccc(-c%10ccccc%10)c89)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(F)cc7)ccc65)ccc4c23)c([2H])c1[2H]]

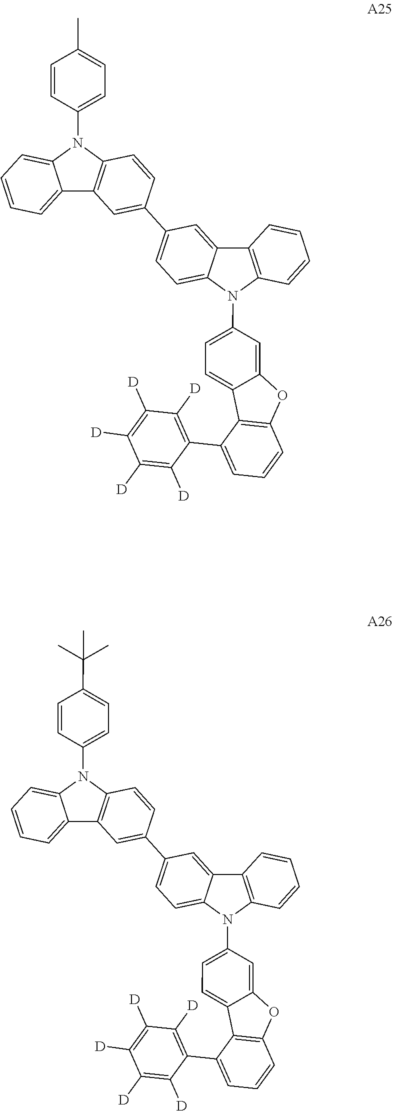 [SMILES: [2H]c1c([2H])c([2H])c(-c2cccc3oc4cc(-n5c6ccccc6c6cc(-c7ccc8c(c7)c7ccccc7n8-c7ccc(C)cc7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7ccc(C(C)(C)C)cc7)ccc65)ccc4c23)c([2H])c1[2H]]

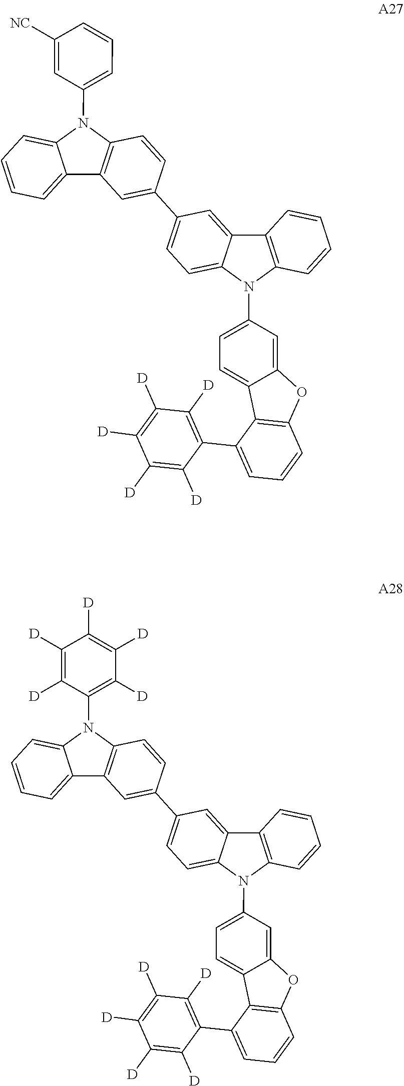 [SMILES: [2H]c1c([2H])c([2H])c(-c2cccc3oc4cc(-n5c6ccccc6c6cc(-c7ccc8c(c7)c7ccccc7n8-c7cccc(C#N)c7)ccc65)ccc4c23)c([2H])c1[2H] ; [2H]c1c([2H])c([2H])c(-c2cccc3oc4cc(-n5c6ccccc6c6cc(-c7ccc8c(c7)c7ccccc7n8-c7c([2H])c([2H])c([2H])c([2H])c7[2H])ccc65)ccc4c23)c([2H])c1[2H]]

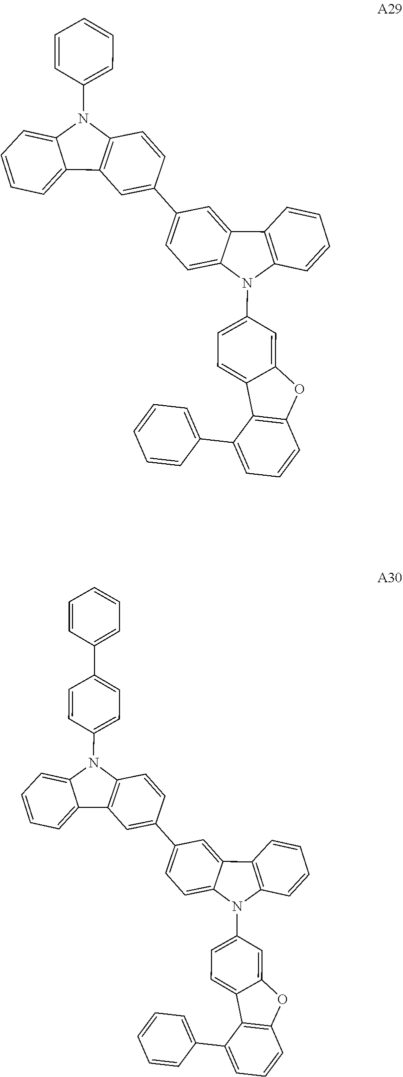 [SMILES: c1ccc(-c2cccc3oc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc6c(c5)oc5cccc(-c7ccccc7)c56)ccc43)cc2)cc1]

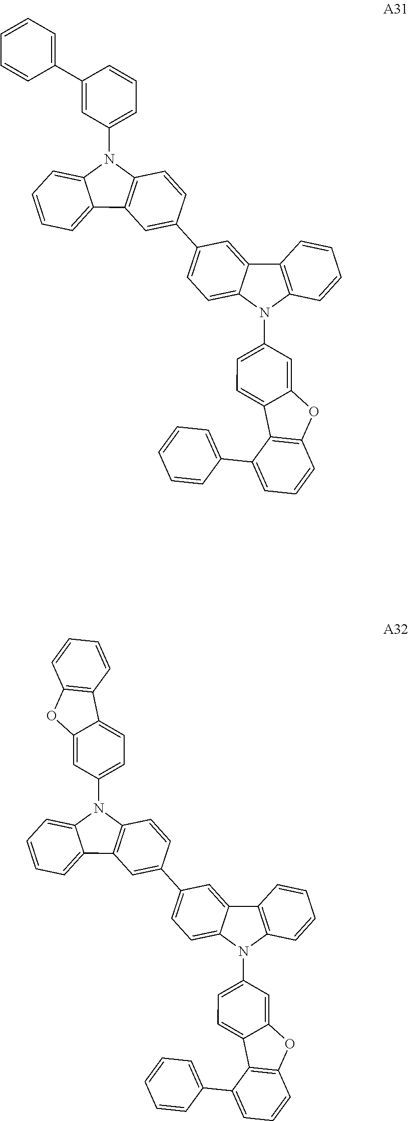 [SMILES: c1ccc(-c2cccc(-n3c4ccccc4c4cc(-c5ccc6c(c5)c5ccccc5n6-c5ccc6c(c5)oc5cccc(-c7ccccc7)c56)ccc43)c2)cc1 ; c1ccc(-c2cccc3oc4cc(-n5c6ccccc6c6cc(-c7ccc8c(c7)c7ccccc7n8-c7ccc8c(c7)oc7ccccc78)ccc65)ccc4c23)cc1]

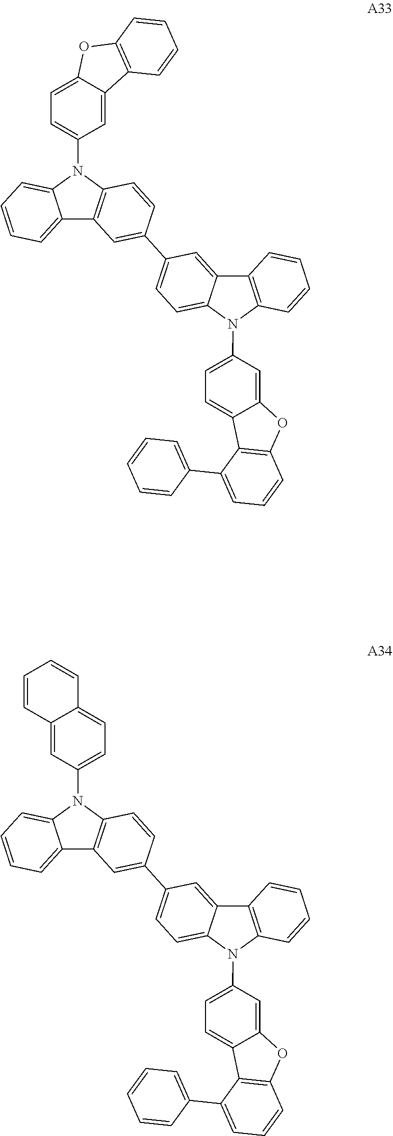 [SMILES: c1ccc(-c2cccc3oc4cc(-n5c6ccccc6c6cc(-c7ccc8c(c7)c7ccccc7n8-c7ccc8oc9ccccc9c8c7)ccc65)ccc4c23)cc1 ; c1ccc(-c2cccc3oc4cc(-n5c6ccccc6c6cc(-c7ccc8c(c7)c7ccccc7n8-c7ccc8ccccc8c7)ccc65)ccc4c23)cc1]

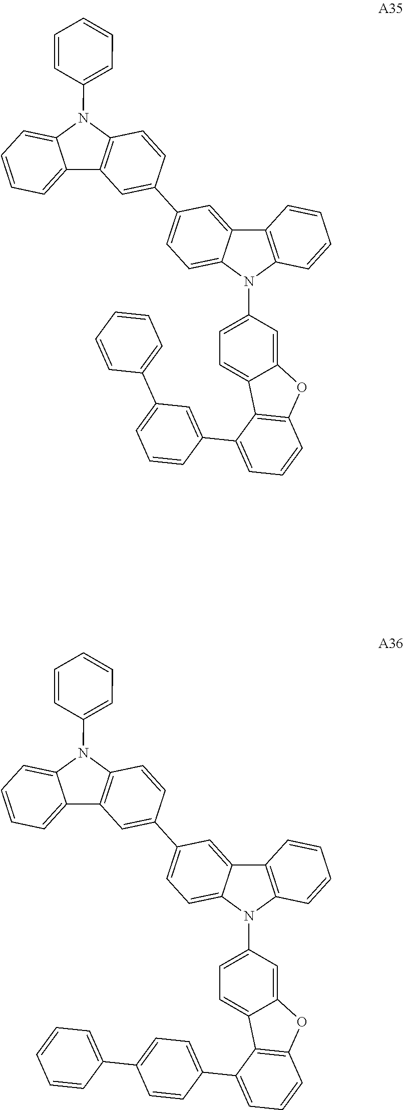 [SMILES: c1ccc(-c2cccc(-c3cccc4oc5cc(-n6c7ccccc7c7cc(-c8ccc9c(c8)c8ccccc8n9-c8ccccc8)ccc76)ccc5c34)c2)cc1 ; c1ccc(-c2ccc(-c3cccc4oc5cc(-n6c7ccccc7c7cc(-c8ccc9c(c8)c8ccccc8n9-c8ccccc8)ccc76)ccc5c34)cc2)cc1]

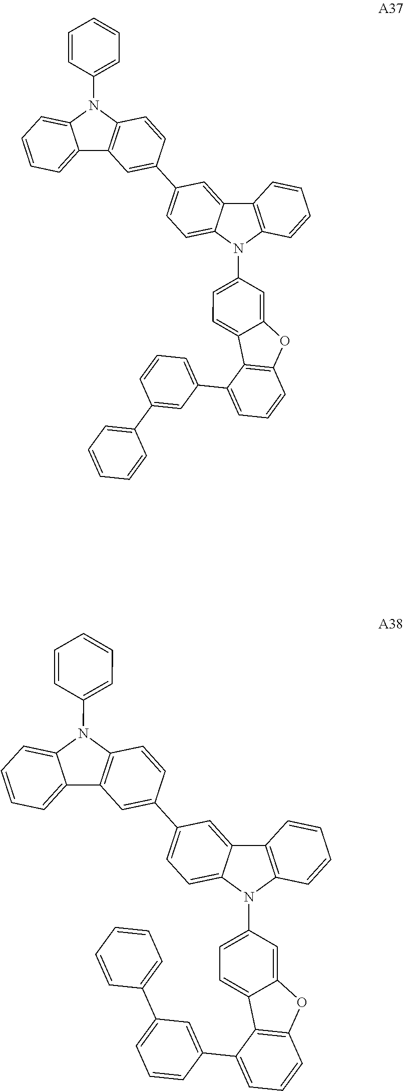 [SMILES: c1ccc(-c2cccc(-c3cccc4oc5cc(-n6c7ccccc7c7cc(-c8ccc9c(c8)c8ccccc8n9-c8ccccc8)ccc76)ccc5c34)c2)cc1 ; c1ccc(-c2cccc(-c3cccc4oc5cc(-n6c7ccccc7c7cc(-c8ccc9c(c8)c8ccccc8n9-c8ccccc8)ccc76)ccc5c34)c2)cc1]

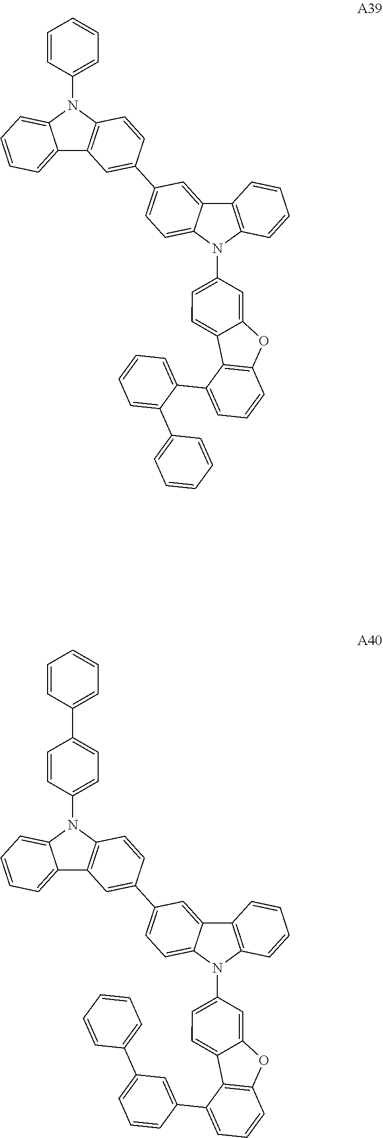 [SMILES: c1ccc(-c2ccccc2-c2cccc3oc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc6c(c5)oc5cccc(-c7cccc(-c8ccccc8)c7)c56)ccc43)cc2)cc1]

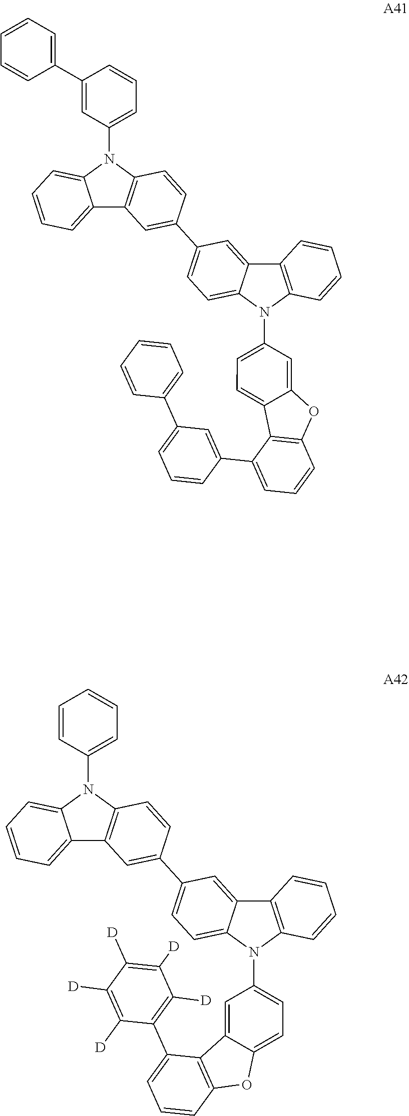 [SMILES: c1ccc(-c2cccc(-c3cccc4oc5cc(-n6c7ccccc7c7cc(-c8ccc9c(c8)c8ccccc8n9-c8cccc(-c9ccccc9)c8)ccc76)ccc5c34)c2)cc1 ; [2H]c1c([2H])c([2H])c(-c2cccc3oc4ccc(-n5c6ccccc6c6cc(-c7ccc8c(c7)c7ccccc7n8-c7ccccc7)ccc65)cc4c23)c([2H])c1[2H]]

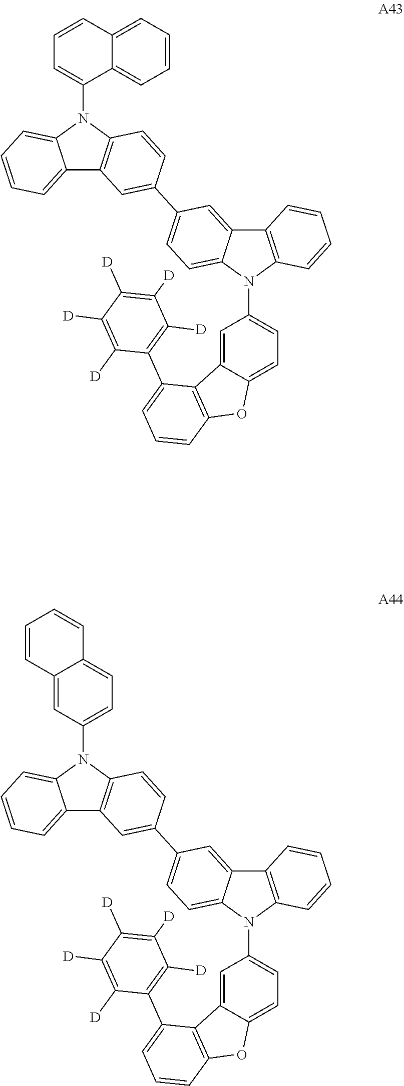 [SMILES: [2H]c1c([2H])c([2H])c(-c2cccc3oc4ccc(-n5c6ccccc6c6cc(-c7ccc8c(c7)c7ccccc7n8-c7cccc8ccccc78)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8ccccc8c7)ccc65)cc4c23)c([2H])c1[2H]]

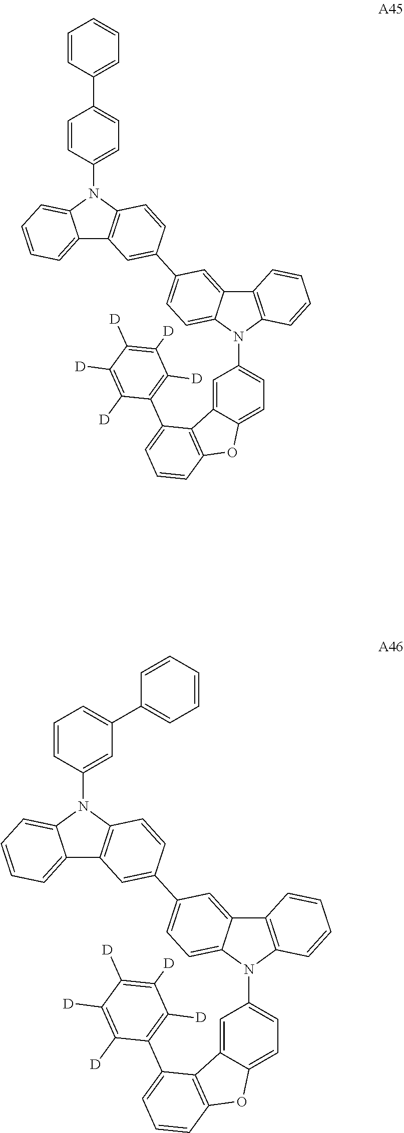 [SMILES: [2H]c1c([2H])c([2H])c(-c2cccc3oc4ccc(-n5c6ccccc6c6cc(-c7ccc8c(c7)c7ccccc7n8-c7ccc(-c8ccccc8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7cccc(-c8ccccc8)c7)ccc65)cc4c23)c([2H])c1[2H]]

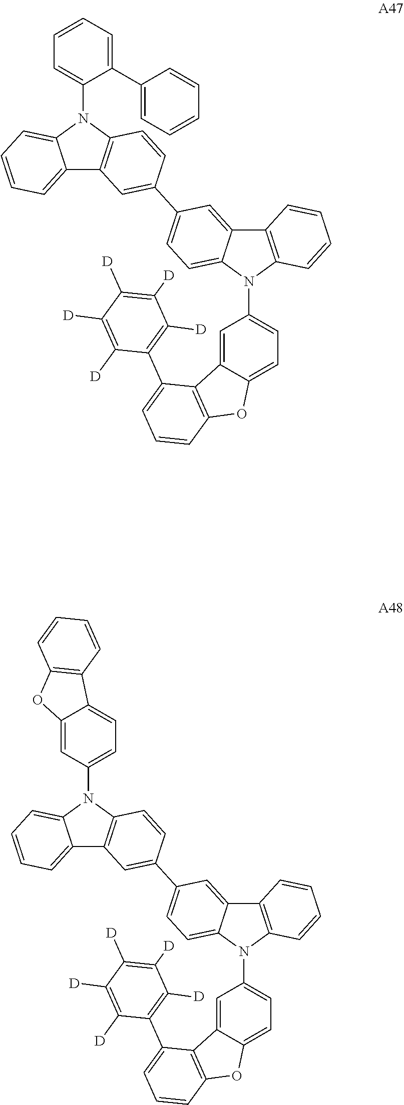 [SMILES: [2H]c1c([2H])c([2H])c(-c2cccc3oc4ccc(-n5c6ccccc6c6cc(-c7ccc8c(c7)c7ccccc7n8-c7ccccc7-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)oc7ccccc78)ccc65)cc4c23)c([2H])c1[2H]]

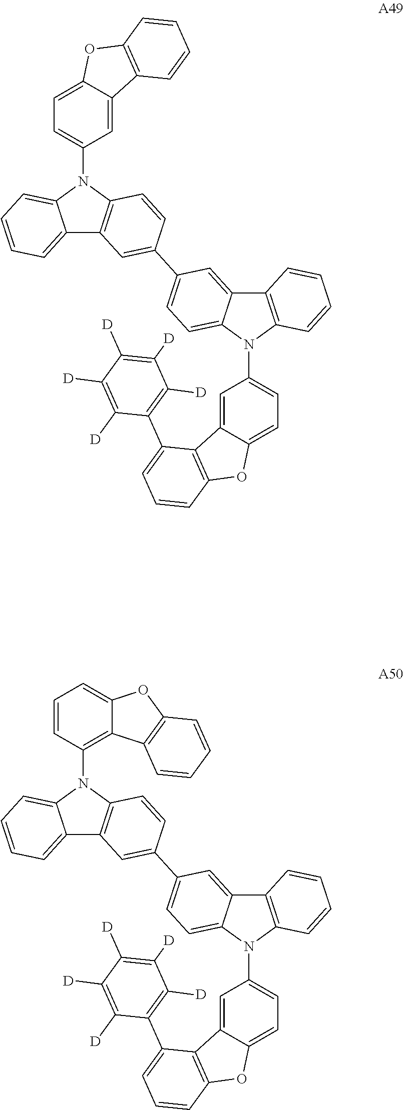 [SMILES: [2H]c1c([2H])c([2H])c(-c2cccc3oc4ccc(-n5c6ccccc6c6cc(-c7ccc8c(c7)c7ccccc7n8-c7ccc8oc9ccccc9c8c7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7cccc8oc9ccccc9c78)ccc65)cc4c23)c([2H])c1[2H]]

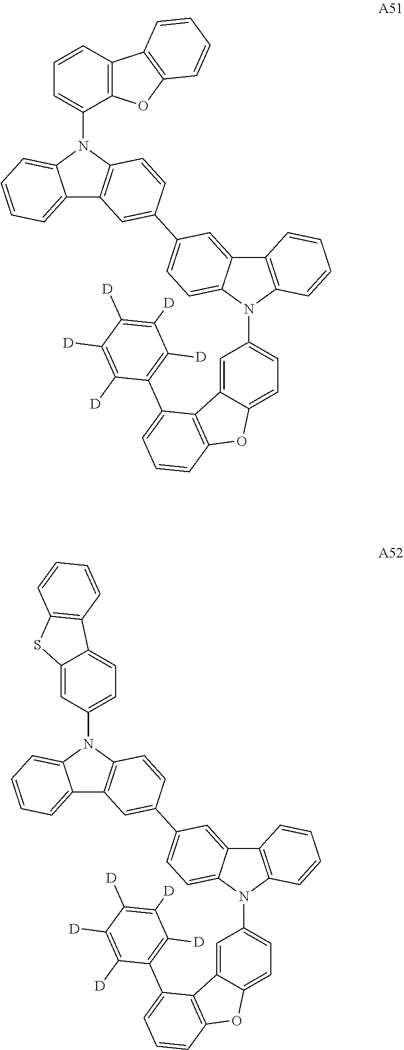 [SMILES: [2H]c1c([2H])c([2H])c(-c2cccc3oc4ccc(-n5c6ccccc6c6cc(-c7ccc8c(c7)c7ccccc7n8-c7cccc8c7oc7ccccc78)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)sc7ccccc78)ccc65)cc4c23)c([2H])c1[2H]]

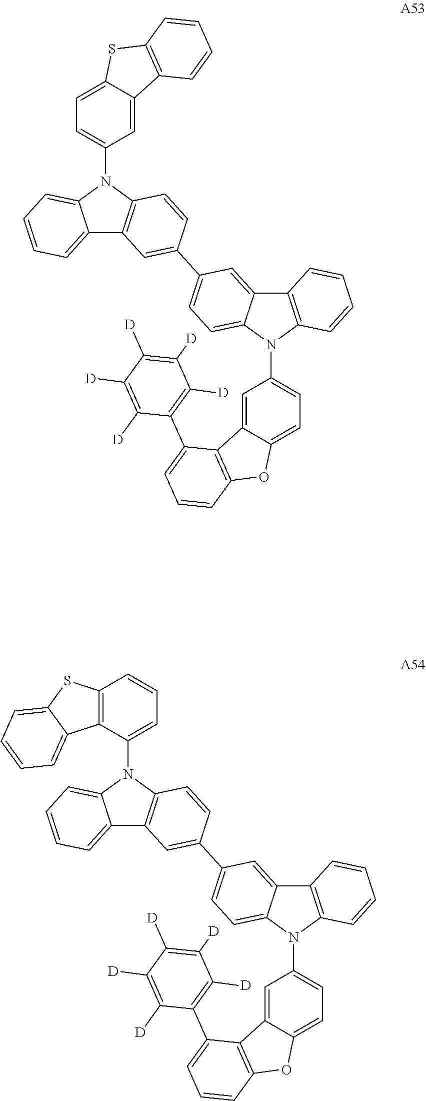 [SMILES: [2H]c1c([2H])c([2H])c(-c2cccc3oc4ccc(-n5c6ccccc6c6cc(-c7ccc8c(c7)c7ccccc7n8-c7ccc8sc9ccccc9c8c7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7cccc8sc9ccccc9c78)ccc65)cc4c23)c([2H])c1[2H]]

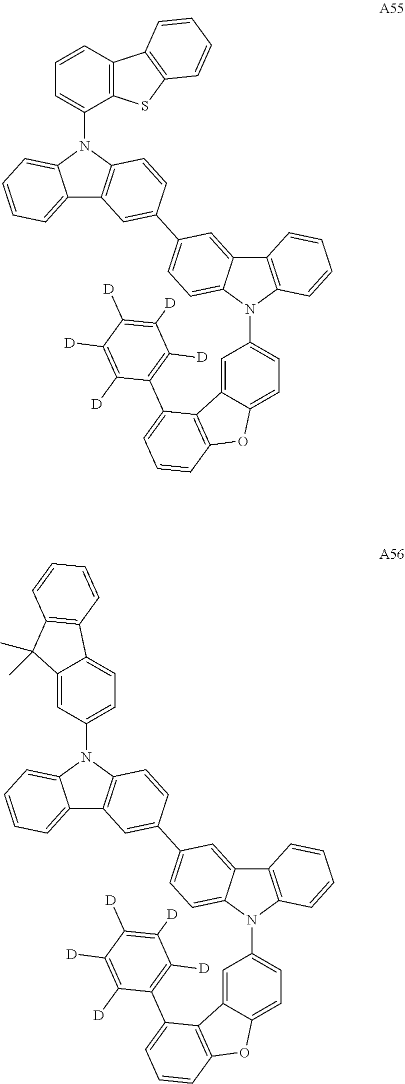 [SMILES: [2H]c1c([2H])c([2H])c(-c2cccc3oc4ccc(-n5c6ccccc6c6cc(-c7ccc8c(c7)c7ccccc7n8-c7cccc8c7sc7ccccc78)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)C(C)(C)c7ccccc7-8)ccc65)cc4c23)c([2H])c1[2H]]

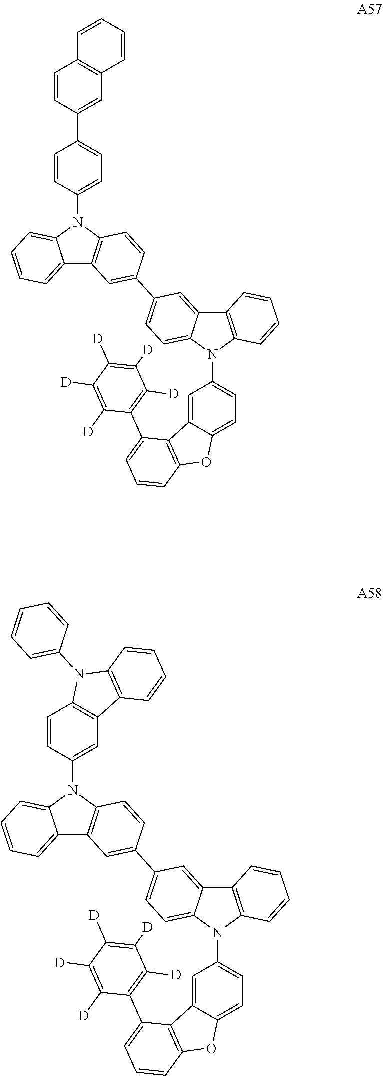 [SMILES: [2H]c1c([2H])c([2H])c(-c2cccc3oc4ccc(-n5c6ccccc6c6cc(-c7ccc8c(c7)c7ccccc7n8-c7ccc(-c8ccc9ccccc9c8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc8c(c7)c7ccccc7n8-c7ccccc7)ccc65)cc4c23)c([2H])c1[2H]]

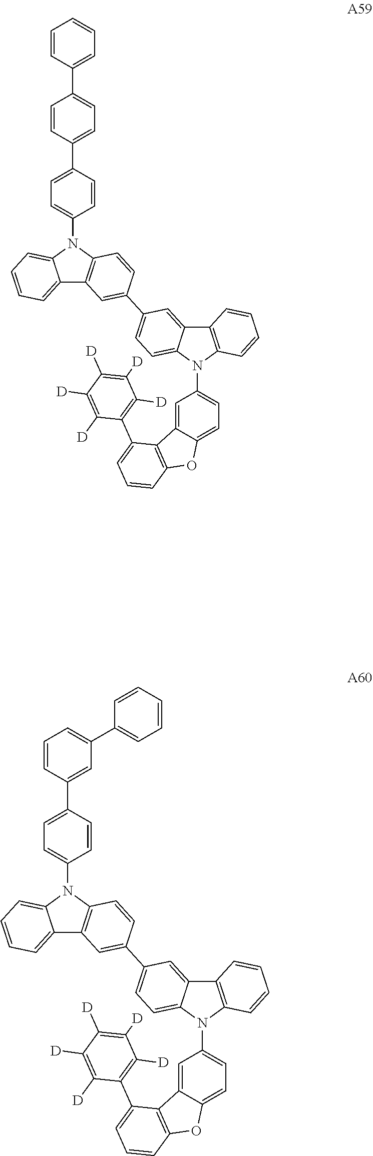 [SMILES: [2H]c1c([2H])c([2H])c(-c2cccc3oc4ccc(-n5c6ccccc6c6cc(-c7ccc8c(c7)c7ccccc7n8-c7ccc(-c8ccc(-c9ccccc9)cc8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(-c8cccc(-c9ccccc9)c8)cc7)ccc65)cc4c23)c([2H])c1[2H]]

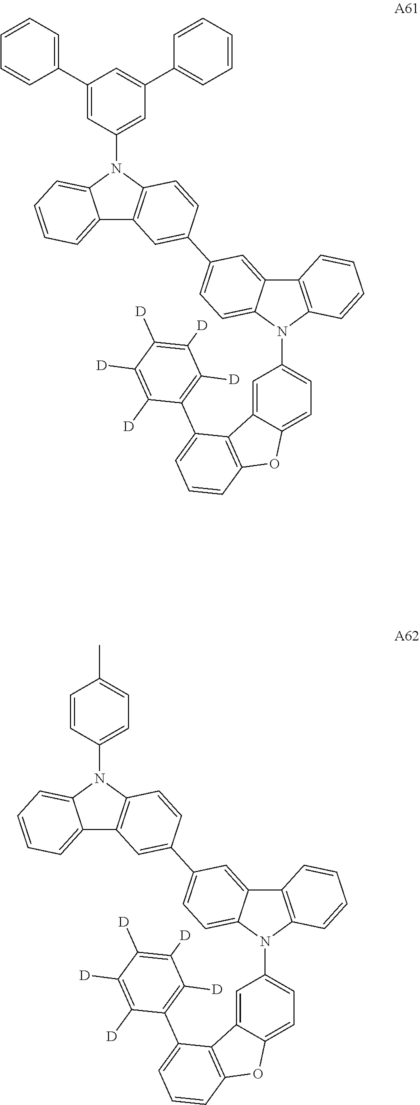 [SMILES: [2H]c1c([2H])c([2H])c(-c2cccc3oc4ccc(-n5c6ccccc6c6cc(-c7ccc8c(c7)c7ccccc7n8-c7cc(-c8ccccc8)cc(-c8ccccc8)c7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(C)cc7)ccc65)cc4c23)c([2H])c1[2H]]

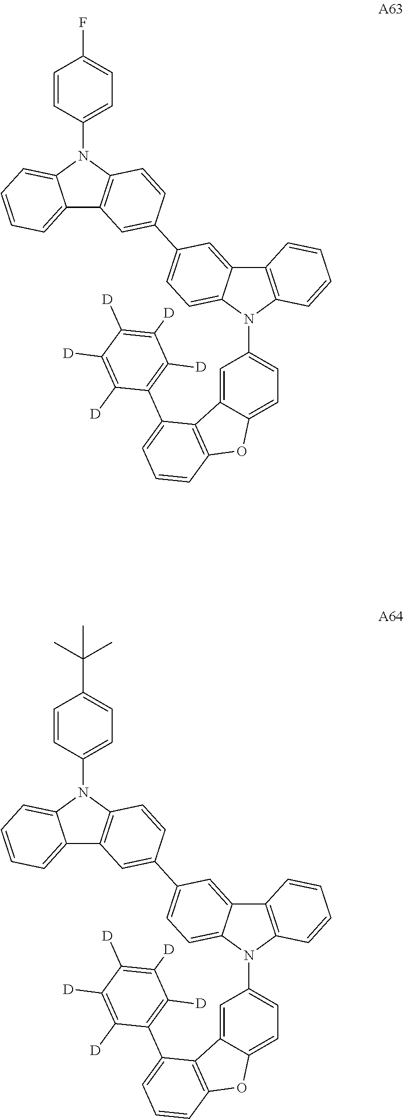 [SMILES: [2H]c1c([2H])c([2H])c(-c2cccc3oc4ccc(-n5c6ccccc6c6cc(-c7ccc8c(c7)c7ccccc7n8-c7ccc(F)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(C(C)(C)C)cc7)ccc65)cc4c23)c([2H])c1[2H]]

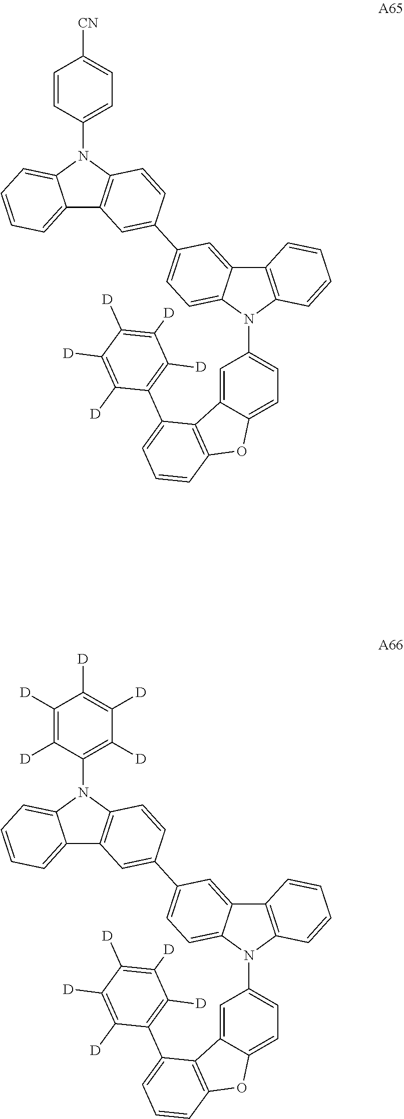 [SMILES: [2H]c1c([2H])c([2H])c(-c2cccc3oc4ccc(-n5c6ccccc6c6cc(-c7ccc8c(c7)c7ccccc7n8-c7ccc(C#N)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7c([2H])c([2H])c([2H])c([2H])c7[2H])ccc65)cc4c23)c([2H])c1[2H]]

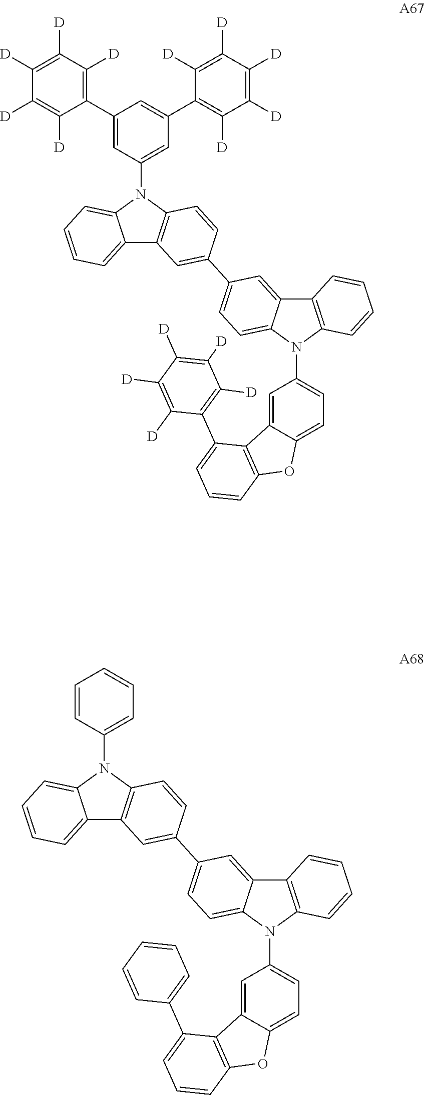 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccc6oc7cccc(-c8c([2H])c([2H])c([2H])c([2H])c8[2H])c7c6c5)ccc43)c2)c([2H])c1[2H] ; c1ccc(-c2cccc3oc4ccc(-n5c6ccccc6c6cc(-c7ccc8c(c7)c7ccccc7n8-c7ccccc7)ccc65)cc4c23)cc1]

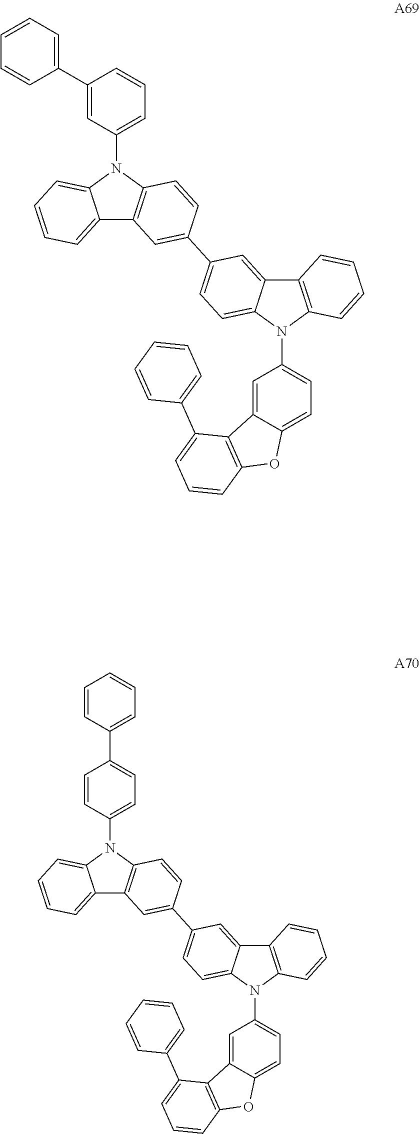 [SMILES: c1ccc(-c2cccc(-n3c4ccccc4c4cc(-c5ccc6c(c5)c5ccccc5n6-c5ccc6oc7cccc(-c8ccccc8)c7c6c5)ccc43)c2)cc1 ; c1ccc(-c2ccc(-n3c4ccccc4c4cc(-c5ccc6c(c5)c5ccccc5n6-c5ccc6oc7cccc(-c8ccccc8)c7c6c5)ccc43)cc2)cc1]

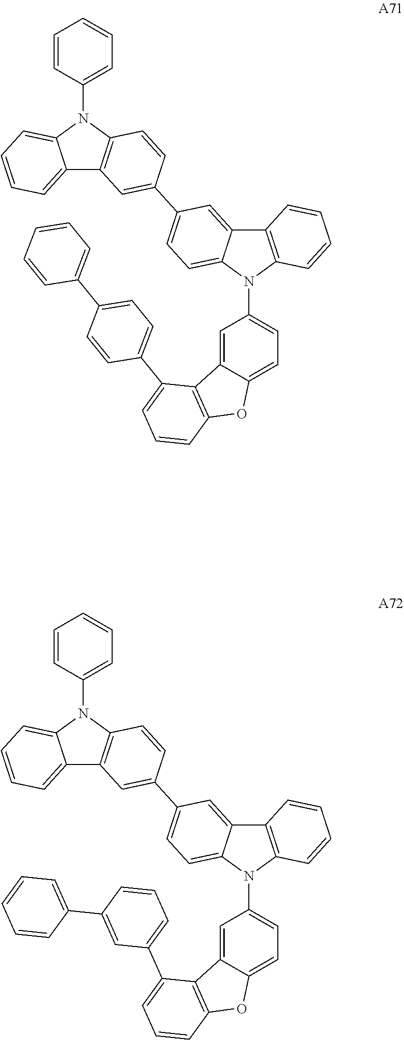 [SMILES: c1ccc(-c2ccc(-c3cccc4oc5ccc(-n6c7ccccc7c7cc(-c8ccc9c(c8)c8ccccc8n9-c8ccccc8)ccc76)cc5c34)cc2)cc1 ; c1ccc(-c2cccc(-c3cccc4oc5ccc(-n6c7ccccc7c7cc(-c8ccc9c(c8)c8ccccc8n9-c8ccccc8)ccc76)cc5c34)c2)cc1]

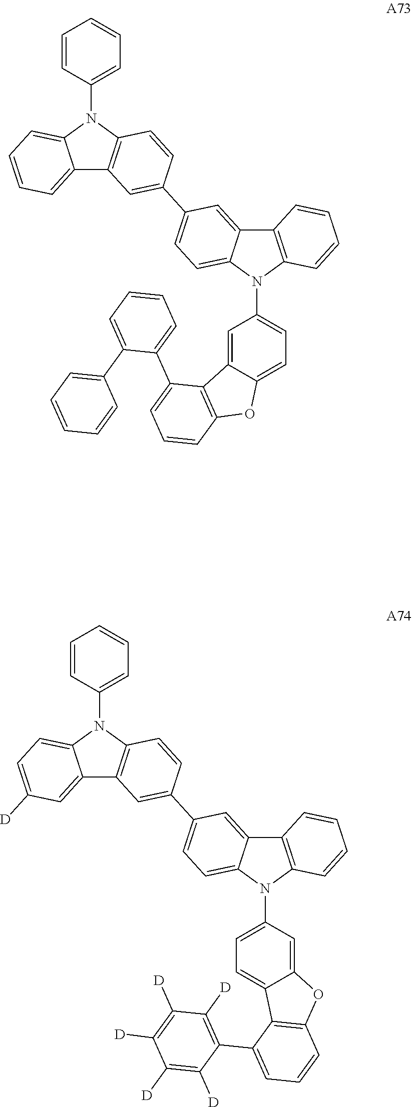 [SMILES: c1ccc(-c2ccccc2-c2cccc3oc4ccc(-n5c6ccccc6c6cc(-c7ccc8c(c7)c7ccccc7n8-c7ccccc7)ccc65)cc4c23)cc1 ; [2H]c1ccc2c(c1)c1cc(-c3ccc4c(c3)c3ccccc3n4-c3ccc4c(c3)oc3cccc(-c5c([2H])c([2H])c([2H])c([2H])c5[2H])c34)ccc1n2-c1ccccc1]

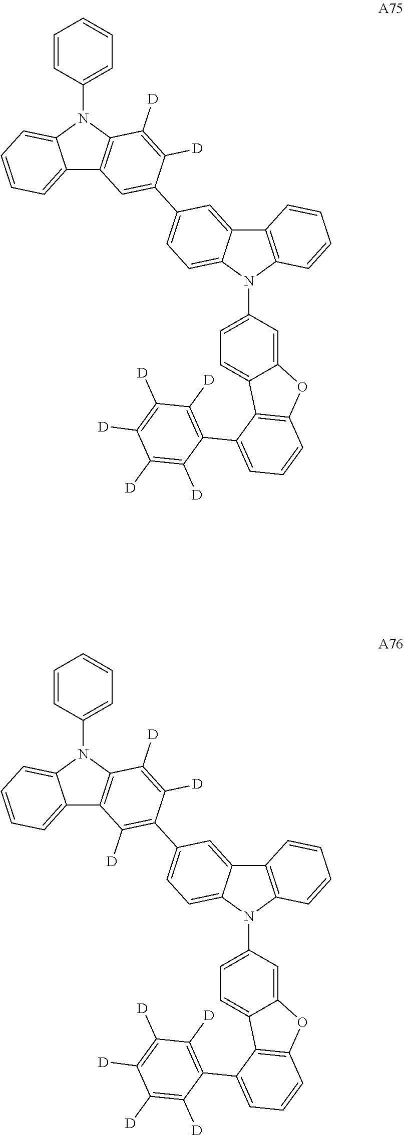 [SMILES: [2H]c1c([2H])c([2H])c(-c2cccc3oc4cc(-n5c6ccccc6c6cc(-c7cc8c9ccccc9n(-c9ccccc9)c8c([2H])c7[2H])ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7ccccc7)ccc65)ccc4c23)c([2H])c1[2H]]

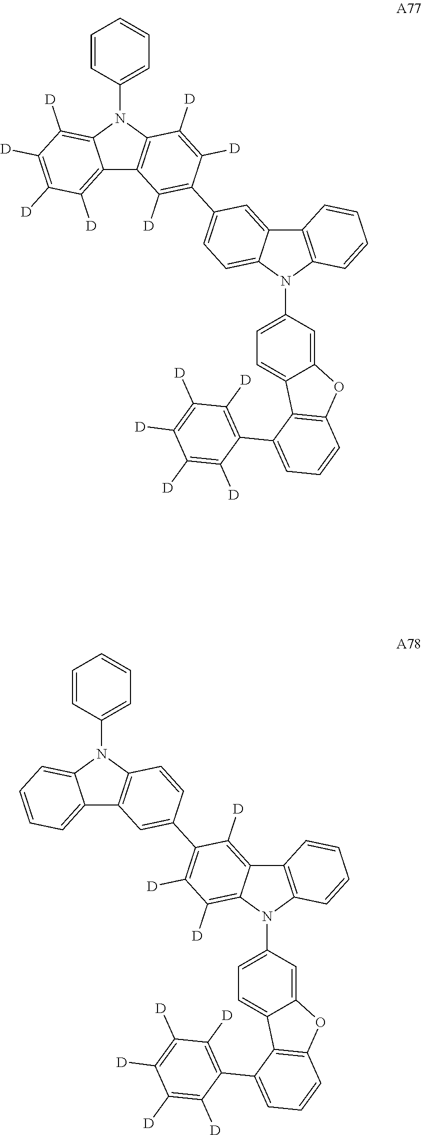 [SMILES: [2H]c1c([2H])c([2H])c(-c2cccc3oc4cc(-n5c6ccccc6c6cc(-c7c([2H])c([2H])c8c(c7[2H])c7c([2H])c([2H])c([2H])c([2H])c7n8-c7ccccc7)ccc65)ccc4c23)c([2H])c1[2H] ; [2H]c1c([2H])c([2H])c(-c2cccc3oc4cc(-n5c6ccccc6c6c([2H])c(-c7ccc8c(c7)c7ccccc7n8-c7ccccc7)c([2H])c([2H])c65)ccc4c23)c([2H])c1[2H]]

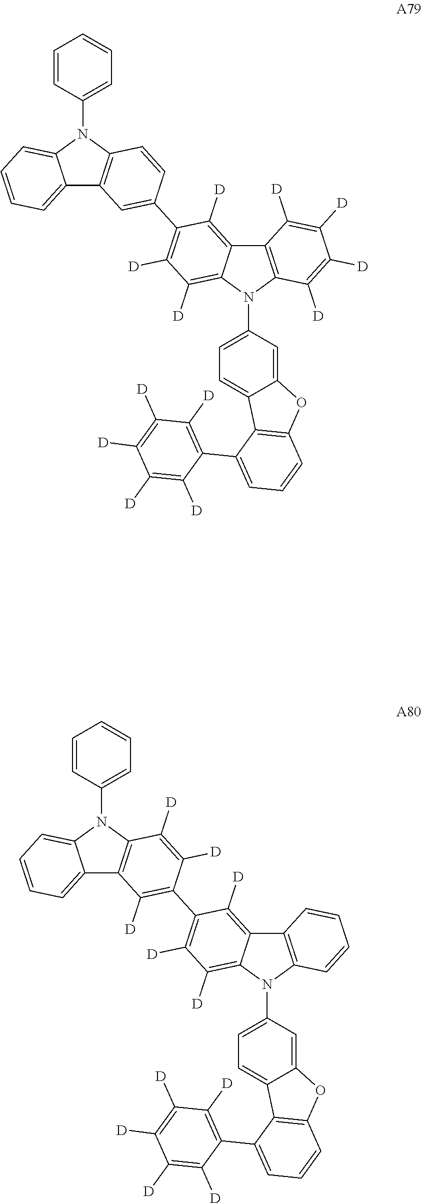 [SMILES: [2H]c1c([2H])c([2H])c(-c2cccc3oc4cc(-n5c6c([2H])c([2H])c([2H])c([2H])c6c6c([2H])c(-c7ccc8c(c7)c7ccccc7n8-c7ccccc7)c([2H])c([2H])c65)ccc4c23)c([2H])c1[2H] ; [2H]c1c([2H])c([2H])c(-c2cccc3oc4cc(-n5c6ccccc6c6c([2H])c(-c7c([2H])c([2H])c8c(c7[2H])c7ccccc7n8-c7ccccc7)c([2H])c([2H])c65)ccc4c23)c([2H])c1[2H]]

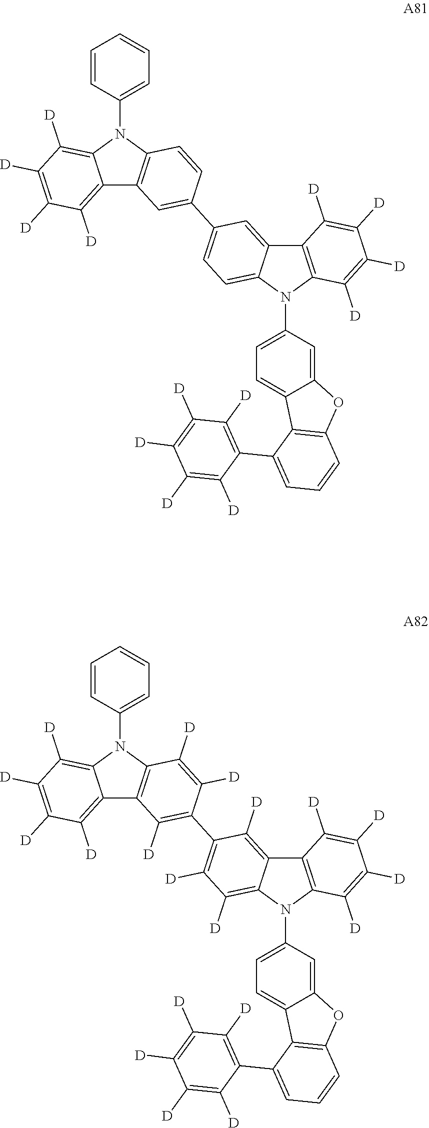 [SMILES: [2H]c1c([2H])c([2H])c(-c2cccc3oc4cc(-n5c6ccc(-c7ccc8c(c7)c7c([2H])c([2H])c([2H])c([2H])c7n8-c7ccccc7)cc6c6c([2H])c([2H])c([2H])c([2H])c65)ccc4c23)c([2H])c1[2H] ; [2H]c1c([2H])c([2H])c(-c2cccc3oc4cc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)ccc4c23)c([2H])c1[2H]]

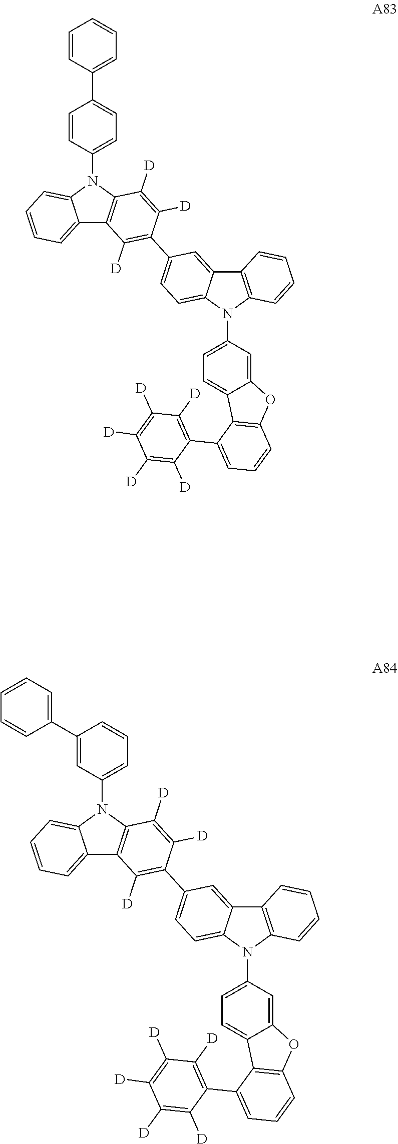 [SMILES: [2H]c1c([2H])c([2H])c(-c2cccc3oc4cc(-n5c6ccccc6c6cc(-c7c([2H])c([2H])c8c(c7[2H])c7ccccc7n8-c7ccc(-c8ccccc8)cc7)ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7cccc(-c8ccccc8)c7)ccc65)ccc4c23)c([2H])c1[2H]]

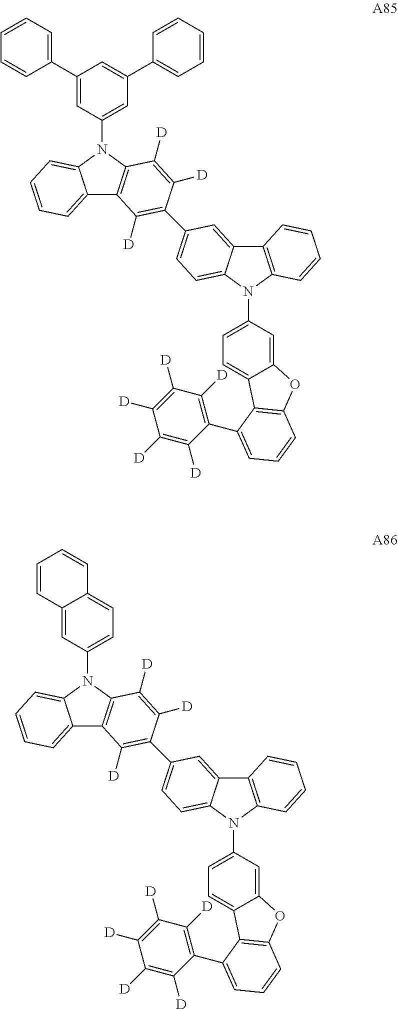 [SMILES: [2H]c1c([2H])c([2H])c(-c2cccc3oc4cc(-n5c6ccccc6c6cc(-c7c([2H])c([2H])c8c(c7[2H])c7ccccc7n8-c7cc(-c8ccccc8)cc(-c8ccccc8)c7)ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7ccc8ccccc8c7)ccc65)ccc4c23)c([2H])c1[2H]]

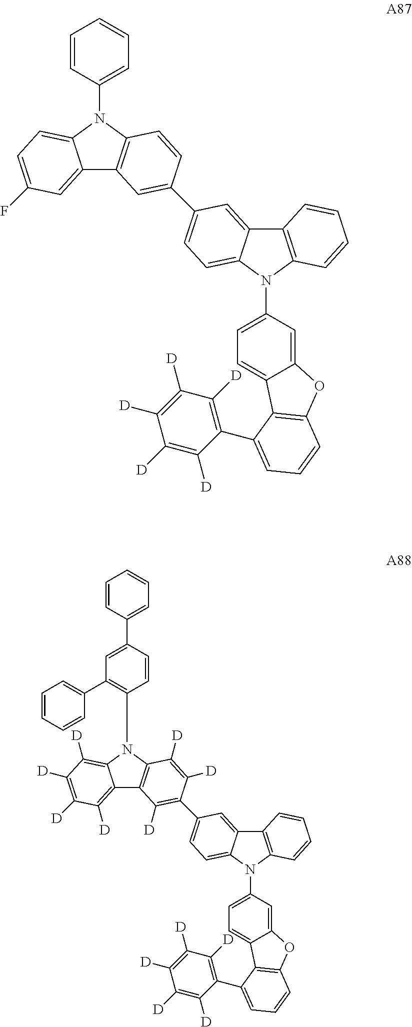 [SMILES: [2H]c1c([2H])c([2H])c(-c2cccc3oc4cc(-n5c6ccccc6c6cc(-c7ccc8c(c7)c7cc(F)ccc7n8-c7ccccc7)ccc65)ccc4c23)c([2H])c1[2H] ; [2H]c1c([2H])c([2H])c(-c2cccc3oc4cc(-n5c6ccccc6c6cc(-c7c([2H])c([2H])c8c(c7[2H])c7c([2H])c([2H])c([2H])c([2H])c7n8-c7ccc(-c8ccccc8)cc7-c7ccccc7)ccc65)ccc4c23)c([2H])c1[2H]]

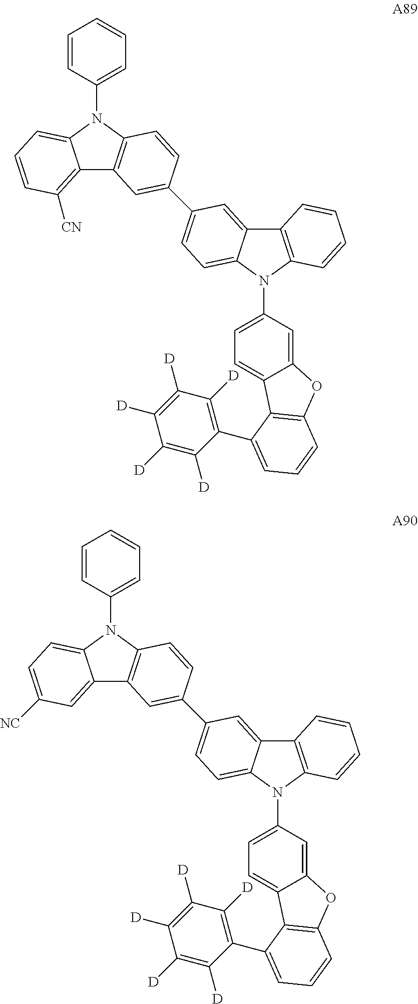 [SMILES: [2H]c1c([2H])c([2H])c(-c2cccc3oc4cc(-n5c6ccccc6c6cc(-c7ccc8c(c7)c7c(C#N)cccc7n8-c7ccccc7)ccc65)ccc4c23)c([2H])c1[2H] ; [2H]c1c([2H])c([2H])c(-c2cccc3oc4cc(-n5c6ccccc6c6cc(-c7ccc8c(c7)c7cc(C#N)ccc7n8-c7ccccc7)ccc65)ccc4c23)c([2H])c1[2H]]

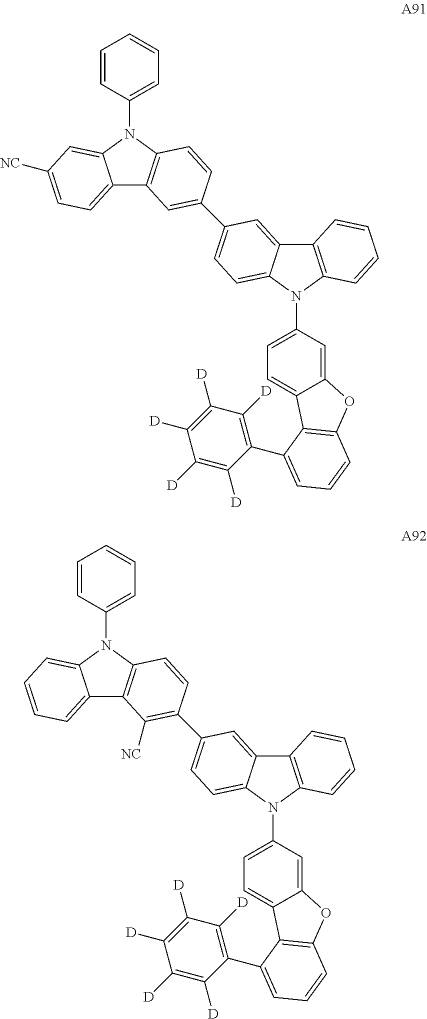 [SMILES: [2H]c1c([2H])c([2H])c(-c2cccc3oc4cc(-n5c6ccccc6c6cc(-c7ccc8c(c7)c7ccc(C#N)cc7n8-c7ccccc7)ccc65)ccc4c23)c([2H])c1[2H] ; [2H]c1c([2H])c([2H])c(-c2cccc3oc4cc(-n5c6ccccc6c6cc(-c7ccc8c(c7C#N)c7ccccc7n8-c7ccccc7)ccc65)ccc4c23)c([2H])c1[2H]]

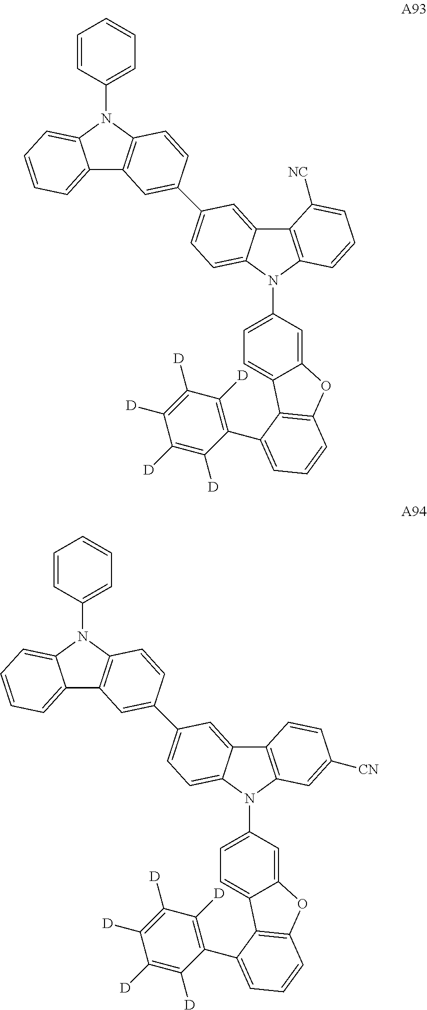 [SMILES: [2H]c1c([2H])c([2H])c(-c2cccc3oc4cc(-n5c6ccc(-c7ccc8c(c7)c7ccccc7n8-c7ccccc7)cc6c6c(C#N)cccc65)ccc4c23)c([2H])c1[2H] ; [2H]c1c([2H])c([2H])c(-c2cccc3oc4cc(-n5c6ccc(-c7ccc8c(c7)c7ccccc7n8-c7ccccc7)cc6c6ccc(C#N)cc65)ccc4c23)c([2H])c1[2H]]

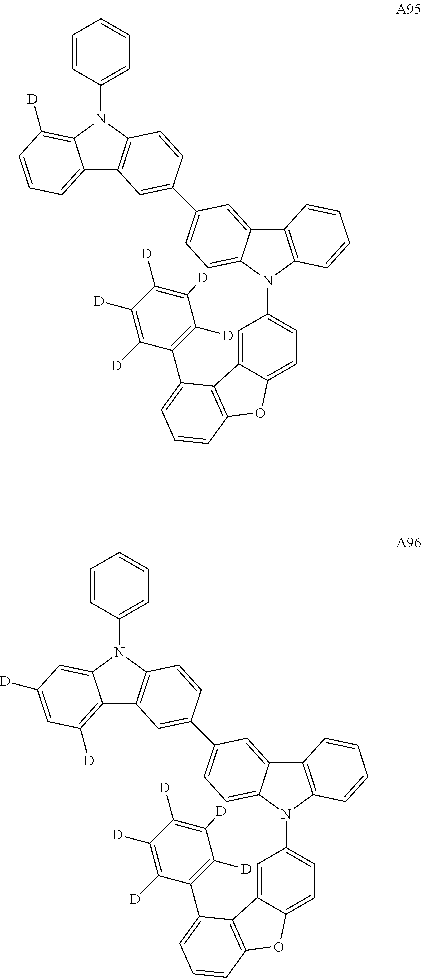 [SMILES: [2H]c1c([2H])c([2H])c(-c2cccc3oc4ccc(-n5c6ccccc6c6cc(-c7ccc8c(c7)c7cccc([2H])c7n8-c7ccccc7)ccc65)cc4c23)c([2H])c1[2H] ; [2H]c1cc([2H])c2c3cc(-c4ccc5c(c4)c4ccccc4n5-c4ccc5oc6cccc(-c7c([2H])c([2H])c([2H])c([2H])c7[2H])c6c5c4)ccc3n(-c3ccccc3)c2c1]

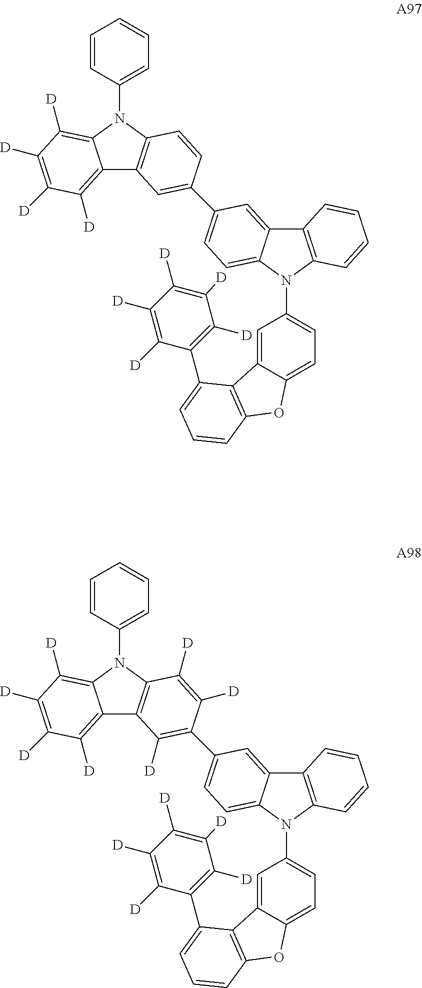 [SMILES: [2H]c1c([2H])c([2H])c(-c2cccc3oc4ccc(-n5c6ccccc6c6cc(-c7ccc8c(c7)c7c([2H])c([2H])c([2H])c([2H])c7n8-c7ccccc7)ccc65)cc4c23)c([2H])c1[2H] ; [2H]c1c([2H])c([2H])c(-c2cccc3oc4ccc(-n5c6ccccc6c6cc(-c7c([2H])c([2H])c8c(c7[2H])c7c([2H])c([2H])c([2H])c([2H])c7n8-c7ccccc7)ccc65)cc4c23)c([2H])c1[2H]]

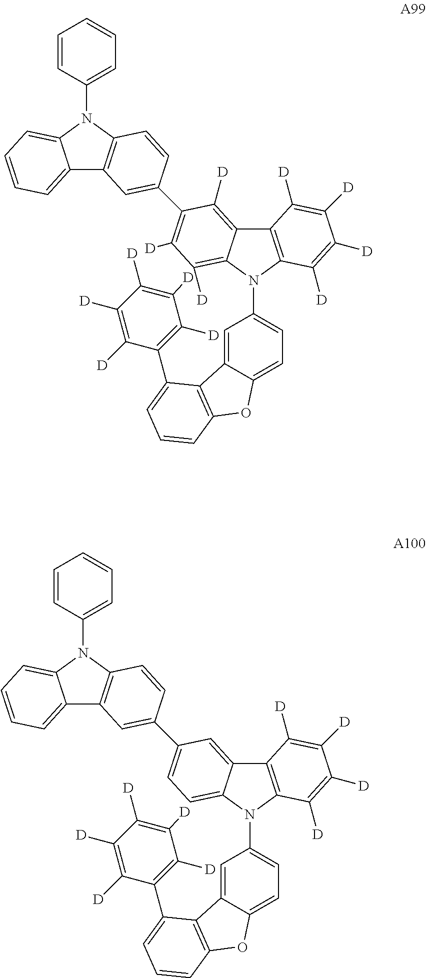 [SMILES: [2H]c1c([2H])c([2H])c(-c2cccc3oc4ccc(-n5c6c([2H])c([2H])c([2H])c([2H])c6c6c([2H])c(-c7ccc8c(c7)c7ccccc7n8-c7ccccc7)c([2H])c([2H])c65)cc4c23)c([2H])c1[2H] ; [2H]c1c([2H])c([2H])c(-c2cccc3oc4ccc(-n5c6ccc(-c7ccc8c(c7)c7ccccc7n8-c7ccccc7)cc6c6c([2H])c([2H])c([2H])c([2H])c65)cc4c23)c([2H])c1[2H]]

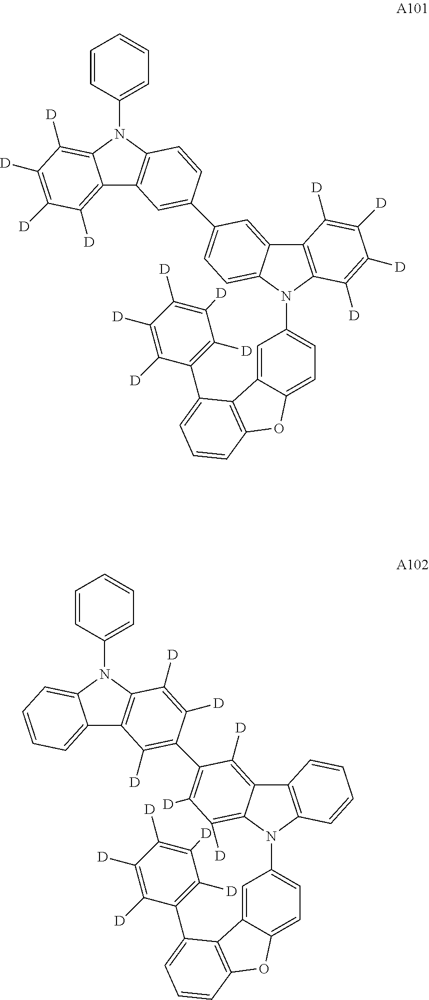 [SMILES: [2H]c1c([2H])c([2H])c(-c2cccc3oc4ccc(-n5c6ccc(-c7ccc8c(c7)c7c([2H])c([2H])c([2H])c([2H])c7n8-c7ccccc7)cc6c6c([2H])c([2H])c([2H])c([2H])c65)cc4c23)c([2H])c1[2H] ; [2H]c1c([2H])c([2H])c(-c2cccc3oc4ccc(-n5c6ccccc6c6c([2H])c(-c7c([2H])c([2H])c8c(c7[2H])c7ccccc7n8-c7ccccc7)c([2H])c([2H])c65)cc4c23)c([2H])c1[2H]]

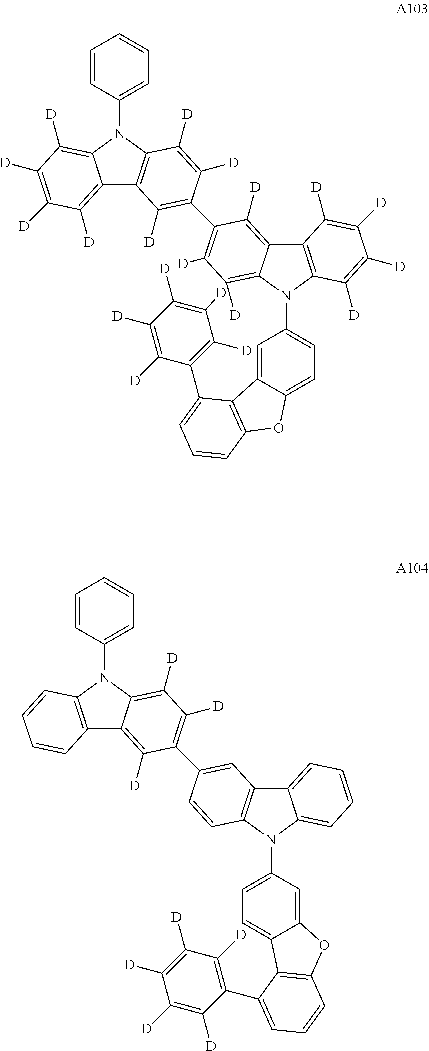 [SMILES: [2H]c1c([2H])c([2H])c(-c2cccc3oc4ccc(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)cc4c23)c([2H])c1[2H] ; [2H]c1c([2H])c([2H])c(-c2cccc3oc4cc(-n5c6ccccc6c6cc(-c7c([2H])c([2H])c8c(c7[2H])c7ccccc7n8-c7ccccc7)ccc65)ccc4c23)c([2H])c1[2H]]

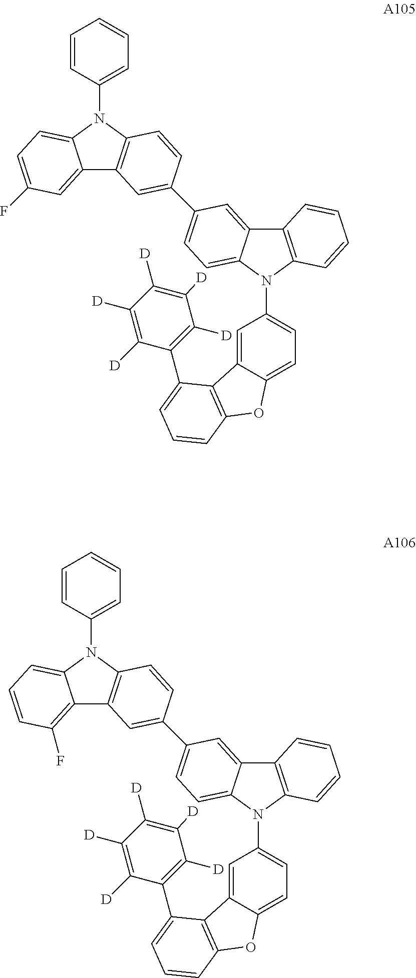 [SMILES: [2H]c1c([2H])c([2H])c(-c2cccc3oc4ccc(-n5c6ccccc6c6cc(-c7ccc8c(c7)c7cc(F)ccc7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7c(F)cccc7n8-c7ccccc7)ccc65)cc4c23)c([2H])c1[2H]]

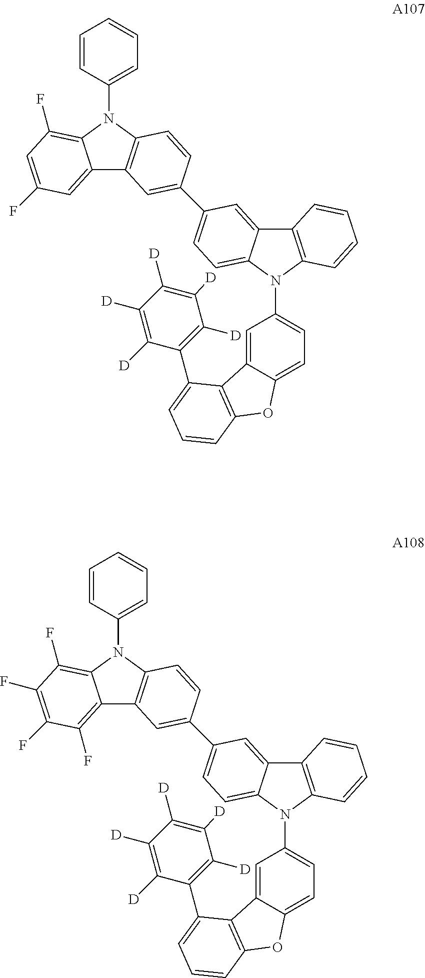 [SMILES: [2H]c1c([2H])c([2H])c(-c2cccc3oc4ccc(-n5c6ccccc6c6cc(-c7ccc8c(c7)c7cc(F)cc(F)c7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7c(F)c(F)c(F)c(F)c7n8-c7ccccc7)ccc65)cc4c23)c([2H])c1[2H]]

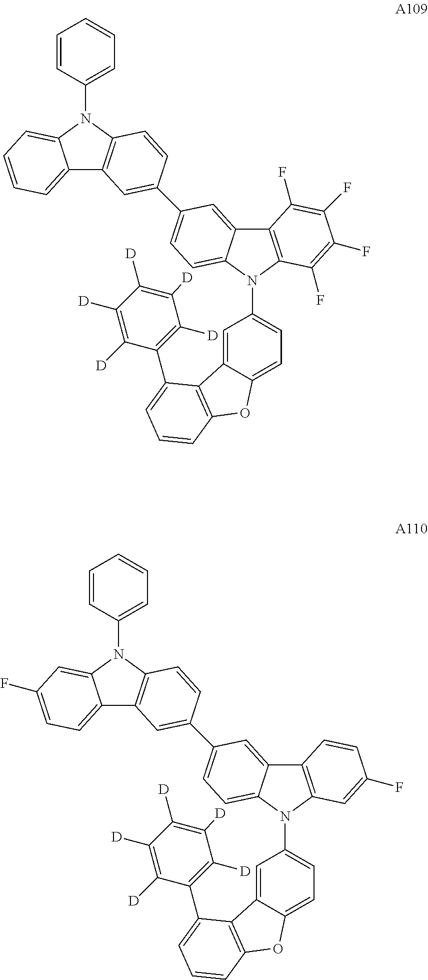 [SMILES: [2H]c1c([2H])c([2H])c(-c2cccc3oc4ccc(-n5c6ccc(-c7ccc8c(c7)c7ccccc7n8-c7ccccc7)cc6c6c(F)c(F)c(F)c(F)c65)cc4c23)c([2H])c1[2H] ; [2H]c1c([2H])c([2H])c(-c2cccc3oc4ccc(-n5c6ccc(-c7ccc8c(c7)c7ccc(F)cc7n8-c7ccccc7)cc6c6ccc(F)cc65)cc4c23)c([2H])c1[2H]]

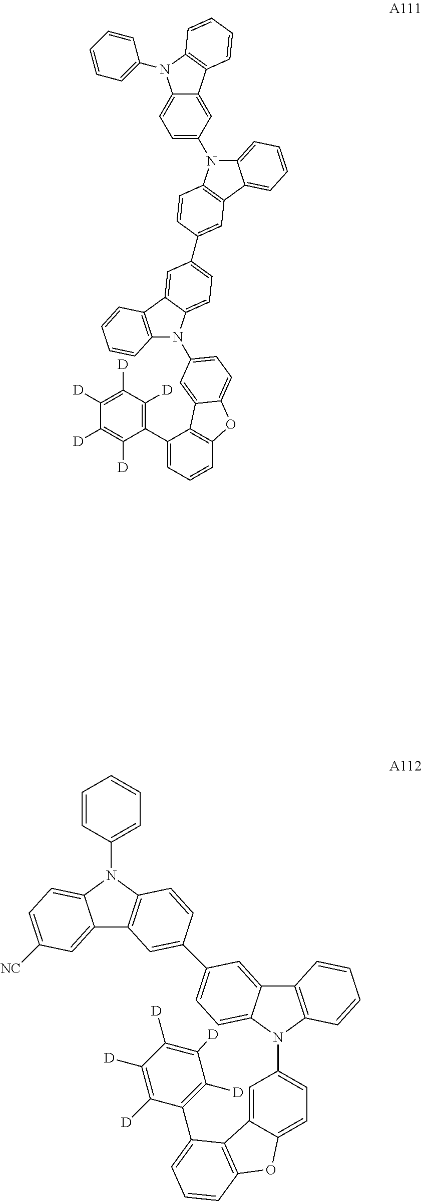 [SMILES: [2H]c1c([2H])c([2H])c(-c2cccc3oc4ccc(-n5c6ccccc6c6cc(-c7ccc8c(c7)c7ccccc7n8-c7ccc8c(c7)c7ccccc7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7cc(C#N)ccc7n8-c7ccccc7)ccc65)cc4c23)c([2H])c1[2H]]

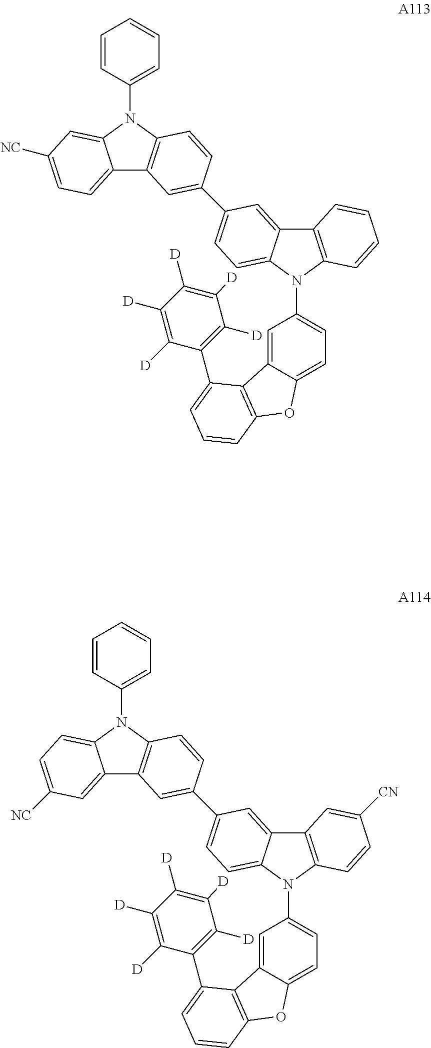 [SMILES: [2H]c1c([2H])c([2H])c(-c2cccc3oc4ccc(-n5c6ccccc6c6cc(-c7ccc8c(c7)c7ccc(C#N)cc7n8-c7ccccc7)ccc65)cc4c23)c([2H])c1[2H] ; [2H]c1c([2H])c([2H])c(-c2cccc3oc4ccc(-n5c6ccc(C#N)cc6c6cc(-c7ccc8c(c7)c7cc(C#N)ccc7n8-c7ccccc7)ccc65)cc4c23)c([2H])c1[2H]]

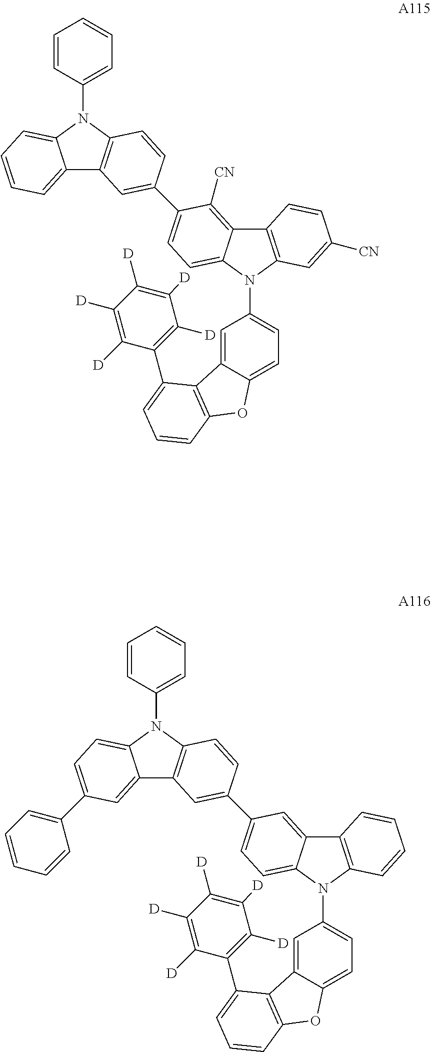 [SMILES: [2H]c1c([2H])c([2H])c(-c2cccc3oc4ccc(-n5c6cc(C#N)ccc6c6c(C#N)c(-c7ccc8c(c7)c7ccccc7n8-c7ccccc7)ccc65)cc4c23)c([2H])c1[2H] ; [2H]c1c([2H])c([2H])c(-c2cccc3oc4ccc(-n5c6ccccc6c6cc(-c7ccc8c(c7)c7cc(-c9ccccc9)ccc7n8-c7ccccc7)ccc65)cc4c23)c([2H])c1[2H]]

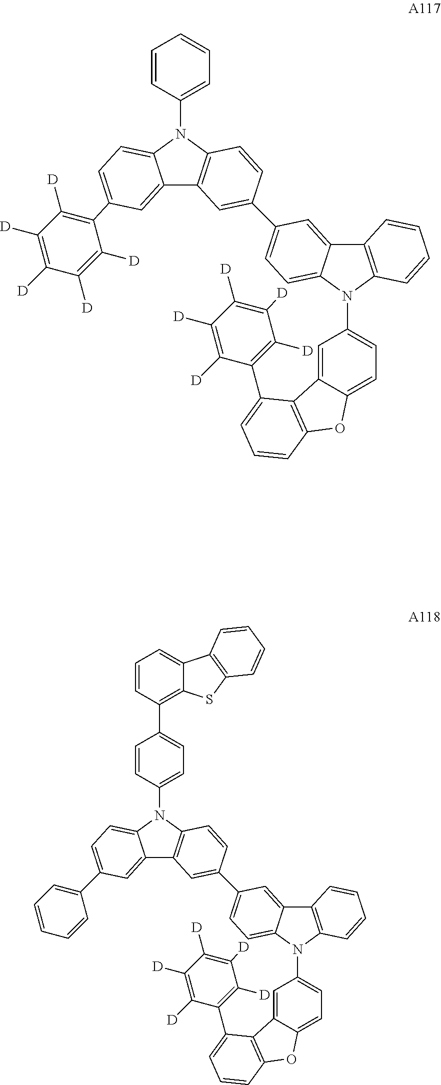 [SMILES: [2H]c1c([2H])c([2H])c(-c2ccc3c(c2)c2cc(-c4ccc5c(c4)c4ccccc4n5-c4ccc5oc6cccc(-c7c([2H])c([2H])c([2H])c([2H])c7[2H])c6c5c4)ccc2n3-c2ccccc2)c([2H])c1[2H] ; [2H]c1c([2H])c([2H])c(-c2cccc3oc4ccc(-n5c6ccccc6c6cc(-c7ccc8c(c7)c7cc(-c9ccccc9)ccc7n8-c7ccc(-c8cccc9c8sc8ccccc89)cc7)ccc65)cc4c23)c([2H])c1[2H]]

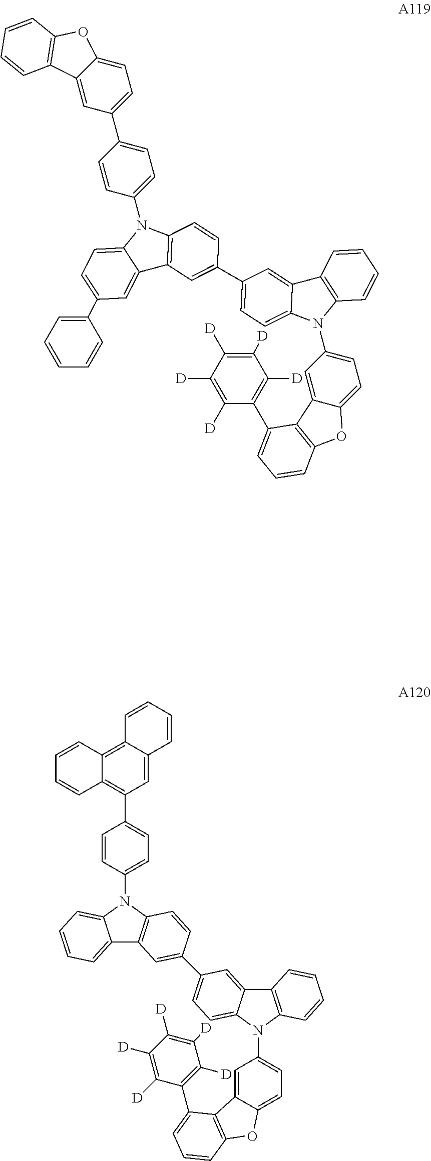 [SMILES: [2H]c1c([2H])c([2H])c(-c2cccc3oc4ccc(-n5c6ccccc6c6cc(-c7ccc8c(c7)c7cc(-c9ccccc9)ccc7n8-c7ccc(-c8ccc9oc%10ccccc%10c9c8)cc7)ccc65)cc4c23)c([2H])c1[2H] ; [2H]c1c([2H])c([2H])c(-c2cccc3oc4ccc(-n5c6ccccc6c6cc(-c7ccc8c(c7)c7ccccc7n8-c7ccc(-c8cc9ccccc9c9ccccc89)cc7)ccc65)cc4c23)c([2H])c1[2H]]

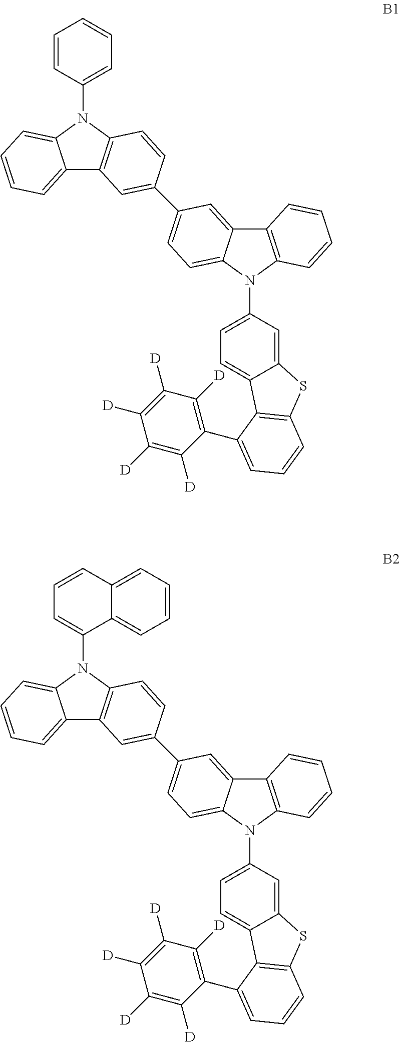 [SMILES: [2H]c1c([2H])c([2H])c(-c2cccc3sc4cc(-n5c6ccccc6c6cc(-c7ccc8c(c7)c7ccccc7n8-c7ccccc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc8ccccc78)ccc65)ccc4c23)c([2H])c1[2H]]

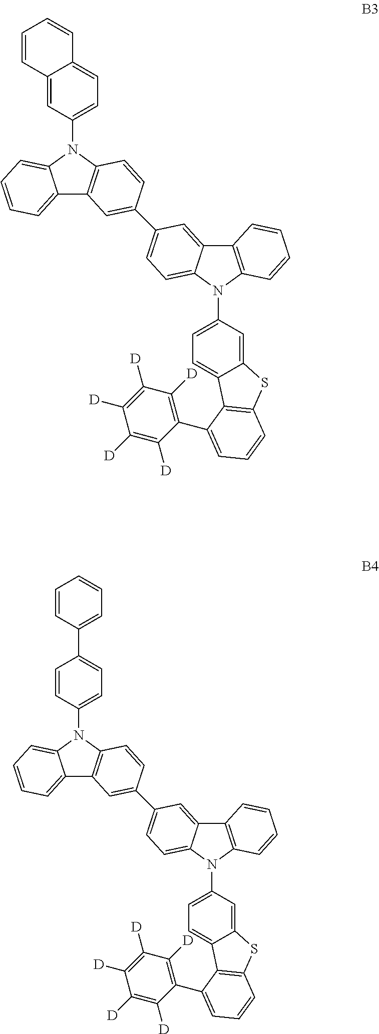 [SMILES: [2H]c1c([2H])c([2H])c(-c2cccc3sc4cc(-n5c6ccccc6c6cc(-c7ccc8c(c7)c7ccccc7n8-c7ccc8ccccc8c7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc(-c8ccccc8)cc7)ccc65)ccc4c23)c([2H])c1[2H]]

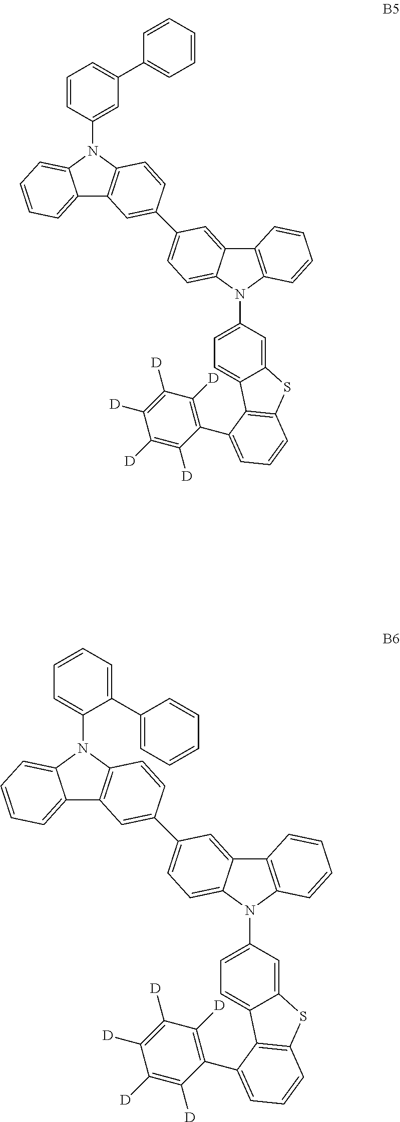 [SMILES: [2H]c1c([2H])c([2H])c(-c2cccc3sc4cc(-n5c6ccccc6c6cc(-c7ccc8c(c7)c7ccccc7n8-c7cccc(-c8ccccc8)c7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccccc7-c7ccccc7)ccc65)ccc4c23)c([2H])c1[2H]]

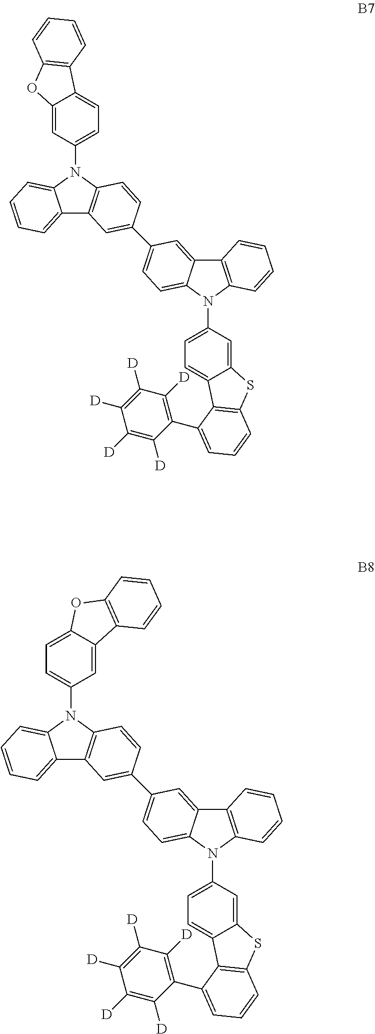 [SMILES: [2H]c1c([2H])c([2H])c(-c2cccc3sc4cc(-n5c6ccccc6c6cc(-c7ccc8c(c7)c7ccccc7n8-c7ccc8c(c7)oc7ccccc78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc8oc9ccccc9c8c7)ccc65)ccc4c23)c([2H])c1[2H]]

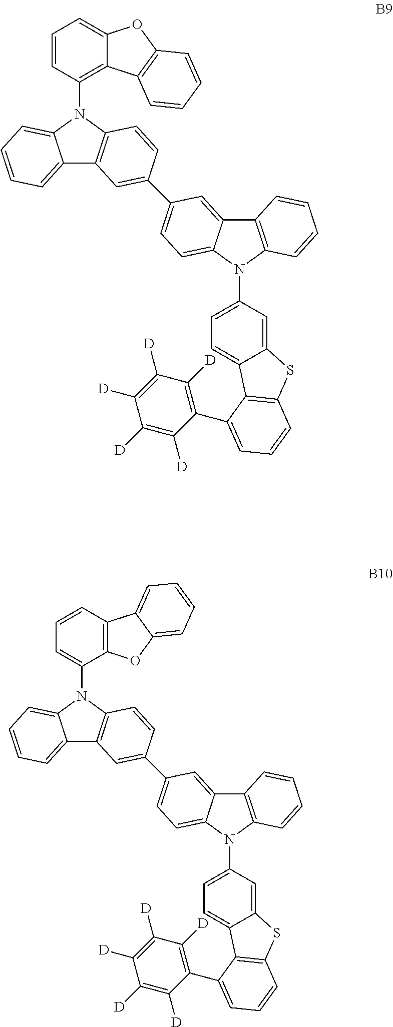 [SMILES: [2H]c1c([2H])c([2H])c(-c2cccc3sc4cc(-n5c6ccccc6c6cc(-c7ccc8c(c7)c7ccccc7n8-c7cccc8oc9ccccc9c78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc8c7oc7ccccc78)ccc65)ccc4c23)c([2H])c1[2H]]

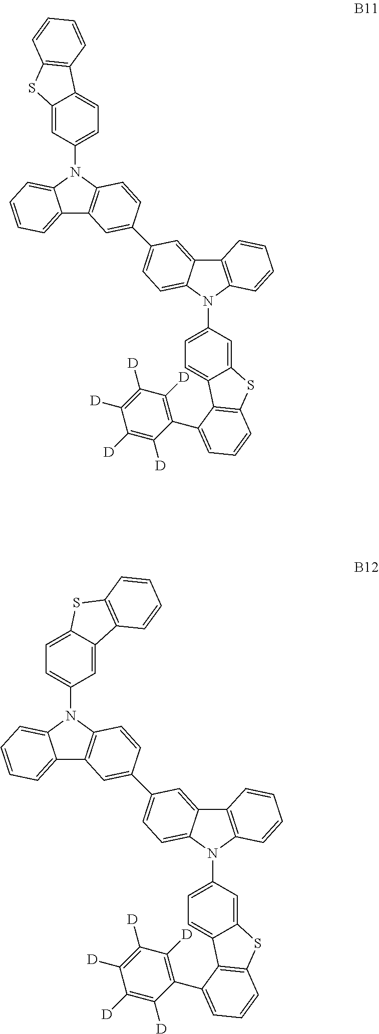 [SMILES: [2H]c1c([2H])c([2H])c(-c2cccc3sc4cc(-n5c6ccccc6c6cc(-c7ccc8c(c7)c7ccccc7n8-c7ccc8c(c7)sc7ccccc78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc8sc9ccccc9c8c7)ccc65)ccc4c23)c([2H])c1[2H]]

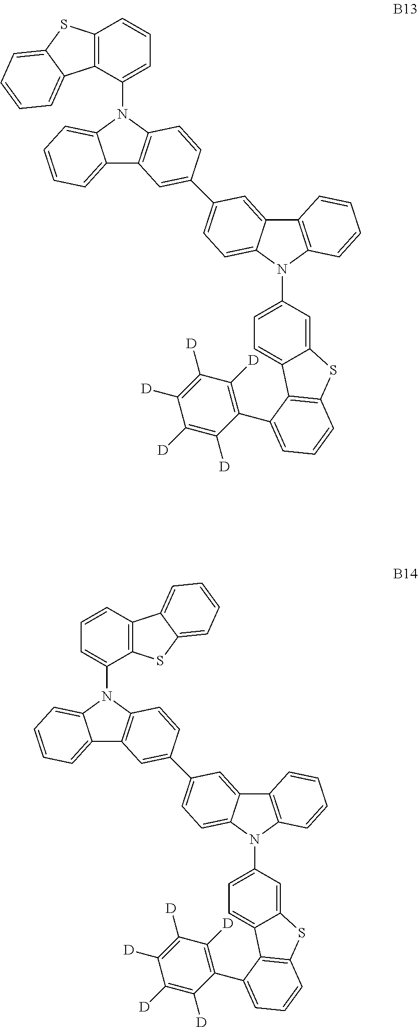 [SMILES: [2H]c1c([2H])c([2H])c(-c2cccc3sc4cc(-n5c6ccccc6c6cc(-c7ccc8c(c7)c7ccccc7n8-c7cccc8sc9ccccc9c78)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc8c7sc7ccccc78)ccc65)ccc4c23)c([2H])c1[2H]]

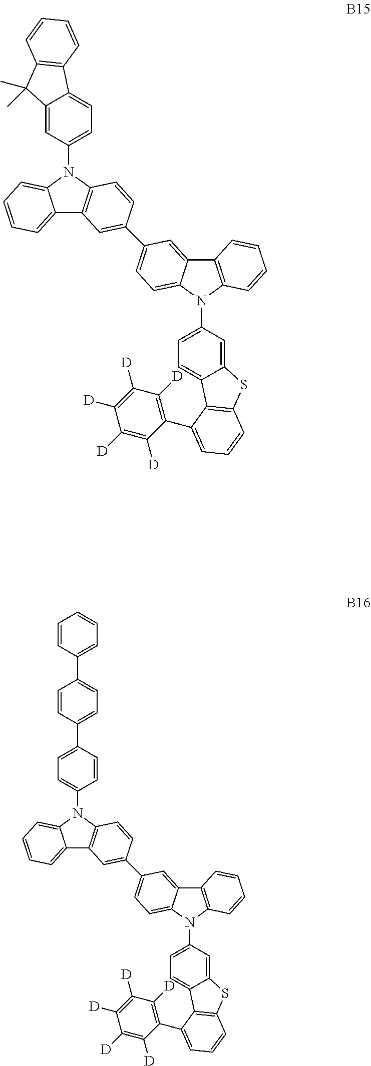 [SMILES: [2H]c1c([2H])c([2H])c(-c2cccc3sc4cc(-n5c6ccccc6c6cc(-c7ccc8c(c7)c7ccccc7n8-c7ccc8c(c7)C(C)(C)c7ccccc7-8)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc(-c8ccc(-c9ccccc9)cc8)cc7)ccc65)ccc4c23)c([2H])c1[2H]]

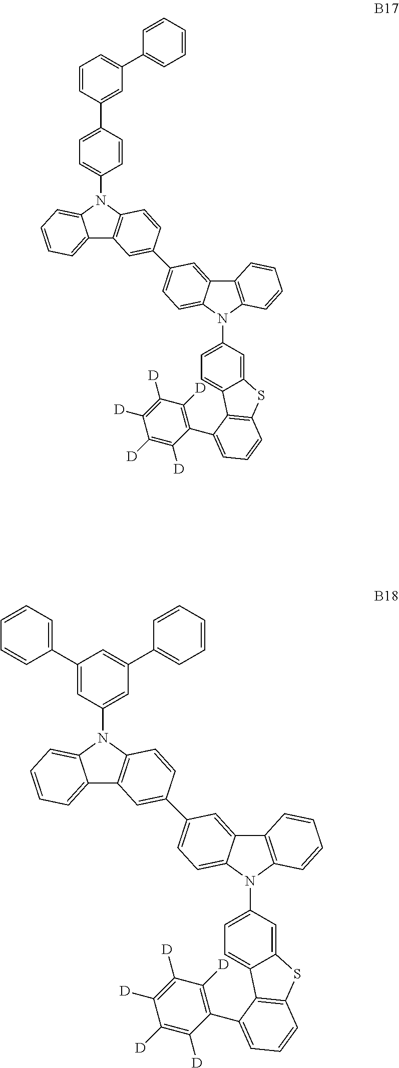 [SMILES: [2H]c1c([2H])c([2H])c(-c2cccc3sc4cc(-n5c6ccccc6c6cc(-c7ccc8c(c7)c7ccccc7n8-c7ccc(-c8cccc(-c9ccccc9)c8)cc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cc(-c8ccccc8)cc(-c8ccccc8)c7)ccc65)ccc4c23)c([2H])c1[2H]]

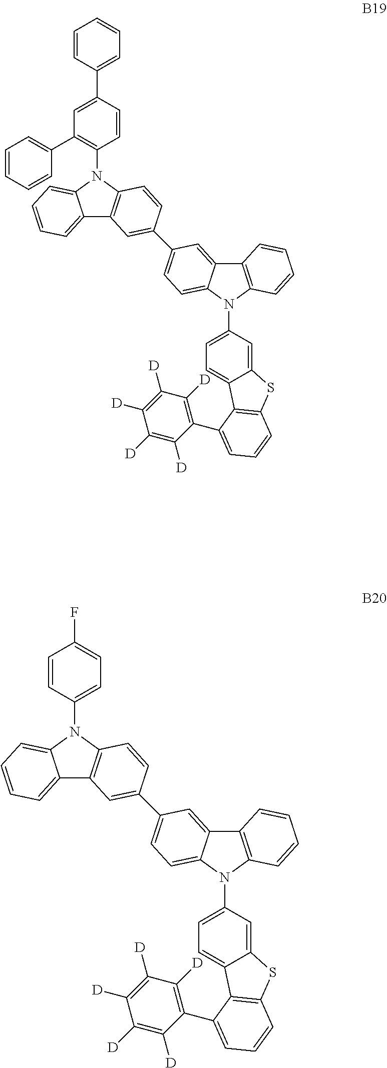 [SMILES: [2H]c1c([2H])c([2H])c(-c2cccc3sc4cc(-n5c6ccccc6c6cc(-c7ccc8c(c7)c7ccccc7n8-c7ccc(-c8ccccc8)cc7-c7ccccc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7ccc(F)cc7)ccc65)ccc4c23)c([2H])c1[2H]]

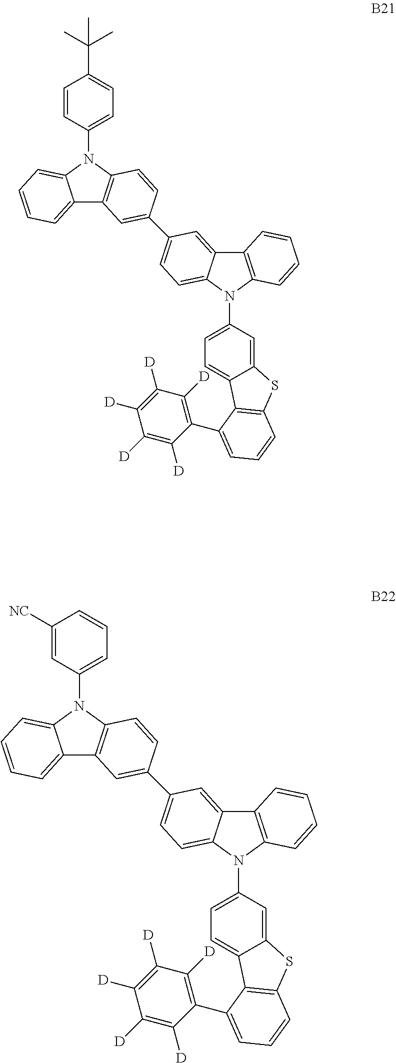 [SMILES: [2H]c1c([2H])c([2H])c(-c2cccc3sc4cc(-n5c6ccccc6c6cc(-c7ccc8c(c7)c7ccccc7n8-c7ccc(C(C)(C)C)cc7)ccc65)ccc4c23)c([2H])c1[2H] ; [2H]c1c([2H])c([2H])c(-c2cccc3sc4cc(-n5c6ccccc6c6cc(-c7ccc8c(c7)c7ccccc7n8-c7cccc(C#N)c7)ccc65)ccc4c23)c([2H])c1[2H]]

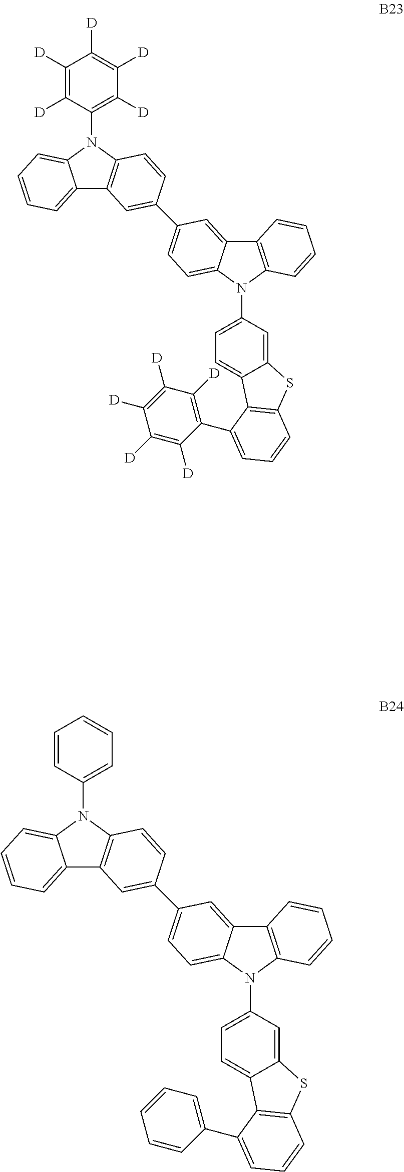 [SMILES: [2H]c1c([2H])c([2H])c(-c2cccc3sc4cc(-n5c6ccccc6c6cc(-c7ccc8c(c7)c7ccccc7n8-c7c([2H])c([2H])c([2H])c([2H])c7[2H])ccc65)ccc4c23)c([2H])c1[2H] ; c1ccc(-c2cccc3sc4cc(-n5c6ccccc6c6cc(-c7ccc8c(c7)c7ccccc7n8-c7ccccc7)ccc65)ccc4c23)cc1]

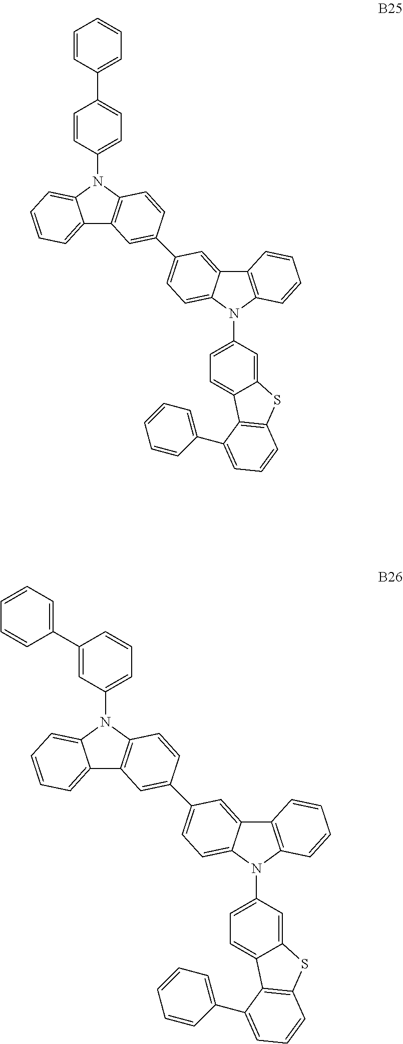 [SMILES: c1ccc(-c2ccc(-n3c4ccccc4c4cc(-c5ccc6c(c5)c5ccccc5n6-c5ccc6c(c5)sc5cccc(-c7ccccc7)c56)ccc43)cc2)cc1 ; c1ccc(-c2cccc(-n3c4ccccc4c4cc(-c5ccc6c(c5)c5ccccc5n6-c5ccc6c(c5)sc5cccc(-c7ccccc7)c56)ccc43)c2)cc1]

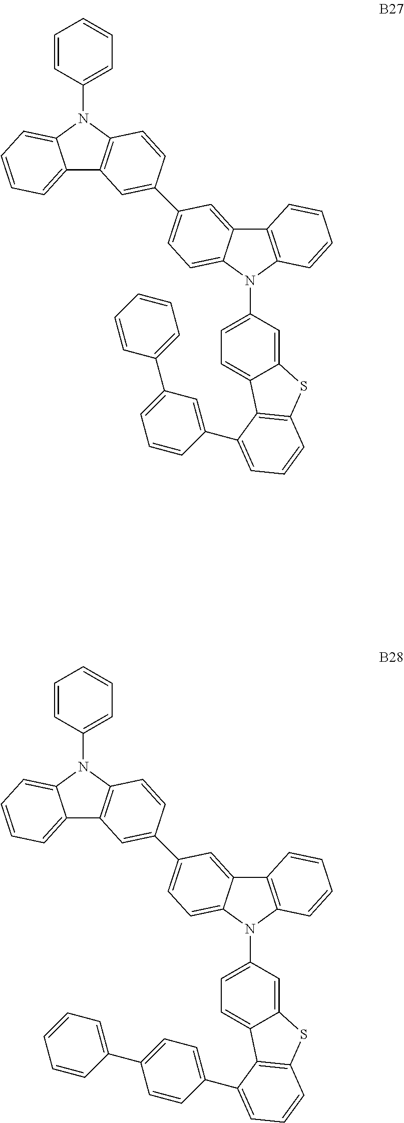 [SMILES: c1ccc(-c2cccc(-c3cccc4sc5cc(-n6c7ccccc7c7cc(-c8ccc9c(c8)c8ccccc8n9-c8ccccc8)ccc76)ccc5c34)c2)cc1 ; c1ccc(-c2ccc(-c3cccc4sc5cc(-n6c7ccccc7c7cc(-c8ccc9c(c8)c8ccccc8n9-c8ccccc8)ccc76)ccc5c34)cc2)cc1]

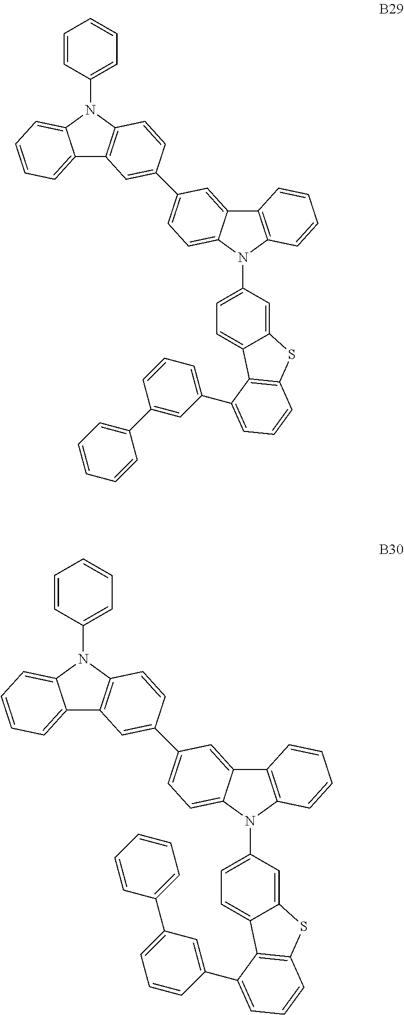 [SMILES: c1ccc(-c2cccc(-c3cccc4sc5cc(-n6c7ccccc7c7cc(-c8ccc9c(c8)c8ccccc8n9-c8ccccc8)ccc76)ccc5c34)c2)cc1 ; c1ccc(-c2cccc(-c3cccc4sc5cc(-n6c7ccccc7c7cc(-c8ccc9c(c8)c8ccccc8n9-c8ccccc8)ccc76)ccc5c34)c2)cc1]

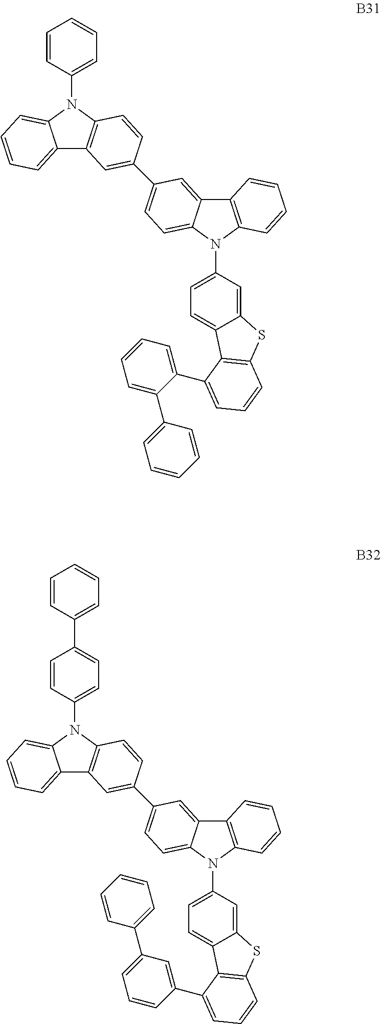 [SMILES: c1ccc(-c2ccccc2-c2cccc3sc4cc(-n5c6ccccc6c6cc(-c7ccc8c(c7)c7ccccc7n8-c7ccccc7)ccc65)ccc4c23)cc1 ; c1ccc(-c2ccc(-n3c4ccccc4c4cc(-c5ccc6c(c5)c5ccccc5n6-c5ccc6c(c5)sc5cccc(-c7cccc(-c8ccccc8)c7)c56)ccc43)cc2)cc1]

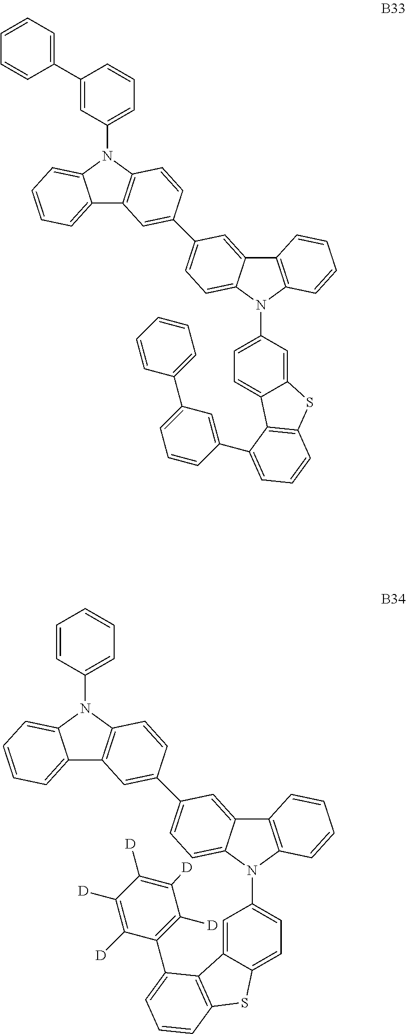 [SMILES: c1ccc(-c2cccc(-c3cccc4sc5cc(-n6c7ccccc7c7cc(-c8ccc9c(c8)c8ccccc8n9-c8cccc(-c9ccccc9)c8)ccc76)ccc5c34)c2)cc1 ; [2H]c1c([2H])c([2H])c(-c2cccc3sc4ccc(-n5c6ccccc6c6cc(-c7ccc8c(c7)c7ccccc7n8-c7ccccc7)ccc65)cc4c23)c([2H])c1[2H]]

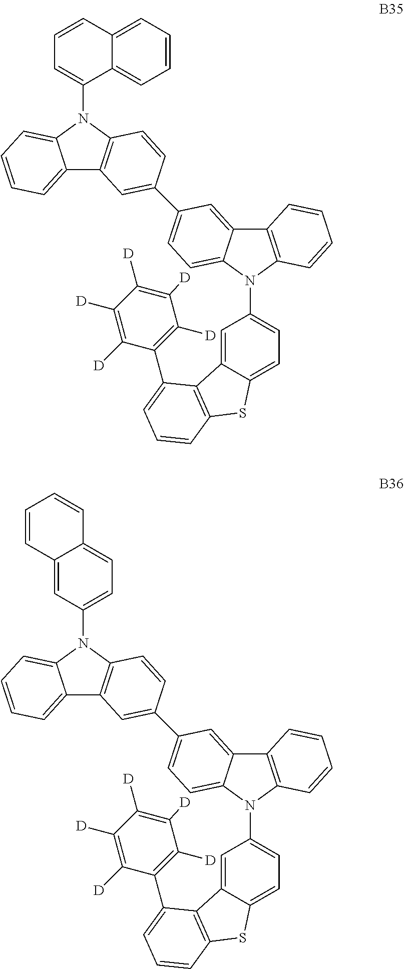 [SMILES: [2H]c1c([2H])c([2H])c(-c2cccc3sc4ccc(-n5c6ccccc6c6cc(-c7ccc8c(c7)c7ccccc7n8-c7cccc8ccccc78)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8ccccc8c7)ccc65)cc4c23)c([2H])c1[2H]]

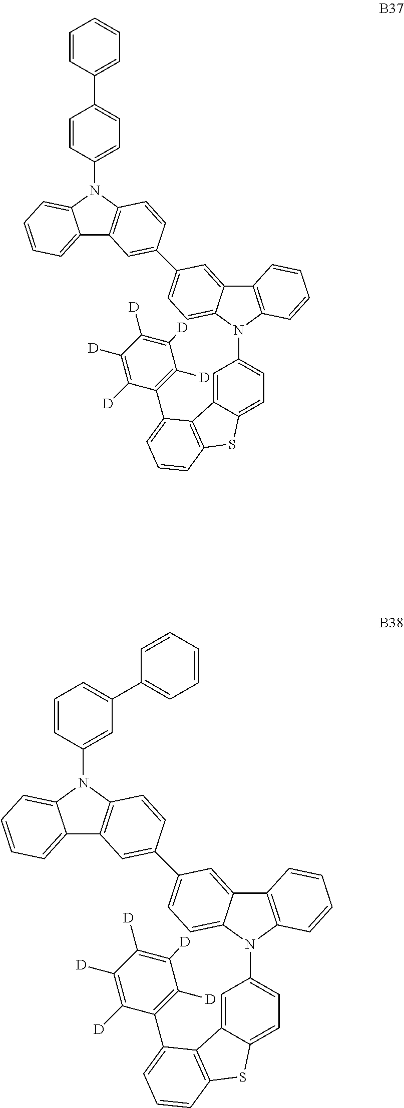 [SMILES: [2H]c1c([2H])c([2H])c(-c2cccc3sc4ccc(-n5c6ccccc6c6cc(-c7ccc8c(c7)c7ccccc7n8-c7ccc(-c8ccccc8)cc7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7cccc(-c8ccccc8)c7)ccc65)cc4c23)c([2H])c1[2H]]

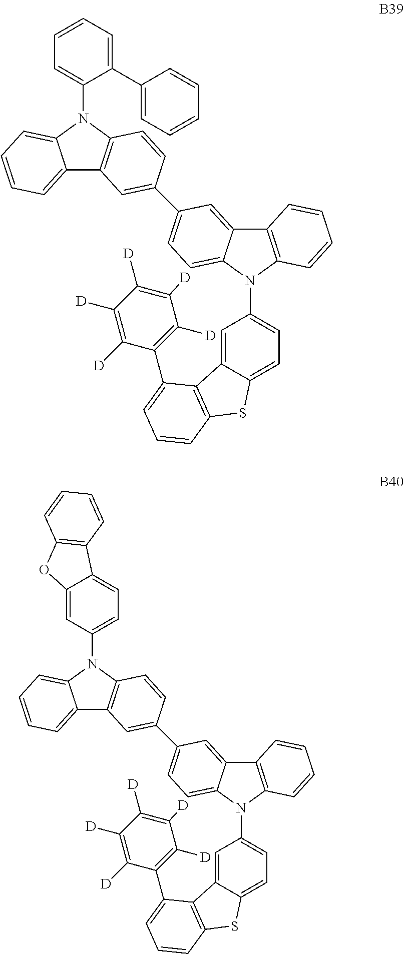 [SMILES: [2H]c1c([2H])c([2H])c(-c2cccc3sc4ccc(-n5c6ccccc6c6cc(-c7ccc8c(c7)c7ccccc7n8-c7ccccc7-c7ccccc7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8c(c7)oc7ccccc78)ccc65)cc4c23)c([2H])c1[2H]]

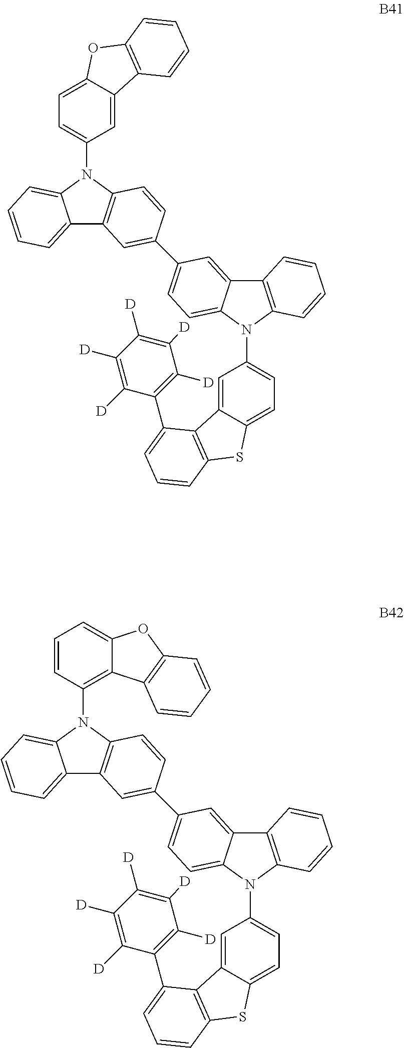 [SMILES: [2H]c1c([2H])c([2H])c(-c2cccc3sc4ccc(-n5c6ccccc6c6cc(-c7ccc8c(c7)c7ccccc7n8-c7ccc8oc9ccccc9c8c7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7cccc8oc9ccccc9c78)ccc65)cc4c23)c([2H])c1[2H]]

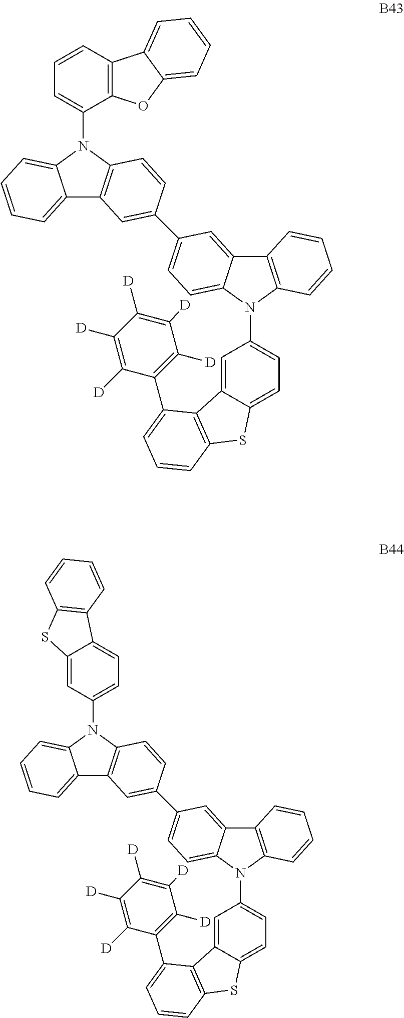 [SMILES: [2H]c1c([2H])c([2H])c(-c2cccc3sc4ccc(-n5c6ccccc6c6cc(-c7ccc8c(c7)c7ccccc7n8-c7cccc8c7oc7ccccc78)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8c(c7)sc7ccccc78)ccc65)cc4c23)c([2H])c1[2H]]

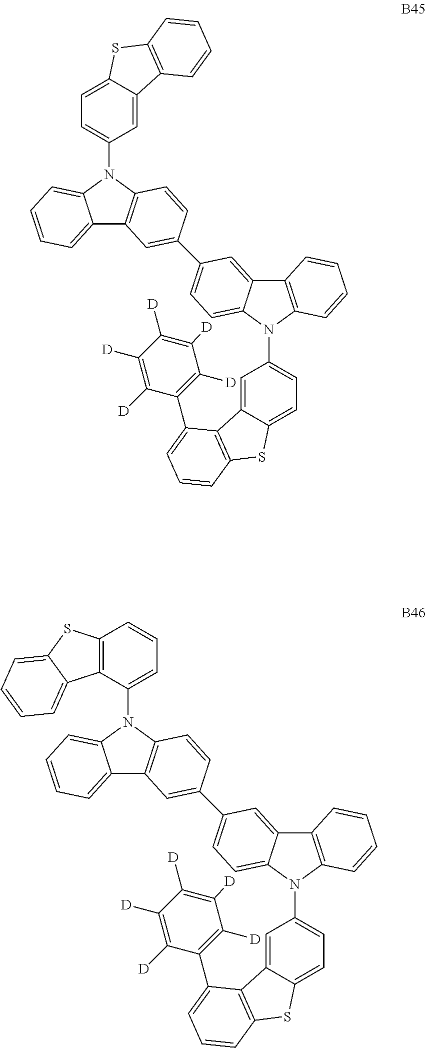 [SMILES: [2H]c1c([2H])c([2H])c(-c2cccc3sc4ccc(-n5c6ccccc6c6cc(-c7ccc8c(c7)c7ccccc7n8-c7ccc8sc9ccccc9c8c7)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7cccc8sc9ccccc9c78)ccc65)cc4c23)c([2H])c1[2H]]

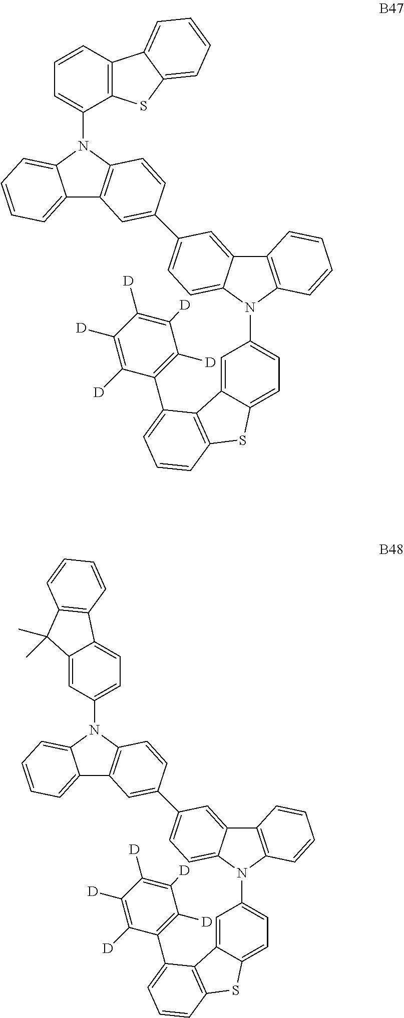 [SMILES: [2H]c1c([2H])c([2H])c(-c2cccc3sc4ccc(-n5c6ccccc6c6cc(-c7ccc8c(c7)c7ccccc7n8-c7cccc8c7sc7ccccc78)ccc65)cc4c23)c([2H])c1[2H] ; [2H]c1c([2H])c([2H])c(-c2cccc3sc4ccc(-n5c6ccccc6c6cc(-c7ccc8c(c7)c7ccccc7n8-c7ccc8c(c7)C(C)(C)c7ccccc7-8)ccc65)cc4c23)c([2H])c1[2H]]

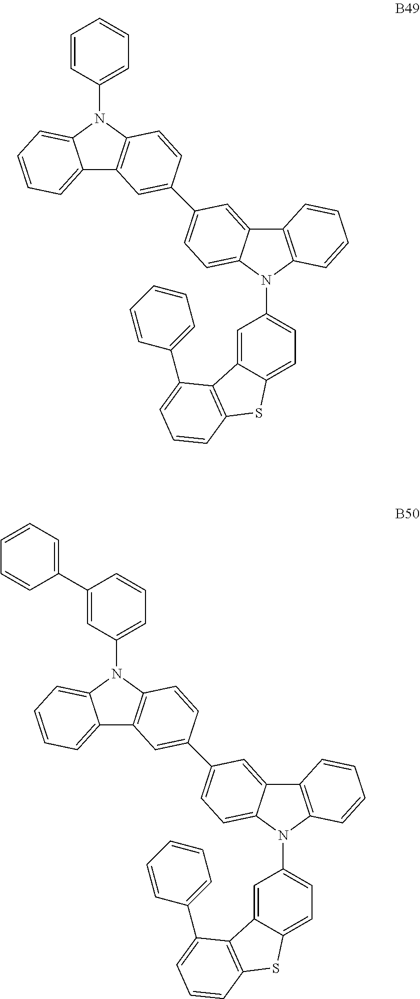 [SMILES: c1ccc(-c2cccc3sc4ccc(-n5c6ccccc6c6cc(-c7ccc8c(c7)c7ccccc7n8-c7ccccc7)ccc65)cc4c23)cc1 ; c1ccc(-c2cccc(-n3c4ccccc4c4cc(-c5ccc6c(c5)c5ccccc5n6-c5ccc6sc7cccc(-c8ccccc8)c7c6c5)ccc43)c2)cc1]

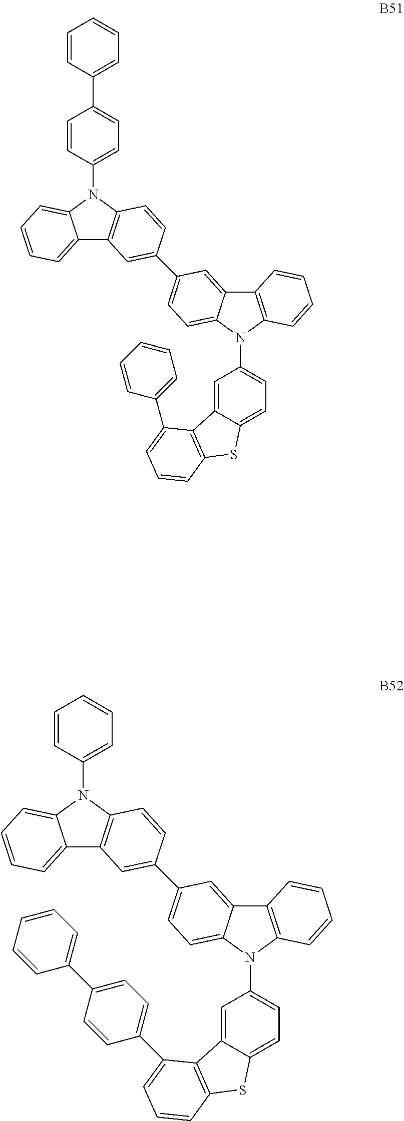 [SMILES: c1ccc(-c2ccc(-n3c4ccccc4c4cc(-c5ccc6c(c5)c5ccccc5n6-c5ccc6sc7cccc(-c8ccccc8)c7c6c5)ccc43)cc2)cc1 ; c1ccc(-c2ccc(-c3cccc4sc5ccc(-n6c7ccccc7c7cc(-c8ccc9c(c8)c8ccccc8n9-c8ccccc8)ccc76)cc5c34)cc2)cc1]

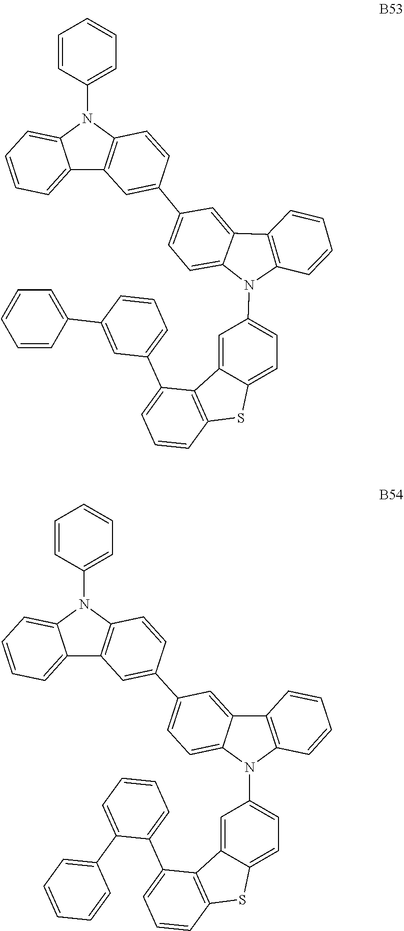 [SMILES: c1ccc(-c2cccc(-c3cccc4sc5ccc(-n6c7ccccc7c7cc(-c8ccc9c(c8)c8ccccc8n9-c8ccccc8)ccc76)cc5c34)c2)cc1 ; c1ccc(-c2ccccc2-c2cccc3sc4ccc(-n5c6ccccc6c6cc(-c7ccc8c(c7)c7ccccc7n8-c7ccccc7)ccc65)cc4c23)cc1]

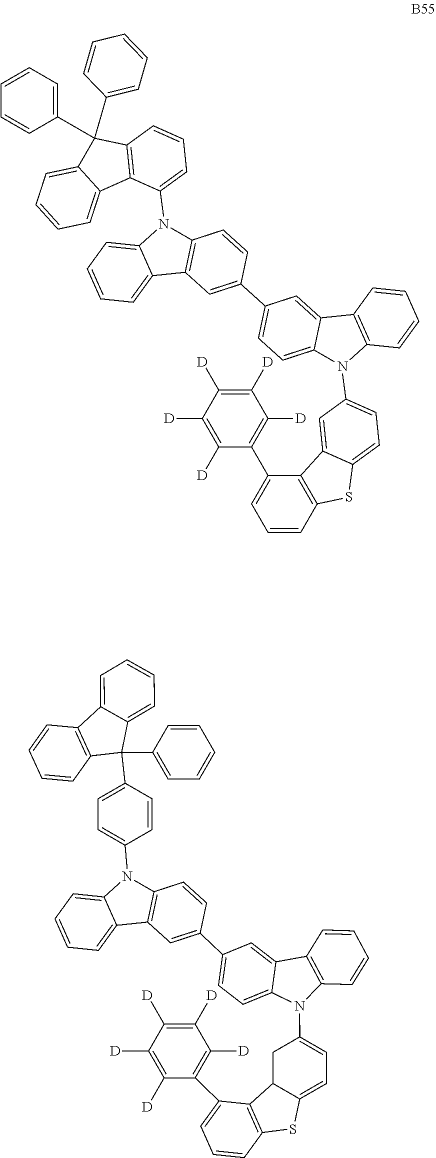 [SMILES: [2H]c1c([2H])c([2H])c(-c2cccc3sc4ccc(-n5c6ccccc6c6cc(-c7ccc8c(c7)c7ccccc7n8-c7cccc8c7-c7ccccc7C8(c7ccccc7)c7ccccc7)ccc65)cc4c23)c([2H])c1[2H] ; [2H]c1c([2H])c([2H])c(-c2cccc3c2C2CC(n4c5ccccc5c5cc(-c6ccc7c(c6)c6ccccc6n7-c6ccc(C7(c8ccccc8)c8ccccc8-c8ccccc87)cc6)ccc54)=CC=C2S3)c([2H])c1[2H]]

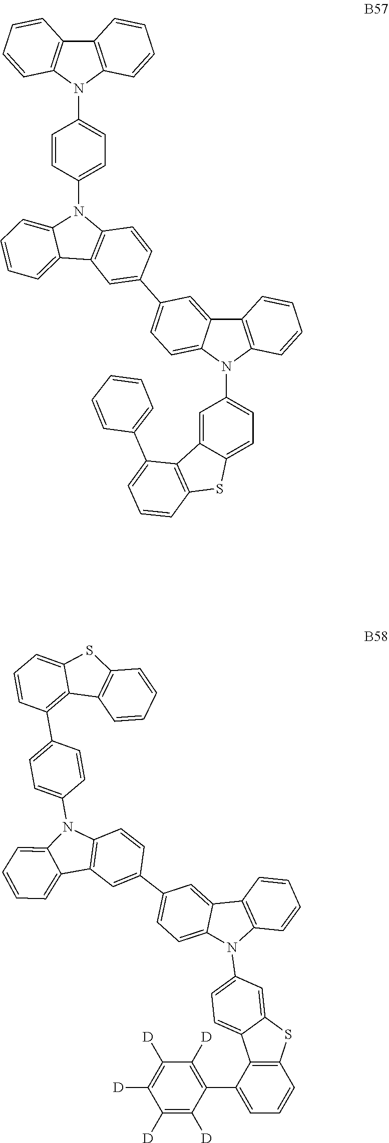 [SMILES: c1ccc(-c2cccc3sc4ccc(-n5c6ccccc6c6cc(-c7ccc8c(c7)c7ccccc7n8-c7ccc(-n8c9ccccc9c9ccccc98)cc7)ccc65)cc4c23)cc1 ; [2H]c1c([2H])c([2H])c(-c2cccc3sc4cc(-n5c6ccccc6c6cc(-c7ccc8c(c7)c7ccccc7n8-c7ccc(-c8cccc9sc%10ccccc%10c89)cc7)ccc65)ccc4c23)c([2H])c1[2H]]

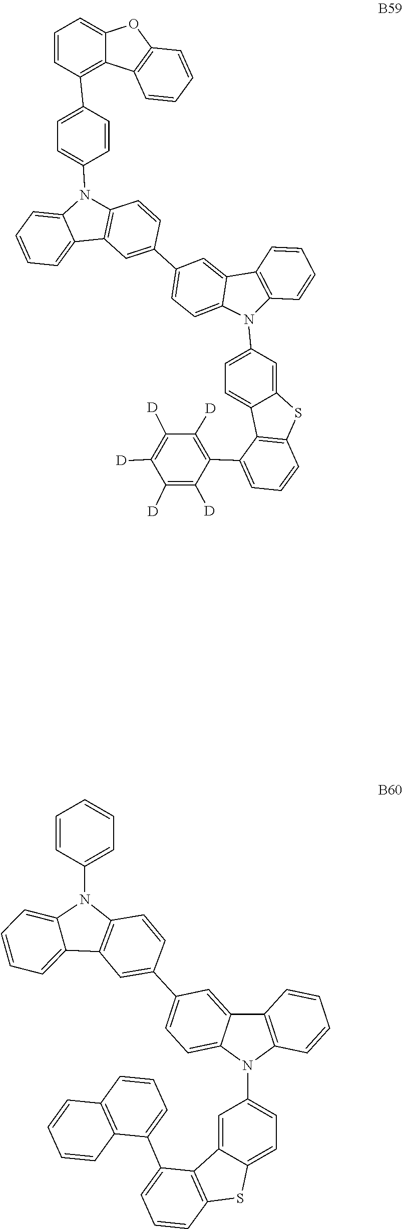 [SMILES: [2H]c1c([2H])c([2H])c(-c2cccc3sc4cc(-n5c6ccccc6c6cc(-c7ccc8c(c7)c7ccccc7n8-c7ccc(-c8cccc9oc%10ccccc%10c89)cc7)ccc65)ccc4c23)c([2H])c1[2H] ; c1ccc(-n2c3ccccc3c3cc(-c4ccc5c(c4)c4ccccc4n5-c4ccc5sc6cccc(-c7cccc8ccccc78)c6c5c4)ccc32)cc1]

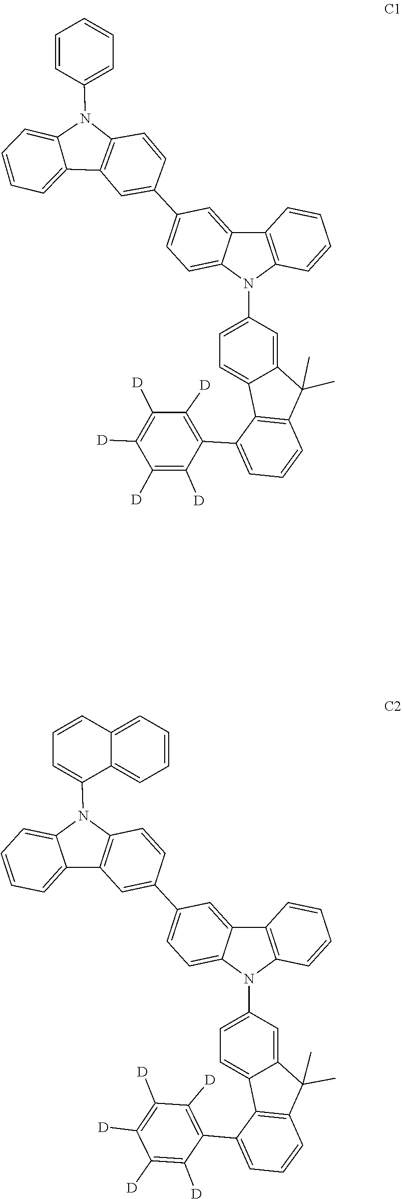 [SMILES: [2H]c1c([2H])c([2H])c(-c2cccc3c2-c2ccc(-n4c5ccccc5c5cc(-c6ccc7c(c6)c6ccccc6n7-c6ccccc6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6cccc7ccccc67)ccc54)cc2C3(C)C)c([2H])c1[2H]]

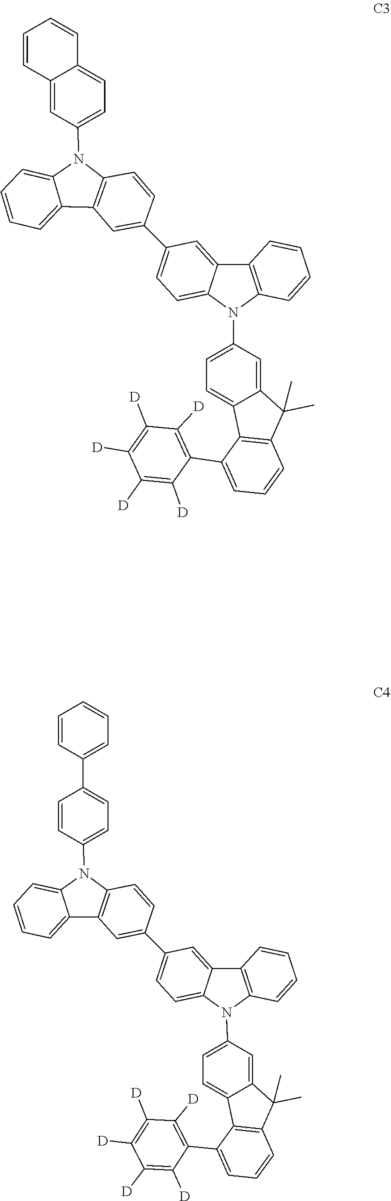 [SMILES: [2H]c1c([2H])c([2H])c(-c2cccc3c2-c2ccc(-n4c5ccccc5c5cc(-c6ccc7c(c6)c6ccccc6n7-c6ccc7ccccc7c6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc(-c7ccccc7)cc6)ccc54)cc2C3(C)C)c([2H])c1[2H]]

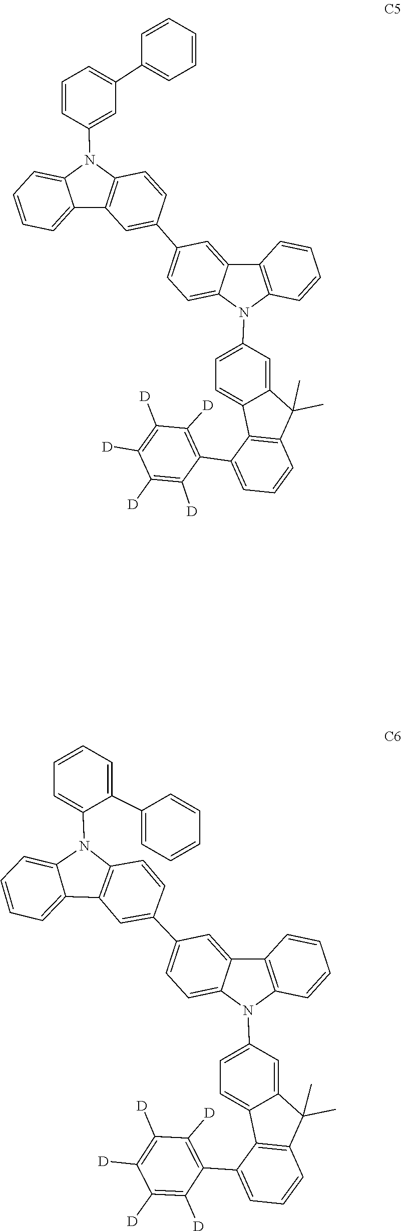 [SMILES: [2H]c1c([2H])c([2H])c(-c2cccc3c2-c2ccc(-n4c5ccccc5c5cc(-c6ccc7c(c6)c6ccccc6n7-c6cccc(-c7ccccc7)c6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccccc6-c6ccccc6)ccc54)cc2C3(C)C)c([2H])c1[2H]]

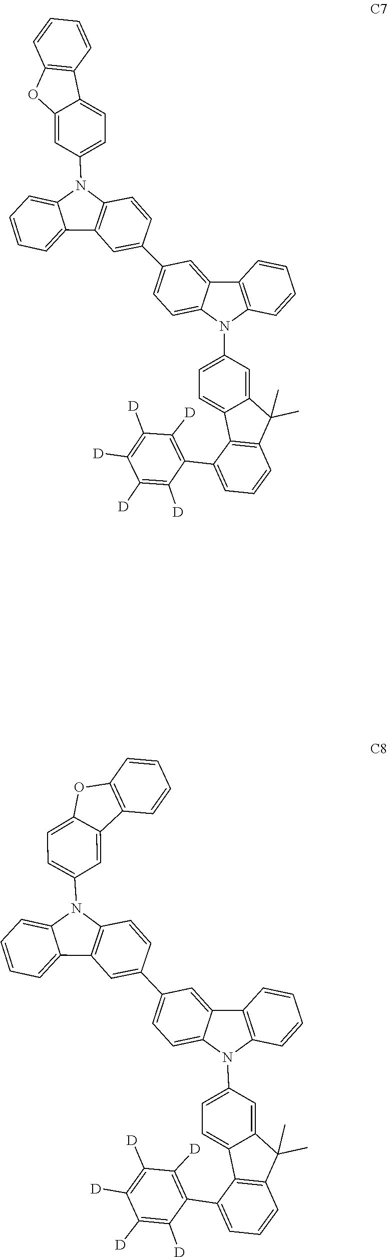 [SMILES: [2H]c1c([2H])c([2H])c(-c2cccc3c2-c2ccc(-n4c5ccccc5c5cc(-c6ccc7c(c6)c6ccccc6n7-c6ccc7c(c6)oc6ccccc67)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc7oc8ccccc8c7c6)ccc54)cc2C3(C)C)c([2H])c1[2H]]

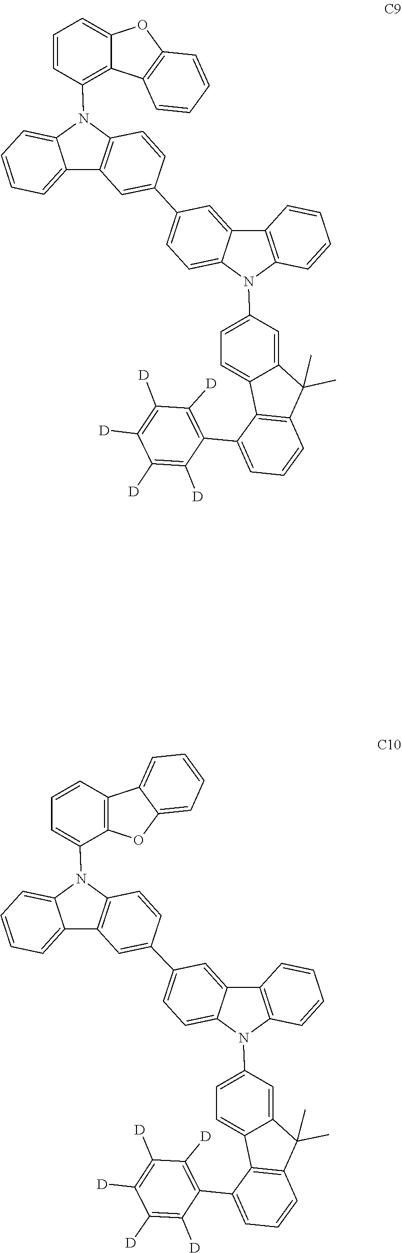 [SMILES: [2H]c1c([2H])c([2H])c(-c2cccc3c2-c2ccc(-n4c5ccccc5c5cc(-c6ccc7c(c6)c6ccccc6n7-c6cccc7oc8ccccc8c67)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6cccc7c6oc6ccccc67)ccc54)cc2C3(C)C)c([2H])c1[2H]]

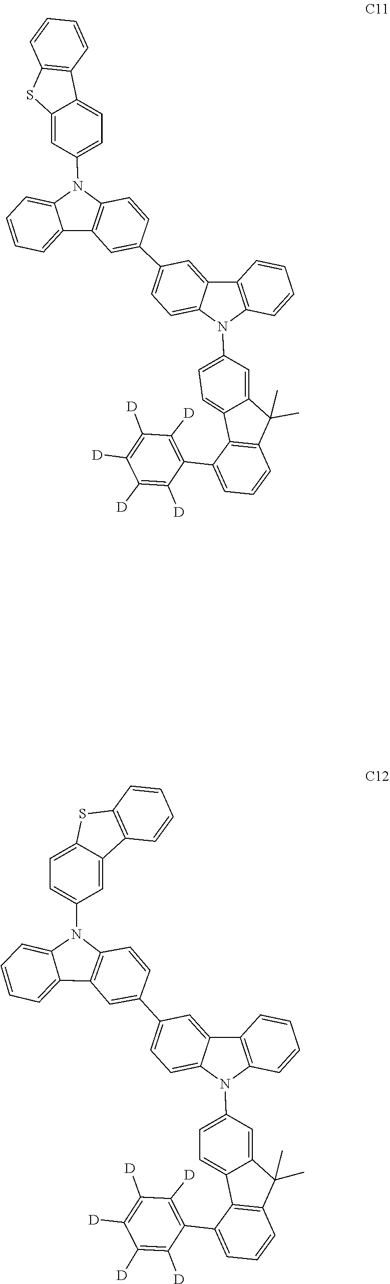 [SMILES: [2H]c1c([2H])c([2H])c(-c2cccc3c2-c2ccc(-n4c5ccccc5c5cc(-c6ccc7c(c6)c6ccccc6n7-c6ccc7c(c6)sc6ccccc67)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6ccc7sc8ccccc8c7c6)ccc54)cc2C3(C)C)c([2H])c1[2H]]

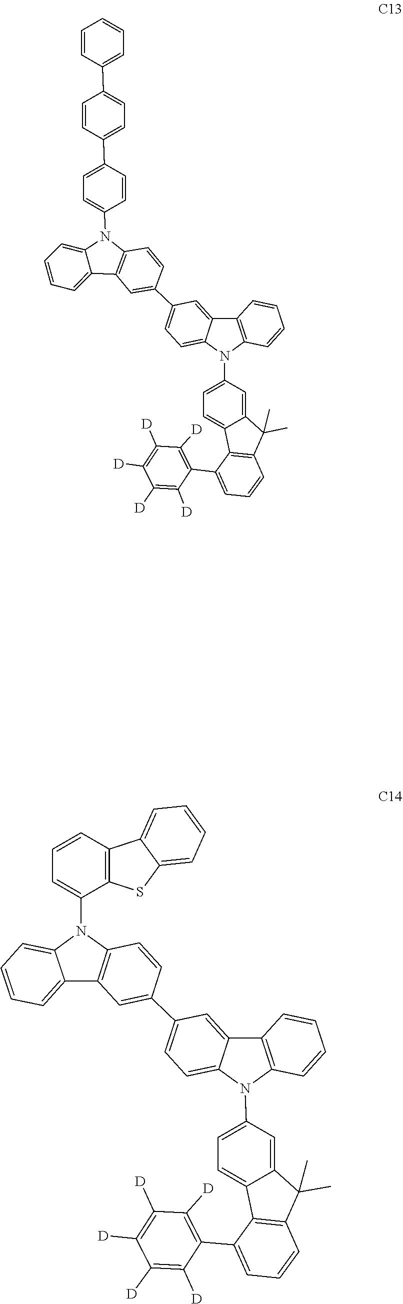 [SMILES: [2H]c1c([2H])c([2H])c(-c2cccc3c2-c2ccc(-n4c5ccccc5c5cc(-c6ccc7c(c6)c6ccccc6n7-c6ccc(-c7ccc(-c8ccccc8)cc7)cc6)ccc54)cc2C3(C)C)c([2H])c1[2H] ; [2H]c1c([2H])c([2H])c(-c2cccc3c2-c2ccc(-n4c5ccccc5c5cc(-c6ccc7c(c6)c6ccccc6n7-c6cccc7c6sc6ccccc67)ccc54)cc2C3(C)C)c([2H])c1[2H]]

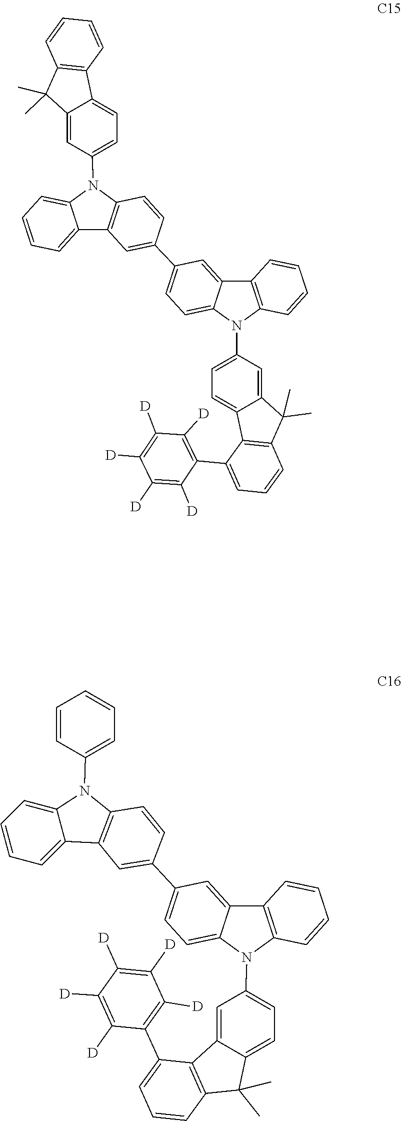

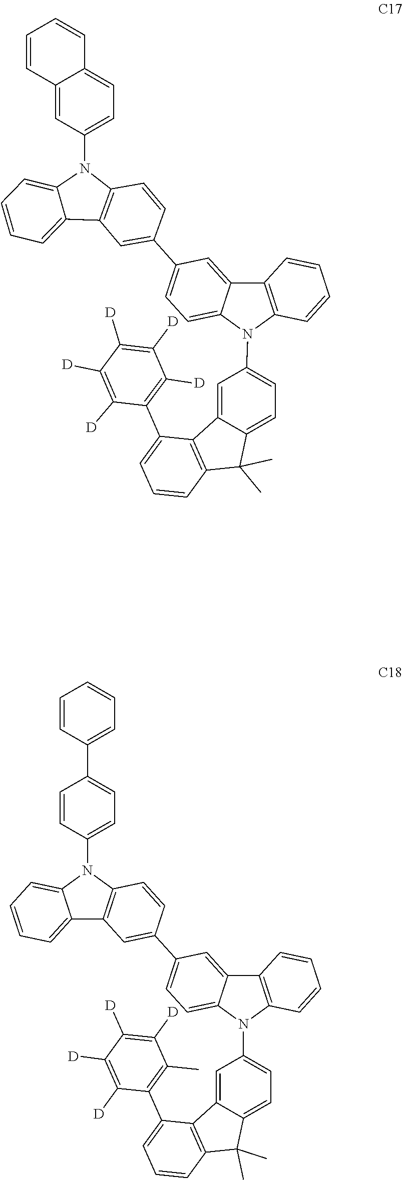 [SMILES: [2H]c1c([2H])c([2H])c(-c2cccc3c2-c2cc(-n4c5ccccc5c5cc(-c6ccc7c(c6)c6ccccc6n7-c6ccc7ccccc7c6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccc(-c7ccccc7)cc6)ccc54)ccc2C3(C)C)c(C)c1[2H]]

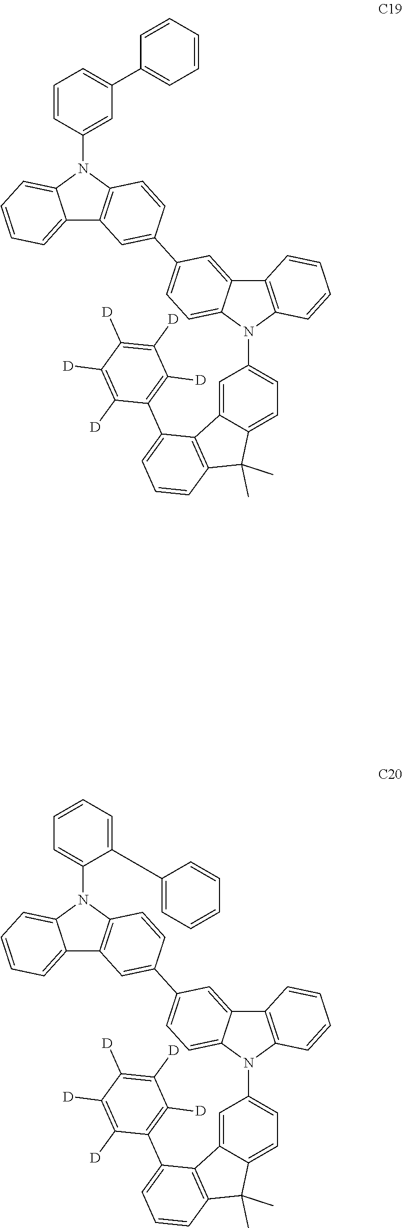 [SMILES: [2H]c1c([2H])c([2H])c(-c2cccc3c2-c2cc(-n4c5ccccc5c5cc(-c6ccc7c(c6)c6ccccc6n7-c6cccc(-c7ccccc7)c6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccccc6-c6ccccc6)ccc54)ccc2C3(C)C)c([2H])c1[2H]]

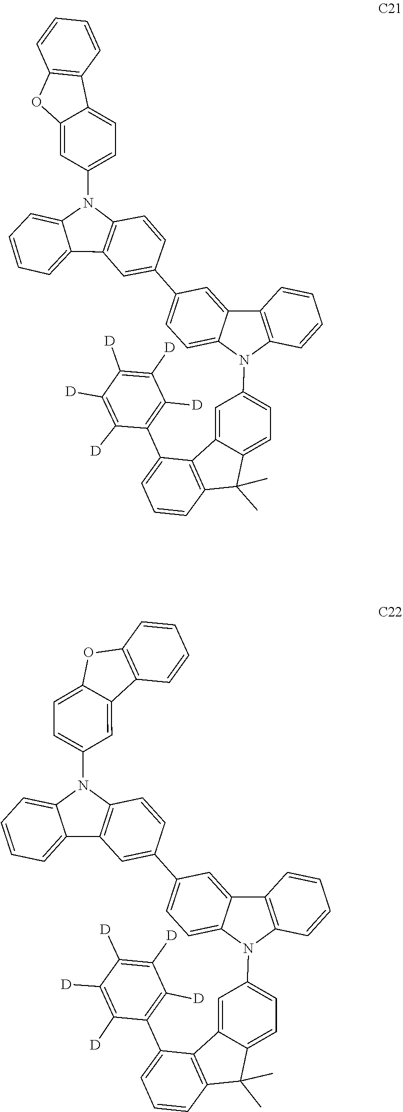 [SMILES: [2H]c1c([2H])c([2H])c(-c2cccc3c2-c2cc(-n4c5ccccc5c5cc(-c6ccc7c(c6)c6ccccc6n7-c6ccc7c(c6)oc6ccccc67)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccc7oc8ccccc8c7c6)ccc54)ccc2C3(C)C)c([2H])c1[2H]]

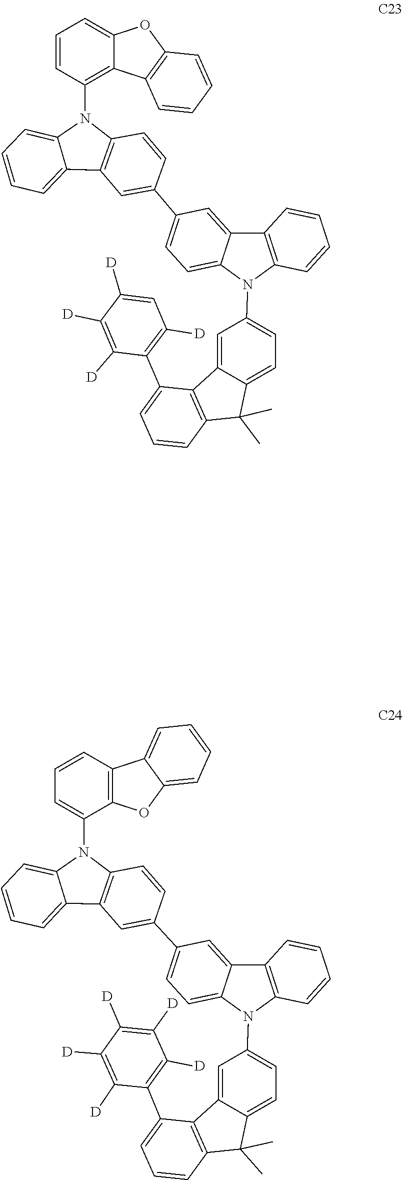 [SMILES: [2H]c1cc([2H])c(-c2cccc3c2-c2cc(-n4c5ccccc5c5cc(-c6ccc7c(c6)c6ccccc6n7-c6cccc7oc8ccccc8c67)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6cccc7c6oc6ccccc67)ccc54)ccc2C3(C)C)c([2H])c1[2H]]

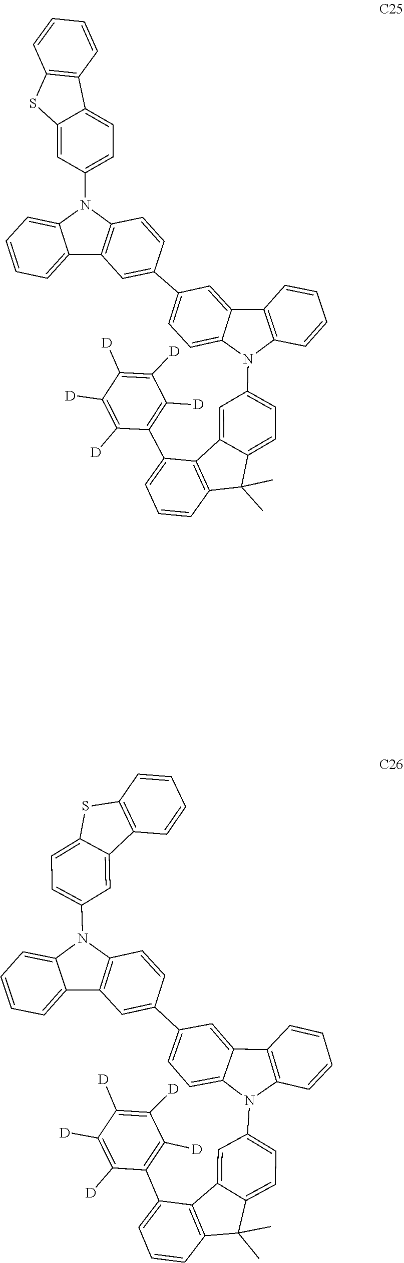 [SMILES: [2H]c1c([2H])c([2H])c(-c2cccc3c2-c2cc(-n4c5ccccc5c5cc(-c6ccc7c(c6)c6ccccc6n7-c6ccc7c(c6)sc6ccccc67)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6ccc7sc8ccccc8c7c6)ccc54)ccc2C3(C)C)c([2H])c1[2H]]

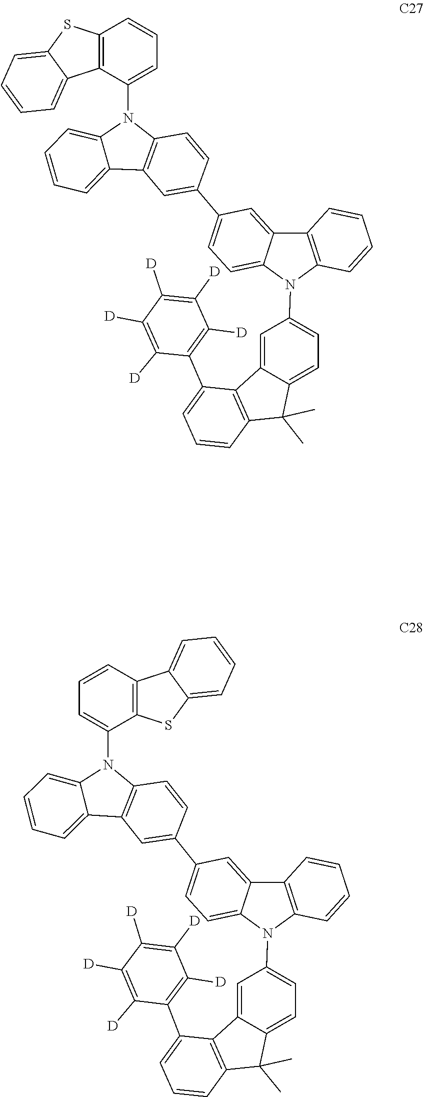 [SMILES: [2H]c1c([2H])c([2H])c(-c2cccc3c2-c2cc(-n4c5ccccc5c5cc(-c6ccc7c(c6)c6ccccc6n7-c6cccc7sc8ccccc8c67)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccccc6n7-c6cccc7c6sc6ccccc67)ccc54)ccc2C3(C)C)c([2H])c1[2H]]

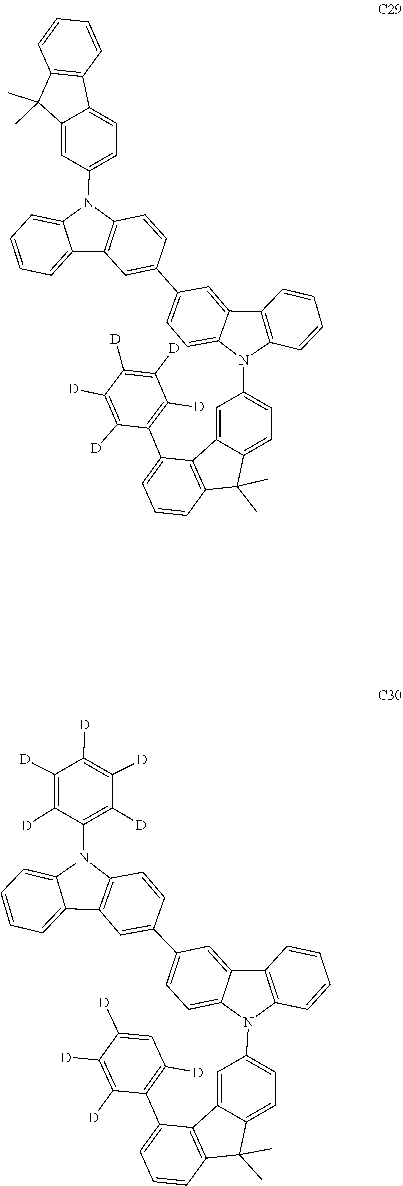 [SMILES: [2H]c1c([2H])c([2H])c(-c2cccc3c2-c2cc(-n4c5ccccc5c5cc(-c6ccc7c(c6)c6ccccc6n7-c6ccc7c(c6)C(C)(C)c6ccccc6-7)ccc54)ccc2C3(C)C)c([2H])c1[2H] ; [2H]c1cc([2H])c(-c2cccc3c2-c2cc(-n4c5ccccc5c5cc(-c6ccc7c(c6)c6ccccc6n7-c6c([2H])c([2H])c([2H])c([2H])c6[2H])ccc54)ccc2C3(C)C)c([2H])c1[2H]]

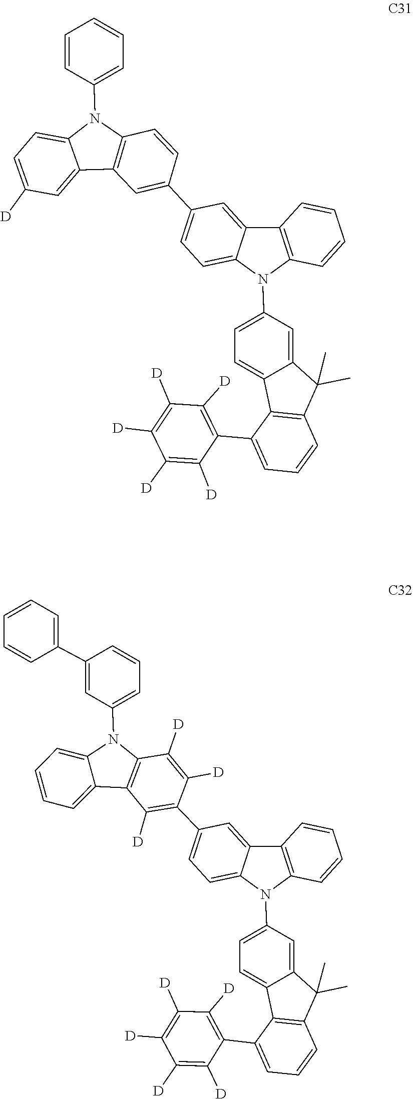 [SMILES: [2H]c1ccc2c(c1)c1cc(-c3ccc4c(c3)c3ccccc3n4-c3ccc4c(c3)C(C)(C)c3cccc(-c5c([2H])c([2H])c([2H])c([2H])c5[2H])c3-4)ccc1n2-c1ccccc1 ; [2H]c1c([2H])c([2H])c(-c2cccc3c2-c2ccc(-n4c5ccccc5c5cc(-c6c([2H])c([2H])c7c(c6[2H])c6ccccc6n7-c6cccc(-c7ccccc7)c6)ccc54)cc2C3(C)C)c([2H])c1[2H]]

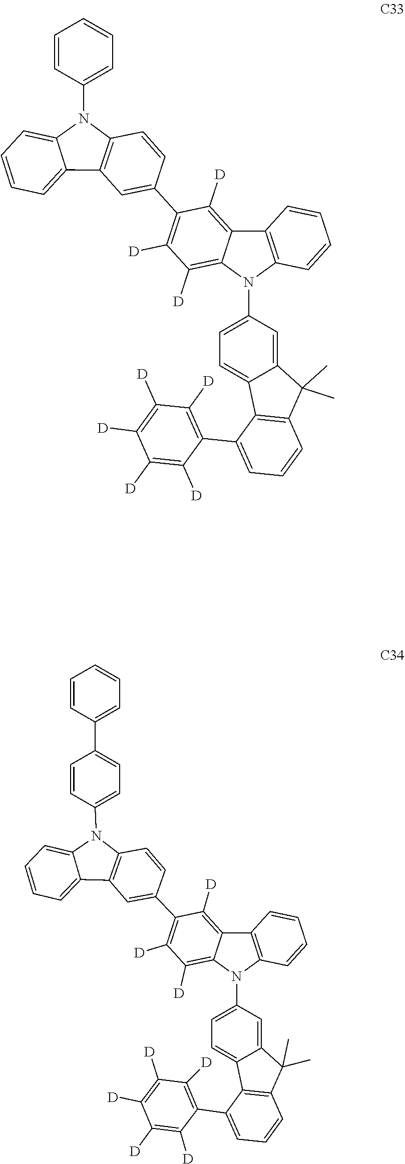 [SMILES: [2H]c1c([2H])c([2H])c(-c2cccc3c2-c2ccc(-n4c5ccccc5c5c([2H])c(-c6ccc7c(c6)c6ccccc6n7-c6ccccc6)c([2H])c([2H])c54)cc2C3(C)C)c([2H])c1[2H] ; [2H]c1c([2H])c([2H])c(-c2cccc3c2-c2ccc(-n4c5ccccc5c5c([2H])c(-c6ccc7c(c6)c6ccccc6n7-c6ccc(-c7ccccc7)cc6)c([2H])c([2H])c54)cc2C3(C)C)c([2H])c1[2H]]

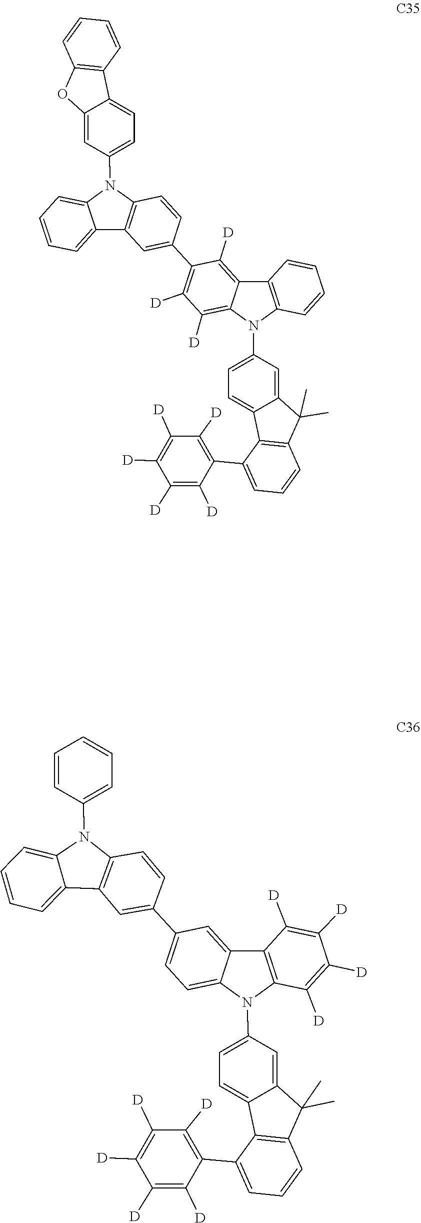 [SMILES: [2H]c1c([2H])c([2H])c(-c2cccc3c2-c2ccc(-n4c5ccccc5c5c([2H])c(-c6ccc7c(c6)c6ccccc6n7-c6ccc7c(c6)oc6ccccc67)c([2H])c([2H])c54)cc2C3(C)C)c([2H])c1[2H] ; [2H]c1c([2H])c([2H])c(-c2cccc3c2-c2ccc(-n4c5ccc(-c6ccc7c(c6)c6ccccc6n7-c6ccccc6)cc5c5c([2H])c([2H])c([2H])c([2H])c54)cc2C3(C)C)c([2H])c1[2H]]

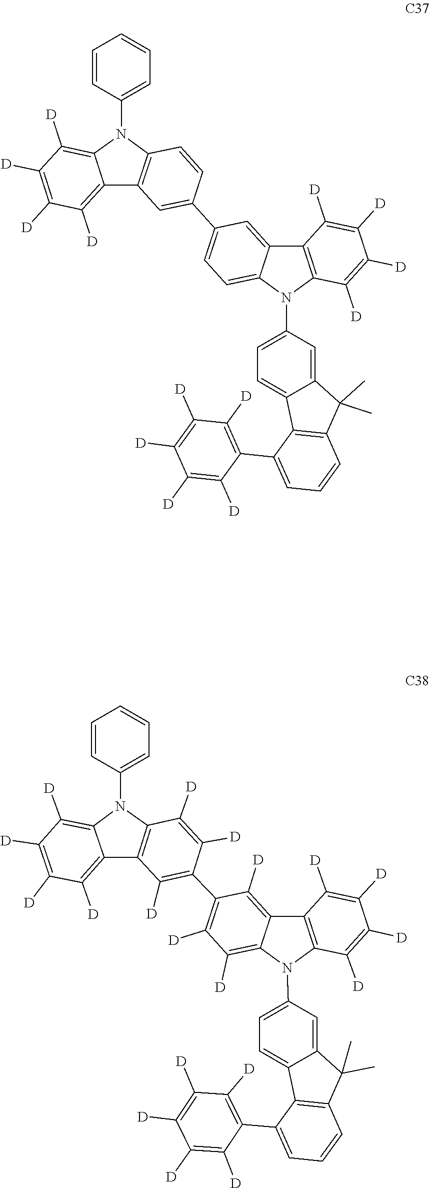 [SMILES: [2H]c1c([2H])c([2H])c(-c2cccc3c2-c2ccc(-n4c5ccc(-c6ccc7c(c6)c6c([2H])c([2H])c([2H])c([2H])c6n7-c6ccccc6)cc5c5c([2H])c([2H])c([2H])c([2H])c54)cc2C3(C)C)c([2H])c1[2H] ; [2H]c1c([2H])c([2H])c(-c2cccc3c2-c2ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)cc2C3(C)C)c([2H])c1[2H]]

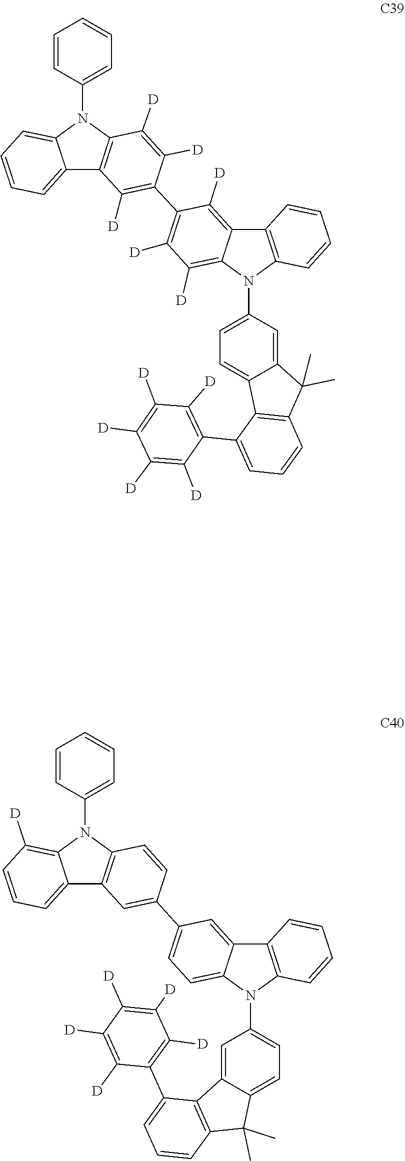 [SMILES: [2H]c1c([2H])c([2H])c(-c2cccc3c2-c2ccc(-n4c5ccccc5c5c([2H])c(-c6c([2H])c([2H])c7c(c6[2H])c6ccccc6n7-c6ccccc6)c([2H])c([2H])c54)cc2C3(C)C)c([2H])c1[2H] ; [2H]c1c([2H])c([2H])c(-c2cccc3c2-c2cc(-n4c5ccccc5c5cc(-c6ccc7c(c6)c6cccc([2H])c6n7-c6ccccc6)ccc54)ccc2C3(C)C)c([2H])c1[2H]]

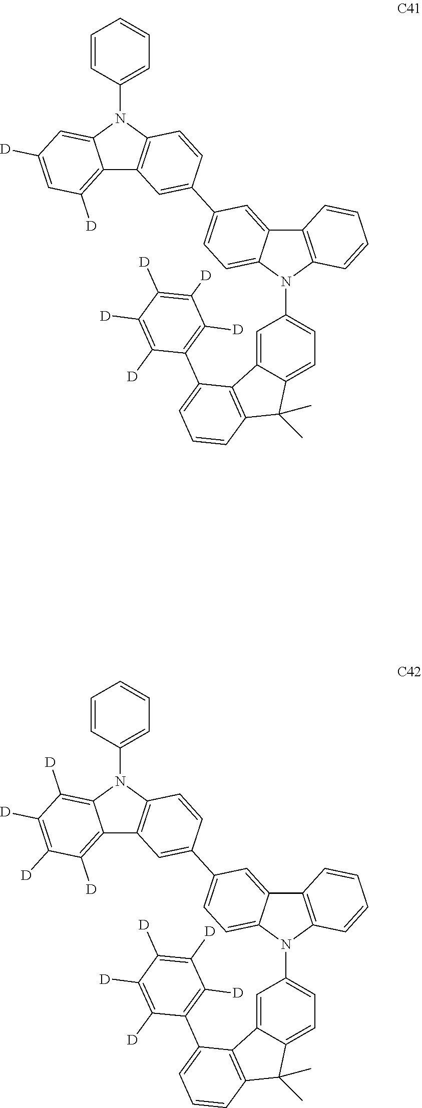 [SMILES: [2H]c1cc([2H])c2c3cc(-c4ccc5c(c4)c4ccccc4n5-c4ccc5c(c4)-c4c(-c6c([2H])c([2H])c([2H])c([2H])c6[2H])cccc4C5(C)C)ccc3n(-c3ccccc3)c2c1 ; [2H]c1c([2H])c([2H])c(-c2cccc3c2-c2cc(-n4c5ccccc5c5cc(-c6ccc7c(c6)c6c([2H])c([2H])c([2H])c([2H])c6n7-c6ccccc6)ccc54)ccc2C3(C)C)c([2H])c1[2H]]

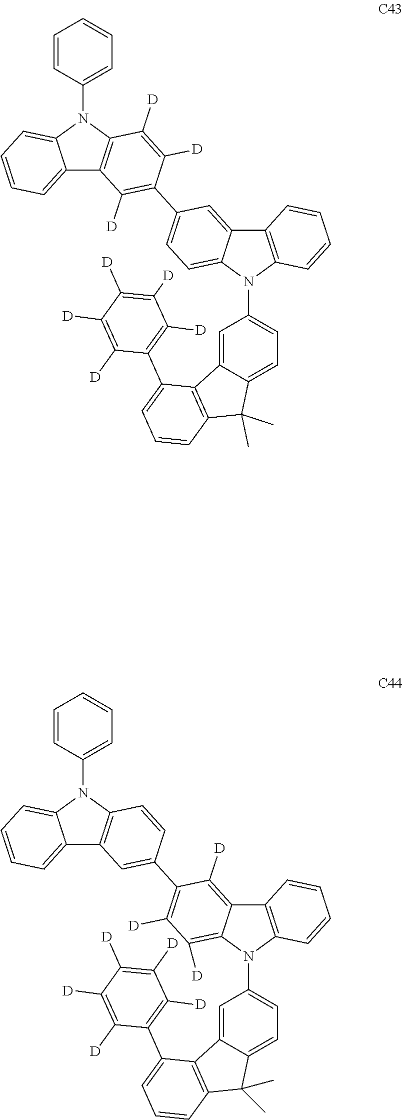 [SMILES: [2H]c1c([2H])c([2H])c(-c2cccc3c2-c2cc(-n4c5ccccc5c5cc(-c6c([2H])c([2H])c7c(c6[2H])c6ccccc6n7-c6ccccc6)ccc54)ccc2C3(C)C)c([2H])c1[2H] ; [2H]c1c([2H])c([2H])c(-c2cccc3c2-c2cc(-n4c5ccccc5c5c([2H])c(-c6ccc7c(c6)c6ccccc6n7-c6ccccc6)c([2H])c([2H])c54)ccc2C3(C)C)c([2H])c1[2H]]

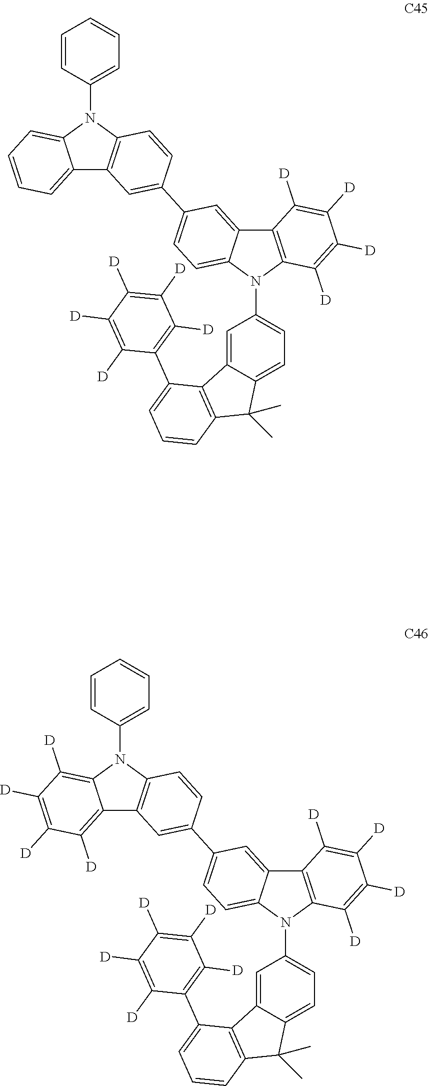 [SMILES: [2H]c1c([2H])c([2H])c(-c2cccc3c2-c2cc(-n4c5ccc(-c6ccc7c(c6)c6ccccc6n7-c6ccccc6)cc5c5c([2H])c([2H])c([2H])c([2H])c54)ccc2C3(C)C)c([2H])c1[2H] ; [2H]c1c([2H])c([2H])c(-c2cccc3c2-c2cc(-n4c5ccc(-c6ccc7c(c6)c6c([2H])c([2H])c([2H])c([2H])c6n7-c6ccccc6)cc5c5c([2H])c([2H])c([2H])c([2H])c54)ccc2C3(C)C)c([2H])c1[2H]]

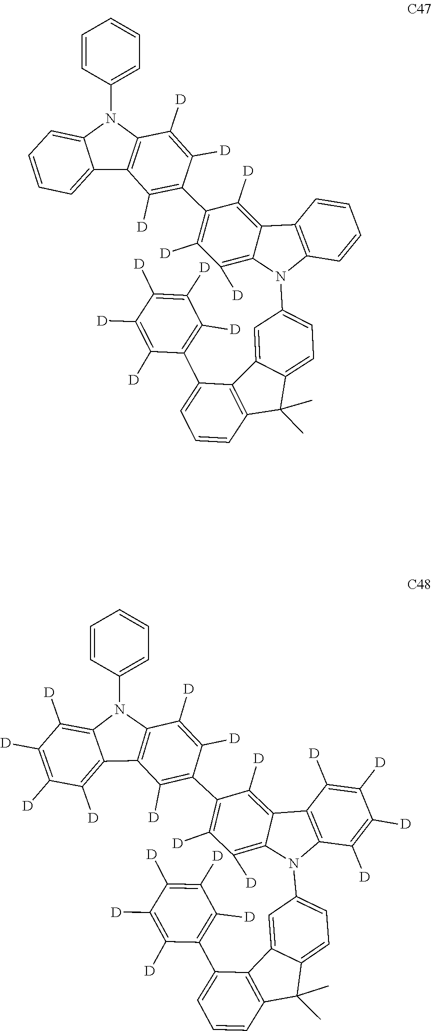 [SMILES: [2H]c1c([2H])c([2H])c(-c2cccc3c2-c2cc(-n4c5ccccc5c5c([2H])c(-c6c([2H])c([2H])c7c(c6[2H])c6ccccc6n7-c6ccccc6)c([2H])c([2H])c54)ccc2C3(C)C)c([2H])c1[2H] ; [2H]c1c([2H])c([2H])c(-c2cccc3c2-c2cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)ccc2C3(C)C)c([2H])c1[2H]]

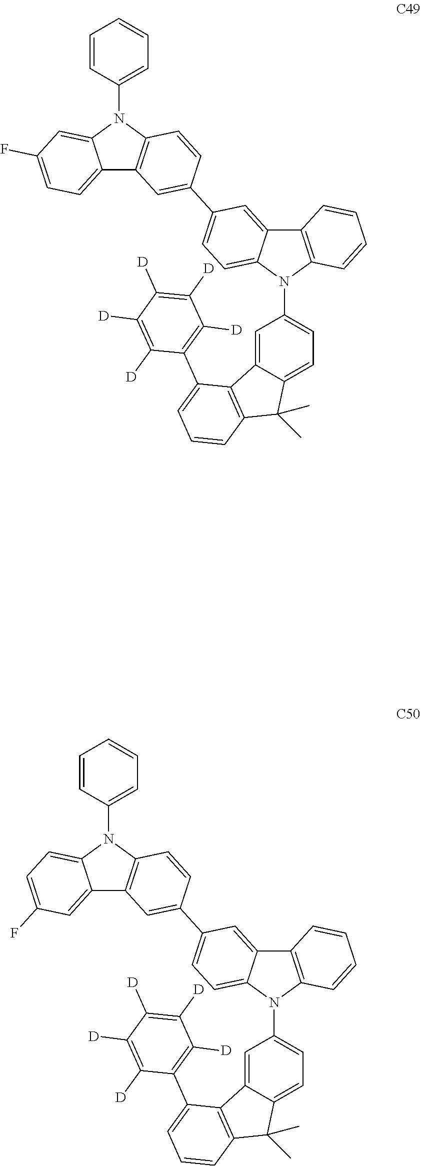 [SMILES: [2H]c1c([2H])c([2H])c(-c2cccc3c2-c2cc(-n4c5ccccc5c5cc(-c6ccc7c(c6)c6ccc(F)cc6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6cc(F)ccc6n7-c6ccccc6)ccc54)ccc2C3(C)C)c([2H])c1[2H]]

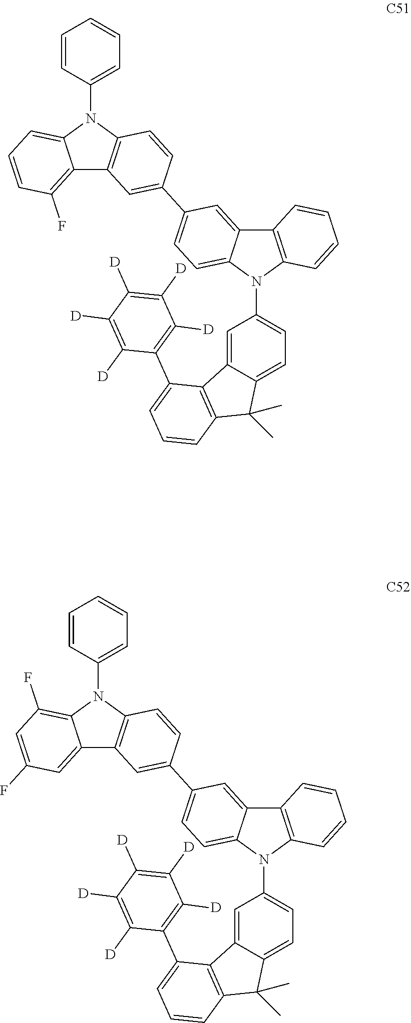 [SMILES: [2H]c1c([2H])c([2H])c(-c2cccc3c2-c2cc(-n4c5ccccc5c5cc(-c6ccc7c(c6)c6c(F)cccc6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6cc(F)cc(F)c6n7-c6ccccc6)ccc54)ccc2C3(C)C)c([2H])c1[2H]]

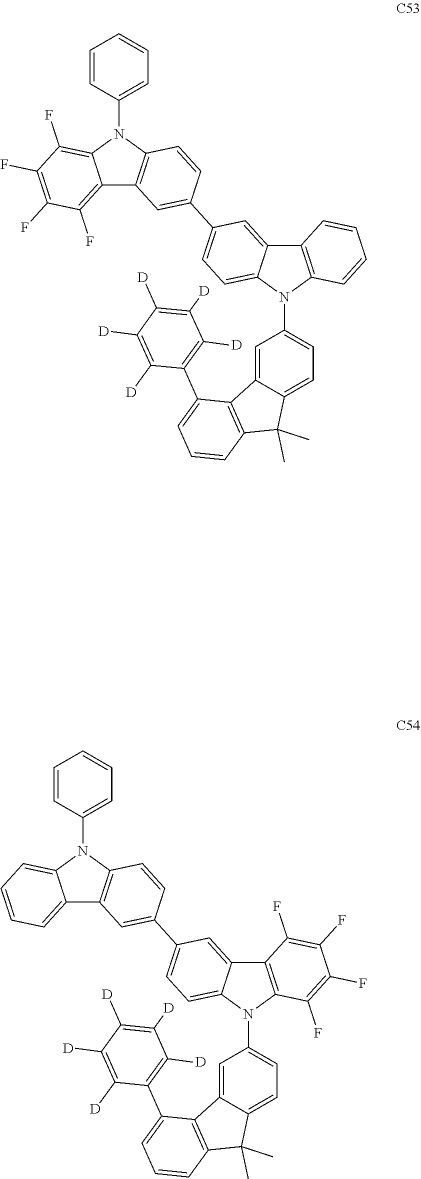 [SMILES: [2H]c1c([2H])c([2H])c(-c2cccc3c2-c2cc(-n4c5ccccc5c5cc(-c6ccc7c(c6)c6c(F)c(F)c(F)c(F)c6n7-c6ccccc6)ccc54)ccc2C3(C)C)c([2H])c1[2H] ; [2H]c1c([2H])c([2H])c(-c2cccc3c2-c2cc(-n4c5ccc(-c6ccc7c(c6)c6ccccc6n7-c6ccccc6)cc5c5c(F)c(F)c(F)c(F)c54)ccc2C3(C)C)c([2H])c1[2H]]

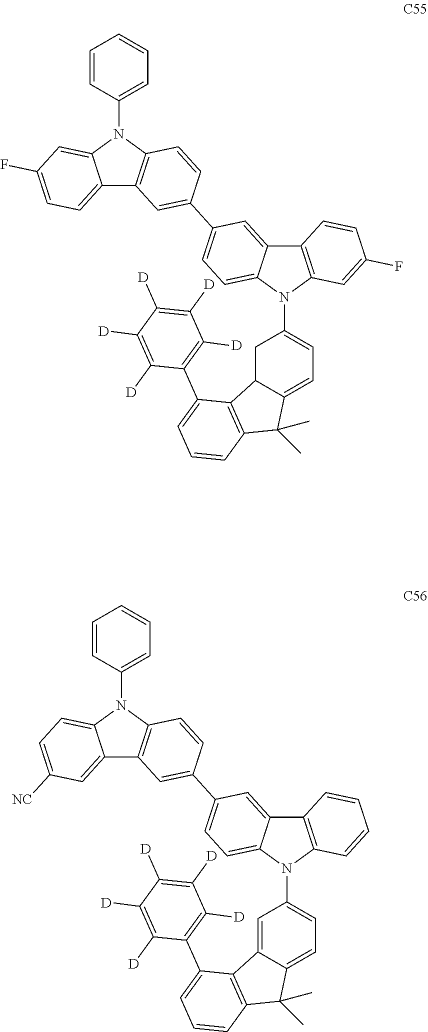 [SMILES: [2H]c1c([2H])c([2H])c(-c2cccc3c2C2CC(n4c5ccc(-c6ccc7c(c6)c6ccc(F)cc6n7-c6ccccc6)cc5c5ccc(F)cc54)=CC=C2C3(C)C)c([2H])c1[2H] ; [2H]c1c([2H])c([2H])c(-c2cccc3c2-c2cc(-n4c5ccccc5c5cc(-c6ccc7c(c6)c6cc(C#N)ccc6n7-c6ccccc6)ccc54)ccc2C3(C)C)c([2H])c1[2H]]

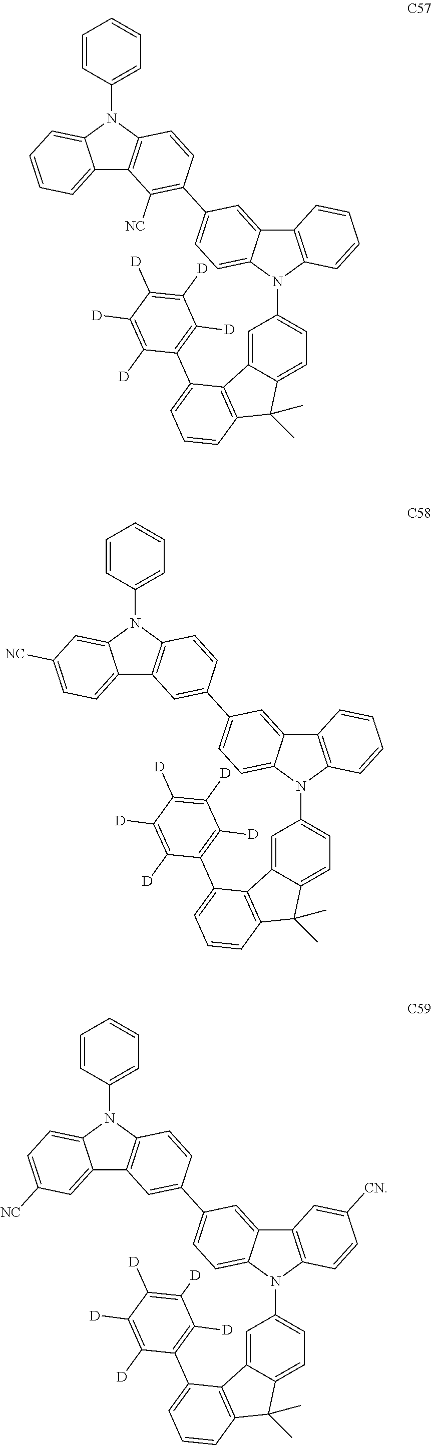 [SMILES: [2H]c1c([2H])c([2H])c(-c2cccc3c2-c2cc(-n4c5ccccc5c5cc(-c6ccc7c(c6C#N)c6ccccc6n7-c6ccccc6)ccc54)ccc2C3(C)C)c([2H])c1[2H] ; [2H]c1c([2H])c([2H])c(-c2cccc3c2-c2cc(-n4c5ccccc5c5cc(-c6ccc7c(c6)c6ccc(C#N)cc6n7-c6ccccc6)ccc54)ccc2C3(C)C)c([2H])c1[2H] ; [2H]c1c([2H])c([2H])c(-c2cccc3c2-c2cc(-n4c5ccc(C#N)cc5c5cc(-c6ccc7c(c6)c6cc(C#N)ccc6n7-c6ccccc6)ccc54)ccc2C3(C)C)c([2H])c1[2H]]

14. An organic electroluminescent device, comprising an anode and a cathode which are oppositely disposed, and a functional layer disposed between the anode and the cathode; wherein
the functional layer comprises the organic compound according to claim 1;
preferably, the functional layer comprises an organic light-emitting layer; and
preferably, the organic electroluminescent device is a green organic electroluminescent device.

15. An electronic apparatus, comprising the organic electroluminescent device according to claim 14.
